(19) United States
(12) Patent Application Publication
(10) Pub. No.: US 2018/0230218 A1
(43) Pub. Date: Aug. 16, 2018

(54) MET ANTIBODIES AND IMMUNOCONJUGATES AND USES THEREOF

(71) Applicant: IMMUNOGEN, INC., Waltham, MA (US)

(72) Inventors: Thomas Chittenden, Sudbury, MA (US); Jutta Deckert, Lexington, MA (US); Stuart William Hicks, North Andover, MA (US); Katharine C. Lai, Littleton, MA (US); Peter U. Park, Somerville, MA (US); Lingyun Rui, Weston, MA (US); Daniel J. Tavares, Natick, MA (US); Neeraj Kohli, Arlington, MA (US)

(21) Appl. No.: 15/860,868

(22) Filed: Jan. 3, 2018

Related U.S. Application Data
(60) Provisional application No. 62/477,017, filed on Mar. 27, 2017.
(60) Provisional application No. 62/442,066, filed on Jan. 4, 2017.

(51) Int. Cl. C07K 16/28; A61K 47/68; A61P 35/00; G01N 33/574

(52) U.S. Cl. CPC C07K 16/2863; A61K 47/6849; A61K 47/6803; A61P 35/00; G01N 33/574; A61K 2039/505; C07K 2317/56; C07K 2317/24; C07K 2317/33; C07K 2317/92; C07K 2317/75; C07K 2317/565

(57) ABSTRACT

MET is a receptor tyrosine kinase found on the surface of tumor cells. The present invention includes anti-MET antibodies, forms and fragments, having superior physical and functional properties; immunoconjugates, compositions, diagnostic reagents, methods for inhibiting growth, therapeutic methods, improved antibodies and cell lines; and polynucleotides, vectors and genetic constructs encoding same.

### RELATED APPLICATIONS

This application claims the benefit of the filing date, under 35 U.S.C. § 119(e), of U.S. Provisional Application Ser. No. 62/442,066, filed on Jan. 4, 2017, and U.S. Provisional Application Ser. No. 62/477,017, filed on Mar. 27, 2017. The entire contents of each of the above-referenced applications are incorporated herein by reference.

### BACKGROUND OF THE INVENTION

MET, also known as c-Met, HGFR, RCCP2 or AUTS9, is a glycosylated receptor tyrosine kinase that plays a central role in epithelial morphogenesis and cancer development. It is also referred to as the hepatocyte growth factor or HGF receptor, the scatter factor or SF receptor, the met proto-oncogene tyrosine kinase or proto-oncogene c-Met.

MET is synthesized as a single chain precursor which undergoes co-translational proteolytic cleavage. This generates a mature MET that is a disulfide-linked dimer composed of a 50 kDa extracellular a chain and a 145 kDa transmembrane β chain (Birchmeier, C. et al. Nat. Rev. Mol. Cell Biol. 2003; 4:915; Corso, S. et al. Trends Mol. Med. 2005; 11:284). The extracellular domain (ECD) contains a seven bladed β-propeller sema domain, a cysteine-rich PSI/MRS domain, and four Ig-like E-set domains, while the cytoplasmic region includes the tyrosine kinase domain and an adaptor protein docking site (Gherardi, E. et al. Proc. Natl. Acad. Sci. 2003; 100:12039, Park, M. et al. Proc. Natl. Acad. Sci. 1987; 84:6379). The sema domain, which is formed by both the α and β chains of MET, mediates both ligand binding and receptor dimerization (Gherardi, E. et al. Proc. Natl. Acad. Sci. 2003; 100:12039, Kong-Beltran, M. et al. Cancer Cell 2004; 6:75).

Hepatocyte growth factor (HGF) is the ligand for MET (Gheradi, E. et al. Proc. Natl. Acad. Sci. 2003; 100:12039). HGF is also known as scatter factor (SF) and hepatopoietin A and it belongs to the plasminogen subfamily of S1 peptidases. Human HGF is produced and secreted as an inactive 728 amino acid (AA) single chain propeptide. It is cleaved after the fourth Kringle domain by a serine protease to form the active form of HGF, which is a disulfide-linked heterodimer with a 60 kDa α and 30 kDa β chain.

HGF regulates epithelial morphogenesis by inducing cell scattering and branching tubulogenesis (Maeshima, A. et al. Kid. Int. 2000; 58:1511; Montesano, R. et al. Cell 1991; 67:901). Thus the interaction between MET and HGF plays an important role during mammalian development, tissue growth and repair. However, inappropriate activation of MET can also support tumor cell proliferation and invasion, drive tumor associated angiogenesis and therefore has been implicated in the formation and progression of several types of cancers.

Aberrant signaling by MET can be the result of multiple mechanisms including ligand-independent activation such as through MET overexpression or MET activating mutations and ligand-dependent activation in either paracrine or autocrine manner. Paracrine induction of epithelial cell scattering and branching tubulogenesis results from the stimulation of MET on undifferentiated epithelium by HGF released from neighboring mesenchymal cells (Sonnenberg, E. et al. J. Cell Biol. 1993; 123:223). Autocrine induction is a result of HGF production by MET positive cells.

Dimerization of the MET receptor in the presence or absence of ligand induces tyrosine phosphorylation in the cytoplasmic region, which in turn activates the kinase domain and provides docking sites for multiple SH2 containing molecules (Naldini, L. et al. Mol. Cell. Biol. 1991; 11:1793, Ponzetto, C. et al. Cell 1994; 77:261). This results in activation of downstream signaling pathways involving key signal transducers such as Src, MAPK, PI3K and Akt.

MET may also form non-covalent complexes with a variety of membrane proteins including CD44v6, CD151, EGF R, Fas, Integrin α6/β4, Plexins B1, 2, 3, and MSP R/Ron (Orian Rousseau, V. et al. Genes Dev. 2002; 16:3074; Follenzi, A. et al. Oncogene 2000; 19:3041). Ligation of one complex component triggers activation of the other, followed by cooperative signaling effects. Formation of some of these heteromeric complexes can lead to epithelial cell morphogenesis and tumor cell invasion (Trusolino, L. et al. Cell 2001; 107:643, Giordano, S. et al. Nat. Cell Biol. 2002; 4:720). More recently, activation of the MET pathway has been seen as mechanism of resistance to EGFR inhibitors (Engelman J. A. et al. Science 2001; 316:1039-1043).

Numerous studies have implicated aberrant function of the receptor tyrosine kinase MET in the progression and metastasis of human carcinoma including in pancreatic cancer, gastric cancer, prostate cancer, ovarian cancer, breast cancer, hepatocellular carcinoma (HCC), melanoma, osteosarcoma, and colorectal cancer (CRC), lung cancer including small-cell lung cancer (SCLC) and non-small cell lung cancer (NSCLC), head and neck squamous cell carcinoma (HNSCC), kidney cancer and thyroid cancer.

Gene amplification events, activating mutations in the kinase domain, genetic polymorphisms, chromosomal translocation, overexpression, and additional splicing and proteolytic cleavage of MET have been described in a wide range of cancers (Birchmeier, C. et al. Nat. Rev. Mol. Cell Biol. 2003; 4:915). Notably, activating mutations in MET leading to constitutive activation have been identified in patients with hereditary papillary renal cancer, directly implicating MET in human tumorigenesis (Nat. Genet. 1997; 16:68-73).

In addition, overexpression of both MET receptor and HGF ligand has been documented in cancers such as HNSCC (Cancer Res 2009; 69(7):3021-31), lung cancer (Oncology 1996; 53:392-7), gastric cancer (Apmis 2000; 108:195-200), pancreatic cancer (Cancer Res 1994 5775-8; Jin, Cancer Res 2008; 68:4360-8) and osteosarcoma (Oncogene 1995; 10:739-49). Co-expression of MET receptor and HGF ligand by the same cell or tissue can lead to autocrine signaling and aberrant receptor activation.

Several small molecule inhibitors of MET have been developed in recent years and are currently being tested in clinical trials (for review see: Comoglio P M. et al., Nat Rev Drug Discov 2008; 7, 504-516, Eder J P. et al. Clin Cancer Res 2009; 15: 2207-2214, Wang M H et al., Acta Pharmacologica Sinica 2010; 31: 1181-1188). Another strategy has been to develop neutralizing antibodies to HGF or HGF antagonists to prevent ligand-dependent activation of MET.

The development of therapeutic antibodies against MET has been very difficult since antibodies that compete for HGF-binding typically result in MET receptor dimerizing and therefore act as agonists (Prat M, et al. J Cell Sci 1998; 111 (Pt 2), 237-247). For example an anti-MET antibody, known as 5D5, was described that blocks HGF binding to MET and acts as a potent agonist in divalent antibody form (Schwall, U.S. Pat. No. 5,686,292). In response, 5D5 was engineered to be monovalent in either a Fab version or in a one armed version (OA5D5) and then behaves as an antagonist (Dennis, U.S. Pat. No. 7,476,724). The one armed version was selected for further development in clinical trials and was termed MetMab (Jin H, et al. Cancer Res 2008; 68, 4360-4368). However, this one armed version cannot be considered a full antibody but rather represents an antibody fragment with undesirable properties including a diminished effector function and a reduced half-life.

Other antibodies have been described that can block HGF binding to MET (Morton P. A. US 2004/0166544 and WO 2005/016382). Other anti-MET antibodies, such as 11E1, 224G11, 223C4 and 227H1 disclosed in WO 2009/007427 (Goetsch L.), were selected to prevent MET receptor dimerization.

Antibody-drug conjugates (ADC), are a type of immunoconjugate that comprise a cytotoxic agent covalently linked to an antibody through specialized chemical linker. The use of ADCs for the local delivery of cytotoxic or cytostatic agents, for example, drugs to kill or inhibit tumor cells in the treatment of cancer (see Syrigos and Epenetos (1999) Anticancer Research 19:605-614; Niculescu-Duvaz and Springer (1997) Adv. Drug Del. Rev. 26:151-172; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (eds.), pp. 475-506). Maximal efficacy with minimal toxicity is sought. Both polyclonal antibodies and monoclonal antibodies have sometimes been reported as being useful in this regard. (See Rowland et al., (1986) Cancer Immunol. Immunother., 21:183-87). Drugs that are known to be used in this fashion include daunomycin, doxorubicin, methotrexate, and vindesine (Rowland et al., Cancer Immunol. Immunother. 21:183-87 (1986)). Toxins used in antibody-toxin conjugates include bacterial toxins such as diphtheria toxin, plant toxins, such as ricin, small molecule toxins such as geldanamycin. Kerr et al (1997) Bioconjugate Chem. 8(6):781-784; Mandler et al (2000) Journal of the Nat. Cancer Inst. 92(19):1573-1581; Mandler et al (2000) Bioorganic & Med. Chem. Letters 10: 1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al. (1993) Cancer Res. 53:3336-3342. Toxins may exert cytotoxic and/or cytostatic effects through diverse mechanisms including tubulin binding, DNA binding, or topoisomerase inhibition. Meyer, D. L. and Senter, P. D. “Recent Advances in Antibody Drug Conjugates for Cancer Therapy” in Annual Reports in Medicinal Chemistry, Vol 38 (2003) Chapter 23, 229-237. But many cytotoxic drugs tend to be inactive or less active when conjugated to large antibodies or protein receptor ligands.

Thus, there continues to be a need for the development of improved and superior c-Met targeted therapeutic agents, including antibodies or antibody fragments that exhibit specificity, reduced toxicity, stability and enhanced physical and functional properties over known therapeutic agents. The instant invention addresses those needs.

### SUMMARY OF THE INVENTION

The present invention provides anti-MET immunoconjugates exhibiting specific and potent cytotoxic activity in MET over-expressed, non-amplified settings. Furthermore, even at high concentrations, the anti-MET immunoconjugates of the invention showed marginal levels of cytotoxicity and no specificity in cells having normal MET gene copy number, expressing less than 30,000 cell surface receptors per cell. Together, these properties result in an effective immunoconjugate for the treatment of cancers, e.g., a cMET over-expressed, non-amplified cancer.

Reference will now be made in detail to certain aspects of the invention, examples of which are illustrated in the accompanying structures and formulas. While the invention will be described in conjunction with the enumerated aspects, it will be understood that they are not intended to limit the invention to those aspects. On the contrary, the invention is intended to cover all alternatives, modifications, and equivalents that may be included within the scope of the present invention as defined by the claims. One skilled in the art will recognize many methods and materials similar or equivalent to those described herein, that can be used in the practice of the present invention.

In one aspect, the present invention provides an isolated monoclonal antibody, or antigen-binding fragment thereof, that specifically binds to an epitope in the extracellular region of human cMET, wherein said antibody or antigen-binding fragment thereof comprises light chain complementary determining regions LC CDR1, LC CDR2, and LC CDR3 and heavy chain complementary determining regions HC CDR1, HC CDR2, and HC CDR3 having the sequences selected from the group consisting of:

(a) SEQ ID NOs:4, 5, and 7 and SEQ ID NOs:13, 14, and 15, respectively;

(b) SEQ ID NOs:1, 2, and 3 and SEQ ID NOs:8, 9, and 10, respectively;

(c) SEQ ID NOs: 1, 2, and 3 and SEQ ID NOs: 8, 12, and 10, respectively;

(d) SEQ ID NOs:4, 5, and 6 and SEQ ID NOs:13, 14, and 15, respectively;

(e) SEQ ID NOs:4, 5, and 6 and SEQ ID NOs:13, 17, and 15, respectively;

(f) SEQ ID NOs:4, 5, and 7 and SEQ ID NOs:13, 17, and 15, respectively; and

(g) SEQ ID NOs:4, 5, and 8 and SEQ ID NOs:13, 17, and 15, respectively.

In certain embodiments, the antibody is a murine, non-human mammal, chimeric, humanized, or human antibody. In certain embodiments, the humanized antibody is a CDR-grafted antibody or resurfaced antibody. In certain embodiments, the antibody is a full-length antibody.

In certain embodiments, the antigen-binding fragment is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the antibody or antigen-binding fragment thereof comprises a light chain variable domain (VL) and a heavy chain variable domain (VH) having sequences that are at least 95%, 96%, 97%, 98%, 99%, or 100% identical to sequences selected from the group consisting of:

(a) SEQ ID NO:32 and SEQ ID NO:36, respectively;

(b) SEQ ID NO:18 and SEQ ID NO:19, respectively;

(c) SEQ ID NO:20 and SEQ ID NO:21, respectively;

(d) SEQ ID NO:22 and SEQ ID NO:23, respectively;

(e) SEQ ID NO:24 and SEQ ID NO:25, respectively;

(f) SEQ ID NO:26 and SEQ ID NO:27, respectively;

(g) SEQ ID NO:28 and SEQ ID NO:31, respectively;

(h) SEQ ID NO:29 and SEQ ID NO:31, respectively;

(i) SEQ ID NO:30 and SEQ ID NO:31, respectively;

(j) SEQ ID NO:32 and SEQ ID NO:35, respectively;

(k) SEQ ID NO:32 and SEQ ID NO:36, respectively;

(l) SEQ ID NO:33 and SEQ ID NO:36, respectively;

(m) SEQ ID NO:33 and SEQ ID NO:35, respectively; and

(n) SEQ ID NO:33 and SEQ ID NO:34, respectively.

In certain embodiments, the antibody or antigen-binding fragment thereof comprises a light chain and a heavy chain having the sequences selected from the group consisting of:

(a) SEQ ID NO:49 and SEQ ID NO:54, respectively;

(b) SEQ ID NO:39 and SEQ ID NO:40, respectively;

(c) SEQ ID NO:41 and SEQ ID NO:42, respectively;

(d) SEQ ID NO:43 and SEQ ID NO:44, respectively;

(e) SEQ ID NO:45 and SEQ ID NO:48, respectively;

(f) SEQ ID NO:46 and SEQ ID NO:48, respectively;

(g) SEQ ID NO:47 and SEQ ID NO:48, respectively;

(h) SEQ ID NO:49 and SEQ ID NO:53, respectively;

(i) SEQ ID NO:49 and SEQ ID NO:52, respectively;

(j) SEQ ID NO:49 and SEQ ID NO:51, respectively;

(k) SEQ ID NO:50 and SEQ ID NO:53, respectively;

(l) SEQ ID NO:50 and SEQ ID NO:52, respectively;

(m) SEQ ID NO:50 and SEQ ID NO:51, respectively;

(n) SEQ ID NO:49 and SEQ ID NO:77, respectively;

(o) SEQ ID NO:49 and SEQ ID NO:78, respectively;

(p) SEQ ID NO:49 and SEQ ID NO:79, respectively;

(q) SEQ ID NO:49 and SEQ ID NO:80, respectively;

(r) SEQ ID NO:49 and SEQ ID NO:81, respectively;

(s) SEQ ID NO:49 and SEQ ID NO:82, respectively;

(t) SEQ ID NO:49 and SEQ ID NO:83, respectively; and

(u) SEQ ID NO:49 and SEQ ID NO:84, respectively.

In certain embodiments, the isolated antibody, or antigen-binding fragment thereof is produced by any of hybridomas 247.27.16, 247.2.26, 247.48.38, 247.3.14, 247.22.2, 248.69.4, and 247.16.8.

In certain embodiments, the present invention provides a polypeptide comprising the VL and VH sequences described herein.

In one aspect, the present invention provides a cell producing the antibody or antigen-binding fragment thereof or the polypeptide described herein.

In another aspect, the present invention provides a method of producing the antibody or antigen-binding fragment thereof, or the polypeptide described herein, wherein the method comprises:
(a) culturing the cell producing the antibody or antigen-binding fragment thereof or the polypeptide described herein; and,
(b) isolating the antibody, antigen-binding fragment thereof, or polypeptide from said cultured cell.

In certain embodiments, the cell is a eukaryotic cell.

In another aspect, the present invention provides a diagnostic reagent comprising the antibody or antigen-binding fragment thereof described herein. In certain embodiments, the antibody or antibody fragment is labeled. In certain embodiments, the label is selected from the group consisting of a radiolabel, a fluorophore, a chromophore, an imaging agent and a metal ion.

In another aspect, the present invention provides a polynucleotide encoding the antibody or antigen-binding fragment thereof described herein, wherein the polynucleotide has a sequence selected from the group consisting of SEQ ID NOs:55-72.

In yet another aspect, the present invention provides a vector comprising the polynucleotide described herein. In certain embodiments, the vector is an expression vector.

In yet another aspect, the present invention provides a host cell comprising the expression vector described herein.

The present invention also provides an immunoconjugate represented by the following formula:

CBACy^{L1})_{W}_{_{L}},

wherein:

CBA is the antibody or antigen-binding fragment thereof or the polypeptide described herein that is covalently linked to Cy^{L1} through a lysine residue;

W_{L} is an integer from 1 to 20; and

Cy^{L1} is represented by the following formula:

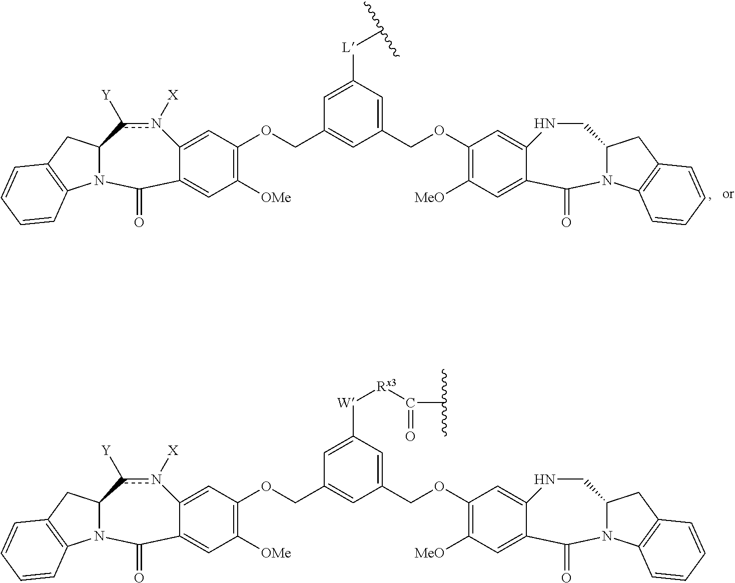

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃H or a pharmaceutically acceptable salt thereof;

W′ is —NR^{e′},

R^{e′} is —(CH₂—CH₂—O)ₙ—R^{k};

n is an integer from 2 to 6;

R^{k} is —H or -Me;

R^{x3} is a (C₁-C₆)alkyl;

L′ is represented by the following formula:

—NR₅—P—C(═O)—(CRₐR_{b})ₘ—C(═O)— (B1′); or

—NR₅—P—C(═O)—(CRₐR_{b})ₘ—S—Z^{S1}— (B2′);

R₅ is —H or a (C₁-C₃)alkyl;

P is an amino acid residue or a peptide containing between 2 to 20 amino acid residues;

Rₐ and R_{b}, for each occurrence, are each independently —H, (C₁-C₃)alkyl, or a charged substituent or an ionizable group Q;

m is an integer from 1 to 6; and

Z^{S1} is selected from any one of the following formulas:

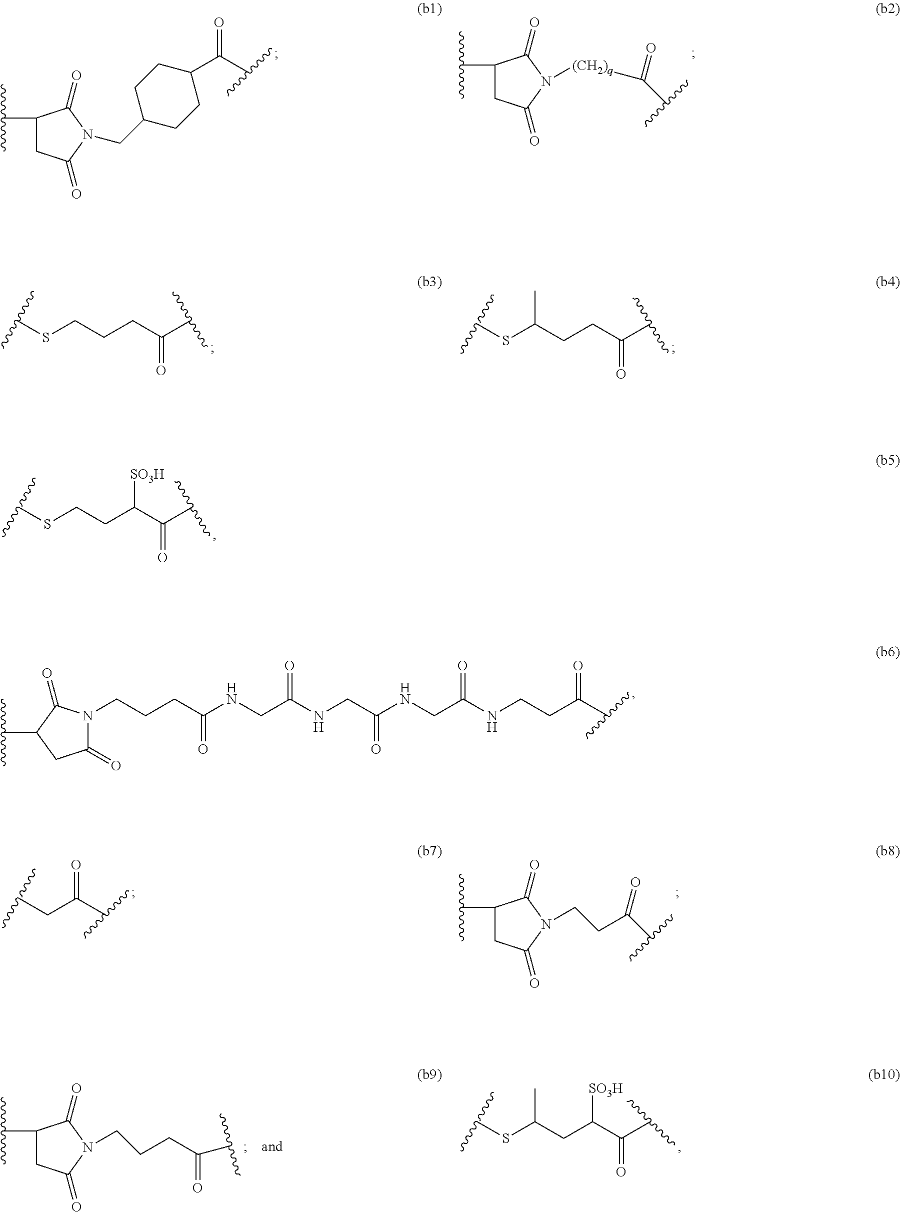

wherein q is an integer from 1 to 5.

In certain embodiments, the present invention provides an immunoconjugate represented by the following formula:

CBACy^{L2})_{W}_{_{L}},

wherein:

CBA is the antibody or antigen-binding fragment thereof or the polypeptide described herein that is covalently linked to Cy^{L2} through a lysine residue;

W_{L} is an integer from 1 to 20; and

Cy^{L2} is represented by the following formula:

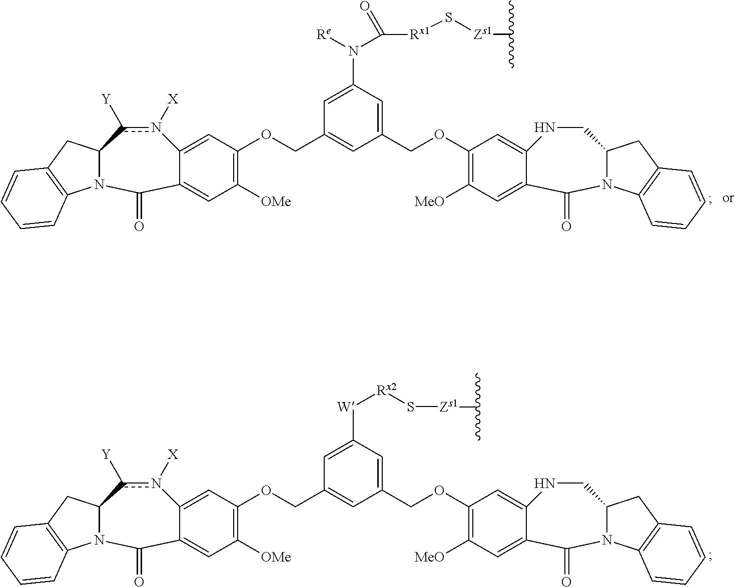

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃H;

R^{x1} and R^{x2} are independently (C₁-C₆)alkyl;

R^{e} is —H or a (C₁-C₆)alkyl;

W′ is —NR^{e′},

R^{e′} is —(CH₂—CH₂—O)ₙ—R^{k};

n is an integer from 2 to 6;

R^{k} is —H or -Me;

Z^{S1} is selected from any one of the following formulas:

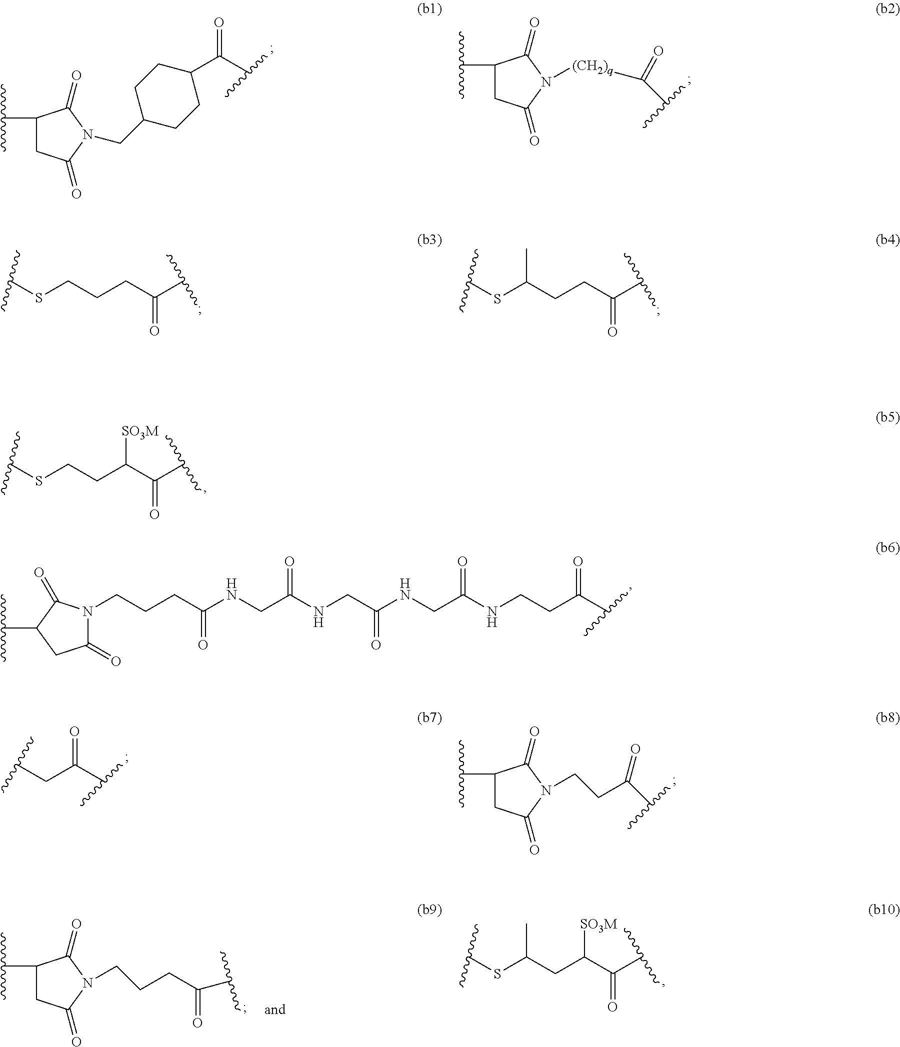

wherein q is an integer from 1 to 5.

In certain embodiments, the immunoconjugates of the present invention is represented by the following formula:

CBACy^{L3})_{W}_{_{L}} (L3),

wherein:

CBA is a MET-binding agent (e.g., an anti-MET antibody or an antibody fragment thereof) described herein above that is covalently linked to Cy^{L3} through a Lys residue;

W_{L} is an integer from 1 to 20;

Cy^{L3} is represented by the following formula:

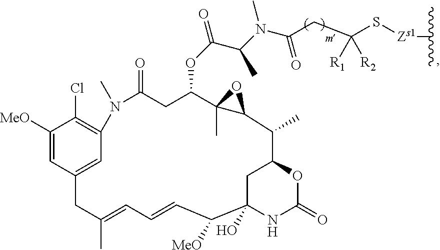

m′ is 1 or 2;

R₁ and R₂, are each independently H or a (C₁-C₃)alkyl; and

Z^{S1} is selected from any one of the following formulas:

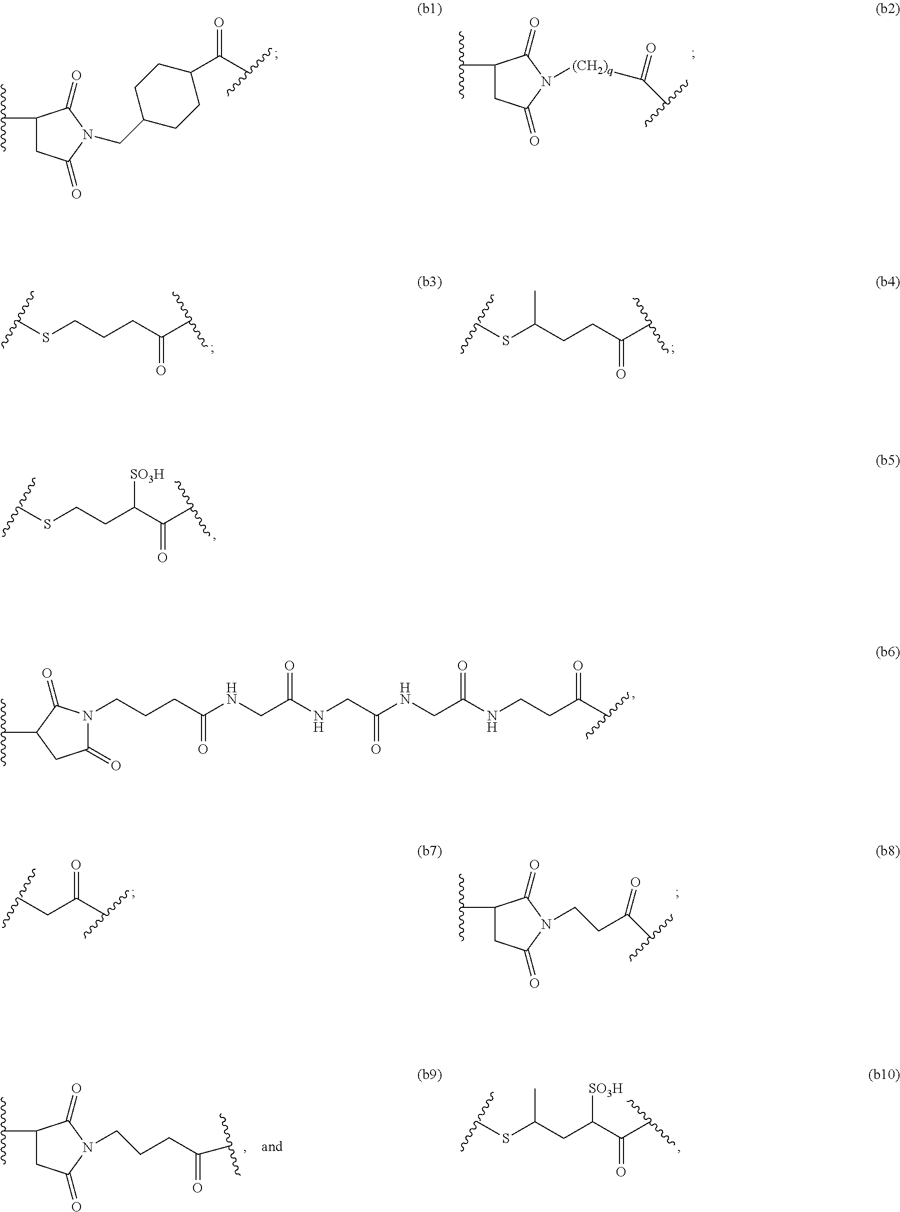

wherein q is an integer from 1 to 5.

In certain embodiments, for immunoconjugates of formula (L3), m′ is 1, and R₁ and R₂ are both H; and the remaining variables are as described above.

In certain embodiments, for immunoconjugates of formula (L3), m′ is 2, and R₁ and R₂ are both Me; and the remaining variables are as described above.

In certain embodiments, the immunoconjugates of the present invention is represented by the following formula:

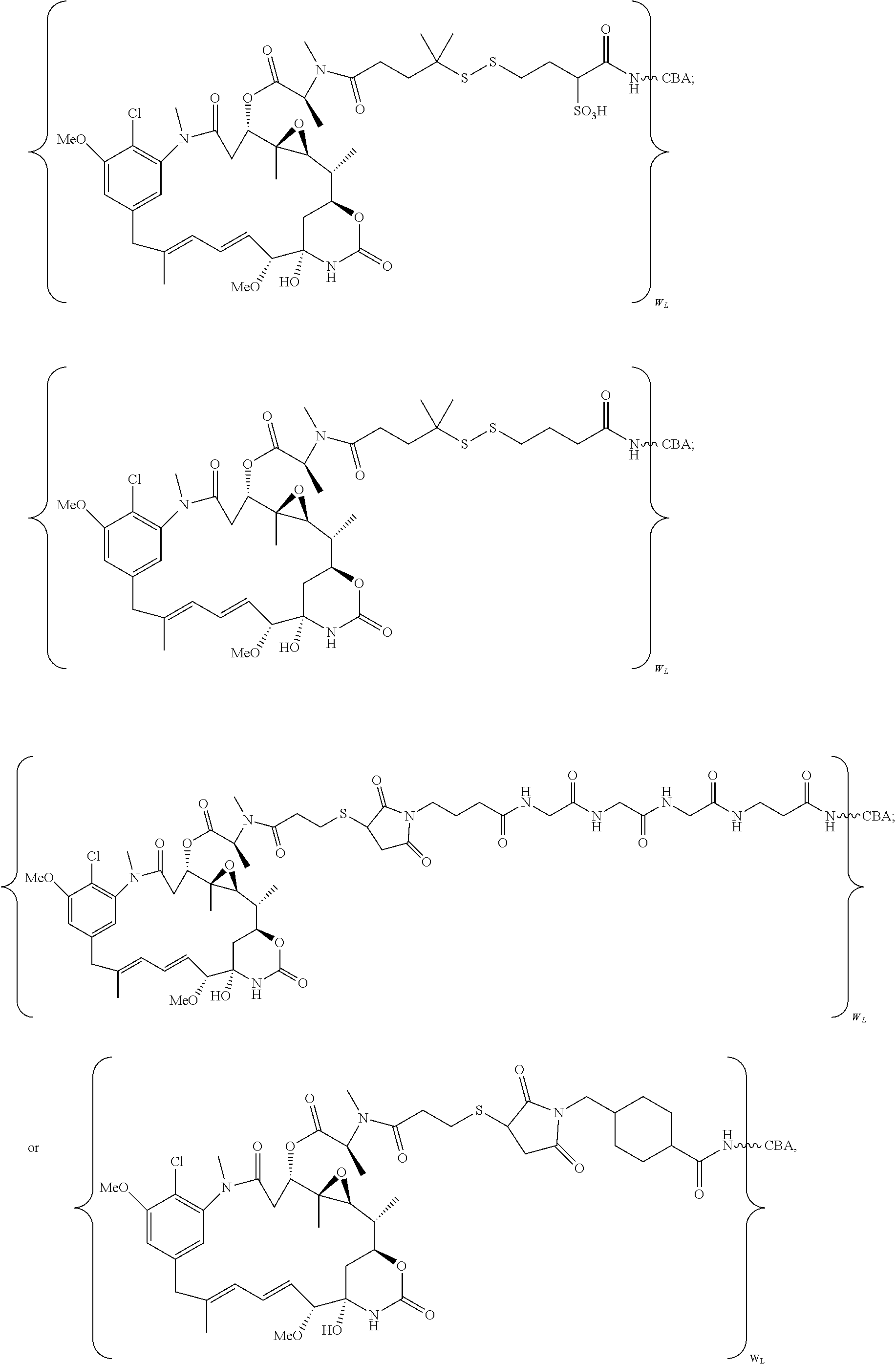

or a pharmaceutically acceptable salt thereof, wherein W_{L} is an integer from 1 to 10.

In certain embodiments, the immunoconjugate of the present invention is represented by the following formula:

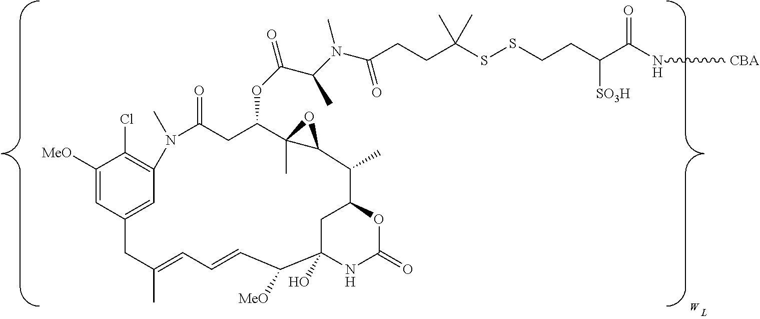

or a pharmaceutically acceptable salt thereof, wherein CBA is the monoclonal antibody, or antigen-binding fragment thereof of claim 1, wherein said antibody or antigen-binding fragment thereof comprises light chain complementary determining regions LC CDR1, LC CDR2, and LC CDR3 and heavy chain complementary determining regions HC CDR1, HC CDR2, and HC CDR3 having the sequences of SEQ ID NOs:4, 5, and 7 and SEQ ID NOs:13, 14, and 15, respectively; and W_{L} is an integer from 1 to 10. In certain embodiments, the isolated monoclonal antibody, or antigen-binding fragment thereof comprises a heavy chain variable domain (VH) and a light chain variable domain (VL) having sequences of SEQ ID NO:32 and SEQ ID NO:36, respectively. In certain embodiments, the isolated monoclonal antibody comprises a heavy chain and a light chain having the sequences of SEQ ID NO:49 and SEQ ID NO:53, respectively. In certain embodiments, the isolated monoclonal antibody comprises a heavy chain and a light chain having the sequences of SEQ ID NO:49 and SEQ ID NO:82, respectively. In certain embodiments, the DAR value for a composition (e.g., pharmaceutical compositions) comprising the immunoconjugate is in the range of 1.0 to 5.0, 1.0 to 4.0, 1.0 to 3.4, 1.0 to 3.0, 1.5 to 2.5, 2.0 to 2.5, or 1.8 to 2.2. In some embodiments, the DAR is less than 4.0, less than 3.8, less than 3.6, less than 3.5, less than 3.0 or less than 2.5.

In certain embodiments, the present invention provides an immunoconjugate represented by the following formula:

CBACy^{C1})_{W}_{_{C}},

wherein:

CBA is the antibody or antigen-binding fragment thereof or the polypeptide described herein that is covalently linked to Cy^{C1} through a cysteine residue;

W_{C} is 1 or 2;

Cy^{C1} is represented by the following formula:

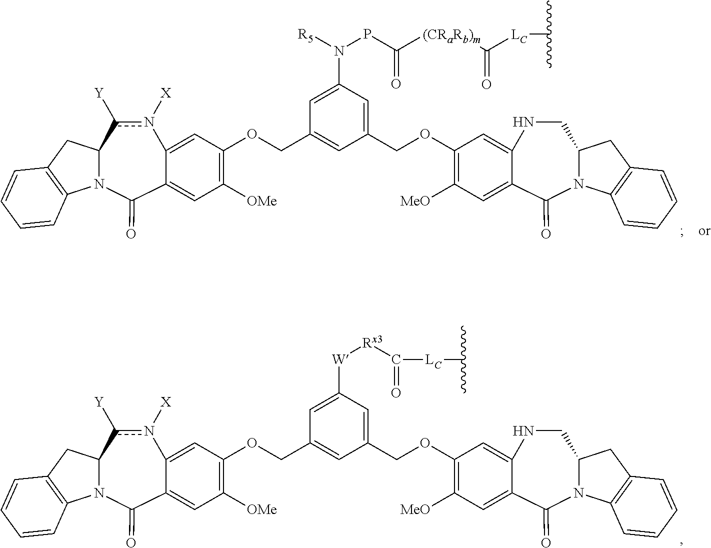

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃H or a pharmaceutically acceptable salt thereof;

R₅ is —H or a (C₁-C₃)alkyl;

P is an amino acid residue or a peptide containing 2 to 20 amino acid residues;

Rₐ and R_{b}, for each occurrence, are independently —H, (C₁-C₃)alkyl, or a charged substituent or an ionizable group Q;

m is an integer from 1 to 6;

W′ is —NR^{e′},

R^{e+} is —(CH₂—CH₂—O)ₙ—R^{k};

n is an integer from 2 to 6;

R^{k} is —H or -Me;

R^{x3} is a (C₁-C₆)alkyl; and,

L_{C} is represented by

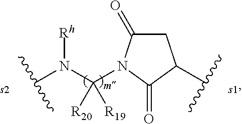

s1 is the site covalently linked to CBA, and s2 is the site covalently linked to the —C(═O)— group on Cy^{C1}; wherein:

R₁₉ and R₂₀, for each occurrence, are independently —H or a (C₁-C₃)alkyl;

m″ is an integer between 1 and 10; and

R^{h} is —H or a (C₁-C₃)alkyl.

In certain embodiments, the present invention provides an immunoconjugate represented by the following formula:

CBACy^{C2})_{WC},

wherein:

CBA is the antibody or antigen-binding fragment thereof or the polypeptide described herein that is covalently linked to Cy^{C2} through a cysteine residue;

W_{C} is 1 or 2;

Cy^{C2} is represented by the following formula:

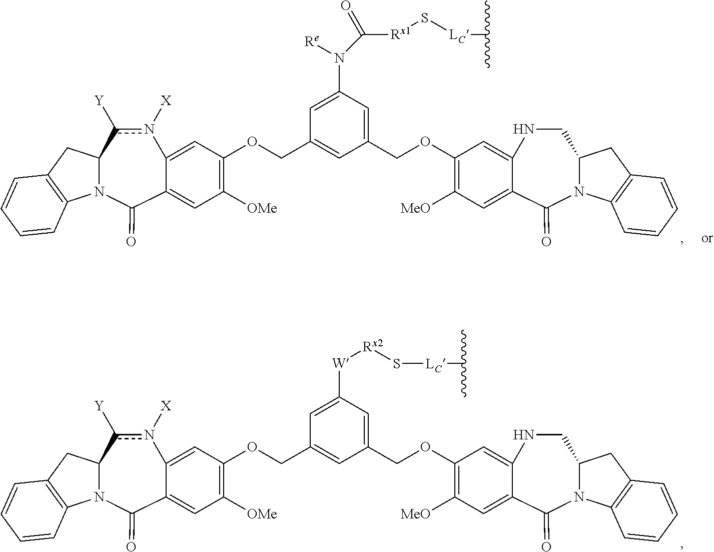

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃H or a pharmaceutically acceptable salt thereof;

R^{x1} is a (C₁-C₆)alkyl;

R^{e} is —H or a (C₁-C₆)alkyl;

W′ is —NH^{e′};

R^{e′} is —(CH₂—CH₂—O)ₙ—R^{k};

n is an integer from 2 to 6;

R^{k} is —H or -Me;

R^{x2} is a (C₁-C₆)alkyl;

L_{C}′ is represented by the following formula:

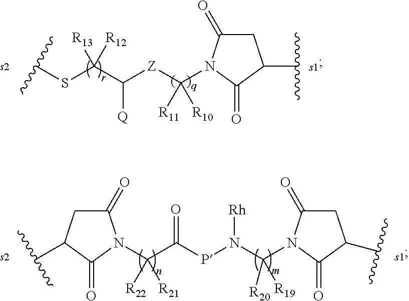

wherein:

s1 is the site covalently linked to the CBA and s2 is the site covalently linked to —S— group on Cy^{C2};

Z is —C(═O)—NR₉—, or —NR₉—C(═O)—;

Q is —H, a charged substituent, or an ionizable group;

R₉, R₁₀, R₁₁, R₁₂, R₁₃, R₁₉, R₂₀, R₂₁ and R₂₂, for each occurrence, are independently —H or a (C₁-C₃)alkyl;

q and r, for each occurrence, are independently an integer between 0 and 10;

m and n are each independently an integer between 0 and 10;

R^{h} is —H or a (C₁-C₃)alkyl; and

P′ is an amino acid residue or a peptide containing 2 to 20 amino acid residues.

In certain embodiments, the immunoconjugate of the present invention is represented by the following formula:

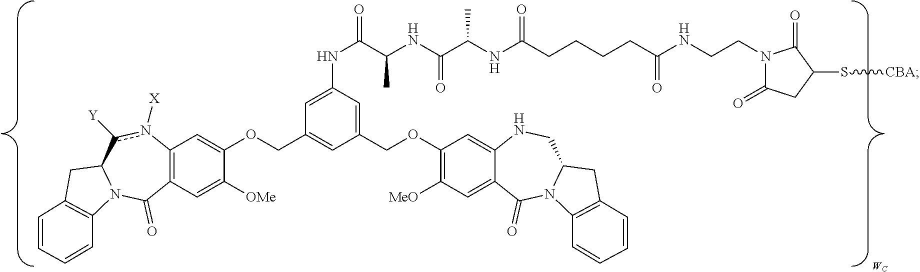

or a pharmaceutically acceptable salt thereof, wherein:

the double line [symbol] between N and C represents a single bond or a double bond, provided that when it is a double bond, X is absent and Y is —H, and when it is a single bond, X is —H, and Y is —SO₃H or a pharmaceutically acceptable salt thereof;

CBA is an isolated monoclonal antibody, or antigen-binding fragment thereof, that specifically binds to an epitope in the extracellular region of human cMET, wherein the antibody or antigen-binding fragment thereof comprises light chain complementary determining regions LC CDR1, LC CDR2, and LC CDR3 and heavy chain complementary determining regions HC CDR1, HC CDR2, and HC CDR3 having the sequences of SEQ ID NOs:4, 5, and 7 and SEQ ID NOs:13, 14, and 15, respectively. In certain embodiments, the isolated monoclonal antibody, or antigen-binding fragment thereof comprises a heavy chain variable domain (VH) and a light chain variable domain (VL) having sequences of SEQ ID NO:32 and SEQ ID NO:36, respectively. In other embodiments, the isolated monoclonal antibody comprises a heavy chain and a light chain having the sequences of SEQ ID NO:49 and SEQ ID NO:54.

The present invention also provides a pharmaceutical composition comprising an antibody or antigen-binding fragment thereof or a polypeptide, or an immunoconjugate described herein and a pharmaceutically acceptable carrier.

The present invention also provides a method for inhibiting aberrant cell proliferation comprising contacting a MET-expressing cell with an isolated monoclonal antibody or antigen-binding fragment thereof or a polypeptide or an immunoconjugate described herein, wherein said contacting inhibits the aberrant proliferation of said cells. In certain embodiments, the contacting induces apoptosis of the cells. In certain embodiments, the MET-expressing cell is a cancer cell. In certain embodiments, the cancer cell is cMet overexpressed, non-amplified. In certain embodiments, the cancer cell is cMet amplified.

Also provided in the present invention is a method for treating a cell proliferation disorder in a patient, comprising administering to the patient a therapeutically effective amount of an isolated, monoclonal antibody or antigen-binding fragment thereof, a polypeptide, an immunoconjugate, or a pharmaceutical composition thereof described herein.

The present invention also provides a use of an isolated, monoclonal antibody or antigen-binding fragment thereof, a polypeptide, an immunoconjugate, or a pharmaceutical composition thereof described herein for treating a cell proliferation disorder in a patient. Also provided in the present invention is a use of an isolated, monoclonal antibody or antigen-binding fragment thereof, a polypeptide, an immunoconjugate, or a pharmaceutical composition thereof for the manufacture of a medicament for treating a cell proliferation disorder in a patient.

In certain embodiments, the patient has been identified having cMet overexpressed, non-amplified. In certain embodiments, the patients has been identified having cMet amplified.

In certain embodiments, the cell proliferation disorder is cancer. In certain embodiments, the cancer is a cancer selected from the group consisting of glioblastoma, pancreatic cancer, gastric cancer, prostate cancer, ovarian cancer, breast cancer, hepatocellular carcinoma (HCC), melanoma, osteosarcoma, and colorectal cancer (CRC), lung cancer including small-cell lung cancer (SCLC), and non-small cell lung cancer (NSCLC), head and neck squamous cell carcinoma (HNSCC), kidney cancer, renal cancer, esophageal cancer, and thyroid cancer. In certain embodiments, the cancer is Met-amplified NSCLC.

### BRIEF DESCRIPTION OF THE DRAWINGS

FIG. 1 depicts the results of HGF-binding assay using MKN45 (solid bars) or BxPC3 cells (open bars) incubated with hybridoma supernatant obtained from fusion 247 containing various anti-MET antibodies.

FIG. 2 depicts the results of HGF-binding assay using MKN45 (solid bars) or BxPC3 cells (open bars) incubated with hybridoma supernatant obtained from fusion 248 containing various anti-MET antibodies.

FIGS. 3A and 3B show sequence alignment of CDR-grafted hucMET-27 constructs.

FIGS. 4A and 4B show positions of backmutations in CDR-grafted hucMET-27 constructs.

FIG. 5 shows binding of hucMET-27 antibodies to NCI-H441 cells expressing human cMET antigen as determined by FACS.

FIGS. 6A, 6B, 6C, and 6D show FACS binding data of huCMET-27 antibodies and conjugates to EBC-1.

FIG. 7 shows pErk stimulation of huCMET-27 antibodies and conjugates in NCI-H441.

FIG. 8 shows pAkt stimulation of huCMET-27 antibodies and conjugates in NCI-H441.

FIG. 9 shows cell proliferation data of huCMET-27 antibodies and conjugates in NCI-H441.

FIGS. 10A, 10B, 10C, and 10D show in vitro cytotoxicity of cMET antibody drug conjugates in EBC-1 and NCI-H441 cell lines.

FIGS. 11A, 11B, 11C, and 11D show in vitro cytotoxicity of cMET antibody drug conjugates in the presence of HGF in EBC-1 cell line.

FIGS. 12A, 12B, and 12C show in vitro cytotoxicity of cMET antibody drug conjugates in gastric cell lines.

FIG. 13 shows in vitro cytotoxicity of cMET antibody drug conjugates in HEP3B cell line.

FIGS. 14A, 14B, 14C, and 14D show in vitro cytotoxicity of cMET SMCC-DM1 antibody drug conjugates in various cell lines.

FIG. 15 shows the anti-tumor activity of cMET SMCC-DM1 antibody drug conjugates in vivo.

FIG. 16 shows the anti-tumor activity of hucMETv1.2-sSPDB-DM4 (5 mg/kg) and hucMETv1.2-DGN549 (lysine-linked; 3 μg/kg and 10 μg/kg, by payload) in the EBC-1 human non-small cell lune squamous cell carcinoma xenograft model.

FIG. 17 shows the anti-tumor activity of hucMETGv1.3-sSPDB-DM4 (5 mg/kg) and hucMETGv1.3-S442C-DGN549 (3 μg/kg and 10 μg/kg, by payload) in HSC2, a HNSCC xenograft model.

FIG. 18 shows the anti-tumor activity of hucMETGv1.3-sSPDB-DM4 (5 mg/kg) and hucMET27Gv1.3-DGN549 (3 μg/kg and 10 μg/kg, by payload) in H1975, a human non-small cell lune squamous cell carcinoma xenograft model.

FIG. 19 shows cell proliferation, pAKT, and pERK stimulation of different cMET reference antibodies, huCMET-27 antibody, and conjugate.

FIG. 20 shows EC₅₀ values of huCMET-27 conjugates and free payload in various cMET over-expressing NSCLC cell lines.

FIG. 21 shows in vitro cytotoxicity of cMET antibody drug conjugates in THLE-2 transformed hepatocytes.

FIG. 22 shows binding of hucMET-27 antibodies and conjugates with and without antibody hinge modifications to EBC-1 cells expressing human cMET antigen as determined by FACS.

FIG. 23 shows cell proliferation of different cMET reference antibodies and huCMET-27 antibodies with and without hinge modifications.

FIG. 24 shows pAKT stimulation of different cMET reference antibodies and huCMET-27 antibodies with and without hinge modifications.

FIG. 25 shows pERK stimulation of different cMET reference antibodies and huCMET-27 antibodies with and without hinge modifications.

FIG. 26 shows in vitro cytotoxicity of cMET antibody drug conjugates with and without antibody hinge modifications in EBC-1 and Hs746T cell lines.

FIG. 27 shows EC₅₀ values of huCMET-27-DM4 conjugates and free payload in cMET over-expressing and cMET amplified cell lines.

### DETAILED DESCRIPTION OF THE INVENTION

### Definitions

To facilitate an understanding of the present invention, a number of terms and phrases are defined below.

As used herein, the term “MET” or “c-MET” or “cMET” or “MET antigen” or HGFR or HGF receptor refers to polypeptides and any variants, isoforms and species homologs of MET that are naturally expressed or are expressed on cells transfected with the HGFR gene, or the like. Human MET is also known as the hepatocyte growth factor or HGF receptor, the scatter factor or SF receptor, and is a member of the receptor tyrosine kinase family. Additional synonyms for MET, as recognized in the art, include HGFR, HGFR antigen, MET receptor, c-MET, c-MET receptor, met proto-oncogene tyrosine kinase or proto-oncogene c-Met, RCCP2 or AUTS9. Two transcript variants encoding different isoforms have been found for human MET. Transcript Variant 1 represents the longer transcript corresponding to GenBank ID (GI) 42741654. It encodes the longer isoform (a) and comprises a 1408 amino acid protein described by GenBank Protein ID 42741655. Transcript Variant 2 uses an alternate in-frame splice junction at the end of an exon compared to variant 1 and corresponds to GenBank ID (GI) 188595715. The resulting isoform (b) comprises a 1390 amino acid protein described by GenBank Protein ID 188595716 and has the same N- and C-termini but is shorter compared to isoform (a).

As used herein, “aberrant MET receptor activation” refers to the dysregulation of MET expression and/or MET signaling including, but not limited to, overexpression of c-Met and/or HGF (e.g., in the presence or absence of gene amplification, e.g., cMET overexpressed-amplified or cMET overexpressed-non-amplified), constitutive kinase activation of c-Met in the presence (i.e., cMET amplified setting) or absence of gene amplification (cMET non-amplified setting), activating mutations of c-Met, and autocrine activation of c-Met by HGF.

For example, “aberrant MET receptor activation” may mean and include any heightened or altered expression or overexpression of MET protein in a tissue, e.g. an increase in the amount of a protein, caused by any means including enhanced expression or translation, modulation of the promoter or a regulator of the protein, amplification of a gene for a protein, or enhanced half-life or stability, such that more of the protein exists or can be detected at any one time, in contrast to a non-overexpressed state. Aberrant MET expression includes and contemplates any scenario or alteration wherein the MET protein expression or post-translational modification is overexpressed, including wherein an altered MET protein, as in mutated MET protein or variant due to sequence alteration, deletion or insertion, or altered folding is expressed.

In one embodiment, “aberrant MET receptor activation” may refer to enhanced MET receptor signaling activity that leads to the activation of key oncogenic signaling pathways including, but not limited to, RAS, PI3 kinase, STAT, β-catenin, Notch, Src, MAPK and Akt signaling pathways. “Aberrant MET receptor activation” may be associated with enhanced angiogenesis and cell metastasis.

In other embodiments, “aberrant MET receptor activation” refers to MET receptor activation, receptor dimerization and associated activation of tyrosine kinase and/or serine/threonine kinase activity.

In another embodiment, “aberrant MET receptor activation” is present when MET receptor associated tyrosine kinase activity is activated. In one aspect, a MET receptor associated tyrosine kinase activity is activated when the MET associated tyrosine kinase activity is detectable.

As used herein, an “antibody” or fragment and the like includes any protein or peptide containing molecule that comprises at least a portion of an immunoglobulin molecule, such as, but not limited to, at least one complementarity determining region (CDR) of a heavy or light chain or a ligand binding portion thereof, a heavy chain variable region or light chain variable region, a heavy chain or light chain constant region, a framework region, or any portion thereof, or at least one portion of an antigen or antigen receptor or binding protein, which can be incorporated into an antibody to MET of the present invention. Such antibody optionally further affects a specific ligand, such as, but not limited to, where such antibody modulates, decreases, increases, antagonizes, agonizes, partially agonizes, partially antagonizes, mitigates, alleviates, blocks, inhibits, abrogates and/or interferes with at least one antigen activity or binding, or with antigen receptor activity or binding, in vitro, in situ, in vivo and ex vivo. As a non-limiting example, various MET specific antibodies are disclosed, wherein a specified portion or variant can bind at least one antigen molecule, or specified portions, variants or domains thereof. A suitable antigen specific antibody, specified portion, or variant can also optionally affect at least one activity or function, such as, but not limited to, ligand binding, receptor dimerization, receptor phosphorylation, receptor signaling, membrane association, cell migration, cell proliferation, receptor binding activity, RNA, DNA or protein production and/or synthesis.

Antibodies are heterotetrameric glycoproteins, composed of two identical light chains (LC) and two identical heavy chains (HC). Typically, each light chain is linked to a heavy chain by one covalent disulfide bond, while the number of disulfide linkages varies between the heavy chains of different immunoglobulin isotypes. Each heavy and light chain also has spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa and lambda, based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

The term “antibody” also includes fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian antigens, such as MET, alone or in combination with other antigens. For example, antibody fragments capable of binding to antigen or portions thereof, include, but are not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the present invention (see, e.g., Colligan, Immunology).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

The term “antibody fragment” refers to a portion of an intact antibody, generally the antigen binding or variable region of an intact antibody. Examples of antibody fragments include, but are not limited to Fab, Fab′, F(ab′)2, single chain (scFv) and Fv fragments, diabodies; linear antibodies; single-chain antibody molecules; single Fab arm “one arm” antibodies and multispecific antibodies formed from antibody fragments, among others.

Antibody fragments include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an antigen or antigen receptor or binding protein, which can be incorporated into an antibody to MET of the present invention.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “epitope” refers to a protein determinant capable of specific binding to an antibody. Epitopes usually consist of chemically active surface groupings of molecules such as amino acids or sugar side chains and usually have specific three dimensional structural characteristics, as well as specific charge characteristics. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Blocking” antibody is one which inhibits or reduces the biological activity of the antigen it binds such as MET. Preferred blocking antibodies substantially or completely inhibit the biological activity of the antigen. Desirably, the biological activity is reduced by 10%, 20%, 30%, 50%, 70%, 80%, 90%, 95%, or even 100%. In one embodiment, the blocking antibody reduces the MET associated tyrosine kinase activity 10%, 20%, 30%, 50%, 70%, 80%, 90%, 95%, or even 100%.

An “isolated” antibody is one separated and/or recovered from its natural environment. Contaminant components of its natural environment are materials which would interfere with diagnostic or therapeutic uses for the antibody, and may include enzymes, hormones, and other proteinaceous or non-proteinaceous solutes. In preferred aspects, the antibody will be purified (1) to greater than 95% by weight of antibody as determined by, for example, the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE (sodium dodecyl sulfate polyacrylamide gel electrophoresis) under reducing or non-reducing conditions using Coomassie blue or, preferably, silver stain. Isolated antibody includes the MET antibody in situ within recombinant cells since at least one component of the antibody's natural environment will not be present. Ordinarily, however, isolated antibody will be prepared by at least one purification step.

A “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

As used herein, the term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc.) with the desired specificity, affinity, and capability while the constant regions are homologous to the sequences in antibodies derived from another (usually human) to avoid eliciting an immune response in that species.

As used herein, the term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539.

As used herein, the term “engineered antibody” or “altered antibody” includes an antibody with significant human frameworks and constant regions (CL, CH domains (e.g., CH1, CH2, CH3), and hinge), and CDRs derived from antigen binding antibodies such as anti-MET antibodies or fragments thereof. Fully human frameworks comprise frameworks that correspond to human germline sequences as well as sequences with somatic mutations. CDRs may be derived from one or more CDRs that associate with or bind to antigen in or outside of the context of any antibody framework. For example, the CDRs of the human engineered antibody of the present invention directed to MET may be derived from CDRs that bind antigen in the context of a mouse antibody framework and then are engineered to bind antigen in the context of a human framework. Often, the human engineered antibody is substantially non-immunogenic in humans.

Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. A human engineered antibody is distinct from a chimeric or humanized antibody.

An engineered antibody can be produced by a non-human animal or prokaryotic or eukaryotic cell that is capable of expressing functionally rearranged human or human engineered immunoglobulin (e.g., heavy chain and/or light chain) genes. Further, when an engineered antibody is a single chain antibody, it can comprise a linker peptide that is not found in native human or non-human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably, human, human engineered, resurfaced or humanized, antibodies that have binding specificities for at least two different antigens such as MET and a non-MET antigen. In the present case, one of the binding specificities is for at least one antigenic protein, the other one is for another antigenic protein. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of about 10 different antibody molecules, of which only one has the correct bispecific structure. The purification of the correct molecule is usually done by affinity chromatography steps or as otherwise described herein Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), U.S. 20090258026, U.S. 20060140946 and U.S. 20070298040, each entirely incorporated herein by reference.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); and antibody-dependent cell-mediated phagocytosis (ADCP).

“Human effector cells” are leukocytes which express one or more FcRs and perform effector functions. In certain aspects, the cells express at least FcyRIII and perform ADCC or ADCP effector function(s). Examples of human leukocytes which mediate ADCC or ADCP include peripheral blood mononuclear cells (PBMC), natural killer (NK) cells, monocytes, cytotoxic T cells and neutrophils. The effector cells may be isolated from a native source, e.g., from blood.

The term “conjugate”, “immunoconjugate” or “ADC” as used herein refers to a compound or a derivative thereof that is linked to a cell binding agent (i.e., an anti-MET antibody or fragment thereof) and is defined by a generic formula: C-L-A, wherein C=compound, L=linker, and A=cell binding agent (CBA) (e.g., an anti-MET antibody or fragment). In some embodiments, the generic formula: D-L-A, wherein D=drug, L=linker and A=cell binding agent (e.g., an anti-MET antibody or fragment), may also be used in the same manner.

A linker is any chemical moiety that is capable of linking a compound, usually a drug, such as a maytansinoid or an indolinobenzodiazepine compounds, to a cell-binding agent such as an anti-MET antibody or a fragment thereof in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

“Abnormal cell growth” or “aberrant cell proliferation”, as used herein, unless otherwise indicated, refers to cell growth that is independent of normal regulatory mechanisms (e.g., loss of contact inhibition). This includes, for example, the abnormal growth of: (1) tumor cells (tumors) that proliferate by expressing a mutated tyrosine kinase or over expression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; (5) benign and malignant cells of other proliferative diseases in which aberrant serine/threonine kinase activation occurs, and (6) benign and malignant cells of other proliferative diseases.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, blastoma, sarcoma, myeloma, leukemia or lymphoid malignancies. The term “cancer” or “cancerous.” as defined herein, includes “pre-cancerous” conditions that, if not treated, can evolve into a cancerous condition.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells).

As used herein, the term “cytotoxic agent” refers to a substance that inhibits or prevents one or more cellular functions and/or causes cell death.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods and compositions of the invention are useful in attempts to delay development of a disease or disorder.

A “therapeutically effective amount” refers to an amount effective, at dosages and for periods of time necessary, to achieve the desired therapeutic result. A “therapeutically effective amount” of a therapeutic agent (e.g., a conjugate or immunoconjugate) may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects.

The term “hepatocyte growth factor” or “HGF”, as used herein, refers, unless indicated otherwise, to any native or variant (whether native or synthetic) HGF polypeptide that is capable of activating the HGF/c-met signaling pathway under conditions that permit such process to occur.

A “therapeutic agent” encompasses both a biological agent such as an antibody, a peptide, a protein, an enzyme, a chemotherapeutic agent, or a conjugate or immunoconjugate.

The terms “polynucleotide” or “nucleic acid”, as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “vector” means a construct, which is capable of delivering, and optionally expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide”, “peptide”, and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The term “identical” or percent “identity”, as known in the art, is a measure of the relationship between two polynucleotides or two polypeptides, as determined by comparing their sequences. Identity or similarity with respect to a sequence is defined herein as the percentage of amino acid residues in the candidate sequence that are identical (i.e., same residue) or similar (i.e., amino acid residue from the same group based on common side-chain properties, see below) to anti-MET antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. Likewise percent similarity can be determined in an analogous manner based on the presence of both identical and similar residues.

The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482 489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are “substantially identical”, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value therebetween, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including, for example, basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies of the invention do not abrogate the binding of the polypeptide or antibody containing the amino acid sequence, to the antigen(s), to which the polypeptide or antibody binds. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

As used herein, “BxPC3 tumor cells” refer to a human pancreatic tumor cell line (ATCC No: CRL-1687; Tan M H, et al. Characterization of a new primary human pancreatic tumor line. Cancer Invest. 4: 15-23, 1986).

As used herein, “MKN45 tumor cells” refer to a human gastric adenocarcinoma cell line (DSMZ no. ACC 409; Naito et al., Virchows Arch B Cell Pathol Incl Mol Pathol 46: 145-154 (1984); Motoyama et al., Acta Pathol Jpn 36: 65-83 (1986); Rege-Cambrin et al., Cancer Genet Cytogenet 64: 170-173 (1992); DSMZ: Deutsche Sammlung von Mikroorganismen and Zellkulturen GmbH (German Collection of Microorganisms and Cell Cultures)).

“Alkyl” as used herein refers to a saturated linear or branched-chain monovalent hydrocarbon radical of one to twenty carbon atoms. Examples of alkyl include, but are not limited to, methyl, ethyl, 1-propyl, 2-propyl, 1-butyl, 2-methyl-1-propyl, —CH₂CH(CH₃)₂), 2-butyl, 2-methyl-2-propyl, 1-pentyl, 2-pentyl 3-pentyl, 2-methyl-2-butyl, 3-methyl-2-butyl, 3-methyl-1-butyl, 2-methyl-1-butyl, 1-hexyl), 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. Preferably, the alkyl has one to ten carbon atoms. More preferably, the alkyl has one to four carbon atoms.

The number of carbon atoms in a group can be specified herein by the prefix “Cₓ₋ₓₓ”, wherein x and xx are integers. For example, “C₁₋₄alkyl” is an alkyl group having from 1 to 4 carbon atoms.

The term “compound” or “cytotoxic compound,” or “cytotoxic agent” are used interchangeably. They are intended to include compounds for which a structure or formula or any derivative thereof has been disclosed in the present invention or a structure or formula or any derivative thereof that has been incorporated by reference. The term also includes, stereoisomers, geometric isomers, tautomers, solvates, metabolites, and salts (e.g., pharmaceutically acceptable salts) of a compound of all the formulae disclosed in the present invention. The term also includes any solvates, hydrates, and polymorphs of any of the foregoing. The specific recitation of “stereoisomers,” “geometric isomers,” “tautomers,” “solvates,” “metabolites,” “salt”, “conjugates,” “conjugates salt,” “solvate,” “hydrate,” or “polymorph” in certain aspects of the invention described in this application shall not be interpreted as an intended omission of these forms in other aspects of the invention where the term “compound” is used without recitation of these other forms.

The term “chiral” refers to molecules that have the property of non-superimposability of the mirror image partner, while the term “achiral” refers to molecules that are superimposable on their mirror image partner.

The term “stereoisomer” refers to compounds that have identical chemical constitution and connectivity, but different orientations of their atoms in space that cannot be interconverted by rotation about single bonds.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as crystallization, electrophoresis and chromatography.

“Enantiomers” refer to two stereoisomers of a compound that are non-superimposable mirror images of one another.

Stereochemical definitions and conventions used herein generally follow S. P. Parker, Ed., McGraw-Hill, Dictionary of Chemical Terms (1984) McGraw-Hill Book Company, New York; and Eliel, E. and Wilen, S., Stereochemistry of Organic Compounds, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and I or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “imine reactive reagent” refers to a reagent that is capable of reacting with an imine group. Examples of imine reactive reagent includes, but is not limited to, sulfites (H₂SO₃, H₂SO₂ or a salt of HSO₃⁻, SO₃²⁻ or HSO₂⁻ formed with a cation), metabisulfite (H₂S₂O₅ or a salt of S₂O₅²⁻ formed with a cation), mono, di, tri, and tetra-thiophosphates (PO₃SH₃, PO₂S₂H₃, POS₃H₃, PS₄H₃ or a salt of PO₃S³⁻, PO₂S₂³⁻, POS₃³⁻ or PS₄³⁻ formed with a cation), thio phosphate esters ((RⁱO)₂PS(ORⁱ), RⁱSH, RⁱSOH, RⁱSO₂H, RⁱSO₃H), various amines (hydroxyl amine (e.g., NH₂OH), hydrazine (e.g., NH₂NH₂), NH₂O—Rⁱ, Rⁱ′NH—Rⁱ, NH₂—Rⁱ), NH₂—CO—NH₂, NH₂—C(═S)—NH₂′thiosulfate (H₂S₂O₃ or a salt of S₂O₃²⁻ formed with a cation), dithionite (H₂S₂O₄ or a salt of S₂O₄²⁻ formed with a cation), phosphorodithioate (P(═S)(OR^{k})(SH)(OH) or a salt thereof formed with a cation), hydroxamic acid (R^{k}C(═O)NHOH or a salt formed with a cation), hydrazide (R^{k}CONHNH₂), formaldehyde sulfoxylate (HOCH₂SO₂H or a salt of HOCH₂SO₂⁻ formed with a cation, such as HOCH₂SO₂⁻Na⁺), glycated nucleotide (such as GDP-mannose), fludarabine or a mixture thereof, wherein Rⁱ and R^{i′} are each independently a linear or branched alkyl having 1 to 10 carbon atoms and are substituted with at least one substituent selected from —N(R^{j})₂, —CO₂H, —SO₃H, and —PO₃H; Rⁱ and R^{i′} can be further optionally substituted with a substituent for an alkyl described herein; R^{j} is a linear or branched alkyl having 1 to 6 carbon atoms; and R^{k} is a linear, branched or cyclic alkyl, alkenyl or alkynyl having 1 to 10 carbon atoms, aryl, heterocyclyl or heteroaryl (preferably, R^{k} is a linear or branched alkyl having 1 to 4 carbon atoms; more preferably, R^{k} is methyl, ethyl or propyl). Preferably, the cation is a monovalent cation, such as Na⁺ or K⁺. Preferably, the imine reactive reagent is selected from sulfites, hydroxyl amine, urea and hydrazine. More preferably, the imine reactive reagent is NaHSO₃ or KHSO₃.

The term “cation” refers to an ion with positive charge. The cation can be monovalent (e.g., Na+, K+, NH4+ etc.), bi-valent (e.g., Ca2+, Mg2+, etc.) or multi-valent (e.g., Al3+ etc.). Preferably, the cation is monovalent.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt can involve the inclusion of another molecule such as an acetate ion, a succinate ion or other counter ion. The counter ion can be any organic or inorganic moiety that stabilizes the charge on the parent compound. Furthermore, a pharmaceutically acceptable salt can have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt can be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt can be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

As used herein, the term “solvate” means a compound that further includes a stoichiometric or non-stoichiometric amount of solvent such as water, isopropanol, acetone, ethanol, methanol, DMSO, ethyl acetate, acetic acid, and ethanolamine dichloromethane, 2-propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

A “metabolite” or “catabolite” is a product produced through metabolism or catabolism in the body of a specified compound, a derivative thereof, or a conjugate thereof, or salt thereof. Metabolites of a compound, a derivative thereof, or a conjugate thereof, can be identified using routine techniques known in the art and their activities determined using tests such as those described herein. Such products can result for example from the oxidation, hydroxylation, reduction, hydrolysis, amidation, deamidation, esterification, deesterification, enzymatic cleavage, and the like, of the administered compound. Accordingly, the invention includes metabolites of compounds, a derivative thereof, or a conjugate thereof, of the invention, including compounds, a derivative thereof, or a conjugate thereof, produced by a process comprising contacting a compound, a derivative thereof, or a conjugate thereof, of this invention with a mammal for a period of time sufficient to yield a metabolic product thereof.

The phrase “pharmaceutically acceptable” indicates that the substance or composition must be compatible chemically and/or toxicologically, with the other ingredients comprising a formulation, and/or the mammal being treated therewith.

The term “protecting group” or “protecting moiety” refers to a substituent that is commonly employed to block or protect a particular functionality while reacting other functional groups on the compound, a derivative thereof, or a conjugate thereof. For example, an “amine-protecting group” or an “amino-protecting moiety” is a substituent attached to an amino group that blocks or protects the amino functionality in the compound. Such groups are well known in the art (see for example P. Wuts and T. Greene, 2007, Protective Groups in Organic Synthesis, Chapter 7, J. Wiley & Sons, NJ) and exemplified by carbamates such as methyl and ethyl carbamate, FMOC, substituted ethyl carbamates, carbamates cleaved by 1,6-β-elimination (also termed “self immolative”), ureas, amides, peptides, alkyl and aryl derivatives. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see P. G. M. Wuts & T. W. Greene, Protective Groups in Organic Synthesis, John Wiley & Sons, New York, 2007.

The term “amino acid” refers to naturally occurring amino acids or non-naturally occurring amino acid. In one embodiment, the amino acid is represented by NH₂—C(R^{aa′}R^{aa})—C(═O)OH, wherein R^{aa} and R^{aa′} are each independently H, an optionally substituted linear, branched or cyclic alkyl, alkenyl or alkynyl having 1 to 10 carbon atoms, aryl, heteroaryl or heterocyclyl or R^{aa} and the N-terminal nitrogen atom can together form a heteroycyclic ring (e.g., as in proline). The term “amino acid residue” refers to the corresponding residue when one hydrogen atom is removed from the amine and/or carboxy end of the amino acid, such as —NH—C(R^{aa′}R^{aa})—C(═O)O—.

The term “peptide” refers to short chains of amino acid monomers linked by peptide (amide) bonds. In some embodiments, the peptides contain 2 to 20 amino acid residues. In other embodiments, the peptides contain 2 to 10 amino acid residus. In yet other embodiments, the peptides contain 2 to 5 amino acid residues. As used herein, when a peptide is a portion of a cytotoxic agent or a linker described herein represented by a specific sequence of amino acids, the peptide can be connected to the rest of the cytotoxic agent or the linker in both directions. For example, a dipeptide X1-X2 includes X1-X2 and X2-X1. Similarly, a tripeptide X1-X2-X3 includes X1-X2-X3 and X3-X2-X1 and a tetrapeptide X1-X2-X3-X4 includes X1-X2-X3-X4 and X4-X2-X3-X1. X1, X2, X3 and X4 represents an amino acid residue.

The term “reactive ester group” refers to a group an ester group that can readily react with an amine group to form amide bond. Exemplary reactive ester groups include, but are not limited to, N-hydroxysuccinimide esters, N-hydroxyphthalimide esters, N-hydroxy sulfo-succinimide esters, para-nitrophenyl esters, dinitrophenyl esters, pentafluorophenyl esters and their derivatives, wherein said derivatives facilitate amide bond formation. In certain embodiments, the reactive ester group is a N-hydroxysuccinimide ester or a N-hydroxy sulfo-succinimide ester.

The term “amine reactive group” refers to a group that can react with an amine group to form a covalent bond. Exemplary amine reactive groups include, but are not limited to, reactive ester groups, acyl halides, sulfonyl halide, imidoester, or a reactive thioester groups. In certain embodiments, the amine reactive group is a reactive ester group. In one embodiment, the amine reactive group is a N-hydroxysuccinimide ester or a N-hydroxy sulfo-succinimide ester.

The term “thiol-reactive group” refers to a group that can react with a thiol (—SH) group to form a covalent bond. Exemplary thiol-reactive groups include, but are not limited to, maleimide, haloacetyl, aloacetamide, vinyl sulfone, vinyl sulfonamide or vinyal pyridine. In one embodiment, the thiol-reactive group is maleimide.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

### I. Anti-MET Antibodies and Antibody Fragments Thereof

The present invention provides agents that specifically bind MET. These agents are referred to herein as “MET binding agents.” Full-length amino acid sequences for human MET are known in the art.

In certain embodiments, the MET binding agents are antibodies, antibody fragments, or immunoconjugates. In some embodiments, the MET binding agents are humanized antibodies.

In certain embodiments, the MET-binding agents have one or more of the following effects: inhibit proliferation of tumor cells, reduce the tumorigenicity of a tumor by reducing the frequency of cancer stem cells in the tumor, inhibit tumor growth, trigger cell death of tumor cells, differentiate tumorigenic cells to a non-tumorigenic state, or prevent metastasis of tumor cells.

In certain embodiments the MET-binding agents are bivalent anti-MET antibodies. In certain embodiments the MET-binding agents are bivalent anti-MET antibodies, antibody fragments, or immunoconjugates that inhibit HGF binding to MET expressing cells.

In certain embodiments the MET-binding agents are bivalent anti-MET antibodies, antibody fragments, or immunoconjugates that inhibit proliferation.

In certain embodiments the MET-binding agents are bivalent anti-MET antibodies, antibody fragments, or immunoconjugates that are capable of inhibiting HGF-induced proliferation, while not inducing proliferation of MET-expressing cells in the absence of HGF. In certain embodiments the MET-binding agents are bivalent anti-MET antibodies, antibody fragments, or immunoconjugates that are capable of inhibiting HGF binding to MET expressing cells and inhibiting HGF-induced proliferation, while not inducing proliferation in the absence of HGF.

In one embodiment, a “c-MET binding agent” may be a c-MET binding polypeptide identified using recombinant procedures, for example, phage display or two hybrid screening and the like.

A. Exemplary Anti-MET Antibodies

Preferred antigen-specific MET antibodies of the invention are described below. Preferred antibodies are polypeptides comprised of one of the individual variable light chains or variable heavy chains described herein. Antibodies and polypeptides can also comprise both a variable light chain and a variable heavy chain. The variable light chain and variable heavy chain sequences of murine anti-MET antibodies are, for example, produced by hybridomas 247.27.16, 247.2.26, 247.48.38, 247.3.14, 247.22.2, 248.69.4, and 247.16.8.

Also provided are humanized (by resurfacing methods and CDR-grafting methods) antibodies.

Also provided are polypeptides that comprise: (a) a polypeptide having at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99% sequence identity to the amino acid sequence of any of the heavy chain variable regions of the antibodies produced by hybridomas 247.27.16, 247.2.26, 247.48.38, 247.3.14, 247.22.2, 248.69.4, and 247.16.8 and/or (b) a polypeptide having at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99% sequence identity to the amino acid sequence of any of the light chain variable regions of the antibodies produced by hybridomas 247.27.16, 247.2.26, 247.48.38, 247.3.14, 247.22.2, 248.69.4, and 247.16.8. In certain embodiments, the polypeptide comprises a polypeptide having at least about 95%, at least about 96%, at least about 97%, at least about 98%, or at least about 99% sequence identity to the amino acid sequence of any of the heavy chain variable regions of the antibodies produced by hybridomas 247.27.16, 247.2.26, 247.48.38, 247.3.14, 247.22.2, 248.69.4, and 247.16.8. Thus, in certain embodiments, the polypeptide comprises (a) a polypeptide having at least about 95% sequence identity to the amino acid sequence of any of the heavy chain variable regions of the antibodies produced by hybridomas 247.27.16, 247.2.26, 247.48.38, 247.3.14, 247.22.2, 248.69.4, and 247.16.8, and/or (b) a polypeptide having at least about 95% sequence identity to the amino acid sequence of any of the light chain variable regions of the antibodies produced by hybridomas 247.27.16, 247.2.26, 247.48.38, 247.3.14, 247.22.2, 248.69.4, and 247.16.8. In certain embodiments, the polypeptide comprises (a) a polypeptide having the amino acid sequence of any of the heavy chain variable regions of the antibodies produced by hybridomas 247.27.16, 247.2.26, 247.48.38, 247.3.14, 247.22.2, 248.69.4, and 247.16.8; and/or (b) a polypeptide having the amino acid sequence of any of the light chain variable regions of the antibodies produced by hybridomas 247.27.16, 247.2.26, 247.48.38, 247.3.14, 247.22.2, 248.69.4, and 247.16.8. In certain embodiments, the polypeptide is an antibody and/or the polypeptide specifically binds MET. In certain embodiments, the polypeptide is a murine, chimeric, or humanized or re-surfaced antibody that specifically binds MET. In certain embodiments, the polypeptide having a certain percentage of sequence identity to the amino acid sequence of any of the heavy chain variable regions or the light chain variable regions of the antibodies produced by hybridomas 247.27.16, 247.2.26, 247.48.38, 247.3.14, 247.22.2, 248.69.4, and 247.16.8 differs from the amino acid sequence of any of the heavy chain variable regions or the light chain variable regions of the antibodies produced by hybridomas 247.27.16, 247.2.26, 247.48.38, 247.3.14, 247.22.2, 248.69.4, and 247.16.8. by conservative amino acid substitutions.

Preferred antibodies are polypeptides containing one of the CDR sequences described herein. For example, an antigen specific antibody of the invention includes one of the light chain CDR sequences (i.e., LC CDR1, LC CDR2, and LC CDR3) and/or one of the heavy chain CDR sequences (i.e., HC CDR1, HC CDR2, and HC CDR3) shown below in Table 1.

TABLE 1
CDR sequences for exemplary c-MET-22 and c-MET-27 antibodies

| | LC CDR1 | LC CDR2 | LC CDR3 |
|---|---|---|---|
| 247.22.2 | RASENIYSTLA (SEQ | AATNLAD (SEQ ID | QHFWGTPYT (SEQ |
| (cMET-22) | ID NO: 1) | NO: 2) | ID NO: 3) |
| | (Kabat, ABM, Chothia | (Kabat, ABM, Chothia | (Kabat, ABM, Chothia |
| | definition) | definition) | definition) |
| 247.27.16 | RASESVDSYGNSFIH | RASNLES (SEQ ID NO: 5) | murine |
| (cMET-27) | (SEQ ID NO: 4) | (Kabat, ABM, Chothia | QQSNEDPLT (SEQ |
| | (Kabat, ABM, Chothia | definition) | ID NO: 6) |
| | definition) | | (Kabat definition) |
| | | | resurfaced |
| | | | v1.0 QQSNEDPLT |
| | | | (SEQ ID NO: 6) |
| | | | (Kabat definition) |
| | | | v1.2 QQSNEEPLT |
| | | | (SEQ ID NO: 7) |
| | | | (Kabat definition) |
| | | | v1.3 QQSNENPLT |
| | | | (SEQ ID NO: 8) (Kabat |
| | | | definition) |
| | | | CDR-grafted |
| | | | QQSNEEPLT (SEQ |
| | | | ID NO: 7) |
| | | | (Kabat definition) |
| | HC CDR1 | HC CDR2 | HC CDR3 |
| 247.22.2 | DYNMD (SEQ ID | DLNPNNGATIYNQKFKG | GNYYGNYYYLMDY |
| (cMET-22) | NO: 8) | (SEQ ID NO: 9) (Kabat | (SEQ ID NO: 10) |
| | (Kabat definition) | definition) | (Kabat, ABM, Chothia |
| | GYTFTDYNMD | DLNPNNGATI (SEQ ID | definition) |
| | (SEQ ID NO: 11) | NO: 12) | |
| | (ABM definition) | (ABM definition) | |
| 247.27.16 | SYDMS (SEQ ID | TINSNGVSIYYPDSVKG | EEITTEMDY (SEQ |
| (cMET-27) | NO: 13) | (SEQ ID NO: 14) (Kabat | ID NO: 15) |
| | (Kabat definition) | definition) | (Kabat, ABM, Chothia |
| | GFTFSSYDMS (SEQ | TINSNGVSIY (SEQ ID | definition) |
| | ID NO: 16) | NO: 17) | |
| | (ABM definition) | (ABM definition) | |

In particular embodiments, the anti-MET antibodies or anti-MET antibody fragment thereof comprises a LC CDR1, a LC CDR2, and a LC CDR3 and a HC CDR1, a HC CDR2, and a HC CDR3 having the sequences selected from the group consisting of:

(a) SEQ ID NOs:1, 2, and 3 and SEQ ID NOs:8, 9, and 10, respectively;

(b) SEQ ID NOs: 1, 2, and 3 and SEQ ID NOs: 8, 12, and 10, respectively;

(c) SEQ ID NOs:4, 5, and 6 and SEQ ID NOs:13, 14, and 15, respectively;

(d) SEQ ID NOs:4, 5, and 6 and SEQ ID NOs:13, 17, and 15, respectively;

(e) SEQ ID NOs:4, 5, and 7 and SEQ ID NOs:13, 17, and 15, respectively;

(f) SEQ ID NOs:4, 5, and 8 and SEQ ID NOs:13, 17, and 15, respectively; and

(g) SEQ ID NOs:4, 5, and 7 and SEQ ID NOs:13, 14, and 15, respectively. In certain embodiments, the anti-MET antibody or anti-MET antibody fragment thereof comprises a LC CDR1, a LC CDR2, and a LC CDR3 and a HC CDR1, a HC CDR2, and a HC CDR3 having the sequence of SEQ ID NOs:4, 5, and 7 and SEQ ID NOs:13, 14, and 15, respectively.

Also provided are humanized antibodies that comprise one of the individual variable light chains or variable heavy chains described herein. The humanized antibodies can also comprise both a variable light chain and a variable heavy chain. The variable light chain and variable heavy chain sequences of chimeric and humanized cMET-22 and cMET-27 antibodies are found in Table 2 below.

TABLE 2
Variable light chain and heavy chain sequences for exemplary cMET-22
and cMET-27 antibodies

| Name | Sequence | SEQ ID |
|---|---|---|
| ch247.22.2 | DIVMTQSPASLSVSVGETVTITCRASENIYSTLAWYQQKQGK | 18 |
| VL | SPQLLVYAATNLADGVPSRFSGSGSGTQYSLKINSLQSEDFG | |
| | SYYCQHFWGTPYTFGGGTKLEIKRT | |
| ch247.22.2 | EVQLEESGPELVKPGASVKIPCKASGYTFTDYNMDWVRQSH | 19 |
| VH | GKSLEWIGDLNPNNGATIYNQKFKGKATLTVDMSSSTAYME | |
| | LRSLTSEDTAVYYCARGNYYGNYYYLMDYWGQGTSVTVSS | |
| hu247.22.2 | DIQMTQSPSSLSVSVGERVTITCRASENIYSTLAWYQQKPGK | 20 |
| VL1.0 | SPKLLVYAATNLADGVPSRFSGSGSGTEYSLKINSLQPDDFG | |
| (resurfaced) | SYYCQHFWGTPYTFGGGTKLEIKR | |
| hu247.22.2 | EVQLVQSGAEVVKPGASVKIPCKASGYTFTDYNMDWVRQS | 21 |
| VH1.0 | PGKSLEWIGDLNPNNGATIYNEKFQGKATLTVDTSSSTAYM | |
| (resurfaced) | ELRSLTSEDTAVYYCARGNYYGNYYYLMDYWGQGTSVTVS | |
| | S | |
| hucMet- | DIQMTQSPSSLSASVGDRVTITCRASENIYSTLAWYQQKPGK | 22 |
| 22_VLGv2 | APKLLVYAATNLADGVPSRFSGSGSGTDYTLTISSLQPEDFA | |
| (CDR | TYYCQHFWGTPYTFGQGTKVEIKR | |
| grafted) | | |
| hucMet- | QVQLVQSGAEVKKPGASVKVSCKASGYTFTDYNMDWVRQ | 23 |
| 22_VHGv2 | APGQRLEWIGDLNPNNGATIYNQKFKGRATLTVDMSASTAY | |
| (CDR | MELSSLRSEDTAVYYCARGNYYGNYYYLMDYWGQGTSVT | |
| grafted) | VSS | |
| muCMET- | DIVLTQSPASLAVSLGQRATISCRASESVDSYGNSFIHWYQQ | 24 |
| 27 VL | KPGQPPKLLIYRASNLESGIPARFSGSGSRTDFTLTINPVEADD | |
| | VATYYCQQSNEDPLTFGAGTKLELKR | |
| muCMET- | EVQLVESGGGLVQPGGSLKLSCAASGFTFSSYDMSWVRQTP | 25 |
| 27 VH | DKRLELVATINSNGVSIYYPDSVKGRFTISRDIAKNTLYLQMS | |
| | SLKSEDTAMYYCAREEITTEMDYWGQGTSVTVSS | |
| ch247.27.16 | DIVMTQSPASLAVSLGQRATISCRASESVDSYGNSFIHWYQQ | 26 |
| VL | KPGQPPKLLIYRASNLESGIPARFSGSGSRTDFTLTINPVEADD | |
| | VATYYCQQSNEDPLTFGAGTKLELKRT | |
| ch247.27.16 | EVQLEESGGGLVQPGGSLKLSCAASGFTFSSYDMSWVRQTP | 27 |
| VH | DKRLELVATINSNGVSIYYPDSVKGRFTISRDIAKNTLYLQMS | |
| | SLKSEDTAMYYCAREEITTEMDYWGQGTSVTVSS | |
| hu247.27.16 | DIVLTQSPASLAVSPGQRATISCRASESVDSYGNSFIHWYQQ | 28 |
| VL1.0 | KPGQPPKLLIYRASNLESGIPARFSGSGSRTDFTLTINPVEAND | |
| (resurfaced) | VATYYCQQSNEDPLTFGGGTKLELK | |
| hu247.27.16 | DIVLTQSPASLAVSPGQRATISCRASESVDSYGNSFIHWYQQ | 29 |
| VL1.2 | KPGQPPKLLIYRASNLESGIPARFSGSGSRTDFTLTINPVEAND | |
| (resurfaced) | VATYYCQQSNEEPLTFGGGTKLELK | |
| hu247.27.16 | DIVLTQSPASLAVSPGQRATISCRASESVDSYGNSFIHWYQQ | 30 |
| VL1.3 | KPGQPPKLLIYRASNLESGIPARFSGSGSRTDFTLTINPVEAND | |
| (resurfaced) | VATYYCQQSNENPLTFGGGTKLELK | |
| hu247.27.16 | EVQLVESGGGLVQPGGSLRLSCAASGFTFSSYDMSWVRQTP | 31 |
| VH1.0 | GKGLELVATINSNGVSIYYPDSVKGRFTISRDIAKNTLYLQMS | |
| (resurfaced) | SLRAEDTAMYYCAREEITTEMDYWGQGTSVTVSS | |
| huCMET-27 | EIVLTQSPATLSLSPGERATLSCRASESVDSYGNSFIHWYQQK | 32 |
| VLGv1 | PGQAPRLLIYRASNLESGIPARFSGSGSGTDFTLTISSLEPEDF | |
| (CDR- | AVYYCQQSNEEPLTFGQGTKVELKR | |
| grafted) | | |
| huCMET-27 | EIVLTQSPATLSLSPGERATLSCRASESVDSYGNSFIHWYQQK | 33 |
| VLGv2 | PGQAPRLLIYRASNLESGIPARFSGSGSRTDFTLTISSLEPEDF | |
| (CDR- | AVYYCQQSNEEPLTFGQGTKVELKR | |
| grafted) | | |
| huCMET-27 | EVQLVESGGGLVQPGGSLRLSCAASGFTFSSYDMSWVRQAP | 34 |
| VHGv1 | GKGLEWVSTINSNGVSIYYPDSVKGRFTISRDNAKNSLYLQM | |
| (CDR- | NSLRAEDTAVYYCAREEITTEMDYWGQGTLVTVSS | |
| grafted) | | |
| huCMET-27 | EVQLVESGGGLVQPGGSLRLSCAASGFTFSSYDMSWVRQAP | 35 |
| VHGv2 | GKGLELVATINSNGVSIYYPDSVKGRFTISRDIAKNSLYLQM | |
| (CDR- | NSLRAEDTAVYYCAREEITTEMDYWGQGTLVTVSS | |
| grafted) | | |
| huCMET-27 | EVQLVESGGGLVQPGGSLRLSCAASGFTFSSYDMSWVRQAP | 36 |
| VHGv3 | GKGLEWVATINSNGVSIYYPDSVKGRFTISRDNAKNSLYLQ | |
| (CDR- | MNSLRAEDTAVYYCAREEITTEMDYWGQGTLVTVSS | |
| grafted) | | |

TABLE 3
Framework donor sequences for humanization by CDR grafting methods

| | | |
|---|---|---|
| human | EIVLTQSPATLSLSPGERATLSCRASQSVSSYLAWYQQKPGQ | 37 |
| germline | APRLLIYDASNRATGIPARFSGSGSGTDFTLTISSLEPEDFAVY | |
| IGKV3- | YCQQRSNWP | |
| 11*01 | | |
| (framework donor | | |
| for the light chain | | |
| in CDR-grafting) | | |
| human | EVQLVESGGGLVQPGGSLRLSCAASGFTFSSYEMNWVRQAP | 38 |
| germline | GKGLEWVSYISSSGSTIYYADSVKGRFTISRDNAKNSLYLQM | |
| IGHV3- | NSLRAEDTAVYYCAR | |
| 48*03 | | |
| (framework donor | | |
| for the heavy | | |
| chain in CDR- | | |
| grafting) | | |

In some embodiments, the anti-MET antibodies or fragment thereof comprises a variable light chain (VL) and a variable heavy chain (VH) having sequences that are at least about 95%, at least about 96%, at least about 97%, at least about 98%, at least about 99%, or 100% sequence identity to the sequences as follows:

(a) SEQ ID NO:18 and SEQ ID NO:19, respectively;

(b) SEQ ID NO:20 and SEQ ID NO:21, respectively;

(c) SEQ ID NO:22 and SEQ ID NO:23, respectively;

(d) SEQ ID NO:24 and SEQ ID NO:25, respectively;

(e) SEQ ID NO:26 and SEQ ID NO:27, respectively;

(f) SEQ ID NO:28 and SEQ ID NO:31, respectively;

(g) SEQ ID NO:29 and SEQ ID NO:31, respectively;

(h) SEQ ID NO:30 and SEQ ID NO:31, respectively;

(i) SEQ ID NO:32 and SEQ ID NO:36, respectively;

(j) SEQ ID NO:32 and SEQ ID NO:35, respectively;

(k) SEQ ID NO:32 and SEQ ID NO:34, respectively;

(l) SEQ ID NO:33 and SEQ ID NO:36, respectively;

(m) SEQ ID NO:33 and SEQ ID NO:35, respectively; and

(n) SEQ ID NO:33 and SEQ ID NO:34, respectively. In particular embodiments, the anti-MET antibody or fragment thereof comprises a VL and VH having the sequences of SEQ ID NO:32 and SEQ ID NO:36.

In certain embodiments, the polypeptide is an antibody and/or the polypeptide specifically binds MET. In certain embodiments, the polypeptide is a murine, chimeric, or humanized (by resurfacing methods or by CDR-grafting methods) antibody that specifically binds MET. In certain embodiments, the polypeptide having a certain percentage of sequence identity to SEQ ID NOs:18-36 by conservative amino acid substitutions.

Also provided are polypeptides that comprise one of the individual light chains or heavy chains described herein. These can also comprise both a light chain and a heavy chain. The light chain and heavy chain sequences of humanized cMET-22 and cMET-27 antibodies are below in Table 4.

TABLE 4
Full-length light chain and heavy chain sequences for exemplary
c-MET-22 and cMET-27 antibodies

| Name | Sequence | SEQ ID |
|---|---|---|
| hu247.22.2 | DIQMTQSPSSLSVSVGERVTITCRASENIYSTLAWYQQKPGK | 39 |
| LC1.0 | SPKLLVYAATNLADGVPSRFSGSGSGTEYSLKINSLQPDDFG | |
| (resurfaced) | SYYCQHFWGTPYTFGGGTKLEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| hu247.22.2 | EVQLVQSGAEVVKPGASVKIPCKASGYTFTDYNMDWVRQS | 40 |
| HC1.0 | PGKSLEWIGDLNPNNGATIYNEKFQGKATLTVDTSSSTAYM | |
| (resurfaced) | ELRSLTSEDTAVYYCARGNYYGNYYYLMDYWGQGTSVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCDKTHTCPPCPAPELLGGPSVFLF | |
| | PPKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSRDELTKNQVSLTC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| hucMet- | DIQMTQSPSSLSASVGDRVTITCRASENIYSTLAWYQQKPGK | 41 |
| 22_LC_Gv2.0 | APKLLVYAATNLADGVPSRFSGSGSGTDYTLTISSLQPEDFA | |
| (CDR | TYYCQHFWGTPYTFGQGTKVEIKRTVAAPSVFIFPPSDEQLK | |
| grafted) | SGTASVVCLLNNFYPREAKVQWKVDNALQSGNSQESVTEQ | |
| | DSKDSTYSLSSTLTLSKADYEKHKVYACEVTHQGLSSPVTKS | |
| | FNRGEC | |
| hucMet- | QVQLVQSGAEVKKPGASVKVSCKASGYTFTDYNMDWVRQ | 42 |
| 22_HC_Gv2.0 | APGQRLEWIGDLNPNNGATIYNQKFKGRATLTVDMSASTAY | |
| (CDR | MELSSLRSEDTAVYYCARGNYYGNYYYLMDYWGQGTSVT | |
| grafted) | VSSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKCK | |
| | VSNKALPAPIEKTISKAKGQPREPQVYTLPPSRDELTKNQVSL | |
| | TCLVKGFYPSDIAVEWESNGQPENNYKTTPPVLDSDGSFFLY | |
| | SKLTVDKSRWQQGNVFSCSVMHEALHNHYTQKSLSLSPG | |
| muCMET- | DIVLTQSPASLAVSLGQRATISCRASESVDSYGNSFIHWYQQ | 43 |
| 27 LC | KPGQPPKLLIYRASNLESGIPARFSGSGSRTDFTLTINPVEADD | |
| | VATYYCQQSNEDPLTFGAGTKLELKRADAAPTVSIFPPSSEQ | |
| | LTSGGASVVCFLNNFYPKDINVKWKIDGSERQNGVLNSWTD | |
| | QDSKDSTYSMSSTLTLTKDEYERHNSYTCEATHKTSTSPIVK | |
| | SFNRNEC | |
| muCMET- | EVQLVESGGGLVQPGGSLKLSCAASGFTFSSYDMSWVRQTP | 44 |
| 27 HC | DKRLELVATINSNGVSIYYPDSVKGRFTISRDIAKNTLYLQMS | |
| | SLKSEDTAMYYCAREEITTEMDYWGQGTSVTVSSAKTTPPS | |
| | VYPLAPGSAAQTNSMVTLGCLVKGYFPEPVTVTWNSGSLSS | |
| | GVHTFPAVLESDLYTLSSSVTVPSSPRPSETVTCNVAHPASST | |
| | KVDKKIVPRDCGCKPCICTVPEVSSVFIFPPKPKDVLTITLTPK | |
| | VTCVVVDISKDDPEVQFSWFVDDVEVHTAQTQPREEQFNST | |
| | FRSVSELPIMHQDWLNGKEFKCRVNSAAFPAPIEKTISKTKG | |
| | RPKAPQVYTIPPPKEQMAKDKVSLTCMITDFFPEDITVEWQW | |
| | NGQPAENYKNTQPIMNTNGSYFVYSKLNVQKSNWEAGNTF | |
| | TCSVLHEGLHNHHTEKSLSHSPGK | |
| hu247.27.16 | DIVLTQSPASLAVSPGQRATISCRASESVDSYGNSFIHWYQQ | 45 |
| LC1.0 | KPGQPPKLLIYRASNLESGIPARFSGSGSRTDFTLTINPVEAND | |
| (resurfaced) | VATYYCQQSNEDPLTFGGGTKLELKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| hu247.27.16 | DIVLTQSPASLAVSPGQRATISCRASESVDSYGNSFIHWYQQ | 46 |
| LC1.2 | KPGQPPKLLIYRASNLESGIPARFSGSGSRTDFTLTINPVEAND | |
| (resurfaced) | VATYYCQQSNEEPLTFGGGTKLELKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| hu247.27.16 | DIVLTQSPASLAVSPGQRATISCRASESVDSYGNSFIHWYQQ | 47 |
| LC1.3 | KPGQPPKLLIYRASNLESGIPARFSGSGSRTDFTLTINPVEAND | |
| (resurfaced) | VATYYCQQSNENPLTFGGGTKLELKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| hu247.27.16 | EVQLVESGGGLVQPGGSLRLSCAASGFTFSSYDMSWVRQTP | 48 |
| HC1.0 | GKGLELVATINSNGVSIYYPDSVKGRFTISRDIAKNTLYLQMS | |
| (resurfaced) | SLRAEDTAMYYCAREEITTEMDYWGQGTSVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCDKTHTCPPCPAPELLGGPSVFLFPPKPKDTL | |
| | MISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLTCLVKGFYP | |
| | SDIAVEWESNGQPENNYKTTPPVLDSDGSFFLYSKLTVDKSR | |
| | WQQGNVFSCSVMHEALHNHYTQKSLSLSPG | |
| huCMET-27 | EIVLTQSPATLSLSPGERATLSCRASESVDSYGNSFIHWYQQK | 49 |
| LC_G v1 | PGQAPRLLIYRASNLESGIPARFSGSGSGTDFTLTISSLEPEDF | |
| (CDR- | AVYYCQQSNEEPLTFGQGTKVELKRTVAAPSVFIFPPSDEQL | |
| grafted) | KSGTASVVCLLNNFYPREAKVQWKVDNALQSGNSQESVTE | |
| | QDSKDSTYSLSSTLTLSKADYEKHKVYACEVTHQGLSSPVT | |
| | KSFNRGEC | |
| huCMET-27 | EIVLTQSPATLSLSPGERATLSCRASESVDSYGNSFIHWYQQK | 50 |
| LC_Gv2 | PGQAPRLLIYRASNLESGIPARFSGSGSRTDFTLTISSLEPEDF | |
| (CDR | AVYYCQQSNEEPLTFGQGTKVELKRTVAAPSVFIFPPSDEQL | |
| grafted) | KSGTASVVCLLNNFYPREAKVQWKVDNALQSGNSQESVTE | |
| | QDSKDSTYSLSSTLTLSKADYEKHKVYACEVTHQGLSSPVT | |
| | KSFNRGEC | |
| huCMET-27 | EVQLVESGGGLVQPGGSLRLSCAASGFTFSSYDMSWVRQAP | 51 |
| HC_G v1 | GKGLEWVSTINSNGVSIYYPDSVKGRFTISRDNAKNSLYLQM | |
| (CDR | NSLRAEDTAVYYCAREEITTEMDYWGQGTLVTVSSASTKGP | |
| grafted) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPG | |
| huCMET-27 | EVQLVESGGGLVQPGGSLRLSCAASGFTFSSYDMSWVRQAP | 52 |
| HC_G v2 | GKGLELVATINSNGVSIYYPDSVKGRFTISRDIAKNSLYLQM | |
| (CDR- | NSLRAEDTAVYYCAREEITTEMDYWGQGTLVTVSSASTKGP | |
| grafted) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPG | |
| huCMET-27 | EVQLVESGGGLVQPGGSLRLSCAASGFTFSSYDMSWVRQAP | 53 |
| HC_G v3 | GKGLEWVATINSNGVSIYYPDSVKGRFTISRDNAKNSLYLQ | |
| (CDR- | MNSLRAEDTAVYYCAREEITTEMDYWGQGTLVTVSSASTK | |
| grafted) | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPG | |
| huCMET-27 | EVQLVESGGGLVQPGGSLRLSCAASGFTFSSYDMSWVRQAP | 54 |
| HC_Gv3 | GKGLEWVATINSNGVSIYYPDSVKGRFTISRDNAKNSLYLQ | |
| Cysmab | MNSLRAEDTAVYYCAREEITTEMDYWGQGTLVTVSSASTK | |
| (CDR | GPSVFPLAPSSKSTSGGTAALGCLVKDYFPEPVTVSWNSGAL | |
| grafted) | TSGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLCLSPG | |
| huCMET-27 | EVQLVESGGGLVQPGGSLRLSCAASGFTFSSYDMSWVRQAP | 77 |
| HC_Gv3 | GKGLEWVATINSNGVSIYYPDSVKGRFTISRDNAKNSLYLQ | |
| Cysmab- | MNSLRAEDTAVYYCAREEITTEMDYWGQGTLVTVSSASTK | |
| IgG2 hinge | GPSVFPLAPSSKSTSGGTAALGCLVKDYFPEPVTVSWNSGAL | |
| (CDR | TSGVHTFPAVLQSSGLYSLSSVVTVPSSSLGTQTYICNVNHKP | |
| grafted) | SNTKVDKKVERKCCVECPPCPAPELLGGPSVFLFPPKPKDTL | |
| | MISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLTCLVKGFYP | |
| | SDIAVEWESNGQPENNYKTTPPVLDSDGSFFLYSKLTVDKSR | |
| | WQQGNVFSCSVMHEALHNHYTQKSLCLSPG | |
| huCMET-27 | EVQLVESGGGLVQPGGSLRLSCAASGFTFSSYDMSWVRQAP | 78 |
| HC_Gv3 | GKGLEWVATINSNGVSIYYPDSVKGRFTISRDNAKNSLYLQ | |
| Cysmab- | MNSLRAEDTAVYYCAREEITTEMDYWGQGTLVTVSSASTK | |
| IgG2 hinge- | GPSVFPLAPCSKSTSGGTAALGCLVKDYFPEPVTVSWNSGAL | |
| S127C | TSGVHTFPAVLQSSGLYSLSSVVTVPSSSLGTQTYICNVNHKP | |
| (CDR | SNTKVDKKVERKCCVECPPCPAPELLGGPSVFLFPPKPKDTL | |
| grafted) | MISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLTCLVKGFYP | |
| | SDIAVEWESNGQPENNYKTTPPVLDSDGSFFLYSKLTVDKSR | |
| | WQQGNVFSCSVMHEALHNHYTQKSLCLSPG | |
| HuCMET-27 | EVQLVESGGGLVQPGGSLRLSCAASGFTFSSYDMSWVRQAP | 79 |
| HC_Gv3- | GKGLEWVATINSNGVSIYYPDSVKGRFTISRDNAKNSLYLQ | |
| IgG2 hinge | MNSLRAEDTAVYYCAREEITTEMDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVERKCCVECPPCPAPELLGGPSVFLFPPKPKDTL | |
| | MISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLTCLVKGFYP | |
| | SDIAVEWESNGQPENNYKTTPPVLDSDGSFFLYSKLTVDKSR | |
| | WQQGNVFSCSVMHEALHNHYTQKSLSLSPG | |
| huCMET-27 | EVQLVESGGGLVQPGGSLRLSCAASGFTFSSYDMSWVRQAP | 80 |
| HC_Gv3- | GKGLEWVATINSNGVSIYYPDSVKGRFTISRDNAKNSLYLQ | |
| IgG2 hinge- | MNSLRAEDTAVYYCAREEITTEMDYWGQGTLVTVSSASTK | |
| S127C | GPSVFPLAPCSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVERKCCVECPPCPAPELLGGPSVFLFPPKPKDTL | |
| | MISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLTCLVKGFYP | |
| | SDIAVEWESNGQPENNYKTTPPVLDSDGSFFLYSKLTVDKSR | |
| | WQQGNVFSCSVMHEALHNHYTQKSLSLSPG | |
| huCMET-27 | EVQLVESGGGLVQPGGSLRLSCAASGFTFSSYDMSWVRQAP | 81 |
| HC_Gv3- | GKGLEWVATINSNGVSIYYPDSVKGRFTISRDNAKNSLYLQ | |
| Cysmab- | MNSLRAEDTAVYYCAREEITTEMDYWGQGTLVTVSSASTK | |
| hinge#28 | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPKSCDCHCPPCPAPELLGGPSVFLFPPKPKDT | |
| | LMISRTPEVTCVVVDVSHEDPEVKFNWYVDGVEVHNAKTK | |
| | PREEQYNSTYRVVSVLTVLHQDWLNGKEYKCKVSNKALPA | |
| | PIEKTISKAKGQPREPQVYTLPPSRDELTKNQVSLTCLVKGFY | |
| | PSDIAVEWESNGQPENNYKTTPPVLDSDGSFFLYSKLTVDKS | |
| | RWQQGNVFSCSVMHEALHNHYTQKSLCLSPG | |
| huCMET-27 | EVQLVESGGGLVQPGGSLRLSCAASGFTFSSYDMSWVRQAP | 82 |
| HC_Gv3- | GKGLEWVATINSNGVSIYYPDSVKGRFTI | |
| hinge#28 | SRDNAKNSLYLQMNSLRAEDTAVYYCAREEITTEMDYWGQ | |
| | GTLVTVSSASTKGPSVFPLAPSSKSTSGGTAALGCLVKDYFP | |
| | EPVTVSWNSGALTSGVHTFPAVLQSSGLYSLSSVVTVPSSSL | |
| | GTQTYICNVNHKPSNTKVDKRVEPKSCDCHCPPCPAPELLGG | |
| | PSVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSRDELTKN | |
| | QVSLTCLVKGFYPSDIAVEWESNGQPENNYKTTPPVLDSDGS | |
| | FFLYSKLTVDKSRWQQGNVFSCSVMHEALHNHYTQKSLSLS | |
| | PG | |
| huCMET-27 | EVQLVESGGGLVQPGGSLRLSCAASGFTFSSYDMSWVRQAP | 83 |
| HC_Gv3- | GKGLEWVATINSNGVSIYYPDSVKGRFTISRDNAKNSLYLQ | |
| Cysmab- | MNSLRAEDTAVYYCAREEITTEMDYWGQGTLVTVSSASTK | |
| hinge#26 | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPRDCGCKPCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLCLSPG | |
| huCMET-27 | EVQLVESGGGLVQPGGSLRLSCAASGFTFSSYDMSWVRQAP | 84 |
| HC_Gv3- | GKGLEWVATINSNGVSIYYPDSVKGRFTISRDNAKNSLYLQ | |
| hinge#26 | MNSLRAEDTAVYYCAREEITTEMDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKRVEPRDCGCKPCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPG | |

In some embodiments, the anti-MET antibodies or fragment thereof comprises a light chain and heavy chain sequence having at least about 95%, at least about 96%, at least about 97%, at least about 98%, at least about 99%, or 100% sequence identity to the sequences as follows:

(a) SEQ ID NO:39 and SEQ ID NO:40, respectively;

(b) SEQ ID NO:41 and SEQ ID NO:42, respectively;

(c) SEQ ID NO:43 and SEQ ID NO:44, respectively;

(d) SEQ ID NO:45 and SEQ ID NO:48, respectively;

(e) SEQ ID NO:46 and SEQ ID NO:48, respectively;

(f) SEQ ID NO:47 and SEQ ID NO:48, respectively;

(g) SEQ ID NO:49 and SEQ ID NO:54, respectively;

(h) SEQ ID NO:49 and SEQ ID NO:53, respectively;

(i) SEQ ID NO:49 and SEQ ID NO:52, respectively;

(j) SEQ ID NO:49 and SEQ ID NO:51, respectively;

(k) SEQ ID NO:50 and SEQ ID NO:53, respectively;

(l) SEQ ID NO:50 and SEQ ID NO:52, respectively;

(m) SEQ ID NO:50 and SEQ ID NO:51, respectively;

(n) SEQ ID NO:49 and SEQ ID NO:77, respectively;

(o) SEQ ID NO:49 and SEQ ID NO:78, respectively;

(p) SEQ ID NO:49 and SEQ ID NO:79, respectively;

(q) SEQ ID NO:49 and SEQ ID NO:80, respectively;

(r) SEQ ID NO:49 and SEQ ID NO:81, respectively;

(s) SEQ ID NO:49 and SEQ ID NO:82, respectively;

(t) SEQ ID NO:49 and SEQ ID NO:83, respectively; and

(u) SEQ ID NO:49 and SEQ ID NO:84, respectively. In particular embodiments, the anti-MET antibody or fragment thereof comprises a light chain and heavy chain having the sequences of SEQ ID NO:49 and SEQ ID NO:54. In some embodiments, the anti-MET antibody or fragment thereof comprises a light chain and heavy chain having the sequences of SEQ ID NO:49 and SEQ ID NO:53. In some embodiments, the anti-MET antibody or fragment thereof comprises a light chain and heavy chain having the sequences of SEQ ID NO:49 and SEQ ID NO:82.

In certain embodiments, the polypeptide is an antibody and/or the polypeptide specifically binds MET. In certain embodiments, the polypeptide is a murine, chimeric, or humanized (by resurfacing methods or CDR-grafting methods) antibody that specifically binds MET. In certain embodiments, the polypeptide having a certain percentage of sequence identity to SEQ ID NOs:39-54 by conservative amino acid substitutions.

One having ordinary skill in the art understands that the sequences in the present application are non-limiting examples.

In certain embodiments, the anti-MET antibodies of the invention include a hinge region modification to reduce agonistic activity of the antibody, where the modification includes an amino acid sequence selected from the group consisting of SEQ ID NOs: 85-108. In particular embodiments, the anti-MET antibodies or anti-MET antibody fragment thereof comprises a LC CDR1, a LC CDR2, and a LC CDR3 and a HC CDR1, a HC CDR2, and a HC CDR3 having the sequences of SEQ ID NOs:4, 5, and 7 and SEQ ID NOs:13, 14, and 15, respectively, where the antibody or anti-MET antibody fragment thereof are further characterized in that the antibody or fragment thereof also comprise a hinge region modification including an amino acid sequence as disclosed in Table 13.

TABLE 13
Hinge region sequences

| | | SEQ |
|---|---|---|
| Name | Sequence | ID |
| Hinge#26 | PRDCGCKPCPPCP | 85 |
| Hinge#28 | PKSCDCHCPPCP | 86 |
| Hinge#22 | RKCCVECPPCP | 87 |
| Hinge#23 | PRDCGCKPCICT | 88 |
| Hinge#24 | PKSCGCKPCICT | 89 |
| Hinge#25 | PKSCGCKPCICP | 90 |
| Hinge#25 | PRDCGCHTCPPCP | 91 |
| Hinge#27 | PRDCGCHTCPPCP | 92 |
| Hinge#58 | CKSCDKTHTCPPCP | 93 |
| Hinge#59 | PCSCDKTHTCPPCP | 94 |
| Hinge#60 | PKCCDKTHTCPPCP | 95 |
| Hinge#61 | PKSCCKTHTCPPCP | 96 |
| Hinge#62 | PKSCDCTHTCPPCP | 97 |
| Hinge#63 | PKSCDKCHTCPPCP | 98 |
| Hinge#64 | PKSCDKTHCCPPCP | 99 |
| Hinge#65 | KCDKTHTCPPCP | 100 |
| Hinge#66 | PKSCDCHTCPPCP | 101 |
| Hinge#67 | PKSCDCTHCPPCP | 102 |
| Hinge#68 | PCSCKHTCPPCP | 103 |
| Hinge#69 | PSCCTHTCPPCP | 104 |
| Hinge#70 | PSCDKHCCPPCP | 105 |
| Hinge#71 | PKSCTCPPCP | 106 |
| Hinge#72 | PKSCDKCVECPPCP | 107 |
| IgG2 hinge | RKCCVECPPCP | 108 |

B. Engineered Anti-MET Antibodies

The anti-MET antibodies and fragments thereof, conjugates, compositions and methods of the invention can be mutant antibodies and the like. The anti-MET antibody can be an “engineered antibody” or an altered antibody such as an amino acid sequence variant of the anti-MET antibody wherein one or more of the amino acid residues of the anti-MET antibody have been modified. The modifications to the amino acid sequence of the anti-MET antibody include, for example, modifications to the polypeptide and/or polynucleotide sequence to improve affinity or avidity of the antibody or fragment for its antigen, improve stability, and/or modifications to the polypeptide and/or polynucleotide sequence to improve production of the antibody, and/or modifications to the Fc portion of the antibody to improve effector function unless otherwise indicated herein or known. The modifications may be made to any known anti-MET antibodies or anti-MET antibodies identified as described herein. Such altered antibodies necessarily have less than 100% sequence identity or similarity with a reference anti-MET antibody. In a preferred aspect, the altered antibody will have an amino acid sequence having at least 20%, 25%, 35%, 45%, 55%, 65%, or 75% amino acid sequence identity or similarity with the amino acid sequence of either the heavy or light chain variable domain of the anti-MET antibody, more preferably at least 80%, more preferably at least 85%, more preferably at least 90%, and most preferably at least 95%. In a preferred aspect, the altered antibody will have an amino acid sequence having at least 25%, 35%, 45%, 55%, 65%, or 75% amino acid sequence identity or similarity with the amino acid sequence of the heavy chain CDR1, CDR2, or CDR3 of the anti-MET antibody, more preferably at least 80%, more preferably at least 85%, more preferably at least 90%, and most preferably at least 95%. In a preferred aspect, the altered antibody will have an amino acid sequence having at least 25%, 35%, 45%, 55%, 65%, or 75% amino acid sequence identity or similarity with the amino acid sequence of light chain CDR1, CDR2, or CDR3 of the anti-MET antibody, more preferably at least 80%, more preferably at least 85%, more preferably at least 90%, and most preferably at least 95%, 96%, 97%, 98%, 99%. In a preferred aspect, the altered antibody will maintain human MET binding capability. In certain aspects, the anti-MET antibody of the invention comprises a heavy chain that is about 10%, 15%, 20%, 25%, 30%, 35%, 40%, 45%, 50%, 55%, 60%, 65%, 70%, 75%, 80%, 85%, 90%, 95%, 96%, 97%, 98%, 99% or more identical to the amino acid sequences of the amino acid sequences of the heavy chain variable regions of the antibodies produced by hybridoma 247.27.16, 247.2.26, 247.48.38, 247.3.14, 247.22.2, 248.69.4, or 247.16.8. In certain aspects, the anti-MET antibody of the invention comprises a light chain that is about 10%, 15%, 20%, 25%, 30%, 35%, 40%, 45%, 50%, 55%, 60%, 65%, 70%, 75%, 80%, 85%, 90%, 95%, 96%, 97%, 98%, 99% or more identical to the amino acid sequences of the amino acid sequences of the light chain variable regions of the antibodies produced by hybridoma 247.27.16, 247.2.26, 247.48.38, 247.3.14, 247.22.2, 248.69.4, or 247.16.8.

In some embodiments of the invention, the anti-MET antibody can be an “engineered antibody” or an altered antibody such as an amino acid sequence variant of the anti-MET antibody wherein one or more of the amino acid residues of the anti-MET antibody have been modified. In a preferred aspect, the altered antibody will have an amino acid sequence having at least 1-5, 1-3, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 conservative amino acid substitutions when compared with the amino acid sequence of either the heavy or light chain variable domain of the anti-MET antibody. In a preferred aspect, the altered antibody will have an amino acid sequence having at least 1-20, 1-15, 1-10, 1-5, 1-3, 20, 19, 18, 17, 16, 15, 14,13, 12, 11, 10, 9, 8, 7, 6, 5, 4, 3, 2 or 1 conservative amino acid substitutions when compared with the amino acid sequence of the heavy chain CDR1, CDR2, or CDR3 of the anti-MET antibody. In a preferred aspect, the altered antibody will have an amino acid sequence having at least 1-20, 1-15, 1-10, 1-5, 1-3, 20, 19, 18, 17, 16, 15, 14,13, 12, 11, 10, 9,8,7,6, 5, 4, 3, 2 or 1 conservative amino acid substitutions when compared with the amino acid sequence of the light chain CDR1, CDR2, or CDR3 of the anti-MET antibody produced by hybridoma 247.27.16, 247.2.26, 247.48.38, 247.3.14, 247.22.2, 248.69.4, or 247.16.8. In a preferred aspect, the altered antibody will maintain human MET binding capability. In certain aspects, the anti-MET antibody of the invention comprises a heavy chain having an amino acid sequence that has about 1-20, 1-15, 1-10, 1-5, 1-3, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 conservative amino acid substitutions when compared with the amino acid sequence of the amino acid sequences of the heavy chain variable regions of the antibodies produced by hybridoma 247.27.16, 247.2.26, 247.48.38, 247.3.14, 247.22.2, 248.69.4, or 247.16.8. In certain aspects, the anti-MET antibody of the invention comprises a light chain having an amino acid sequence that has about 1-20, 1-15, 1-10, 1-5, 1-3, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 conservative amino acid substitutions when compared with the amino acid sequence of the light chain variable regions of the antibodies produced by hybridoma 247.27.16, 247.2.26, 247.48.38, 247.3.14, 247.22.2, 248.69.4, or 247.16.8. In a preferred aspect, the altered antibody will have an amino acid sequence having at least 1-20, 1-15, 1-10, 1-5, 1-3, 20, 19, 18, 17, 16, 15, 14,13, 12, 11, 10, 9,8,7,6, 5, 4, 3, 2 or 1 conservative amino acid substitutions when compared with the amino acid sequence of the heavy chain CDR1, CDR2, or CDR3 of the anti-MET antibody produced by hybridoma 247.27.16, 247.2.26, 247.48.38, 247.3.14, 247.22.2, 248.69.4, or 247.16.8.

To generate an altered antibody, one or more amino acid alterations (e.g., substitutions) are introduced in one or more of the hypervariable regions of an antibody. Alternatively, or in addition, one or more alterations (e.g., substitutions) of framework region residues may be introduced in an anti-MET antibody where these result in an improvement in the binding affinity of the antibody mutant for the antigen. Examples of framework region residues to modify include those which non-covalently bind antigen directly (Amit et al., Science, 233:747-753 (1986)); interact with/effect the conformation of a CDR (Chothia et al., J. Mol. Biol., 196:901-917 (1987)); and/or participate in the VL VH interface. In certain aspects, modification of one or more of such framework region residues results in an enhancement of the binding affinity of the antibody for the antigen. For example, from about one to about five framework residues (e.g., 1, 2, 3, 4 or 5) may be altered in this aspect of the invention. Sometimes, this may be sufficient to yield an antibody with an enhancement of the binding affinity, even where none of the hypervariable region residues have been altered. Normally, however, an altered antibody will comprise additional hypervariable region alteration(s).

The hypervariable region residues which are altered may be changed randomly, especially where the starting binding affinity of an anti-MET antibody for the antigen is such that such randomly produced altered antibody can be readily screened.

One useful procedure for generating such an altered antibody is called “alanine scanning mutagenesis” (Cunningham and Wells, Science, 244:1081-1085 (1989)). One or more of the hypervariable region residue(s) are replaced by alanine or polyalanine residue(s) to affect the interaction of the amino acids with the antigen. Those hypervariable region residue(s) demonstrating functional sensitivity to the substitutions then are refined by introducing additional or other mutations at or for the sites of substitution. Thus, while the site for introducing an amino acid sequence variation is predetermined, the nature of the mutation per se need not be predetermined. The Ala-mutants produced this way are screened for their biological activity as described herein and/or as known in the art.

Another procedure for generating such an altered antibody involves affinity maturation using phage display (Hawkins et al., J. Mol. Biol., 254:889-896 (1992) and Lowman et al., Biochemistry, 30(45):10832-10837 (1991)).

Mutations in antibody sequences may include substitutions, deletions, including internal deletions, additions, including additions yielding fusion proteins, or conservative substitutions of amino acid residues within and/or adjacent to the amino acid sequence, but that result in a “silent” change, in that the change produces a functionally equivalent anti-MET antibody or fragment. Conservative amino acid substitutions may be made on the basis of similarity in polarity, charge, solubility, hydrophobicity, hydrophilicity, and/or the amphipathic nature of the residues involved. For example, non-polar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues can influence chain orientation. Non-conservative substitutions will entail exchanging a member of one of these classes for a member of another class. Furthermore, if desired, non-classical amino acids or chemical amino acid analogs can be introduced as a substitution or addition into the antibody sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs generally.

Any technique for mutagenesis known in the art can be used to modify individual nucleotides in a DNA sequence, for purposes of making amino acid substitution(s) in the antibody sequence, or for creating/deleting restriction sites to facilitate further manipulations. Such techniques include, but are not limited to, chemical mutagenesis, in vitro site-directed mutagenesis (Kunkel, Proc. Natl. Acad. Sci. USA, 82:488 (1985); Hutchinson, C. et al., J. Biol. Chem., 253:6551 (1978)), oligonucleotide-directed mutagenesis (Smith, Ann. Rev. Genet., 19:423-463 (1985); Hill et al., Methods Enzymol., 155:558-568 (1987)), PCR-based overlap extension (Ho et al., Gene, 77:51-59 (1989)), PCR-based megaprimer mutagenesis (Sarkar et al., Biotechniques, 8:404-407 (1990)), etc. Modifications can be confirmed by double-stranded dideoxy DNA sequencing.

C. Antibody Humanization and Resurfacing

Methods for engineering, humanizing or resurfacing non-human or human antibodies can also be used and are well known in the art. A humanized, resurfaced or similarly engineered antibody may have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence.

Among many available sources, human Ig sequences are disclosed at the following exemplary web pages:

Entrez and IgBlast web pages at the National Center for Biotechnology Information;

ImMunoGeneTics (“IMGT”) web pages at the global ImMunoGeneTics Web Resource for immunoglobulins (IG), T cell receptors (TR) and major histocompatibility complex (MHC) and related proteins of the immune system (RPI). Editor: Marie-Paule Lefranc (LIGM, Universite-Montpellier II, CNRS, Montpellier, France);

The Kabat Database of Sequences of Proteins of Immunological Interest; and

FTP KABAT repository at the National Center for Biotechnology Information.

The contents of each of these resources and citations is hereby incorporated herein in its entirety by reference.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing MET binding. Accordingly, part or all of the non-human or human CDR sequences are maintained while the non-human sequences of the variable and constant regions may be replaced with human or other amino acids.

Antibodies can also optionally be humanized, resurfaced, engineered or human antibodies engineered with retention of high affinity for the antigen MET and other favorable biological properties. To achieve this goal, humanized (or human) or engineered anti-MET antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as MET. In this way, framework (FR) residues can be selected and combined from the consensus and import sequences so that the desired antibody characteristic, such as increased affinity for the target antigen(s), is achieved.

Humanization, resurfacing or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; PCT/: US98/16280; US96/18978; US91/09630; US91/05939; US94/01234; GB89/01334; GB91/01134; GB92/01755; WO90/14443; WO90/14424; WO90/14430; EP 229246; 7,557,189; 7,538,195; and 7,342,110, each of which is entirely incorporated herein by reference, including the references cited therein.

D. Variant Fc Regions and Engineered Effector Function

The present invention provides formulation of proteins comprising a variant Fc region. That is, a non-naturally occurring Fc region, for example an Fc region comprising one or more non-naturally occurring amino acid residues. Also encompassed by the variant Fc regions of the present invention are Fc regions which comprise amino acid deletions, additions and/or modifications.

In certain aspects, the antibody comprises an altered (e.g., mutated) Fc region. For example, in some aspects, the Fc region has been altered to reduce or enhance the effector functions of the antibody, alter serum half life or other functional properties of the antibody. In some aspects, the Fc region is an isotype selected from IgM, IgA, IgG, IgE, or other isotype.

It will be understood that Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 (Cγ2 and Cγ3) and the hinge between Cγ1 (Cγ1) and Cγ2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al., supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc. Polymorphisms have been observed at a number of Fc positions, including, but not limited to, Kabat 270, 272, 312, 315, 356, and 358, and thus slight differences between the presented sequence and sequences in the prior art may exist and would be known to one of skill in the art based on the present teachings.

Fc mutations can be introduced into engineered antibodies to alter their interaction with the neonatal Fc receptor (FcRn) and improve their pharmacokinetic properties. A collection of human Fc variants with improved binding to the FcRn has been described and include, for example, those disclosed in Shields et al., 2001. High resolution mapping of the binding site on human IgG1 for FcγRI, FcγRII, FcγRIII, and FcRn and design of IgG1 variants with improved binding to the FcγR, J. Biol. Chem. 276:6591-6604), which is hereby entirely incorporated by reference.

Thus the serum half-life of proteins comprising Fc regions may be increased by increasing the binding affinity of the Fc region for FcRn. In one aspect, the Fc variant protein has enhanced serum half life relative to comparable molecule.

The Fc hinge region can also be engineered to alter Fab arm flexibility as a means to manipulate antibody binding, effector potency or other functional properties of the antibody. The flexibility of the antibody's Fc hinge is largely a function of its length and the number and placement of cysteine residues. Amino acid changes to the Fc hinge cysteine residues or length have been described which can elicited altered ADCC or CDC activity (Dall'Acqua W F et al., 2006; J Immunol; 177:1129-38), or other antibody binding mediated functional activities (WO 2010064090). It may therefore be desirable to make such amino acid modifications, including amino acid deletions and substitutions, in the Fc hinge region.

It may also be desirable to modify the anti-MET antibody of the invention with respect to effector function, so as to enhance the effectiveness of the antibody in treating a cancer, for example. In vitro assays known in the art can be used to determine whether the anti-MET antibodies, compositions, conjugates and methods of the invention, for example, are capable of mediating effector functions such as ADCC or CDC, such as those described herein.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. High-affinity IgG antibodies, for example, directed to the surface of target cells “arm” the cytotoxic cells and afford such killing. Lysis of the target cell is extracellular, requires direct cell-to-cell contact, and does not involve complement. It is contemplated that, in addition to antibodies, other proteins comprising Fc regions, specifically Fc fusion proteins, having the capacity to bind specifically to an antigen-bearing target cell will be able to effect cell-mediated cytotoxicity. For simplicity, the cell-mediated cytotoxicity resulting from the activity of an Fc fusion protein is also referred to herein as ADCC activity.

The ability of any particular Fc variant protein to mediate lysis of the target cell by ADCC can be assayed. To assess ADCC activity an Fc variant protein of interest is added to target cells in combination with immune effector cells, which may be activated by the antigen antibody complexes resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985, 79:277-282; Bruggemann et al., 1987, J Exp Med, 166:1351-1361; Wilkinson et al., 2001, J Immunol Methods, 258:183-191; and Patel et al., 1995, J Immunol Methods, 184:29-38. Alternatively, or additionally, ADCC activity of the Fc variant protein of interest may be assessed in vivo, e.g., in an animal model such as that disclosed in Clynes et al., 1998, PNAS USA, 95:652-656.

“Complement dependent cytotoxicity” and “CDC” refer to the lysing of a target cell in the presence of complement. The complement activation pathway is initiated by the binding of the first component of the complement system (C1q) to a molecule, an antibody for example, complexed with a cognate antigen. To assess complement activation, a CDC assay, e.g. as described in Gazzano-Santoro et al., 1996, J. Immunol. Methods, 202:163, may be performed.

The Fc region of an antibody of the present invention can be designed with altered effector functions including, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.)

For example, one can generate a variant Fc region of the engineered anti-MET antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other aspects, only one of these activities may be increased, and, optionally, also the other activity reduced (e.g., to generate an Fc region variant with improved ADCC activity, but reduced CDC activity, and vice versa). An exemplary Fc mutant is the triple residue change, S239D, A330L, and I332E (EU numbering system) in which ADCC is enhanced and CDC activity is diminished. Non-limiting methods for designing such mutants can be found, for example, in Lazar et al. (2006, Proc. Natl. Acad. Sci. U.S.A. 103(11): 4005-4010) and Okazaki et al. (2004, J. Mol. Biol. 336(5):1239-49). See also WO 03/074679, WO 2004/029207, WO 2004/099249, WO2006/047350, WO 2006/019447, WO 2006/105338, WO 2007/041635.

Other methods of engineering Fc regions of antibodies so as to alter effector functions are known in the art (e.g., U.S. Patent Publication No. 20040185045 and PCT Publication No. WO 2004/016750, both to Koenig et al., which describe altering the Fc region to enhance the binding affinity for FcγRIIB as compared with the binding affinity for FCγRIIA; see, also, PCT Publication Nos. WO 99/58572 to Armour et al.; WO 99/51642 to Idusogie et al.; and U.S. Pat. No. 6,395,272 to Deo et al.; the disclosures of which are incorporated herein in their entireties). Methods of modifying the Fc region to decrease binding affinity to FcγRIIB are also known in the art (e.g., U.S. Patent Publication No. 20010036459 and PCT Publication No. WO 01/79299, both to Ravetch et al., the disclosures of which are incorporated herein in their entireties). Modified antibodies having variant Fc regions with enhanced binding affinity for FcγRIIIA and/or FcγRIIA as compared with a wild type Fc region are known (e.g., PCT Publication Nos. WO 2004/063351, to Stavenhagen et al.; the disclosure of which is incorporated herein in its entirety).

In additional examples, cysteine residue(s) may be introduced in the Fc region, thereby allowing interchain disulfide bond formation in this region. The homodimeric antibody thus generated may have improved internalization capability and/or increased complement-mediated cell killing and/or antibody-dependent cellular cytotoxicity (ADCC). See, Caron et al., J. Exp Med., 176:1191-1195 (1992) and Shopes, B., J. Immunol., 148:2918-2922 (1992). Homodimeric antibodies with enhanced activity may also be prepared using hetero-bifunctional cross-linkers as described in Wolff et al., Cancer Research, 53:2560-2565 (1993). Alternatively, an antibody can be engineered which has dual Fc regions and may thereby have enhanced complement lysis and ADCC capabilities. See, Stevenson et al., Anti-Cancer Drug Design, 3:219-230 (1989).

The present invention encompasses Fc variant proteins which have altered binding properties for an Fc ligand (e.g., an Fc receptor, C1q) relative to a comparable molecule (e.g., a protein having the same amino acid sequence except having a wild type Fc region). Examples of binding properties include, but are not limited to, binding specificity, equilibrium dissociation constant (K_{D}), dissociation and association rates (K_{off} and Kₒₙ), binding affinity and/or avidity. It is generally understood that a binding molecule (e.g., a Fc variant protein such as an antibody) with a low K_{D} is preferable to a binding molecule with a high K_{D}. However, in some instances the value of the Kₒₙ or K_{off} may be more relevant than the value of the K_{D}. One skilled in the art can determine which kinetic parameter is most important for a given antibody application.

The affinities and binding properties of an Fc domain for its ligand, may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immunoabsorbent assay (ELISA), or radioimmunoassay (RIA)), or kinetics (e.g., BIACORE.™ analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in, for example, Paul, W. E., ed., Fundamental Immunology, 4th Ed., Lippincott-Raven, Philadelphia (1999).

For example, a modification that enhances Fc binding to one or more positive regulators (e.g., FcγRIIIA) while leaving unchanged or even reducing Fc binding to the negative regulator FcγRIIB would be more preferable for enhancing ADCC activity. Alternatively, a modification that reduced binding to one or more positive regulator and/or enhanced binding to FcγRIIB would be preferable for reducing ADCC activity. Accordingly, the ratio of binding affinities (e.g., equilibrium dissociation constants (KD)) can indicate if the ADCC activity of an Fc variant is enhanced or decreased. For example, a decrease in the ratio of FcγRIIIA/FcγRIIB equilibrium dissociation constants (KD), will correlate with improved ADCC activity, while an increase in the ratio will correlate with a decrease in ADCC activity. Additionally, modifications that enhanced binding to C1q would be preferable for enhancing CDC activity while modification that reduced binding to C1q would be preferable for reducing or eliminating CDC activity.

In one aspect, the Fc variants of the invention bind FcγRIIIA with increased affinity relative to a comparable molecule. In another aspect, the Fc variants of the invention bind FcγRIIIA with increased affinity and bind FcγRIIB with a binding affinity that is unchanged relative to a comparable molecule. In still another aspect, the Fc variants of the invention bind FcγRIIIA with increased affinity and bind FcγRIIB with a decreased affinity relative to a comparable molecule. In yet another aspect, the Fc variants of the invention have a ratio of FcγRIIIA/FcγRIIB equilibrium dissociation constants (K_{D}) that is decreased relative to a comparable molecule.

In one aspect, the Fc variant protein has enhanced binding to one or more Fc ligand relative to a comparable molecule. In another aspect, the Fc variant protein has an affinity for an Fc ligand that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. In a specific aspect, the Fc variant protein has enhanced binding to an Fc receptor. In another specific aspect, the Fc variant protein has enhanced binding to the Fc receptor FcγRIIIA In still another specific aspect, the Fc variant protein has enhanced binding to the Fc receptor FcRn. In yet another specific aspect, the Fc variant protein has enhanced binding to C1q relative to a comparable molecule.

In another aspect, an Fc variant of the invention has an equilibrium dissociation constant (KD) that is decreased between about 2 fold and about 10 fold, or between about 5 fold and about 50 fold, or between about 25 fold and about 250 fold, or between about 100 fold and about 500 fold, or between about 250 fold and about 1000 fold relative to a comparable molecule. In another aspect, an Fc variant of the invention has an equilibrium dissociation constant (KD) that is decreased between 2 fold and 10 fold, or between 5 fold and 50 fold, or between 25 fold and 250 fold, or between 100 fold and 500 fold, or between 250 fold and 1000 fold relative to a comparable molecule. In a specific aspect, the Fc variants have an equilibrium dissociation constants (K_{D}) for FcγRIIIA that is reduced by at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or at least 400 fold, or at least 600 fold, relative to a comparable molecule.

In one aspect, an Fc variant protein has enhanced ADCC activity relative to a comparable molecule. In a specific aspect, an Fc variant protein has ADCC activity that is at least 2 fold, or at least 3 fold, or at least 5 fold, or at least 10 fold, or at least 50 fold, or at least 100 fold greater than that of a comparable molecule. In another specific aspect, an Fc variant protein has enhanced binding to the Fc receptor FcγRIIIA and has enhanced ADCC activity relative to a comparable molecule. In other aspects, the Fc variant protein has both enhanced ADCC activity and enhanced serum half life relative to a comparable molecule.

In one aspect, an Fc variant protein has enhanced CDC activity relative to a comparable molecule. In a specific aspect, an Fc variant protein has CDC activity that is at least 2 fold, or at least 3 fold, or at least 5 fold, or at least 10 fold, or at least 50 fold, or at least 100 fold greater than that of a comparable molecule. In other aspects, the Fc variant protein has both enhanced CDC activity and enhanced serum half life relative to a comparable molecule.

In one aspect, the present invention provides formulations, wherein the Fc region comprises a non-naturally occurring amino acid residue at one or more positions selected from the group consisting of 234, 235, 236, 239, 240, 241, 243, 244, 245, 247, 252, 254, 256, 262, 263, 264, 265, 266, 267, 269, 296, 297, 298, 299, 313, 325, 326, 327, 328, 329, 330, 332, 333, and 334 as numbered by the EU index as set forth in Kabat. Optionally, the Fc region may comprise a non-naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217) or as disclosed herein.

In a specific aspect, the present invention provides an Fc variant protein formulation, wherein the Fc region comprises at least one non-naturally occurring amino acid residue selected from the group consisting of 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241L, 241Y, 241E, 241 R. 243W, 243L 243Y, 243R, 243Q, 244H, 245A, 247V, 247G, 252Y, 254T, 256E, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 269H, 269Y, 269F, 269R, 296E, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 313F, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 332D, 332S, 332W, 332F, 332E, 332N, 332Q, 332T, 332H, 332Y, and 332A as numbered by the EU index as set forth in Kabat. Optionally, the Fc region may comprise additional and/or alternative non-naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In another aspect, the present invention provides an Fc variant protein formulation, wherein the Fc region comprises at least a non-naturally occurring amino acid at one or more positions selected from the group consisting of 239, 330 and 332, as numbered by the EU index as set forth in Kabat. In a specific aspect, the present invention provides an Fc variant protein formulation, wherein the Fc region comprises at least one non-naturally occurring amino acid selected from the group consisting of 239D, 330L and 332E, as numbered by the EU index as set forth in Kabat. Optionally, the Fc region may further comprise an additional non-naturally occurring amino acid at one or more positions selected from the group consisting of 252, 254, and 256, as numbered by the EU index as set forth in Kabat. In a specific aspect, the present invention provides an Fc variant protein formulation, wherein the Fc region comprises at least one non-naturally occurring amino acid selected from the group consisting of 239D, 330L and 332E, as numbered by the EU index as set forth in Kabat, and at least one non-naturally occurring amino acid at one or more positions are selected from the group consisting of 252Y, 254T and 256E, as numbered by the EU index as set forth in Kabat.

In one aspect, the Fc variants of the present invention may be combined with other known Fc variants such as those disclosed in Ghetie et al., 1997, Nat. Biotech. 15:637-40; Duncan et al, 1988, Nature 332:563-564; Lund et al., 1991, J. Immunol., 147:2657-2662; Lund et al, 1992, Mol. Immunol., 29:53-59; Alegre et al, 1994, Transplantation 57:1537-1543; Hutchins et al., 1995, Proc Natl. Acad Sci USA, 92:11980-11984; Jefferis et al, 1995, Immunol. Lett., 44:111-117; Lund et al., 1995, Faseb J., 9:115-119; Jefferis et al, 1996, Immunol Lett., 54:101-104; Lund et al, 1996, J. Immunol., 157:4963-4969; Armour et al., 1999, Eur. J. Immunol. 29:2613-2624; Idusogie et al, 2000, J. Immunol., 164:4178-4184; Reddy et al, 2000, J. Immunol., 164:1925-1933; Xu et al., 2000, Cell Immunol., 200:16-26; Idusogie et al, 2001, J. Immunol., 166:2571-2575; Shields et al., 2001, J Biol. Chem., 276:6591-6604; Jefferis et al., 2002, Immunol Lett., 82:57-65; Presta et al., 2002, Biochem. Soc. Trans., 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; and WO 04/063351 which disclose exemplary Fc variants. Also encompassed by the present invention are Fc regions which comprise deletions, additions and/or modifications. Still other modifications/substitutions/additions/deletions of the Fc domain will be readily apparent to one skilled in the art.

Alternatively or additionally, it may be useful to combine amino acid modifications with one or more further amino acid modifications that alter C1q binding and/or the complement dependent cytotoxicity (CDC) function of the Fc region of an antigen binding molecule. The starting polypeptide of particular interest may be one that binds to C1q and displays complement dependent cytotoxicity. Polypeptides with pre-existing C1q binding activity, optionally further having the ability to mediate CDC, may be modified such that one or both of these activities are enhanced. Amino acid modifications that alter C1q and/or modify its complement dependent cytotoxicity function are described, for example, in WO0042072, which is hereby entirely incorporated by reference.

Methods for generating non-naturally occurring Fc regions are known in the art. For example, amino acid substitutions and/or deletions can be generated by mutagenesis methods, including, but not limited to, site-directed mutagenesis (e.g., Kunkel, Proc. Natl. Acad. Sci. USA, 82:488-492 (1985)), PCR mutagenesis (e.g., Higuchi, in “PCR Protocols: A Guide to Methods and Applications”, Academic Press, San Diego, pp. 177-183 (1990)), and cassette mutagenesis (e.g., Wells et al., Gene, 34:315-323 (1985)). Preferably, site-directed mutagenesis is performed by the overlap-extension PCR method (e.g., Higuchi, in “PCR Technology: Principles and Applications for DNA Amplification”, Stockton Press, New York, pp. 61-70 (1989)). Other exemplary methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351, the entire contents of which are incorporated herein by reference).

In some aspects, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by methods disclosed herein and any method known to one skilled in the art, for example by using engineered or variant expression strains, by using growth conditions or media affecting glycosylation, by co-expression with one or more enzymes, for example DI N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al., 1999, Nat. Biotechnol., 17:176-180; Davies et al., 20017 Biotechnol Bioeng., 74:288-294; Shields et al., 2002, J Biol. Chem., 277:26733-26740; Shinkawa et al., 2003, J Biol. Chem., 278:3466-3473) U.S. Pat. No. 6,602,684; U.S. application Ser. No. 10/277,370; U.S. application Ser. No. 10/113,929; PCT WO 00/61739A1; PCT WO 01/292246A1; PCT WO 02/311140A1; PCT WO 02/30954A1; Potelligent™ technology (Biowa, Inc., Princeton, N.J.); GlycoMAb™ glycosylation engineering technology (GLYCART™ biotechnology AG, Zurich, Switzerland). See also, e.g., WO 00061739; EA01229125; US 20030115614; Okazaki et al., 2004, JMB, 336: 1239-49.

In certain aspects, an Fc variant protein with engineered glycoforms contains carbohydrate structures attached to the Fc region that lack fucose. Such variants have improved ADCC function. Examples of publications related to “defucosylated” or “fucose-deficient” antibodies include: US Pat. Appl. No. US 2003/0157108 (Presta, L.) and US 2004/0093621 (Kyowa Hakko Kogyo Co., Ltd); US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al., J. Mol. Biol., 336:1239-1249 (2004); Yamane Ohnuki et al., Biotech. Bioeng., 87: 614 (2004).

Antibodies with a bisecting N-acetylglucosamine (GlcNAc) in the carbohydrate attached to an Fc region of the antibody are referenced in, for example, WO 2003/011878, Jean-Mairet et al. and US Pat. No. 6,602,684, Umana et al. Antibodies with at least one galactose residue in the oligosaccharide attached to an Fc region of the antibody are reported in, for example, WO 1997/30087, Patel et al. See also, WO 1998/58964 and WO 1999/22764 (Raju, S.) concerning antibodies with altered carbohydrate attached to the Fc region thereof. See also, for example, US 2005/0123546 (Umana et al.) regarding antigen-binding molecules with modified glycosylation.

Non-limiting examples of cell lines producing defucosylated antibodies include Lec13 CHO cells deficient in protein fucosylation (Ripka et al. Arch. Biochem. Biophys. 249:533-545 (1986); US Pat. Appl. No. US 2003/0157108 AI, Presta, L; and WO 2004/056312 AI, Adams et al., especially at Example 11), knockout cell lines, such as alpha-1,6-fucosyltransferase gene, FUT8, knockout CHO cells (Yamane-Ohnuki et al., Biotech. Bioeng., 87: 614 (2004)), and through the use of fucosylation pathway inhibitors such as, for example, castanospermine in cell culture media (US Pat. Appl. No. 2009/0041765).

In certain embodiments, the antibody of the present invention is expressed in cells that express beta (1,4)-N-acetylglucosaminyltransferase III (GnT III), such that GnT III adds GlcNAc to the human engineered antigen specific antibody. Methods for producing antibodies in such a fashion are provided in WO/9954342, WO/03011878, patent publication 20030003097A1, and Umana et al., Nature Biotechnology, 17:176-180, February 1999.

Another method to alter the glycosylation pattern of the Fc region of an antibody is through amino acid substitution(s). Glycosylation of an Fc region is, for example, either N-linked or O-linked.

N-linked generally refers to the attachment of the carbohydrate moiety to the side chain of an asparagine residue. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

O-linked glycosylation generally refers to the attachment of one of the sugars N-aceylgalactosamine, galactose, or xylose to a hydroxyamino acid, most commonly serine or threonine, although 5-hydroxyproline or 5-hydroxylysine may also be used.

The glycosylation pattern of an antibody or fragment thereof may be altered, for example, by deleting one or more glycosylation site(s) found in the polypeptide, and/or adding one or more glycosylation site(s) that are not present in the polypeptide. Removal of glycosylation sites in the Fc region of an antibody or antibody fragment is conveniently accomplished by altering the amino acid sequence such that it eliminates one or more of the above-described tripeptide sequences (for N-linked glycosylation sites).

An exemplary glycosylation variant has an amino acid substitution of residue N297 to A297 (EU numbering system) of the heavy chain. The removal of an O-linked glycosylation site may also be achieved by the substitution of one or more glycosylated serine or threonine residues with any amino acid besides serine or threonine.

E. Functional Equivalents, Antibody Variants and Derivatives

Functional equivalents further include fragments of antibodies that have the same, or comparable binding characteristics to those of the whole or intact antibody. Such fragments may contain one or both Fab fragments or the F(ab′)₂ fragment. Preferably the antibody fragments contain all six complementarity determining regions of the whole antibody, although fragments containing fewer than all of such regions, such as one, two, three, four or five CDRs, are also functional. Further, the functional equivalents may be or may combine members of any one of the following immunoglobulin classes: IgG, IgM, IgA, IgD, or IgE, and the subclasses thereof.

In certain aspects of the invention, the anti-MET antibodies can be modified to produce fusion proteins; i.e., the antibody, or a fragment fused to a heterologous protein, polypeptide or peptide. In certain aspects, the protein fused to the portion of an anti-MET antibody is an enzyme component of ADEPT. Examples of other proteins or polypeptides that can be engineered as a fusion protein with an anti-MET antibody include, but are not limited to, toxins such as ricin, abrin, ribonuclease, DNase I, Staphylococcal enterotoxin-A, pokeweed anti-viral protein, gelonin, diphtherin toxin, Pseudomonas exotoxin, and Pseudomonas endotoxin. See, for example, Pastan et al., Cell, 47:641 (1986); and Goldenberg et al., Cancer Journal for Clinicians, 44:43 (1994). Enzymatically active toxins and fragments thereof which can be used include, but are not limited to, diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. Non-limiting examples are included in, for example, WO 93/21232 published Oct. 28, 1993 incorporated entirely herein by reference.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of the antibodies or fragments thereof (e.g., an antibody or a fragment thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol., 16(2):76-82; Hansson et al., 1999, J. Mol. Biol., 287:265-76; and Lorenzo and Blasco, 1998, Biotechniques, 24(2):308-313, each of which is hereby incorporated by reference in its entirety. The antibody can further be a binding-domain immunoglobulin fusion protein as described in U.S. Publication 20030118592, U.S. Publication 200330133939, and PCT Publication WO 02/056910, all to Ledbetter et al., which are incorporated herein by reference in their entireties.

Domain Antibodies. The anti-MET antibodies of the compositions and methods of the invention can be domain antibodies, e.g., antibodies containing the small functional binding units of antibodies, corresponding to the variable regions of the heavy (VH) or light (VL) chains of human antibodies. Examples of domain antibodies include, but are not limited to, those available from Domantis Limited (Cambridge, UK) and Domantis Inc. (Cambridge, Mass., USA), that are specific to therapeutic targets (see, for example, WO04/058821; WO04/003019; U.S. Pat. Nos. 6,291,158; 6,582,915; 6,696,245; and 6,593,081). Commercially available libraries of domain antibodies can be used to identify anti-MET domain antibodies. In certain aspects, the anti-MET antibodies of the invention comprise a MET functional binding unit and a Fc gamma receptor functional binding unit.

Diabodies. The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

Vaccibodies. In certain aspects of the invention, the anti-MET antibodies are vaccibodies. Vaccibodies are dimeric polypeptides. Each monomer of a vaccibody consists of a scFv with specificity for a surface molecule on an APC connected through a hinge region and a Cg3 domain to a second scFv. In other aspects of the invention, vaccibodies containing as one of the scFv's an anti-MET antibody fragment may be used to juxtapose B cells to be destroyed and an effector cell that mediates ADCC. For example, see, Bogen et al., U.S. Patent Application Publication No. 20040253238.

Linear Antibodies. In certain aspects of the invention, the anti-MET antibodies are linear antibodies. Linear antibodies comprise a pair of tandem Fd segments (VH-CH1-VH-CH1) which form a pair of antigen-binding regions. Linear antibodies can be bispecific or monospecific. Non-limiting examples of linear antibodies are disclosed in, for example, Zapata et al., Protein Eng., 8(10): 1057-1062 (1995).

Parent Antibody. In certain aspects of the invention, the anti-MET antibody is a parent antibody. A “parent antibody” is an antibody comprising an amino acid sequence which lacks, or is deficient in, one or more amino acid residues in or adjacent to one or more hypervariable regions thereof compared to an altered/mutant antibody as herein disclosed. Thus, the parent antibody has a shorter hypervariable region than the corresponding hypervariable region of an antibody mutant as herein disclosed. The parent polypeptide may comprise a native sequence (i.e., a naturally occurring) antibody (including a naturally occurring allelic variant) or an antibody with pre-existing amino acid sequence modifications (such as other insertions, deletions and/or substitutions) of a naturally occurring sequence. Preferably the parent antibody is a humanized antibody or a human antibody.

Antibody Fragments. “Antibody fragments” comprise a portion of a full-length antibody, generally the antigen binding or variable region thereof. Examples of antibody fragments include Fab, Fab′, F(ab′)2, and Fv fragments; diabodies; linear antibodies; single-chain antibody molecules; single Fab arm “one arm” antibodies and multispecific antibodies formed from antibody fragments, among others.

Traditionally, fragments were derived via proteolytic digestion of intact antibodies (see, e.g., Morimoto et al., Journal of Biochemical and Biophysical Methods, 24:107-117 (1992) and Brennan et al., Science, 229:81 (1985)). However, fragments can now be produced directly by recombinant host cells. For example, the antibody fragments can be isolated from the antibody phage libraries as discussed herein. Alternatively, Fab′-SH fragments can be directly recovered from E. coli and chemically coupled to form F(ab′)2 fragments (Carter et al., Bio Technology, 10:163-167 (1992)). According to another approach, F(ab′)2 fragments can be isolated directly from recombinant host cell culture. Single Fab arm “one arm” antibodies can be made by generating Fc “knob and hole” mutations such that the resulting molecule can be expressed in bacterial or mammalian hosts containing a single Fab arm with a full dimeric Fc region (Merchant et al., Nat. Biotechnol., 1998 Jul. 16(7):677-81, WO 2005/063816 A2). Other techniques for the production of antibody fragments are apparent to the skilled practitioner given the detailed teachings in the present specification. In other aspects, the antibody of choice is a single-chain Fv fragment (scFv). See, for example, WO 93/16185. In certain aspects, the antibody is not a Fab fragment.

Bispecific Antibodies. Bispecific antibodies are antibodies that have binding specificities for at least two different epitopes. Exemplary bispecific antibodies may bind to two different epitopes of MET. Other such antibodies may bind MET and further bind a second antigen. Alternatively, a MET binding arm may be combined with an arm which binds to a triggering molecule on a leukocyte such as a T cell receptor molecule (e.g., CD2 or CD3), or Fc receptors for IgG (FcγR), so as to focus cellular defense mechanisms to the target. Bispecific antibodies may also be used to localize cytotoxic agents to the target. These antibodies possess a cell marker-binding arm and an arm which binds the cytotoxic agent (e.g., saporin, anti-interferons, vinca alkaloid, ricin A chain, methola-exate or radioactive isotope hapten). Bispecific antibodies can be prepared as full-length antibodies or antibody fragments (e.g., F(ab′): bispecific antibodies).

Methods for making bispecific antibodies are known in the art. See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl. Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; EP 03089 and US 2009/0048122.

In certain aspects of the invention, the compositions and methods comprise a bispecific murine antibody or fragment thereof and/or conjugates thereof with specificity for human MET and the CD3 epsilon chain of the T cell receptor, such as the bispecific antibody described by Daniel et al., Blood, 92:4750-4757 (1998). In preferred aspects, where the anti-MET antibody or fragments thereof and/or conjugates thereof of the compositions and methods of the invention is bispecific, the anti-MET antibody is human or humanized and has specificity for human MET and an epitope on a T cell or is capable of binding to a human effector-cell such as, for example, a monocyte/macrophage and/or a natural killer cell to effect cell death.

F. Antibody Binding Affinity

The antibodies of the invention bind human MET, with a wide range of affinities (K_{D}). In a preferred aspect, at least one mAb of the present invention can optionally bind human antigen with high affinity. For example, a human or human engineered or humanized or resurfaced mAb can bind human antigen with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³, 10⁻¹⁴, 10⁻¹⁵ or any range or value therein, as determined by flow cytometry base assays, enzyme-linked immunoabsorbent assay (ELISA), surface plasmon resonance (SPR) or the KinExA® method, as practiced by those of skill in the art. The anti-MET antibodies bind with a Kd of about 10⁻⁹ M or less, more specifically about 10⁻⁹ to 10⁻¹⁰ M.

The affinity or avidity of an antibody for an antigen is determined experimentally using any suitable method well known in the art, e.g. flow cytometry, enzyme-linked immunoabsorbent assay (ELISA), or radioimmunoassay (RIA), or kinetics (e.g., BIACORE™ analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., K_{D} or K_{d}, Kₒₙ, K_{off}) are preferably made with standardized solutions of antibody and antigen, and a standardized buffer, as known in the art and such as the buffer described herein.

In one aspect, binding assays can be performed using flow cytometry on cells expressing the MET antigen on the surface. For example, such MET-positive cells are incubated with varying concentrations of anti-MET antibodies using 1×10⁵ cells per sample in 100 μL FACS buffer (RPMI-1640 medium supplemented with 2% normal goat serum). Then, the cells are pelleted, washed, and incubated for 1 h with 100 μL of FITC-conjugated goat anti-mouse IgG-antibody (such as obtainable from Jackson ImmunoResearch) in FACS buffer. The cells are pelleted again, washed with FACS buffer and resuspended in 200 μL of PBS containing 1% formaldehyde. Samples are acquired, for example, using a FACSCalibur flow cytometer with the HTS multiwell sampler and analyzed using CellQuest Pro (all from BD Biosciences, San Diego, US). For each sample the mean fluorescence intensity for FL1 (MFI) is exported and plotted against the antibody concentration in a semi-log plot to generate a binding curve. A sigmoidal dose-response curve is fitted for binding curves and EC50 values are calculated using programs such as GraphPad Prism v4 with default parameters (GraphPad software, San Diego, CA). EC50 values can be used as a measure for the apparent dissociation constant “Kd” or “KD” for each antibody.

In certain aspects of the invention, the anti-MET antibodies can be modified to alter their binding affinity for the MET and antigenic fragments thereof. Binding properties may be determined by a variety of in vitro assay methods known in the art, e.g. enzyme-linked immunoabsorbent assay (ELISA), or radioimmunoassay (RIA)), or kinetics (e.g., BIACORE™ analysis). It is generally understood that a binding molecule having a low KD is preferred.

In one aspect of the present invention, antibodies or antibody fragments specifically bind MET and antigenic fragments thereof with a dissociation constant or KD or Kd (k_{off}/kₒₙ) of less than 10⁻⁵ M, or of less than 10⁻⁶ M, or of less than 10⁻⁷ M, or of less than 10⁻⁸ M, or of less than 10⁻⁹ M, or of less than 10⁻¹⁰ M, or of less than 10⁻¹¹ M, or of less than 10⁻¹² M, or of less than 10⁻¹³ M.

In another aspect, the antibody or fragment of the invention binds to MET and/or antigenic fragments thereof with a K_{off} of less than 1×10⁻³ s⁻¹, or less than 3×10⁻³ s⁻¹. In other aspects, the antibody binds to HGFR and antigenic fragments thereof with a K_{off} less than 10⁻³ s⁻¹ less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10-8 s⁻¹, less than 5×10⁻⁸ s-¹, less than 10⁻⁹ s⁻¹, less than 5×10⁻⁹ s⁻¹, or less than 10⁻¹⁰ s⁻¹.

In another aspect, the antibody or fragment of the invention binds to MET and/or antigenic fragments thereof with an association rate constant or kₒₙ rate of at least 10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s ⁻¹, or at least 10⁸M⁻¹ s⁻¹, or at least 10⁹ M⁻¹ s⁻¹.

One of skill understands that the conjugates of the invention may have the same properties as those described herein.

G. Antibody pI and Tm

In certain aspects of the invention, the anti-MET antibodies can be modified to alter their isoelectric point (pI). Antibodies, like all polypeptides, have a pI, which is generally defined as the pH at which a polypeptide carries no net charge. It is known in the art that protein solubility is typically lowest when the pH of the solution is equal to the isoelectric point (pI) of the protein. As used herein the pI value is defined as the pI of the predominant charge form. The pI of a protein may be determined by a variety of methods including but not limited to, isoelectric focusing and various computer algorithms (see, e.g., Bjellqvist et al., 1993, Electrophoresis, 14:1023). In addition, the thermal melting temperatures (Tm) of the Fab domain of an antibody, can be a good indicator of the thermal stability of an antibody and may further provide an indication of the shelf-life. A lower Tm indicates more aggregation/less stability, whereas a higher Tm indicates less aggregation/more stability. Thus, in certain aspects antibodies having higher Tm are preferable. Tm of a protein domain (e.g., a Fab domain) can be measured using any standard method known in the art, for example, by differential scanning calorimetry (see, e.g., Vermeer et al., 2000, Biophys. J. 78:394-404; Vermeer et al., 2000, Biophys. J. 79: 2150-2154).

Accordingly, an additional non-exclusive aspect of the present invention includes modified antibodies that have certain preferred biochemical characteristics, such as a particular isoelectric point (pI) or melting temperature (Tm).

### II. Polynucleotides, Vectors, Host Cells and Recombinant Methods

The present invention further provides polynucleotides comprising a nucleotide sequence encoding an antibody of the invention or epitope-binding fragments thereof.

Also provided are polynucleotides encoding such anti-MET antibodies as described above.

Also provided is a polynucleotide having least about 10%, 15%, 20%, 25%, 30%, 35%, 40%, 45%, 50%, 55%, 60%, 65%, 70%, 75%, 80%, 85%, 90%, 95%, 96%, 97%, 98%, or 99% sequence identity to the polynucleotide that encodes for or transcribes the amino acid sequence of any of the heavy chain variable regions of the antibodies produced by hybridomas 247.27.16, 247.2.26, 247.48.38, 247.3.14, 247.22.2, 248.69.4, and 247.16.8 and/or (b) a polynucleotide having at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99% sequence identity to the polynucleotide encoding or transcribing the amino acid sequence of any of the light chain variable regions of the antibodies produced by hybridomas 247.27.16, 247.2.26, 247.48.38, 247.3.14, 247.22.2, 248.69.4, and 247.16.8.

The invention provides a polynucleotide encoding a polypeptide comprising a sequence selected from the group consisting of SEQ ID NOs:55-72. The invention further provides a polynucleotide comprising a humanized variable region DNA sequence selected from those shown in Tables 5 and 6 below.

TABLE 5
Nucleotide sequences encoding variable light chain and heavy chain sequences
of exemplary cMET-22 and cMET-27 antibodies

| | | SEQ |
|---|---|---|
| Name | Sequence | ID |
| hucMET- | GAATTCGCCACCATGGGATGGTCCTGTATTATCCTGTTCCT | 55 |
| 22VLGv2 | GGTCGCTACCGCTACTGGGGTGCATAGTGATATTCAGATG | |
| (CDR | ACTCAGTCCCCTAGTTCACTGTCCGCCTCTGTGGGCGACC | |
| grafted) | GGGTGACCATCACATGCAGAGCCTCTGAGAACATCTACAG | |
| | CACCCTGGCTTGGTATCAGCAGAAGCCAGGCAAGGCCCCC | |
| | AAGCTGCTGGTGTACGCTGCTACCAACCTGGCCGATGGCG | |
| | TGCCATCCCGGTTCAGCGGCTCCGGCTCTGGCACCGACTA | |
| | TACCCTGACAATCAGCTCCCTGCAACCAGAGGATTTCGCC | |
| | ACATACTATTGCCAGCACTTCTGGGGCACCCCATACACCT | |
| | TTGGCCAGGGCACAAAGGTGGAGATCAAGCGTACG | |
| hucMET- | AAGCTTGCCACCATGGGTTGGTCATGTATTATTCTGTTTCT | 56 |
| 22VHGv2 | GGTCGCTACCGCTACCGGGGTCCATAGTCAGGTGCAGCTG | |
| (CDR | GTGCAGTCTGGAGCCGAAGTGAAGAAGCCAGGCGCCAGC | |
| grafted) | GTGAAGGTGTCTTGCAAGGCTTCCGGCTACACCTTCACAG | |
| | ACTATAACATGGATTGGGTGAGGCAGGCTCCAGGACAGC | |
| | GGCTGGAGTGGATCGGCGACCTGAACCCTAACAATGGCG | |
| | CCACCATCTACAATCAGAAGTTTAAGGGCAGGGCTACCCT | |
| | GACAGTGGACATGTCTGCCTCCACCGCTTATATGGAGCTG | |
| | TCCAGCCTGAGAAGCGAGGATACAGCCGTGTACTATTGTG | |
| | CTCGCGGCAACTACTATGGCAATTACTATTATCTGATGGA | |
| | TTACTGGGGACAGGGGACTTCCGTGACCGTGAGTTCCGCC | |
| | TCTACAAAGGGCCC | |
| huCMET-27 | gaattcgccaccatgggatggtcatgcattatcctgtttctggtggccacagctacaggcgtccac | 57 |
| VLGv1 | tccgaaattgtcctcacacagagccccgcaactctctccctctcccctggagaacgggcaacttt | |
| (CDR- | gtcatgtcgcgccagtgagtctgttgattcatacggcaacagttttattcattggtatcaacagaagc | |
| grafted) | caggacaagctccccggttgctgatctacagggccagtaatctggagagtggcatccccgccc | |
| | gattctccggctctggcagcggcaccgactttacattgaccatctctagcctcgaacccgaagact | |
| | tcgctgtgtattattgccagcagtcaaatgaggaacctctgacctttggacagggaaccaaggtg | |
| | gaactgaagcgtacg | |
| huCMET-27 | gaattcgccaccatggggtggtcttgcataatccttttcctggtggcaaccgcaactggcgttcact | 58 |
| VLGv2 | ctgaaatcgtattgacacaaagccccgccacactgtctctgagccccggagaacgagccaccct | |
| (CDR- | gtcttgtagagcttctgaaagcgtggactcctacgggaatagctttatccactggtatcagcaaaa | |
| grafted) | gccaggtcaagcccccaggctcttgatttaccgggcctcaaacctggaatctggcatcccagca | |
| | aggttttctggctccggcagtaggacagacttcacacttacaatcagttccttggagccagaagat | |
| | tttgcagtatattattgtcagcagtcaaatgaggagcctctgaccttcggccagggaactaaagttg | |
| | aattgaagcgtacg | |
| huCMET-27 | aagcttgccaccatgggctggagctgtataatactgttcctggtcgctacagcaaccggcgttcac | 59 |
| VHGv1 | tcagaggtacagcttgtggagtccggtggaggactcgttcaacccgggggctcactgcgtctga | |
| (CDR- | gctgtgctgcaagcgggttcacattttcttcttatgacatgtcatgggtaaggcaagctcccggcaa | |
| grafted) | gggtttggaatgggttagcactatcaattccaatggtgtgtccatctattacccagatagcgttaaa | |
| | ggacgttttactataagcagggataatgccaaaaactcactgtatctgcagatgaactctctccga | |
| | gctgaagacaccgcagtgtattattgtgcacgggaggagattacaactgagatggattattgggg | |
| | acaggggactctcgtaactgtgtcctccgctagtacaaagggccc | |
| huCMET-27 | aagcttgccaccatgggctggagctgtatcatcctgttcctggtcgccaccgcaacaggcgttca | 60 |
| VHGv2 | ctccgaagtacagctcgtggaatctggcggcggccttgtgcagcccggcggctccctgagact | |
| (CDR- | gtcttgtgccgcctccggctttaccttcagcagctacgatatgtcctgggttaggcaagctcccgg | |
| grafted) | aaaaggcttggaactggtcgccacaatcaattctaatggcgtgtctatctattaccccgacagtgtg | |
| | aagggacgcttcacaatcagtagagacattgctaaaaactctctctatttgcagatgaactcactca | |
| | gggctgaagacactgctgtctactactgtgcccgagaggaaattaccaccgaaatggactattgg | |
| | ggtcagggaaccctggttactgtgtcctctgcctctaccaagggccc | |
| huCMET-27 | aagcttgccaccatgggttggtcttgtattatcctgttcctggtcgctactgctaccggcgtccactc | 61 |
| VHGv3 | agaagtccagctggtcgagagcggcgggggtctggtgcagccaggaggctctctgaggctgt | |
| (CDR- | cctgcgccgctagcggcttcaccttttccagctacgacatgagctgggtgagacaggctccagg | |
| grafted) | caagggcctggagtgggtggctaccatcaactctaatggcgtgtccatctactatcctgactctgt | |
| | gaagggcaggttcacaatcagccgggataacgccaagaactccctgtatctgcagatgaactcc | |
| | ctgagagccgaggatacagccgtgtactattgtgctcgcgaggagatcacaaccgaaatggact | |
| | attggggacagggaactctggtgaccgtctcatccgcaagcactaagggccc | |
| hu247.22.2 | gaattcgccaccatgggctggtcatgcattatcctgttcctggtggccacagctaccggcgtccac | 62 |
| VL1.0 | tcagacattcagatgacccaatcaccatcctctctgagcgtgtcagtcggtgaaagggttactatta | |
| (resurfaced) | cctgccgtgcatccgaaaatatttactccactctggcttggtaccaacaaaagccaggcaagtcc | |
| | cctaaactgctggtatatgcagccaccaatttggcagatggagtcccttcccgattttccgggagt | |
| | ggcagtggaactgagtactcactcaaaatcaacagcctccagcccgacgatttcggatcttacta | |
| | ctgtcagcacttctggggcacaccttacaccttcggcgggggaactaagctggagatcaagcgt | |
| | acg | |
| hu247.22.2 | aagcttgccaccatgggctggtcctgcatcatacttttcctggtcgccaccgctacaggtgtgcac | 63 |
| VH1.0 | tcagaggtgcagctggtgcagtcaggcgccgaagtggttaaacccggagcctcagtgaagatc | |
| (resurfaced) | ccttgcaaagcatccggctatacattcaccgactataatatggattgggtaagacagtcccctggg | |
| | aagtctcttgaatggatcggtgaccttaatcccaacaatggtgcaacaatctacaatgaaaaatttc | |
| | aggggaaagctactctgaccgtggatacttcatccagtaccgcctatatggaactgaggtctctta | |
| | catccgaggatactgcagtgtattactgcgcccggggcaactactacggaaattactactatctga | |
| | tggactactgggggcagggcacttctgtgactgtttcctccgcttccacaaagggccc | |
| hu247.27.16 | gaattcgccaccatgggctggtcttgtatcattttgttcctggttgccaccgcaacaggtgtacactc | 64 |
| VL1.0 | tgacatagtccttacacaaagcccagcatcactcgcagtcagcccaggccagagagctacaatc | |
| (resurfaced) | tcctgtcgggcttccgagtccgtcgattcctatggaaacagcttcatacactggtaccagcagaaa | |
| | cctgggcagcccccaaaacttctgatttatcgggcaagtaatttggagtcaggtatccctgccagg | |
| | ttcagtggttctggctcccgaaccgattttacactcactattaaccccgtggaagccaatgacgtcg | |
| | caacttactactgccaacagagtaacgaggaccccttgaccttcggcggcggcactaagctgga | |
| | gctcaagcgtacg | |
| hu247.27.16 | gaattcgccaccatgggttggtcctgcattatcctctttttggtggctacagcaaccggtgttcattct | 65 |
| VL1.2 | gacattgttctcactcagtcacctgcaagtttggccgtcagtccaggacagcgggcaaccatctc | |
| (resurfaced) | ctgtcgcgctagcgaatccgtagatagctatggaaactcctttattcactggtaccagcaaaagcc | |
| | agggcagcctcccaaactgctgatttacagagcctccaacctggaaagtggcatccccgcccg | |
| | gttcagtggctcaggttctcggaccgattttacactcaccattaatcccgtggaagctaacgatgtg | |
| | gctacatactattgtcagcagagcaatgaggaacctctcaccttcggggggggcactaagctgg | |
| | agctgaagcgtacg | |
| hu247.27.16 | gaattcgccaccatgggctggtcttgcatcatactgttcctggtcgcaactgccacaggcgttcac | 66 |
| VL1.3 | tctgatatcgtgttgacccagtcccctgccagcctcgcagtcagccctggccagcgcgctaccat | |
| (resurfaced) | atcttgtcgtgcttctgaaagcgtggattcttacggcaacagtttcatacactggtaccagcagaaa | |
| | cctgggcagccacctaaactgctgatctatcgagcttcaaaccttgaatccggcattcctgcccgg | |
| | ttttcaggctccggctccagaaccgatttcaccttgaccattaatcctgtagaggctaatgacgttg | |
| | ccacctactattgccagcaatccaatgaaaaccctctcacctttggcggcgggacaaagctgga | |
| | gctgaagcgtacg | |
| hu247.27.16 | aagcttgccaccatgggttggtcctgtattatcctgtttttggtggctactgcaaccggcgtacatag | 67 |
| VH1.0 | tgaggtccagttggttgagtccggcggcggtctggtccagcccggcggtagcctgcggctgagt | |
| (resurfaced) | tgcgctgcctcaggctttactttctccagctacgacatgagttgggttcgacagacaccaggcaag | |
| | ggcctggaactcgtggcaacaatcaatagtaacggtgtcagcatatactaccccgacagtgtcaa | |
| | ggggaggtttaccataagtagagatatcgccaaaaacacattgtacctgcagatgtccagtctgc | |
| | gtgccgaggatacagctatgtactactgtgcacgcgaagagatcaccacagagatggactactg | |
| | ggggcagggtacaagcgtcaccgtcagctctgctagtaccaagggccc | |

TABLE 6
Nucleotide sequences encoding full-length light chain and heavy chain
sequences of exemplary cMET-22 and cMET-27 antibodies

| | | SEQ |
|---|---|---|
| Name | Sequence | ID |
| huCMET-27 | gaattcgccaccatgggatggtcatgcattatcctgtttctggtggccacagctacaggcgtccac | 68 |
| LCGv1 | tccgaaattgtcctcacacagagccccgcaactctctccctctcccctggagaacgggcaacttt | |
| (CDR- | gtcatgtcgcgccagtgagtctgttgattcatacggcaacagttttattcattggtatcaacagaagc | |
| grafted) | caggacaagctccccggttgctgatctacagggccagtaatctggagagtggcatccccgccc | |
| | gattctccggctctggcagcggcaccgactttacattgaccatctctagcctcgaacccgaagact | |
| | tcgctgtgtattattgccagcagtcaaatgaggaacctctgacctttggacagggaaccaaggtg | |
| | gaactgaagcgtacggtggctgcaccatctgtcttcatcttcccgccatctgatgagcagttgaaa | |
| | tctggaactgcctctgttgtgtgcctgctgaataacttctatcccagagaggccaaagtacagtgg | |
| | aaggtggataacgccctccaatcgggtaactcccaggagagtgtcacagagcaggacagcaa | |
| | ggacagcacctacagcctcagcagcaccctgacgctgagcaaagcagactacgagaaacaca | |
| | aagtctacgcctgcgaagtcacccatcagggcctgagctcgcccgtcacaaagagcttcaacag | |
| | gggagagtgttag | |
| huCMET-27 | gaattcgccaccatggggtggtcttgcataatccttttcctggtggcaaccgcaactggcgttcact | 69 |
| LCGv2 | ctgaaatcgtattgacacaaagccccgccacactgtctctgagccccggagaacgagccaccct | |
| (CDR- | gtcttgtagagcttctgaaagcgtggactcctacgggaatagctttatccactggtatcagcaaaa | |
| grafted) | gccaggtcaagcccccaggctcttgatttaccgggcctcaaacctggaatctggcatcccagca | |
| | aggttttctggctccggcagtaggacagacttcacacttacaatcagttccttggagccagaagat | |
| | tttgcagtatattattgtcagcagtcaaatgaggagcctctgaccttcggccagggaactaaagttg | |
| | aattgaagcgtacggtggctgcaccatctgtcttcatcttcccgccatctgatgagcagttgaaatc | |
| | tggaactgcctctgttgtgtgcctgctgaataacttctatcccagagaggccaaagtacagtggaa | |
| | ggtggataacgccctccaatcgggtaactcccaggagagtgtcacagagcaggacagcaagg | |
| | acagcacctacagcctcagcagcaccctgacgctgagcaaagcagactacgagaaacacaaa | |
| | gtctacgcctgcgaagtcacccatcagggcctgagctcgcccgtcacaaagagcttcaacagg | |
| | ggagagtgttag | |
| huCMET-27 | aagcttgccaccatgggctggagctgtataatactgttcctggtcgctacagcaaccggcgttcac | 70 |
| HCGv1 | tcagaggtacagcttgtggagtccggtggaggactcgttcaacccgggggctcactgcgtctga | |
| (CDR- | gctgtgctgcaagcgggttcacattttcttcttatgacatgtcatgggtaaggcaagctcccggcaa | |
| grafted) | gggtttggaatgggttagcactatcaattccaatggtgtgtccatctattacccagatagcgttaaa | |
| | ggacgttttactataagcagggataatgccaaaaactcactgtatctgcagatgaactctctccga | |
| | gctgaagacaccgcagtgtattattgtgcacgggaggagattacaactgagatggattattgggg | |
| | acaggggactctcgtaactgtgtcctccgctagtacaaagggcccatcagttttccccttggctcc | |
| | aagttctaaatccacaagcggtggaacagctgcactgggatgcctcgttaaagattatttccctga | |
| | gcctgtgacagtgagctggaatagcggagcattgacttcaggtgtgcacacttttcccgctgtgtt | |
| | gcagtcctccggtctgtactcactgtccagtgtcgtaaccgtcccttctagcagcttgggaaccca | |
| | gacctacatctgtaacgtcaaccataaaccatccaacacaaaggtggataagaaggttgaacca | |
| | aagagctgtgataagacacatacatgccctccttgtcctgcaccagagctcctcggaggtccatct | |
| | gtgttcctgtttccccccaaacccaaggacactcttatgatctctcgtactccagaggtcacctgtgt | |
| | tgttgtcgacgtgagccatgaagatcccgaggttaaattcaactggtacgtggatggagtcgagg | |
| | ttcacaatgccaagaccaagcccagggaggagcaatataattctacatatcgggtagtgagcgtt | |
| | ctgaccgtgctccaccaagattggctcaatggaaaagagtacaagtgcaaggtgtccaacaagg | |
| | ctcttcccgctcccattgagaaaactatctccaaagccaaggggcagccacgggaaccccaggt | |
| | gtatacattgcccccatctagagacgagctgaccaagaaccaggtgagtctcacttgtctggtca | |
| | aggggttttacccttctgacattgctgtagagtgggagtctaacggacagccagaaaacaactac | |
| | aagacaactcccccagtgctggacagcgacgggagcttcttcctctactccaagttgactgtaga | |
| | caagtctagatggcagcaaggaaacgttttctcctgctcagtaatgcatgaggctctgcacaatca | |
| | ctatacccagaaatcactgtcccttagcccagggtgactcgag | |
| huCMET-27 | aagcttgccaccatgggctggagctgtatcatcctgttcctggtcgccaccgcaacaggcgttca | 71 |
| HCGv2 | ctccgaagtacagctcgtggaatctggcggcggccttgtgcagcccggcggctccctgagact | |
| (CDR- | gtcttgtgccgcctccggctttaccttcagcagctacgatatgtcctgggttaggcaagctcccgg | |
| grafted) | aaaaggcttggaactggtcgccacaatcaattctaatggcgtgtctatctattaccccgacagtgtg | |
| | aagggacgcttcacaatcagtagagacattgctaaaaactctctctatttgcagatgaactcactca | |
| | gggctgaagacactgctgtctactactgtgcccgagaggaaattaccaccgaaatggactattgg | |
| | ggtcagggaaccctggttactgtgtcctctgcctctaccaagggcccatcagttttccccttggctc | |
| | caagttctaaatccacaagcggtggaacagctgcactgggatgcctcgttaaagattatttccctg | |
| | agcctgtgacagtgagctggaatagcggagcattgacttcaggtgtgcacacttttcccgctgtgt | |
| | tgcagtcctccggtctgtactcactgtccagtgtcgtaaccgtcccttctagcagcttgggaaccca | |
| | gacctacatctgtaacgtcaaccataaaccatccaacacaaaggtggataagaaggttgaacca | |
| | aagagctgtgataagacacatacatgccctccttgtcctgcaccagagctcctcggaggtccatct | |
| | gtgttcctgtttccccccaaacccaaggacactcttatgatctctcgtactccagaggtcacctgtgt | |
| | tgttgtcgacgtgagccatgaagatcccgaggttaaattcaactggtacgtggatggagtcgagg | |
| | ttcacaatgccaagaccaagcccagggaggagcaatataattctacatatcgggtagtgagcgtt | |
| | ctgaccgtgctccaccaagattggctcaatggaaaagagtacaagtgcaaggtgtccaacaagg | |
| | ctcttcccgctcccattgagaaaactatctccaaagccaaggggcagccacgggaaccccaggt | |
| | gtatacattgcccccatctagagacgagctgaccaagaaccaggtgagtctcacttgtctggtca | |
| | aggggttttacccttctgacattgctgtagagtgggagtctaacggacagccagaaaacaactac | |
| | aagacaactcccccagtgctggacagcgacgggagcttcttcctctactccaagttgactgtaga | |
| | caagtctagatggcagcaaggaaacgttttctcctgctcagtaatgcatgaggctctgcacaatca | |
| | ctatacccagaaatcactgtcccttagcccagggtgactcgag | |
| huCMET-27 | aagcttgccaccatgggttggtcttgtattatcctgttcctggtcgctactgctaccggcgtccactc | 72 |
| HCGv3 | agaagtccagctggtcgagagcggcgggggtctggtgcagccaggaggctctctgaggctgt | |
| (CDR- | cctgcgccgctagcggcttcaccttttccagctacgacatgagctgggtgagacaggctccagg | |
| grafted) | caagggcctggagtgggtggctaccatcaactctaatggcgtgtccatctactatcctgactctgt | |
| | gaagggcaggttcacaatcagccgggataacgccaagaactccctgtatctgcagatgaactcc | |
| | ctgagagccgaggatacagccgtgtactattgtgctcgcgaggagatcacaaccgaaatggact | |
| | attggggacagggaactctggtgaccgtctcatccgcaagcactaagggcccatcagttttcccc | |
| | ttggctccaagttctaaatccacaagcggtggaacagctgcactgggatgcctcgttaaagattat | |
| | ttccctgagcctgtgacagtgagctggaatagcggagcattgacttcaggtgtgcacacttttccc | |
| | gctgtgttgcagtcctccggtctgtactcactgtccagtgtcgtaaccgtcccttctagcagcttgg | |
| | gaacccagacctacatctgtaacgtcaaccataaaccatccaacacaaaggtggataagaaggt | |
| | tgaaccaaagagctgtgataagacacatacatgccctccttgtcctgcaccagagctcctcggag | |
| | gtccatctgtgttcctgtttccccccaaacccaaggacactcttatgatctctcgtactccagaggtc | |
| | acctgtgttgttgtcgacgtgagccatgaagatcccgaggttaaattcaactggtacgtggatgga | |
| | gtcgaggttcacaatgccaagaccaagcccagggaggagcaatataattctacatatcgggtag | |
| | tgagcgttctgaccgtgctccaccaagattggctcaatggaaaagagtacaagtgcaaggtgtcc | |
| | aacaaggctcttcccgctcccattgagaaaactatctccaaagccaaggggcagccacgggaa | |
| | ccccaggtgtatacattgcccccatctagagacgagctgaccaagaaccaggtgagtctcacttg | |
| | tctggtcaaggggttttacccttctgacattgctgtagagtgggagtctaacggacagccagaaaa | |
| | caactacaagacaactcccccagtgctggacagcgacgggagcttcttcctctactccaagttga | |
| | ctgtagacaagtctagatggcagcaaggaaacgttttctcctgctcagtaatgcatgaggctctgc | |
| | acaatcactatacccagaaatcactgtcccttagcccagggtgactcgag | |
| huCMET-27 | ATGGGTTGGTCCTGTATTATTCTGTTTCTGGTCGCTACCGC | 109 |
| HC_Gv3 | TACTGGGGTCCATTCCGAAGTGCAGCTGGTCGAGAGTGGG | |
| Cysmab- | GGAGGGCTGGTGCAGCCTGGCGGAAGCCTGAGACTGTCTT | |
| IgG2 hinge | GCGCCGCTAGTGGCTTCACCTTTTCCAGCTACGACATGAG | |
| (CDR | CTGGGTGCGCCAGGCACCAGGGAAGGGTCTGGAGTGGGT | |
| grafted) | CGCCACTATCAACTCAAATGGCGTGTCCATCTACTATCCC | |
| | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCATCCGTGTTCC | |
| | CACTGGCTCCCTCATCCAAAAGTACTTCAGGGGGTACCGC | |
| | AGCCCTGGGATGTCTGGTGAAGGACTACTTCCCAGAGCCC | |
| | GTCACCGTGTCTTGGAACAGTGGGGCTCTGACCTCCGGTG | |
| | TCCACACATTTCCAGCAGTGCTGCAGAGCTCTGGCCTGTA | |
| | CTCCCTGAGTTCAGTGGTCACAGTGCCCTCCAGCTCTCTGG | |
| | GAACACAGACTTATATCTGCAACGTGAATCATAAGCCTTC | |
| | CAATACTAAAGTCGATAAGAAAGTGGAGCGAAAGTGCTG | |
| | CGTGGAATGCCCCCCTTGTCCTGCACCAGAACTGCTGGGC | |
| | GGACCCTCCGTGTTCCTGTTTCCACCCAAGCCTAAAGACA | |
| | CTCTGATGATTTCCCGGACACCTGAGGTCACTTGCGTGGT | |
| | CGTGGACGTGTCCCACGAGGACCCCGAAGTCAAGTTCAAC | |
| | TGGTACGTGGATGGAGTCGAAGTGCATAATGCTAAGACA | |
| | AAACCTAGAGAGGAACAGTACAACAGTACATATAGAGTC | |
| | GTGTCAGTCCTGACTGTGCTGCACCAGGACTGGCTGAACG | |
| | GGAAGGAGTATAAGTGCAAAGTGAGCAATAAGGCTCTGC | |
| | CCGCACCTATCGAGAAAACCATTTCTAAGGCTAAAGGCCA | |
| | GCCTAGGGAACCACAGGTGTACACACTGCCTCCATCTCGG | |
| | GACGAGCTGACTAAGAACCAGGTCAGTCTGACCTGTCTGG | |
| | TGAAAGGGTTCTATCCATCCGATATCGCAGTGGAGTGGGA | |
| | AAGCAATGGTCAGCCCGAGAACAATTACAAGACTACCCC | |
| | CCCTGTGCTGGACTCAGATGGGTCCTTCTTTCTGTATAGTA | |
| | AGCTGACCGTGGATAAATCAAGGTGGCAGCAGGGTAATG | |
| | TCTTTTCCTGTAGCGTGATGCACGAAGCCCTGCATAACCA | |
| | CTACACTCAGAAAAGCCTGTGCCTGTCCCCTGGA | |
| huCMET-27 | ATGGGTTGGTCCTGTATTATTCTGTTTCTGGTCGCTACCGC | 110 |
| HC_Gv3 | TACTGGGGTCCATTCCGAAGTGCAGCTGGTCGAGAGTGGG | |
| Cysmab- | GGAGGGCTGGTGCAGCCTGGCGGAAGCCTGAGACTGTCTT | |
| IgG2 hinge- | GCGCCGCTAGTGGCTTCACCTTTTCCAGCTACGACATGAG | |
| S127C | CTGGGTGCGCCAGGCACCAGGGAAGGGTCTGGAGTGGGT | |
| (CDR | CGCCACTATCAACTCAAATGGCGTGTCCATCTACTATCCC | |
| grafted) | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCAAGCGTCTTCC | |
| | CCCTGGCTCCATGCTCAAAGTCAACAAGCGGTGGTACTGC | |
| | TGCTCTGGGTTGCCTGGTCAAGGATTATTTTCCCGAGCCTG | |
| | TCACCGTGTCATGGAACTCCGGGGCACTGACCAGCGGTGT | |
| | CCACACATTCCCAGCCGTGCTGCAGTCCAGCGGGCTGTAC | |
| | TCTCTGTCTAGTGTGGTCACTGTGCCATCATCCAGCCTGGG | |
| | TACTCAGACCTATATCTGCAACGTGAATCATAAGCCCTCC | |
| | AATACCAAAGTCGACAAGAAAGTGGAGCGAAAGTGCTGC | |
| | GTGGAATGCCCACCTTGTCCAGCACCAGAACTGCTGGGCG | |
| | GACCATCCGTGTTCCTGTTTCCACCCAAGCCCAAAGACAC | |
| | ACTGATGATTAGCAGGACACCCGAGGTCACTTGCGTGGTC | |
| | GTGGACGTGTCTCACGAGGACCCCGAAGTCAAGTTTAACT | |
| | GGTACGTGGATGGCGTCGAAGTGCATAATGCTAAGACTAA | |
| | ACCCAGGGAGGAACAGTACAACAGTACATATCGGGTCGT | |
| | GTCAGTCCTGACTGTGCTGCACCAGGATTGGCTGAACGGG | |
| | AAGGAGTATAAGTGCAAAGTGAGTAATAAGGCCCTGCCT | |
| | GCTCCAATCGAGAAAACCATTTCCAAGGCTAAAGGCCAGC | |
| | CCAGAGAACCTCAGGTGTACACACTGCCTCCATCACGCGA | |
| | CGAGCTGACTAAGAACCAGGTCTCCCTGACCTGTCTGGTG | |
| | AAAGGCTTCTATCCTTCTGATATCGCAGTGGAGTGGGAAA | |
| | GTAATGGACAGCCAGAGAACAATTACAAGACCACACCCC | |
| | CTGTGCTGGACAGCGATGGCTCTTTCTTTCTGTATTCCAAG | |
| | CTGACAGTCGACAAAAGCAGATGGCAGCAGGGAAACGTG | |
| | TTCTCCTGCAGTGTGATGCACGAAGCCCTGCATAACCATT | |
| | ACACTCAGAAAAGCCTGTGCCTGTCCCCTGGG | |
| huCMET-27 | ATGGGTTGGTCCTGTATTATTCTGTTTCTGGTCGCTACCGC | 111 |
| HC_Gv3- | TACTGGGGTCCATTCCGAAGTGCAGCTGGTCGAGAGTGGG | |
| IgG2 hinge | GGAGGGCTGGTGCAGCCTGGCGGAAGCCTGAGACTGTCTT | |
| | GCGCCGCTAGTGGCTTCACCTTTTCCAGCTACGACATGAG | |
| | CTGGGTGCGCCAGGCACCAGGGAAGGGTCTGGAGTGGGT | |
| | CGCCACTATCAACTCAAATGGCGTGTCCATCTACTATCCC | |
| | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCATCCGTGTTCC | |
| | CACTGGCTCCCTCATCCAAAAGTACTTCAGGGGGTACCGC | |
| | AGCCCTGGGATGTCTGGTGAAGGACTACTTCCCAGAGCCC | |
| | GTCACCGTGTCTTGGAACAGTGGGGCTCTGACCTCCGGTG | |
| | TCCACACATTTCCAGCAGTGCTGCAGAGCTCTGGCCTGTA | |
| | CTCCCTGAGTTCAGTGGTCACAGTGCCCTCCAGCTCTCTGG | |
| | GAACACAGACTTATATCTGCAACGTGAATCATAAGCCTTC | |
| | CAATACTAAAGTCGATAAGAAAGTGGAGCGAAAGTGCTG | |
| | CGTGGAATGCCCCCCTTGTCCTGCACCAGAACTGCTGGGC | |
| | GGACCCTCCGTGTTCCTGTTTCCACCCAAGCCTAAAGACA | |
| | CTCTGATGATTTCCCGGACACCTGAGGTCACTTGCGTGGT | |
| | CGTGGACGTGTCCCACGAGGACCCCGAAGTCAAGTTCAAC | |
| | TGGTACGTGGATGGAGTCGAAGTGCATAATGCTAAGACA | |
| | AAACCTAGAGAGGAACAGTACAACAGTACATATAGAGTC | |
| | GTGTCAGTCCTGACTGTGCTGCACCAGGACTGGCTGAACG | |
| | GGAAGGAGTATAAGTGCAAAGTGAGCAATAAGGCTCTGC | |
| | CCGCACCTATCGAGAAAACCATTTCTAAGGCTAAAGGCCA | |
| | GCCTAGGGAACCACAGGTGTACACACTGCCTCCATCTCGG | |
| | GACGAGCTGACTAAGAACCAGGTCAGTCTGACCTGTCTGG | |
| | TGAAAGGGTTCTATCCATCCGATATCGCAGTGGAGTGGGA | |
| | AAGCAATGGTCAGCCCGAGAACAATTACAAGACTACCCC | |
| | CCCTGTGCTGGACTCAGATGGGTCCTTCTTTCTGTATAGTA | |
| | AGCTGACCGTGGATAAATCAAGGTGGCAGCAGGGTAATG | |
| | TCTTTTCCTGTAGCGTGATGCACGAAGCCCTGCATAACCA | |
| | CTACACTCAGAAAAGCCTGTCCCTGTCCCCTGGA | |
| huCMET-27 | ATGGGTTGGTCCTGTATTATTCTGTTTCTGGTCGCTACCGC | 112 |
| HC_Gv3- | TACTGGGGTCCATTCCGAAGTGCAGCTGGTCGAGAGTGGG | |
| IgG2 hinge- | GGAGGGCTGGTGCAGCCTGGCGGAAGCCTGAGACTGTCTT | |
| S127C | GCGCCGCTAGTGGCTTCACCTTTTCCAGCTACGACATGAG | |
| | CTGGGTGCGCCAGGCACCAGGGAAGGGTCTGGAGTGGGT | |
| | CGCCACTATCAACTCAAATGGCGTGTCCATCTACTATCCC | |
| | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCAAGCGTCTTCC | |
| | CCCTGGCTCCATGCTCAAAGTCAACAAGCGGTGGTACTGC | |
| | TGCTCTGGGTTGCCTGGTCAAGGATTATTTTCCCGAGCCTG | |
| | TCACCGTGTCATGGAACTCCGGGGCACTGACCAGCGGTGT | |
| | CCACACATTCCCAGCCGTGCTGCAGTCCAGCGGGCTGTAC | |
| | TCTCTGTCTAGTGTGGTCACTGTGCCATCATCCAGCCTGGG | |
| | TACTCAGACCTATATCTGCAACGTGAATCATAAGCCCTCC | |
| | AATACCAAAGTCGACAAGAAAGTGGAGCGAAAGTGCTGC | |
| | GTGGAATGCCCACCTTGTCCAGCACCAGAACTGCTGGGCG | |
| | GACCATCCGTGTTCCTGTTTCCACCCAAGCCCAAAGACAC | |
| | ACTGATGATTAGCAGGACACCCGAGGTCACTTGCGTGGTC | |
| | GTGGACGTGTCTCACGAGGACCCCGAAGTCAAGTTTAACT | |
| | GGTACGTGGATGGCGTCGAAGTGCATAATGCTAAGACTAA | |
| | ACCCAGGGAGGAACAGTACAACAGTACATATCGGGTCGT | |
| | GTCAGTCCTGACTGTGCTGCACCAGGATTGGCTGAACGGG | |
| | AAGGAGTATAAGTGCAAAGTGAGTAATAAGGCCCTGCCT | |
| | GCTCCAATCGAGAAAACCATTTCCAAGGCTAAAGGCCAGC | |
| | CCAGAGAACCTCAGGTGTACACACTGCCTCCATCACGCGA | |
| | CGAGCTGACTAAGAACCAGGTCTCCCTGACCTGTCTGGTG | |
| | AAAGGCTTCTATCCTTCTGATATCGCAGTGGAGTGGGAAA | |
| | GTAATGGACAGCCAGAGAACAATTACAAGACCACACCCC | |
| | CTGTGCTGGACAGCGATGGCTCTTTCTTTCTGTATTCCAAG | |
| | CTGACAGTCGACAAAAGCAGATGGCAGCAGGGAAACGTG | |
| | TTCTCCTGCAGTGTGATGCACGAAGCCCTGCATAACCATT | |
| | ACACTCAGAAAAGCCTGAGCCTGTCCCCTGGG | |
| huCMET-27 | ATGGGTTGGTCCTGTATTATTCTGTTTCTGGTCGCTACCGC | 113 |
| HC_Gv3- | TACTGGGGTCCATTCCGAAGTGCAGCTGGTCGAGAGTGGG | |
| Cysmab- | GGAGGGCTGGTGCAGCCTGGCGGAAGCCTGAGACTGTCTT | |
| hinge#28 | GCGCCGCTAGTGGCTTCACCTTTTCCAGCTACGACATGAG | |
| | CTGGGTGCGCCAGGCACCAGGGAAGGGTCTGGAGTGGGT | |
| | CGCCACTATCAACTCAAATGGCGTGTCCATCTACTATCCC | |
| | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCAAGCGTCTTTC | |
| | CACTGGCTCCAAGTTCCAAGTCTACAAGCGGCGGTACTGC | |
| | TGCTCTGGGGTGTCTGGTGAAGGATTATTTCCCTGAACCA | |
| | GTCACCGTGTCATGGAACTCCGGGGCTCTGACCTCCGGTG | |
| | TCCACACATTCCCCGCAGTGCTGCAGTCCAGCGGGCTGTA | |
| | CTCCCTGTCTAGTGTGGTCACTGTGCCTTCATCCAGCCTGG | |
| | GTACTCAGACCTATATCTGTAACGTGAATCACAAGCCAAG | |
| | CAATACCAAGGTCGACAAACGAGTGGAACCCAAATCTTG | |
| | CGATTGTCATTGCCCACCTTGCCCAGCTCCTGAGCTGCTGG | |
| | GCGGACCCAGCGTGTTCCTGTTTCCACCCAAGCCTAAAGA | |
| | CACACTGATGATTAGTAGGACACCCGAAGTCACTTGCGTG | |
| | GTCGTGGACGTGTCCCACGAGGACCCCGAAGTCAAGTTTA | |
| | ACTGGTACGTGGATGGCGTCGAGGTGCATAATGCAAAGA | |
| | CTAAACCAAGGGAGGAACAGTACAACAGTACATATCGGG | |
| | TCGTGTCAGTCCTGACTGTGCTGCATCAGGACTGGCTGAA | |
| | CGGGAAGGAATATAAGTGTAAAGTGAGCAATAAGGCACT | |
| | GCCAGCCCCCATCGAGAAAACCATTTCTAAGGCCAAAGGC | |
| | CAGCCTAGAGAACCACAGGTGTACACACTGCCTCCATCAC | |
| | GCGACGAGCTGACTAAGAACCAGGTCTCCCTGACCTGCCT | |
| | GGTGAAAGGCTTCTATCCTTCTGATATCGCTGTGGAGTGG | |
| | GAAAGTAATGGACAGCCAGAGAACAATTACAAGACCACA | |
| | CCCCCTGTGCTGGACAGCGATGGCTCTTTCTTTCTGTATTC | |
| | CAAGCTGACAGTGGATAAAAGCAGATGGCAGCAGGGAAA | |
| | CGTGTTCTCCTGCAGTGTGATGCACGAAGCCCTGCATAAC | |
| | CATTACACTCAGAAGAGCCTGTGCCTGTCCCCTGGG | |
| huCMET-27 | ATGGGTTGGTCCTGTATTATTCTGTTTCTGGTCGCTACCGC | 114 |
| HC_Gv3- | TACTGGGGTCCATTCCGAAGTGCAGCTGGTCGAGAGTGGG | |
| hinge#28 | GGAGGGCTGGTGCAGCCTGGCGGAAGCCTGAGACTGTCTT | |
| | GCGCCGCTAGTGGCTTCACCTTTTCCAGCTACGACATGAG | |
| | CTGGGTGCGCCAGGCACCAGGGAAGGGTCTGGAGTGGGT | |
| | CGCCACTATCAACTCAAATGGCGTGTCCATCTACTATCCC | |
| | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCAAGCGTGTTCC | |
| | CACTGGCTCCCAGCTCCAAGAGCACCTCCGGAGGAACAGC | |
| | CGCTCTGGGCTGTCTGGTGAAGGACTACTTCCCAGAGCCC | |
| | GTGACCGTGTCCTGGAACTCTGGCGCCCTGACCTCCGGAG | |
| | TGCACACATTTCCAGCTGTGCTGCAGTCTAGCGGCCTGTA | |
| | CTCTCTGTCCTCTGTGGTGACCGTGCCCAGCTCCTCTCTGG | |
| | GCACCCAGACATATATCTGTAACGTGAATCACAAGCCATC | |
| | CAATACAAAGGTGGACAAGCGGGTGGAGCCCAAGTCTTG | |
| | CGATTGTCACTGCCCACCTTGCCCTGCTCCAGAGCTGCTG | |
| | GGCGGCCCTTCCGTGTTCCTGTTTCCACCCAAGCCTAAGG | |
| | ACACCCTGATGATCAGCAGAACCCCCGAGGTGACATGCGT | |
| | GGTGGTGGACGTGTCCCACGAGGACCCCGAGGTGAAGTTT | |
| | AACTGGTACGTGGATGGCGTGGAGGTGCACAATGCTAAG | |
| | ACAAAGCCTCGGGAGGAGCAGTACAACTCTACCTATAGG | |
| | GTGGTGAGCGTGCTGACAGTGCTGCACCAGGACTGGCTGA | |
| | ACGGCAAGGAGTATAAGTGTAAGGTGTCTAATAAGGCCCT | |
| | GCCCGCTCCTATCGAGAAGACCATCAGCAAGGCCAAGGG | |
| | CCAGCCTAGAGAGCCACAGGTGTACACACTGCCTCCATCT | |
| | CGCGACGAGCTGACCAAGAACCAGGTGAGCCTGACATGC | |
| | CTGGTGAAGGGCTTCTATCCTAGCGATATCGCTGTGGAGT | |
| | GGGAGTCCAATGGCCAGCCAGAGAACAATTACAAGACCA | |
| | CACCCCCTGTGCTGGACAGCGATGGCTCCTTCTTTCTGTAT | |
| | TCCAAGCTGACCGTGGATAAGTCTCGGTGGCAGCAGGGCA | |
| | ACGTGTTTTCTTGTAGCGTGATGCACGAGGCTCTGCACAA | |
| | TCACTATACACAGAAGTCCCTGTCTCTGAGCCCCGGC | |
| huCMET-27 | ATGGGTTGGTCCTGTATTATTCTGTTTCTGGTCGCTACCGC | 115 |
| HC_Gv3- | TACTGGGGTCCATTCCGAAGTGCAGCTGGTCGAGAGTGGG | |
| Cysmab- | GGAGGGCTGGTGCAGCCTGGCGGAAGCCTGAGACTGTCTT | |
| hinge#26 | GCGCCGCTAGTGGCTTCACCTTTTCCAGCTACGACATGAG | |
| | CTGGGTGCGCCAGGCACCAGGGAAGGGTCTGGAGTGGGT | |
| | CGCCACTATCAACTCAAATGGCGTGTCCATCTACTATCCC | |
| | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCAAGCGTCTTCC | |
| | CTCTGGCTCCATCAAGCAAATCAACTTCTGGCGGCACAGC | |
| | AGCACTGGGGTGTCTGGTCAAGGACTACTTTCCCGAGCCT | |
| | GTCACCGTGTCATGGAACTCCGGGGCTCTGACCAGCGGTG | |
| | TCCACACATTCCCAGCAGTGCTGCAGTCCAGCGGGCTGTA | |
| | CTCTCTGTCTAGTGTGGTCACTGTGCCATCATCCAGCCTGG | |
| | GTACTCAGACCTATATCTGCAACGTGAATCATAAGCCCTC | |
| | CAATACCAAGGTCGACAAAAGGGTGGAACCCAGGGACTG | |
| | CGGCTGTAAACCTTGCCCACCTTGTCCAGCTCCTGAGCTG | |
| | CTGGGCGGACCATCCGTGTTCCTGTTTCCACCCAAGCCCA | |
| | AAGACACACTGATGATTAGCCGGACACCCGAAGTCACTTG | |
| | CGTGGTCGTGGACGTGTCTCACGAGGACCCCGAAGTCAAG | |
| | TTTAACTGGTACGTGGATGGAGTCGAGGTGCATAATGCAA | |
| | AGACTAAACCTAGAGAGGAACAGTACAACAGTACATATA | |
| | GAGTCGTGTCAGTCCTGACTGTGCTGCACCAGGACTGGCT | |
| | GAACGGGAAGGAATATAAGTGCAAAGTGAGTAATAAGGC | |
| | ACTGCCAGCCCCCATCGAGAAAACCATTTCCAAGGCCAAA | |
| | GGCCAGCCCAGGGAGCCACAGGTGTACACACTGCCTCCAT | |
| | CACGTGACGAGCTGACTAAGAACCAGGTCTCCCTGACCTG | |
| | TCTGGTGAAAGGCTTCTATCCTTCTGATATCGCTGTGGAGT | |
| | GGGAAAGTAATGGACAGCCAGAGAACAATTACAAGACCA | |
| | CACCCCCTGTGCTGGACAGCGATGGCTCTTTCTTTCTGTAT | |
| | TCCAAGCTGACAGTGGATAAAAGCCGCTGGCAGCAGGGA | |
| | AACGTGTTCTCCTGCAGTGTGATGCACGAAGCCCTGCATA | |
| | ACCACTACACTCAGAAGAGCCTGTGCCTGTCCCCTGGC | |
| huCMET-27 | ATGGGTTGGTCCTGTATTATTCTGTTTCTGGTCGCTACCGC | 116 |
| HC_Gv3- | TACTGGGGTCCATTCCGAAGTGCAGCTGGTCGAGAGTGGG | |
| hinge#26 | GGAGGGCTGGTGCAGCCTGGCGGAAGCCTGAGACTGTCTT | |
| | GCGCCGCTAGTGGCTTCACCTTTTCCAGCTACGACATGAG | |
| | CTGGGTGCGCCAGGCACCAGGGAAGGGTCTGGAGTGGGT | |
| | CGCCACTATCAACTCAAATGGCGTGTCCATCTACTATCCC | |
| | GACTCTGTCAAGGGAAGGTTCACCATCTCCAGGGACAACG | |
| | CAAAAAATAGCCTGTACCTGCAGATGAACTCTCTGCGAGC | |
| | CGAAGACACCGCCGTGTACTATTGCGCCCGTGAGGAAATT | |
| | ACCACAGAGATGGATTATTGGGGCCAGGGAACCCTGGTC | |
| | ACAGTGTCTAGTGCCAGCACAAAGGGCCCAAGCGTCTTCC | |
| | CTCTGGCTCCATCAAGCAAATCAACTTCTGGCGGCACAGC | |
| | AGCACTGGGGTGTCTGGTCAAGGACTACTTTCCCGAGCCT | |
| | GTCACCGTGTCATGGAACTCCGGGGCTCTGACCAGCGGTG | |
| | TCCACACATTCCCAGCAGTGCTGCAGTCCAGCGGGCTGTA | |
| | CTCTCTGTCTAGTGTGGTCACTGTGCCATCATCCAGCCTGG | |
| | GTACTCAGACCTATATCTGCAACGTGAATCATAAGCCCTC | |
| | CAATACCAAGGTCGACAAAAGGGTGGAACCCAGGGACTG | |
| | CGGCTGTAAACCTTGCCCACCTTGTCCAGCTCCTGAGCTG | |
| | CTGGGCGGACCATCCGTGTTCCTGTTTCCACCCAAGCCCA | |
| | AAGACACACTGATGATTAGCCGGACACCCGAAGTCACTTG | |
| | CGTGGTCGTGGACGTGTCTCACGAGGACCCCGAAGTCAAG | |
| | TTTAACTGGTACGTGGATGGAGTCGAGGTGCATAATGCAA | |
| | AGACTAAACCTAGAGAGGAACAGTACAACAGTACATATA | |
| | GAGTCGTGTCAGTCCTGACTGTGCTGCACCAGGACTGGCT | |
| | GAACGGGAAGGAATATAAGTGCAAAGTGAGTAATAAGGC | |
| | ACTGCCAGCCCCCATCGAGAAAACCATTTCCAAGGCCAAA | |
| | GGCCAGCCCAGGGAGCCACAGGTGTACACACTGCCTCCAT | |
| | CACGTGACGAGCTGACTAAGAACCAGGTCTCCCTGACCTG | |
| | TCTGGTGAAAGGCTTCTATCCTTCTGATATCGCTGTGGAGT | |
| | GGGAAAGTAATGGACAGCCAGAGAACAATTACAAGACCA | |
| | CACCCCCTGTGCTGGACAGCGATGGCTCTTTCTTTCTGTAT | |
| | TCCAAGCTGACAGTGGATAAAAGCCGCTGGCAGCAGGGA | |
| | AACGTGTTCTCCTGCAGTGTGATGCACGAAGCCCTGCATA | |
| | ACCACTACACTCAGAAGAGCCTGTCCCTGTCCCCTGGC | |

Also provided is a polynucleotide having at least about 95%, at least about 96%, at least about 97%, at least about 98%, or at least about 99% sequence identity to the sequences in Table 5 (SEQ ID NOs:55-67). In particular embodiments, also provided is a polynucleotide having at least about 95%, at least about 96%, at least about 97%, at least about 98%, or at least about 99% sequence identity to the following sequences:

SEQ ID NOs: 55 and/or 56
SEQ ID NOs: 57 and/or 61
SEQ ID NOs: 57 and/or 60
SEQ ID NOs: 57 and/or 59
SEQ ID NOs: 58 and/or 61
SEQ ID NOs: 58 and/or 60
SEQ ID NOs: 58 and/or 59
SEQ ID NOs: 62 and/or 63
SEQ ID NOs: 64 and/or 67
SEQ ID NOs: 65 and/or 67
SEQ ID NOs: 66 and/or 67

Also provided is a polynucleotide having at least about 95%, at least about 96%, at least about 97%, at least about 98%, or at least about 99% sequence identity to the sequences in Table 6 (SEQ ID NOs:68-72). In particular embodiments, also provided is a polynucleotide having at least about 95%, at least about 96%, at least about 97%, at least about 98%, or at least about 99% sequence identity to the following sequences:

SEQ ID NOs: 68 and/or 72
SEQ ID NOs: 68 and/or 71
SEQ ID NO: 68 and/or 70
SEQ ID NO: 68 and/or 109
SEQ ID NO: 68 and/or 110
SEQ ID NO: 68 and/or 111
SEQ ID NO: 68 and/or 112
SEQ ID NO: 68 and/or 113
SEQ ID NO: 68 and/or 114
SEQ ID NO: 68 and/or 115
SEQ ID NO: 68 and/or 116
SEQ ID NO: 69 and/or 72
SEQ ID NO: 69 and/or 71
SEQ ID NO: 69 and/or 70

In one embodiment, the polynucleotide has the sequence of SEQ ID NO:68 and SEQ ID NO:114.

The present invention further provides variants of the hereinabove described polynucleotides encoding, for example, fragments, analogs, and derivatives.

The polynucleotide variants can contain alterations in the coding regions, non-coding regions, or both. In some embodiments the polynucleotide variants contain alterations which produce silent substitutions, additions, or deletions, but do not alter the properties or activities of the encoded polypeptide. In some embodiments, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

The present invention also encompasses polynucleotides encoding a polypeptide that can bind MET and that hybridizes under stringent hybridization conditions to polynucleotides that encode an antibody of the present invention, wherein said stringent hybridization conditions include: pre-hybridization for 2 hours at 60° C. in 6×SSC, 0.5% SDS, 5× Denhardt's solution, and 100 μg/ml heat denatured salmon sperm DNA; hybridization for 18 hours at 60° C.; washing twice in 4×SSC, 0.5% SDS, 0.1% sodium pyrophosphate, for 30 min at 60° C. and twice in 2×SSC, 0.1% SDS for 30 min at 60° C.

The polynucleotides may be obtained, and the nucleotide sequence of the polynucleotides determined, using methods known in the art. For example, if the nucleotide sequence of the antibody is known, a polynucleotide encoding the antibody may be assembled from chemically synthesized oligonucleotides (e.g., as described in Kutmeier et al., 1994, BioTechniques 17:242) which, briefly, involves the synthesis of overlapping oligonucleotides containing portions of the sequence encoding the antibody, annealing and ligation of those oligonucleotides, and then amplification of the ligated oligonucleotides by PCR.

Methods for the construction of recombinant vectors containing antibody coding sequences and appropriate transcriptional and translational control signals are well known in the art. Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In this regard, U.S. Pat. No. 7,538,195 has been referred to in the present disclosure, the teachings of which are hereby incorporated in its entirety by reference.

In another aspect, diverse antibodies and antibody fragments, as well as antibody mimics may be readily produced by mutation, deletion and/or insertion within the variable and constant region sequences that flank a particular set of CDRs. Thus, for example, different classes of antibody are possible for a given set of CDRs by substitution of different heavy chains, whereby, for example, IgG1-4, IgM, IgA1-2, IgD, IgE antibody types and isotypes may be produced. Similarly, artificial antibodies within the scope of the invention may be produced by embedding a given set of CDRs within an entirely synthetic framework. The term “variable” is used herein to describe certain portions of the variable domains that differ in sequence among antibodies and are used in the binding and specificity of each particular antibody for its antigen. However, the variability is not usually evenly distributed through the variable domains of the antibodies. It is typically concentrated in three segments called complementarity determining regions (CDRs) or hypervariable regions both in the light chain and the heavy chain variable domains. The more highly conserved portions of the variable domains are called the framework (FR). The variable domains of heavy and light chains each comprise four framework regions, largely adopting a beta-sheet configuration, connected by three CDRs, which form loops connecting, and in some cases forming part of the beta-sheet structure. The CDRs in each chain are held together in close proximity by the FR regions and, with the CDRs from the other chain, contribute to the formation of the antigen binding site of antibodies (see, for example, E. A. Kabat et al. Sequences of Proteins of Immunological Interest, fifth edition, 1991, NIH). The constant domains are not involved directly in binding an antibody to an antigen, but exhibit various effector functions, such as participation of the antibody in antibody-dependent cellular toxicity.

Humanized antibodies, or antibodies adapted for non-rejection by other mammals, may be produced using several technologies such as resurfacing and CDR grafting. In the resurfacing technology, molecular modeling, statistical analysis and mutagenesis are combined to adjust the non-CDR surfaces of variable regions to resemble the surfaces of known antibodies of the target host. Strategies and methods for the resurfacing of antibodies, and other methods for reducing immunogenicity of antibodies within a different host, are disclosed in, for example, U.S. Pat. No. 5,639,641, which is hereby incorporated in its entirety by reference. In the CDR grafting technology, the murine heavy and light chain CDRs are grafted into a fully human framework sequence.

The invention also includes functional equivalents of the antibodies described in this specification. Functional equivalents have binding characteristics that are comparable to those of the antibodies, and include, for example, chimerized, humanized and single chain antibodies as well as fragments thereof. Exemplary methods of producing such functional equivalents are disclosed in PCT Application WO 93/21319, European Patent Application No. 239,400; PCT Application WO 89/09622; European Patent Application 338,745; and European Patent Application EP 332,424, which are incorporated in their respective entireties by reference.

Functional equivalents include polypeptides with amino acid sequences substantially the same as the amino acid sequence of the variable or hypervariable regions of the antibodies of the invention. “Substantially the same” as applied to an amino acid sequence is defined herein as a sequence with at least about 90%, and more preferably at least about 95%, 96%, 97%, 98%, and 99% sequence identity to another amino acid sequence, as determined by the FASTA search method in accordance with Pearson and Lipman, Proc. Natl. Acad. Sci. USA 85, 2444-2448 (1988).

Chimeric antibodies preferably can have constant regions derived substantially or exclusively from human antibody constant regions and variable regions derived substantially or exclusively from the sequence of the variable region from a mammal other than a human. Humanized forms of the antibodies can be made by substituting the complementarity determining regions of, for example, a mouse antibody, into a human framework domain, e.g., PCT Pub. No. WO92/22653. Humanized antibodies preferably can have constant regions and variable regions other than the complementarity determining regions (CDRs) derived substantially or exclusively from the corresponding human antibody regions and CDRs derived substantially or exclusively from a mammal other than a human.

Functional equivalents also include single-chain antibody fragments, also known as single-chain antibodies (scFvs). These fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the (V_{L}) and (V_{H}) domains occurs once they are linked so as to maintain the target molecule binding-specificity of the whole antibody from which the single-chain antibody fragment is derived. Generally, the carboxyl terminus of the (V_{L}) or (V_{H}) sequence may be covalently linked by such a peptide linker to the amino acid terminus of a complementary (V_{L}) and (V_{H}) sequence. Single-chain antibody fragments may be generated by molecular cloning, antibody phage display library or similar techniques. These proteins may be produced either in eukaryotic cells or prokaryotic cells, including bacteria.

Single-chain antibody fragments may contain amino acid sequences having at least one of the variable or complementarity determining regions (CDRs) of the intact antibodies described in this specification, but are lacking some or all of the constant domains of those antibodies. These constant domains are not necessary for antigen binding, but constitute a major portion of the structure of intact antibodies. Single-chain antibody fragments may therefore overcome some of the problems associated with the use of antibodies containing a part or all of a constant domain. For example, single-chain antibody fragments tend to be free of undesired interactions between biological molecules and the heavy-chain constant region, or other unwanted biological activity. Additionally, single-chain antibody fragments are considerably smaller than intact or whole antibodies and may therefore have greater capillary permeability than intact antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than intact antibodies.

The knowledge of the amino acid and nucleic acid sequences for the anti-MET antibody and its resurfaced or humanized variants, which are described herein, can be used to develop many antibodies which also bind to human MET. Several studies have surveyed the effects of introducing one or more amino acid changes at various positions in the sequence of an antibody, based on the knowledge of the primary antibody sequence, on its properties such as binding and level of expression (e.g., Yang, W. P. et al., 1995, J. Mol. Biol., 254, 392-403; Rader, C. et al., 1998, Proc. Natl. Acad. Sci. USA, 95, 8910-8915; Vaughan, T. J. et al., 1998, Nature Biotechnology, 16, 535-539).

In these studies, variants of the primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli (Vaughan, T. J., et al., 1998, Nature Biotechnology, 16, 535-539; Adey, N. B. et al., 1996, Chapter 16, pp. 277-291, in “Phage Display of Peptides and Proteins”, Eds. Kay, B. K. et al., Academic Press). These methods of changing the sequence of the primary antibody have resulted in improved affinities of the secondary antibodies (e.g., Gram, H. et al., 1992, Proc. Natl. Acad. Sci. USA, 89, 3576-3580; Boder, E. T. et al., 2000, Proc. Natl. Acad. Sci. USA, 97, 10701-10705; Davies, J. and Riechmann, L., 1996, Immunotechnolgy, 2, 169-179; Thompson, J. et al., 1996, J. Mol. Biol., 256, 77-88; Short, M. K. et al., 2002, J. Biol. Chem., 277, 16365-16370; Furukawa, K. et al., 2001, J. Biol. Chem., 276, 27622-27628).

By a similar directed strategy of changing one or more amino acid residues of the antibody, the antibody sequences described in this invention can be used to develop anti-MET antibodies with improved functions, such as those methods described in patent application publication 20090246195, the contents of which is incorporated in its entirety herein by reference.

### III. Immunoconjugates

In one aspect, the present invention relates to immunoconjugates comprising a MET-binding agent (e.g., an anti-MET antibody or an antibody fragment thereof) described herein conjugated or covalently linked to a cytotoxic agent described herein. Cytotoxic agents include any agent that is detrimental to cells such as, for example, Pseudomonas exotoxin, Diptheria toxin, a botulinum toxin A through F, ricin abrin, saporin, and cytotoxic fragments of such agents. Cytotoxic agents also include any agent having a therapeutic effect to prophylactically or therapeutically treat a disorder. Such therapeutic agents may be may be chemical therapeutic agents, protein or polypeptide therapeutic agents, and include therapeutic agents that possess a desired biological activity and/or modify a given biological response. Examples of therapeutic agents include alkylating agents, angiogenesis inhibitors, anti-mitotic agents, hormone therapy agents, and antibodies useful for the treatment of cell proliferative disorders. In certain embodiments, the therapeutic agents are maytansinoid compounds, such as those described in U.S. Pat. Nos. 5,208,020 and 7,276,497, incorporated herein by reference in its entirety. In certain embodiments, the therapeutic agents are benzodiazepine compounds, such as pyrrolobenzodiazepine (PBD) (such as those described in WO2010/043880, WO2011/130616, WO2009/016516, WO 2013/177481 and WO 2012/112708) and indolinobenzodiazepine (IGN) compounds (such as those described in WO/2010/091150, and WO 2012/128868 and U.S. application Ser. No. 15/195,269, filed on Jun. 28, 2016, entitled “CONJUGATES OF CYSTEINE ENGINEERED ANTIBODIES”). The entire teachings of all of these patents, patent publications and applications are incorporate herein by reference in their entireties.

As used herein, a “pyrrolobenzodiazepine” (PBD) compound is a compound having a pyrrolobenzodiazepine core structure. The pyrrolobenzodiazepine can be substituted or unsubstituted. It also includes a compound having two pyrrolobenzodiazepine core linked by a linker. The imine functionality (—C═N—) as part of indolinobenzodiazepine core can be reduced.

In certain embodiments, the pyrrolobenzodiazepine compound comprises a core structure represented by

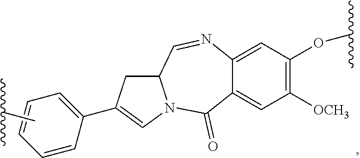

which can be optionally substituted.

In certain embodiments, the pyrrolobenzodiazepine compounds comprises a core structure represented by

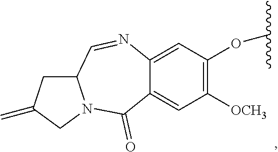 [SMILES: *Oc1cc2c(cc1OC)C(=O)N1CC(=C)CC1C=N2]

which can be optionally substituted.

As used herein, a “indolinobenzodiazepine” (IGN) compound is a compound having an indolinobenzodiazepine core structure. The indolinobenzodiazepine can be substituted or unsubstituted. It also includes a compound having two indolinobenzodiazepine core linked by a linker. The imine functionality (—C═N—) as part of indolinobenzodiazepine core can be reduced.

In certain embodiments, the indolinobenzodiazepine compound comprises a core structure represented by

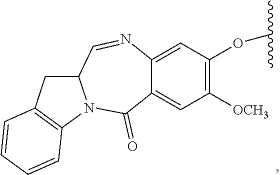 [SMILES: *Oc1cc2c(cc1OC)C(=O)N1c3ccccc3CC1C=N2]

which can be optionally substituted.

In some embodiments, the indolinobenzodiazepine compound comprises a core structure represented by

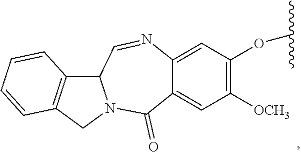 [SMILES: *Oc1cc2c(cc1OC)C(=O)N1Cc3ccccc3C1C=N2]

which can be further substituted.

The cytotoxic agent may be coupled or conjugated either directly to the MET-binding agent or indirectly, through a linker using techniques known in the art to produce an “immunoconjugate,” “conjugate,” or “ADC.”

A. Exemplary Immunoconjugates

In a first embodiment, the immunoconjugate of the present invention comprises a MET-binding agent (e.g., an anti-MET antibody or an antibody fragment thereof) described herein covalently linked to a cytotoxic agent described herein through the ε-amino group of one or more lysine residues located on the MET-binding agent.

In a 1^{st} specific embodiment of the first embodiment, the immunoconjugate of the present invention is represented by the following formula:

CBA+Cy^{L1})_{W}_{_{L}} (L1),

wherein:

CBA is a MET-binding agent (e.g., an anti-MET antibody or an antibody fragment thereof) described herein above that is covalently linked to Cy^{L1} through a lysine residue;

W_{L} is an integer from 1 to 20; and

Cy^{L1} is a cytotoxic compound represented by the following formula:

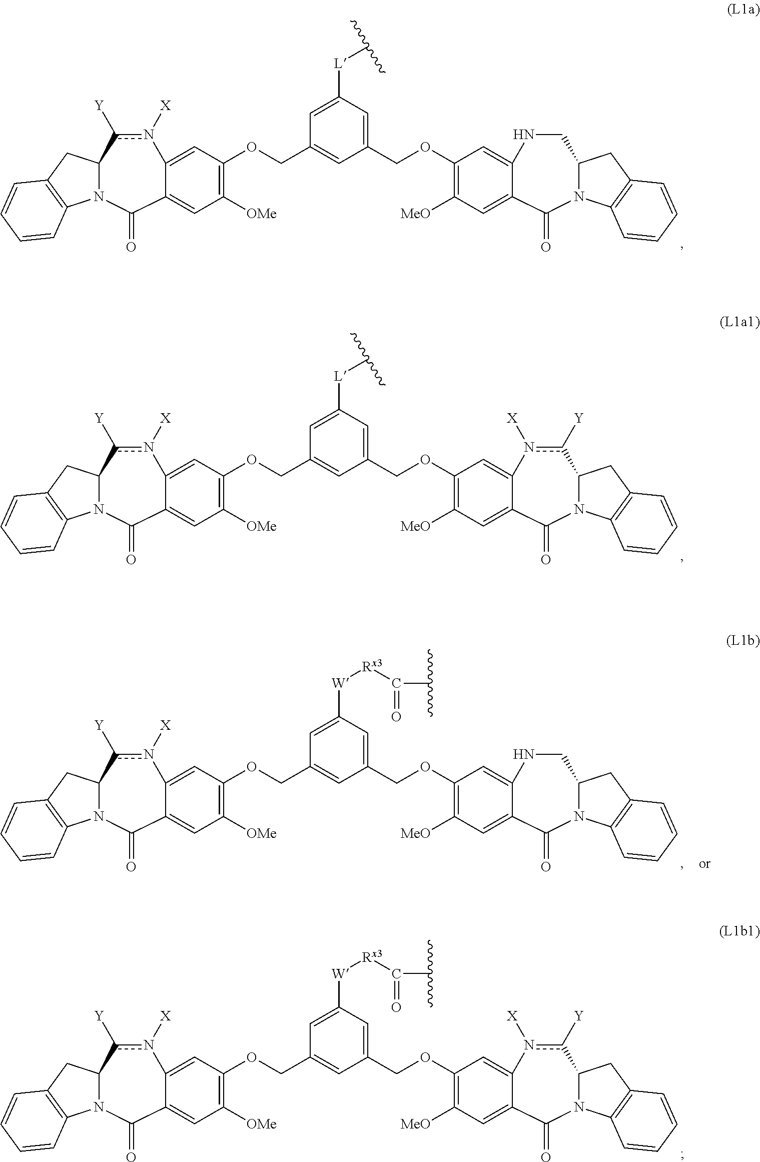

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃H or a pharmaceutically acceptable salt thereof;

W′ is —NR^{e′},

R^{e′} is —(CH₂—CH₂—O)ₙ—R^{k};

n is an integer from 2 to 6;

R^{k} is —H or -Me;

R^{x3} is a (C₁-C₆)alkyl;

L′ is represented by the following formula:

—NR₅—P—C(═O)—(CRₐR_{b})ₘ—C(═O)— (B1′); or

—NR₅—P—C(═O)—(CRₐR_{b})ₘ—S—Z^{S1}— (B2′);

R₅ is —H or a (C₁-C₃)alkyl;

P is an amino acid residue or a peptide containing between 2 to 20 amino acid residues;

Rₐ and R_{b}, for each occurrence, are each independently —H, (C₁-C₃)alkyl, or a charged substituent or an ionizable group Q;

m is an integer from 1 to 6; and

Z^{S1} is selected from any one of the following formulas:

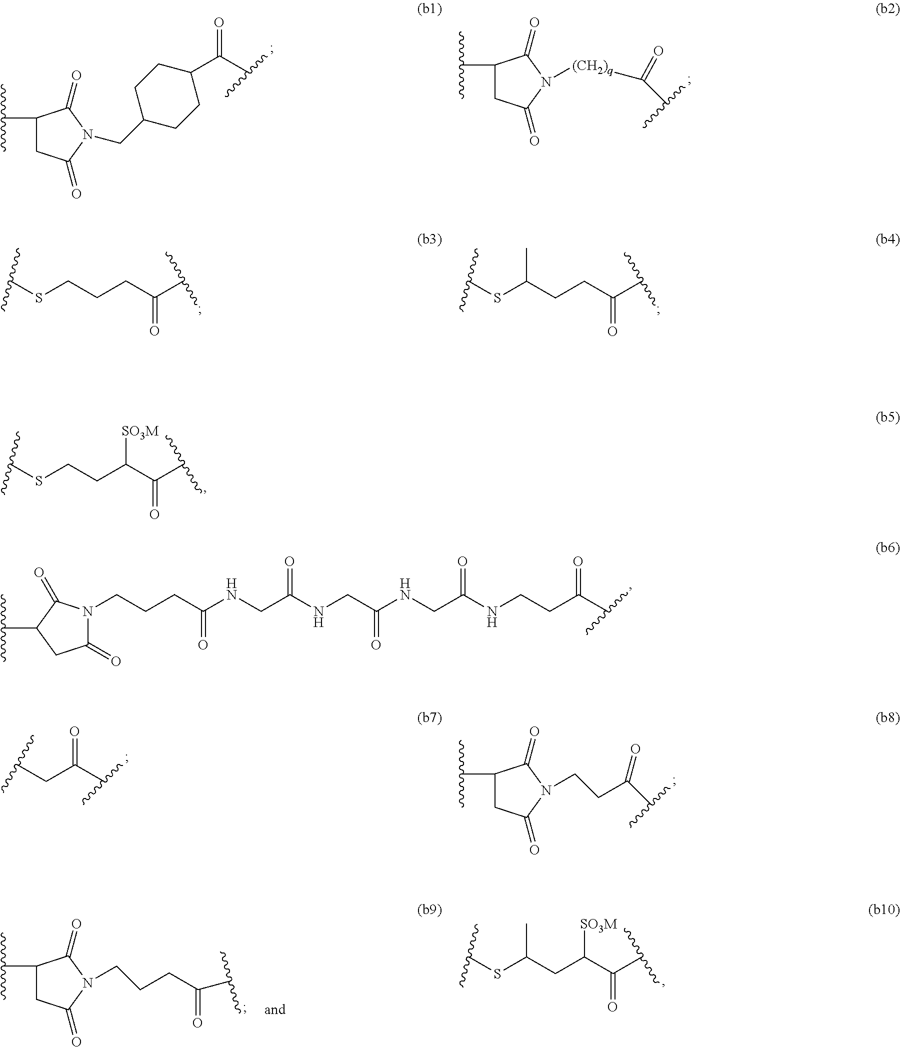

wherein q is an integer from 1 to 5.

In a 2^{nd} specific embodiment, for conjugates of formula (L1), Cy^{L1} is represented by formula (L1a) or (L1a1); and the remaining variables are as described above in the 1^{st} specific embodiment.

In a 3^{rd} specific embodiment, for conjugates of formula (L1), Cy^{L1} is represented by formula (L1b) or (L1b1); and the remaining variables are as described above in the 1^{st} specific embodiment. More specifically, R^{x3} is a (C₂-C₄)alkyl.

In a 4^{th} specific embodiment, for conjugates of formula (L1), Cy^{L1} is represented by formula (L1a); Rₐ and R_{b} are both H; R₅ is H or Me, and the remaining variables are as described above in the 1^{st} specific embodiment.

In a 5^{th} specific embodiment, P is a peptide containing 2 to 5 amino acid residues; and the remaining variables are described above in the 1^{st}, 2^{nd} or 4^{th} specific embodiment. In a more specific embodiment, P is selected from the group consisting of Gly-Gly-Gly, Ala-Val, Val-Ala, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:74), β-Ala-Leu-Ala-Leu (SEQ ID NO:75), Gly-Phe-Leu-Gly (SEQ ID NO:76), Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. More specifically, P is Gly-Gly-Gly, Ala-Val, Ala-Ala, Ala-D-Ala, D-Ala-Ala, or D-Ala-D-Ala.

In a 6^{th} specific embodiment, Q is —SO₃H or a pharmaceutically acceptable salt thereof; and the remaining variables are as described above in the 1^{st}, 2^{nd}, 4^{th} or 5^{th} specific embodiment or any more specific embodiments described therein.

In a 7^{th} specific embodiment, the immunoconjugate of the first embodiment is represented by the following formula:

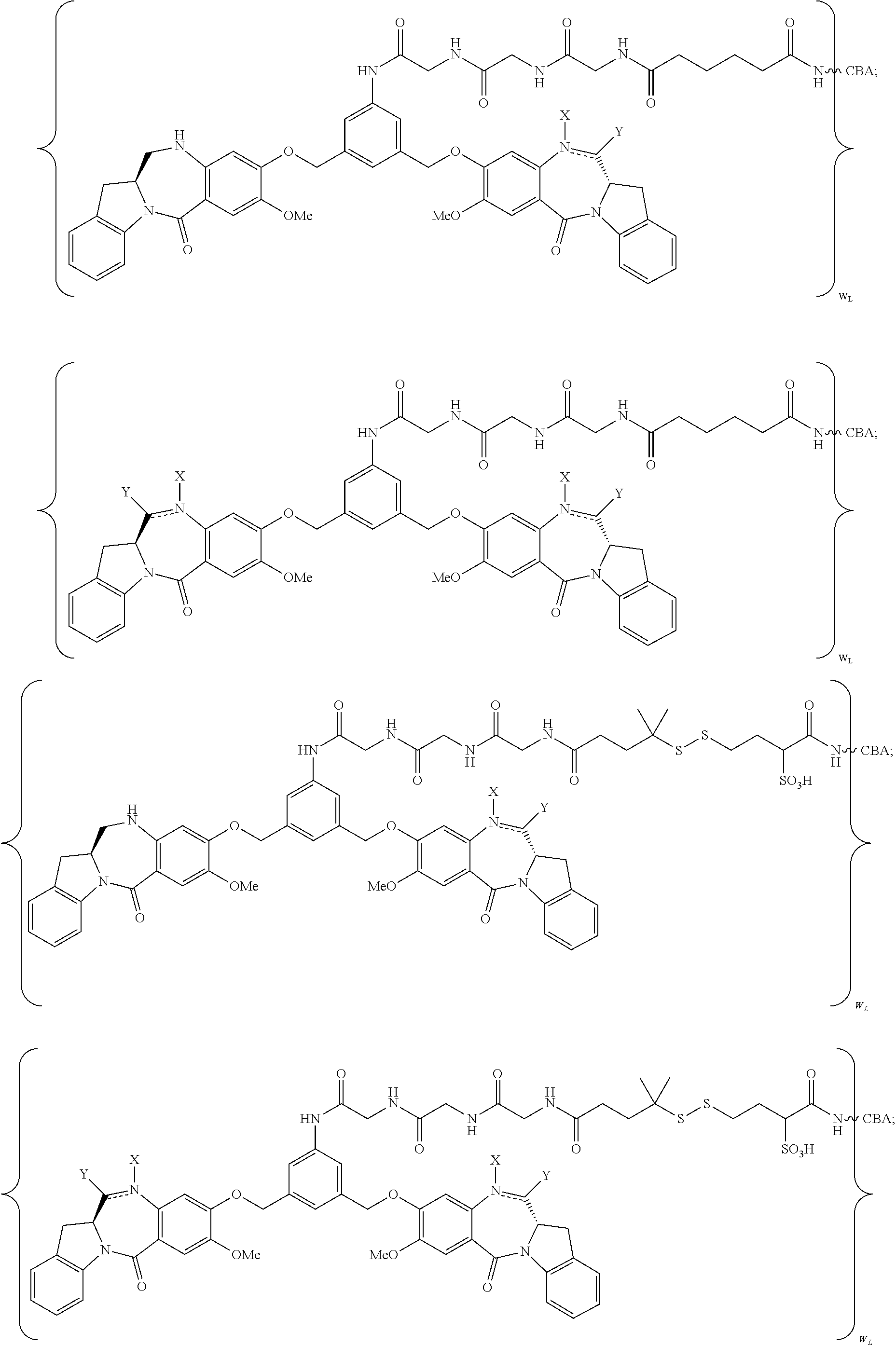

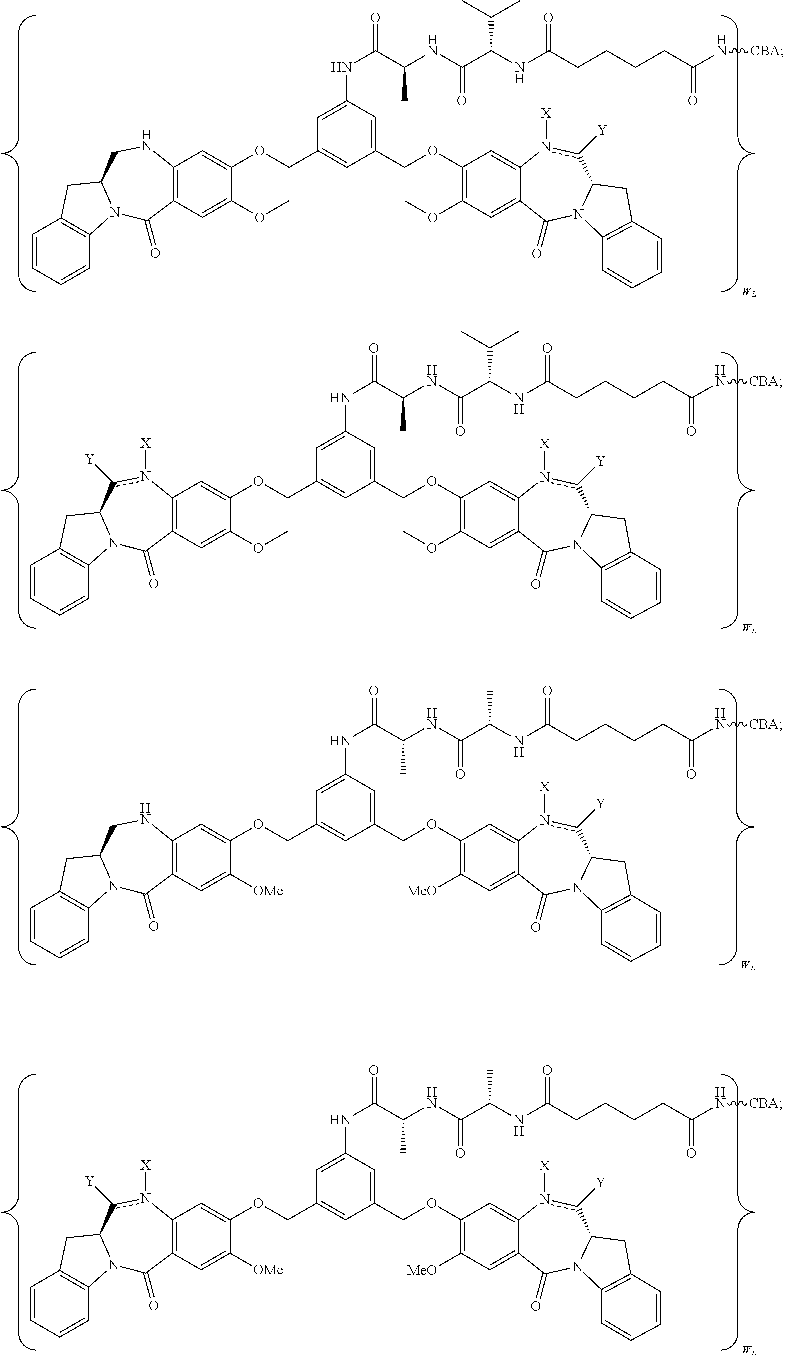

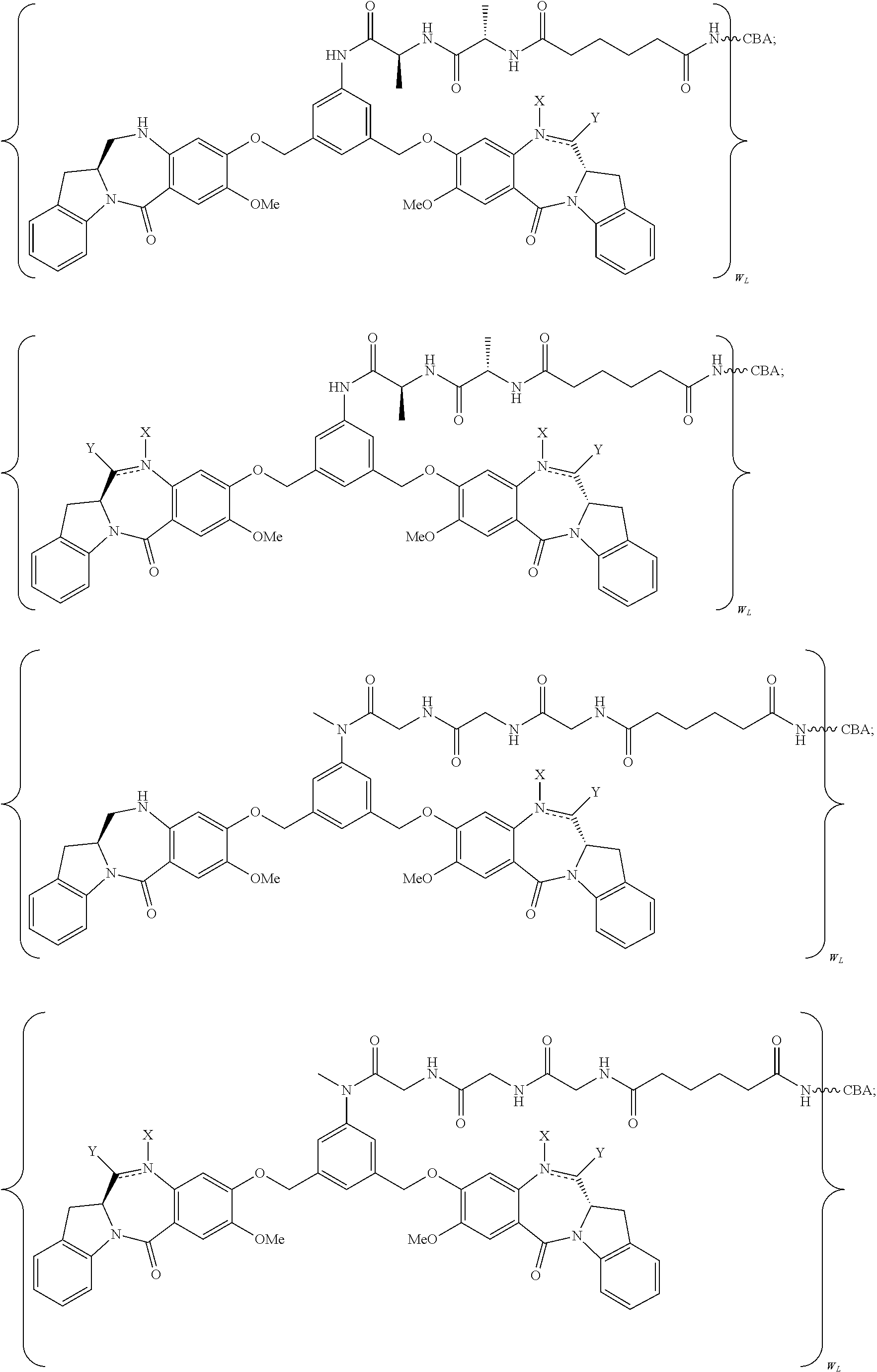

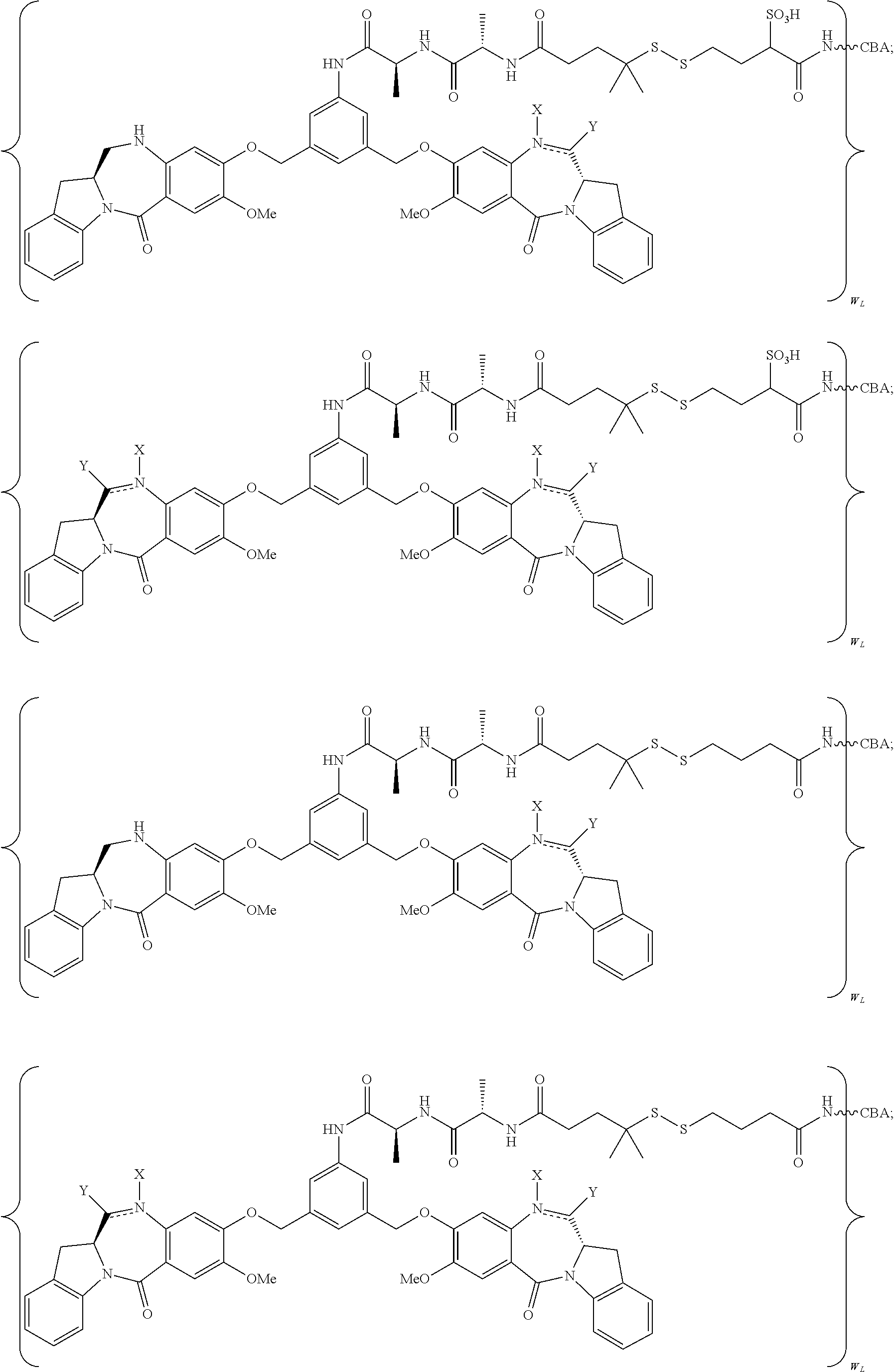

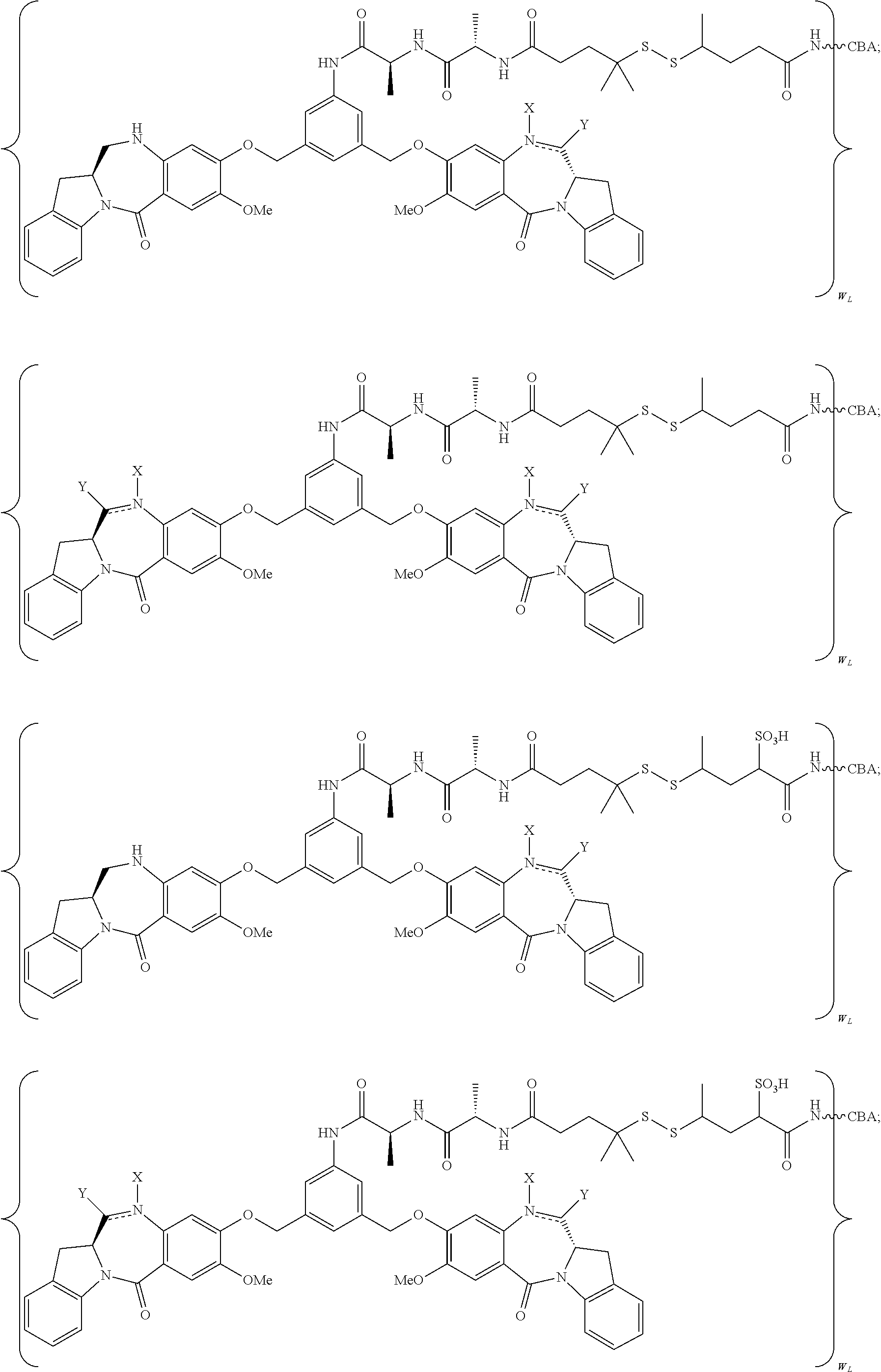

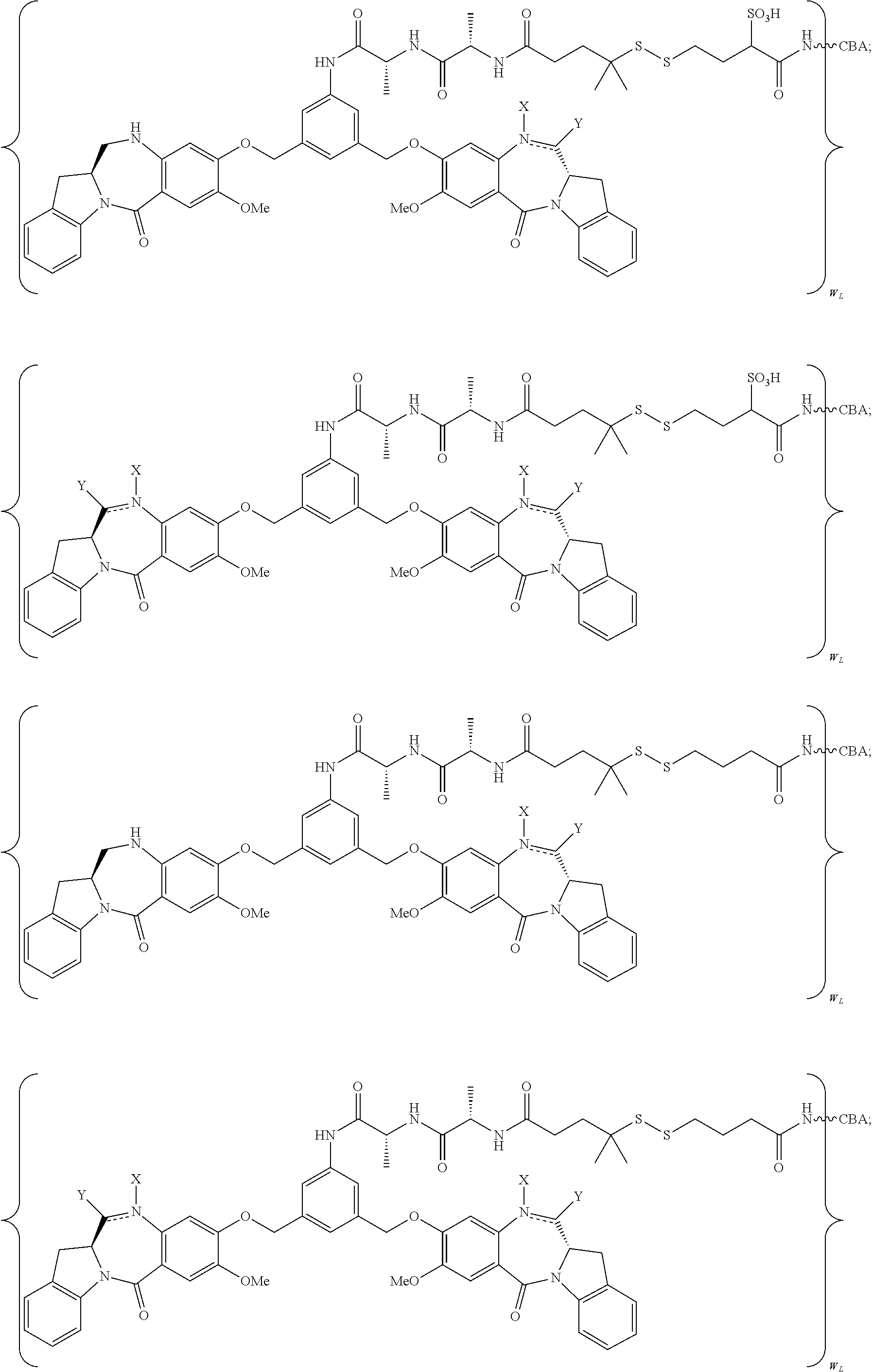

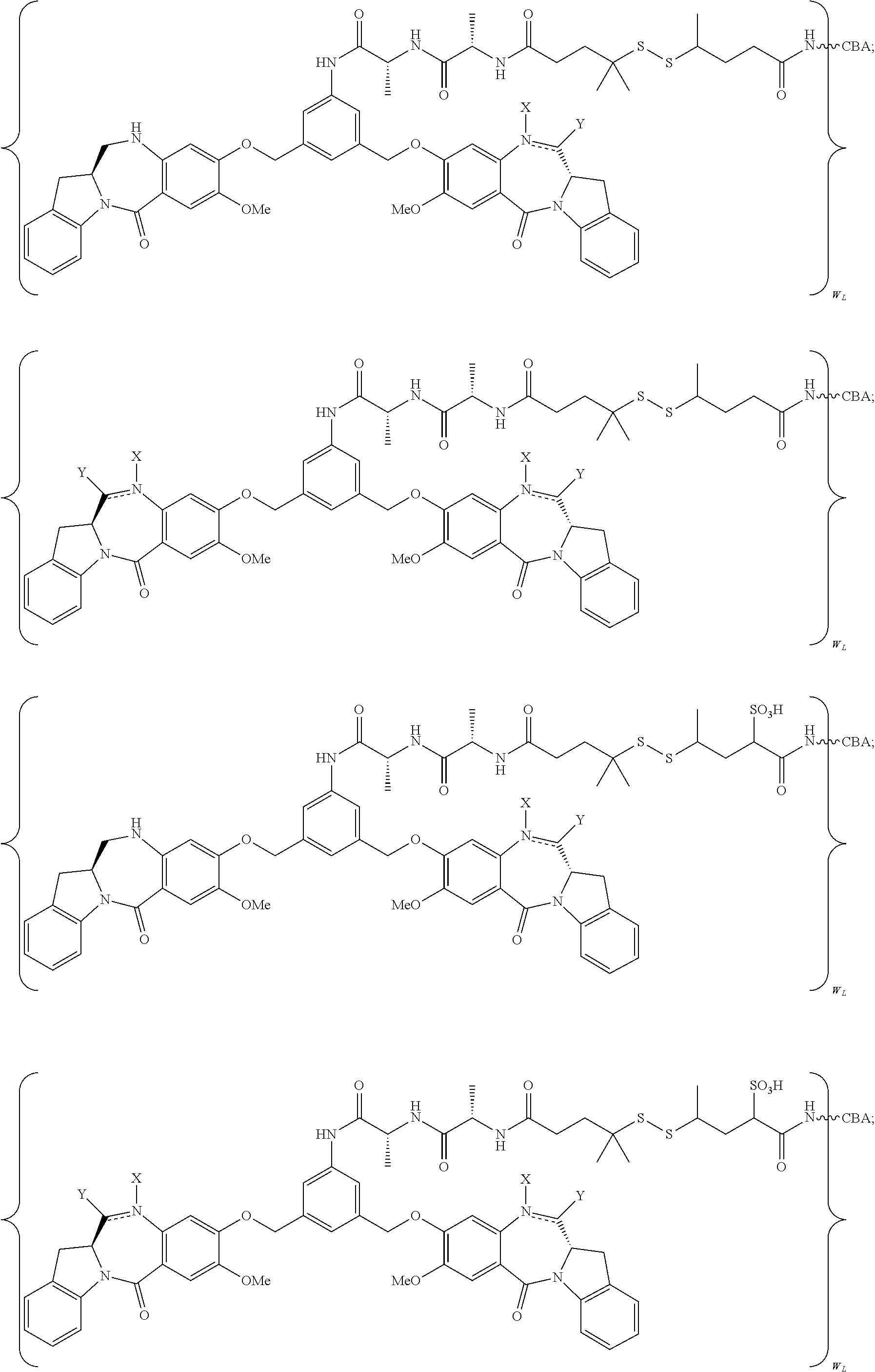

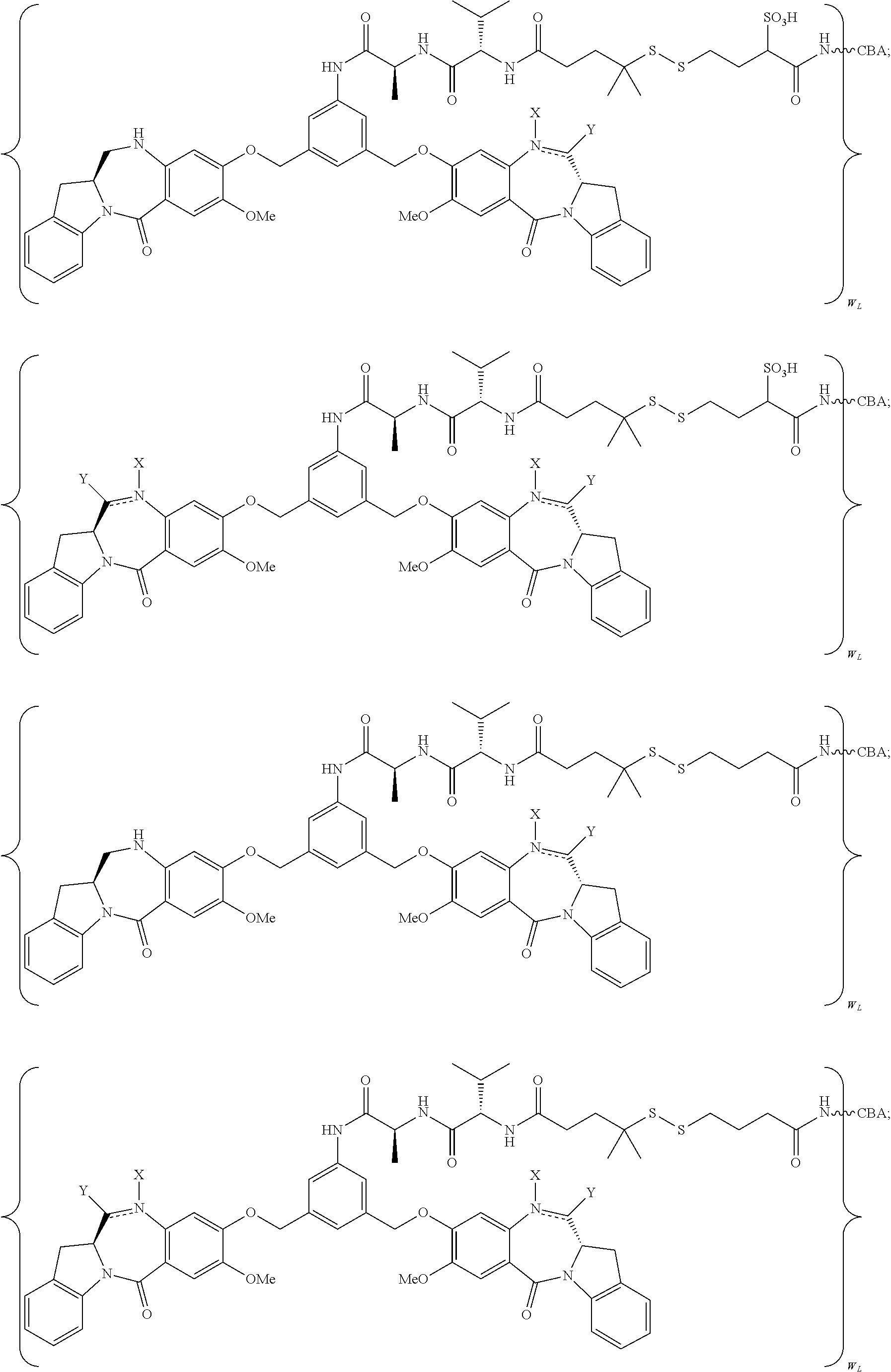

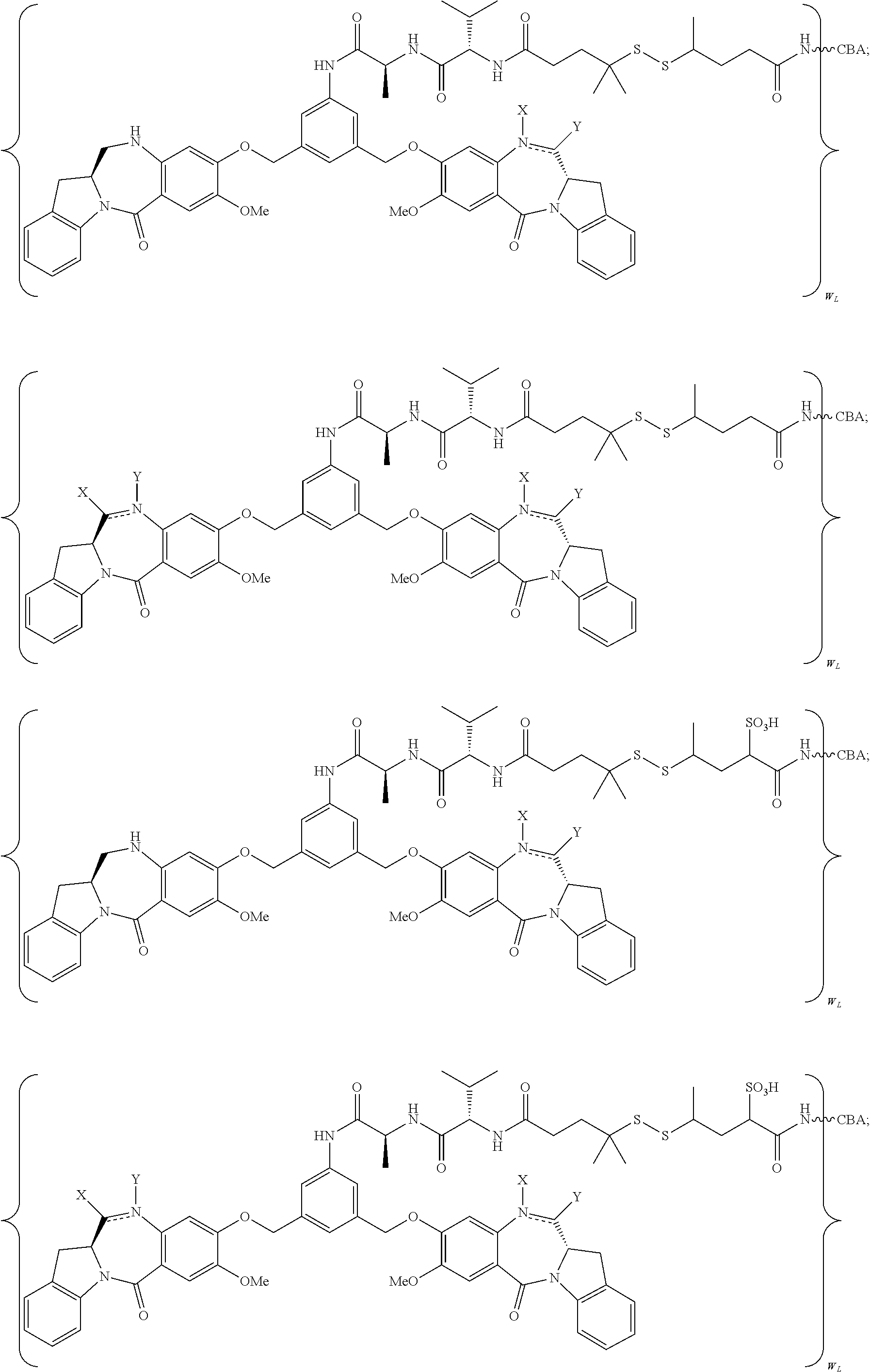

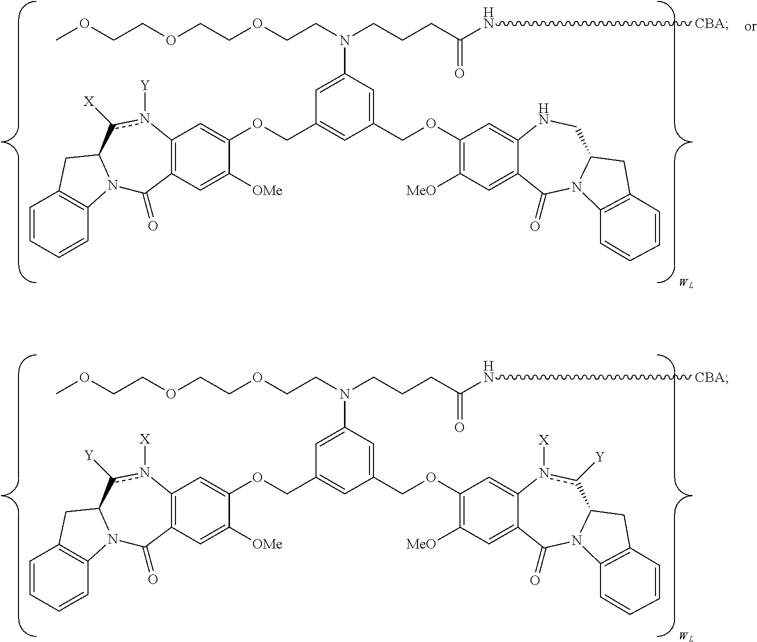

or a pharmaceutically acceptable salt thereof, wherein W_{L} is an integer from 1 to 10; the double line [symbol] between N and C represents a single bond or a double bond, provided that when it is a double bond, X is absent and Y is —H; and when it is a single bond, X is —H, and Y is —OH or —SO₃H or a pharmaceutically acceptable salt thereof. In a more specific embodiment, the double line [symbol] between N and C represents a double bond, X is absent and Y is —H. In another more specific embodiment, the double line [symbol] between N and C represents a single bond, X is —H and Y is —SO₃H or a pharmaceutically acceptable salt thereof.

In a 8^{th} specific embodiment, the immunoconjugate of the first embodiment is represented by the following formula:

CBACy^{L2})_{W}_{_{L}} (L2),

wherein:

CBA is a MET-binding agent (e.g., an anti-MET antibody or an antibody fragment thereof) described herein above that is covalently linked to Cy^{L2} through a lysine residue;

W_{L} is an integer from 1 to 20; and

Cy^{L2} is a cytotoxic compound represented by the following formula:

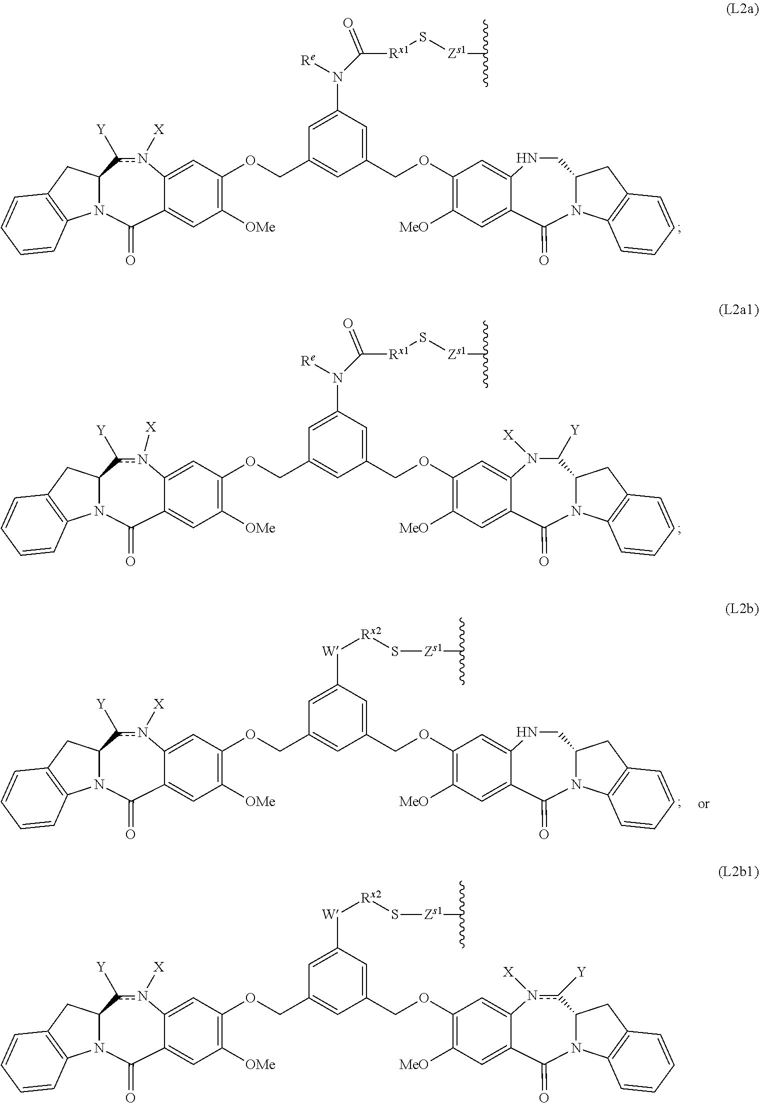

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃H or a pharmaceutically acceptable salt thereof;

R^{x1} and R^{x2} are independently (C₁-C₆)alkyl;

R^{e} is —H or a (C₁-C₆)alkyl;

W′ is —NR^{e′},

R^{e′} is —(CH₂—CH₂—O)ₙ—R^{k};

n is an integer from 2 to 6;

R^{k} is —H or -Me;

Z^{S1} is selected from any one of the following formulas:

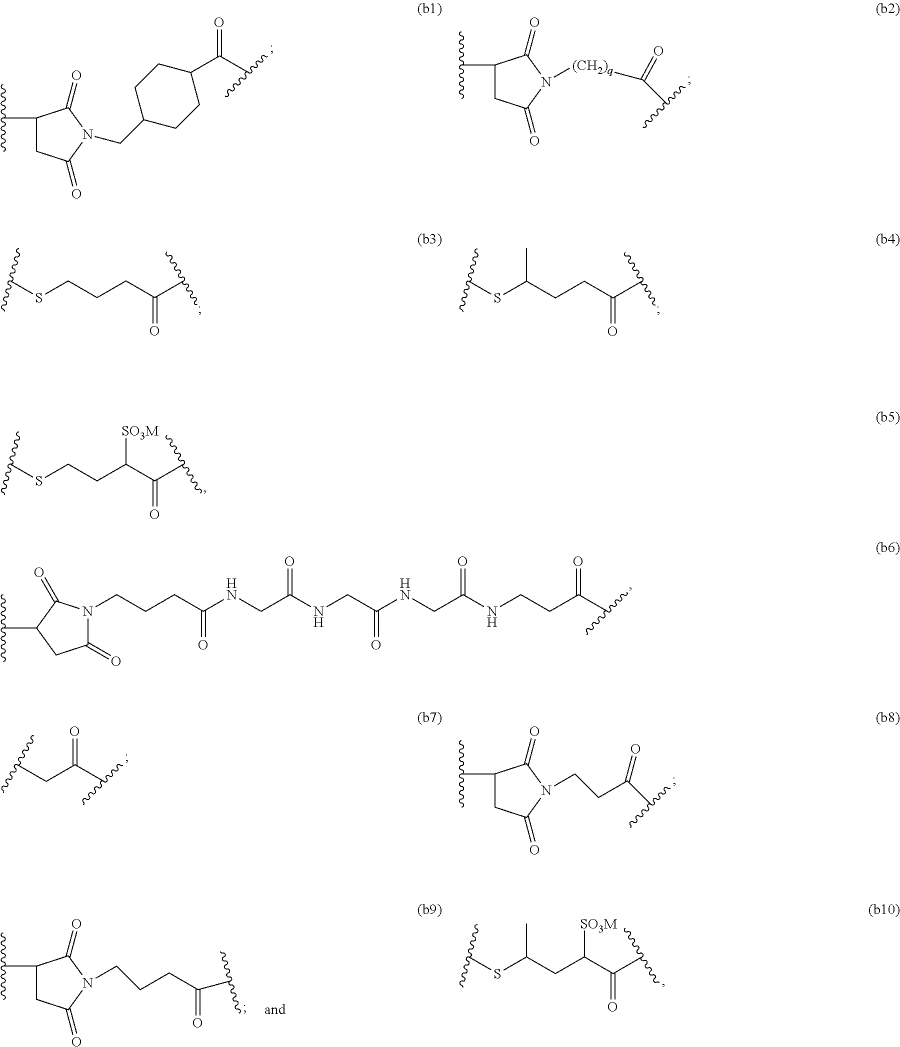

wherein q is an integer from 1 to 5.

In a 9^{th} specific embodiment, for immunoconjugates of formula (L2), Cy^{L2} is represented by formula (L2a) or (L2a1); and the remaining variables are as described above in the 8^{th} specific embodiment.

In a 10^{th} specific embodiment, for immunoconjugates of formula (L2), Cy^{L2} is represented by formula (L2b) or (L2b1); and the remaining variables are as described above in the 8^{th} specific embodiment.

In a 11^{th} specific embodiment, for immunoconjugates of formula (L2), R^{e} is H or Me; R^{x1} and R^{x2} are independently —(CH₂)ₚ—(CR^{f}R^{g})—, wherein R^{f} and R^{g} are each independently —H or a (C₁-C₄)alkyl; and p is 0, 1, 2 or 3; and the remaining variables are as described above in the 8^{th}, 9^{th} or 10^{th} specific embodiment. More specifically, R^{f} and R^{g} are the same or different, and are selected from —H and -Me.

In a 12^{th} specific embodiment, the immunoconjugate of the first embodiment is represented by the following formula:

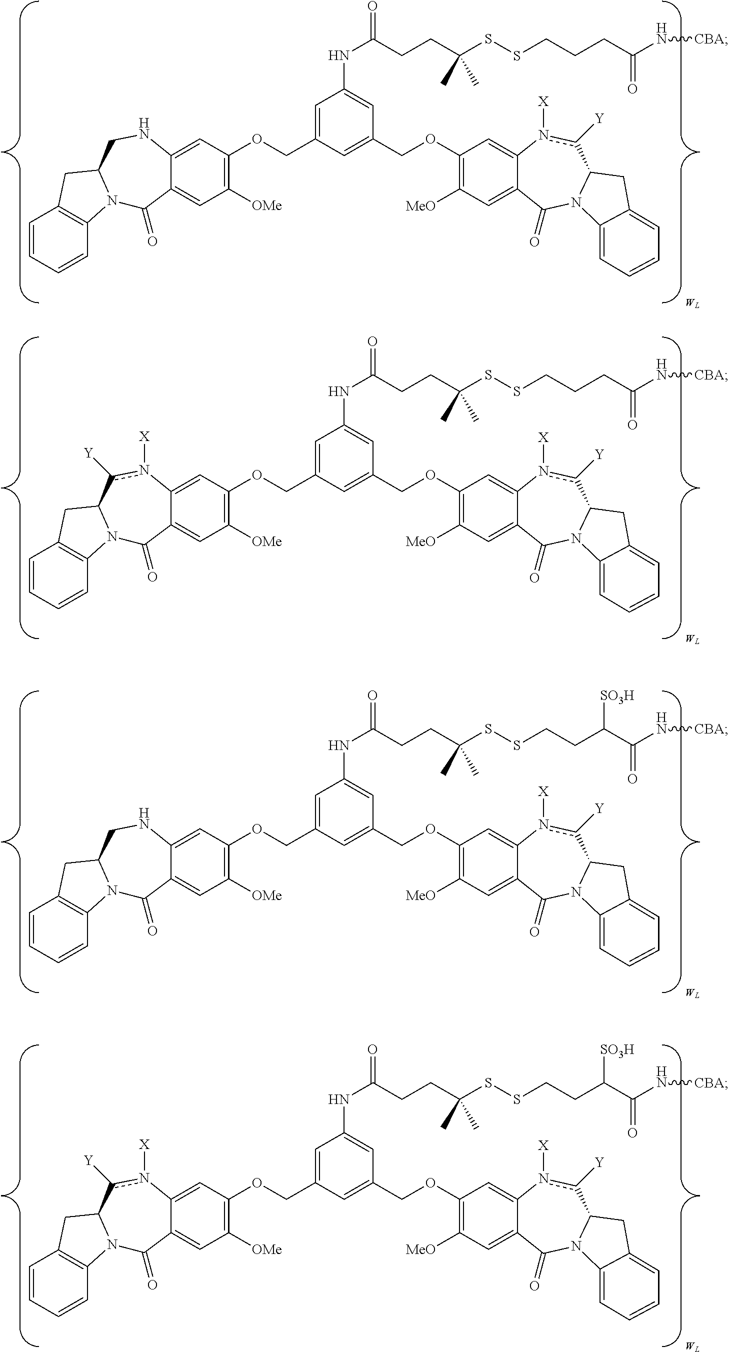

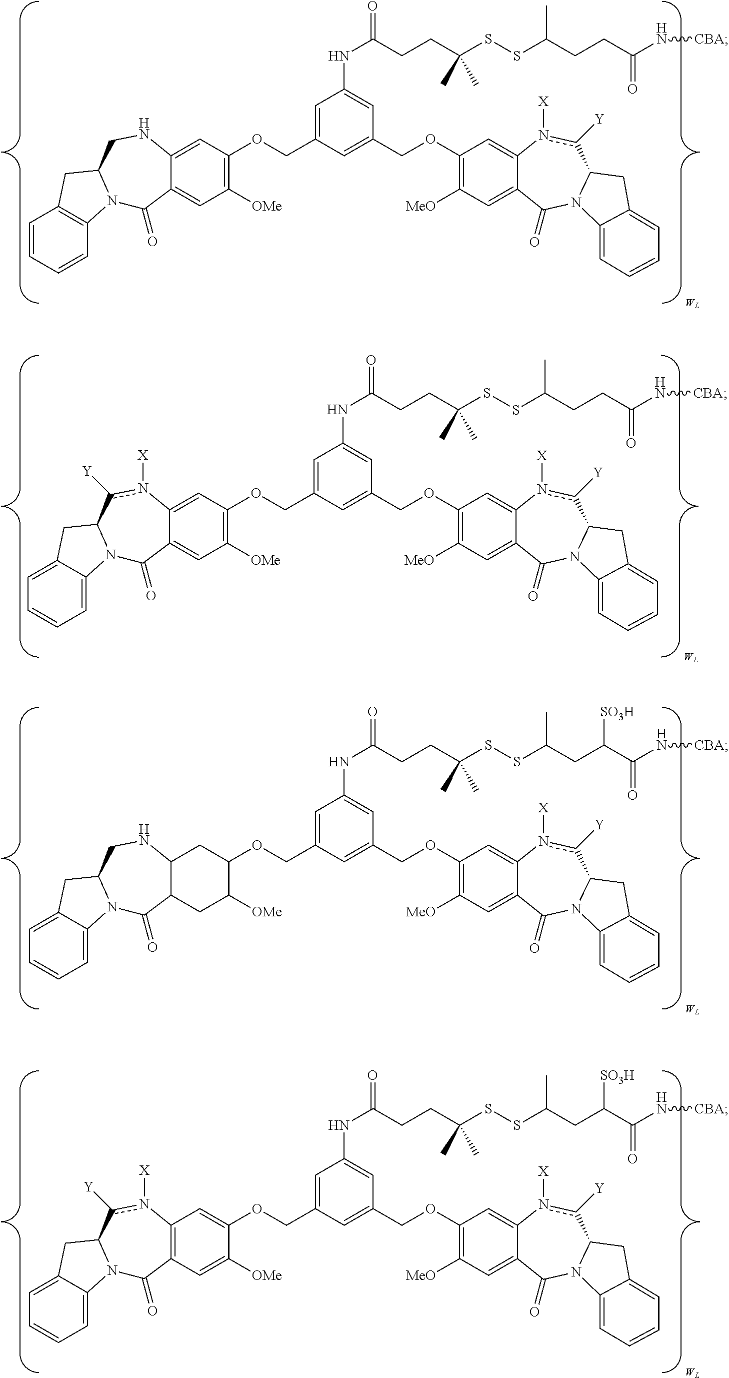

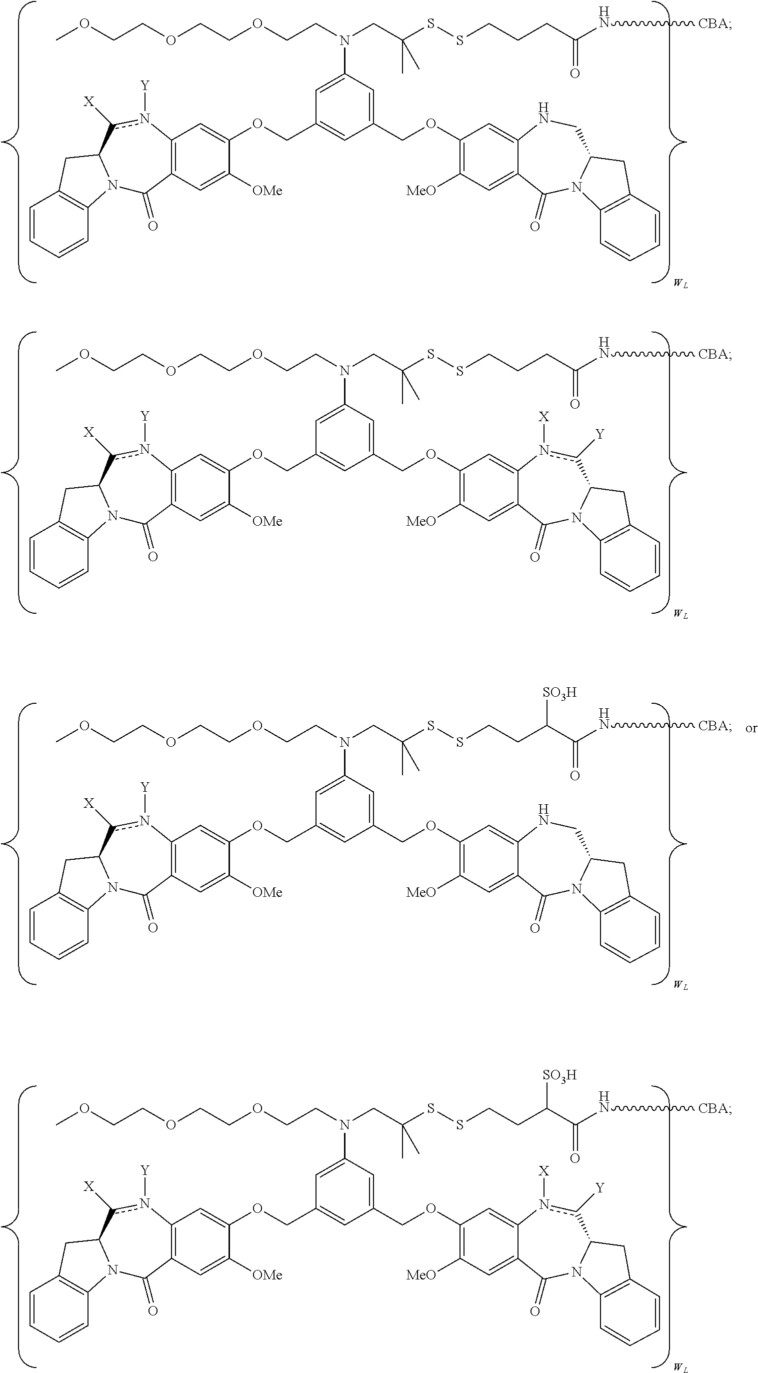

or a pharmaceutically acceptable salt thereof, wherein W_{L} is an integer from 1 to 10; the double line [symbol] between N and C represents a single bond or a double bond, provided that when it is a double bond, X is absent and Y is —H; and when it is a single bond, X is —H and Y is —OH or —SO₃H or a pharmaceutically acceptable salt thereof. In a more specific embodiment, the double line [symbol] between N and C represents a double bond. In another more specific embodiment, the double line [symbol] between N and C represents a single bond, X is —H and Y is —SO₃H or a pharmaceutically acceptable salt thereof.

In a 13^{th} specific embodiment, the immunoconjugates of the first embodiment is represented by the following formula:

CBACy^{L3})_{W}_{_{L}} (L3),

wherein:

CBA is a MET-binding agent (e.g., an anti-MET antibody or an antibody fragment thereof) described herein above that is covalently linked to Cy^{L3} through a Lys residue;

W_{L} is an integer from 1 to 20;

Cy^{L3} is represented by the following formula:

m′ is 1 or 2;

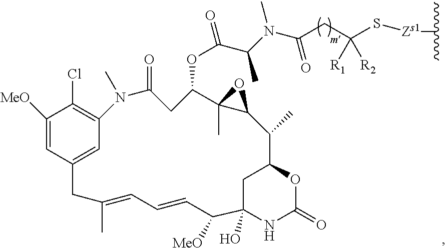

R₁ and R₂, are each independently H or a (C₁-C₃)alkyl; and

Z^{S1} is selected from any one of the following formulas:

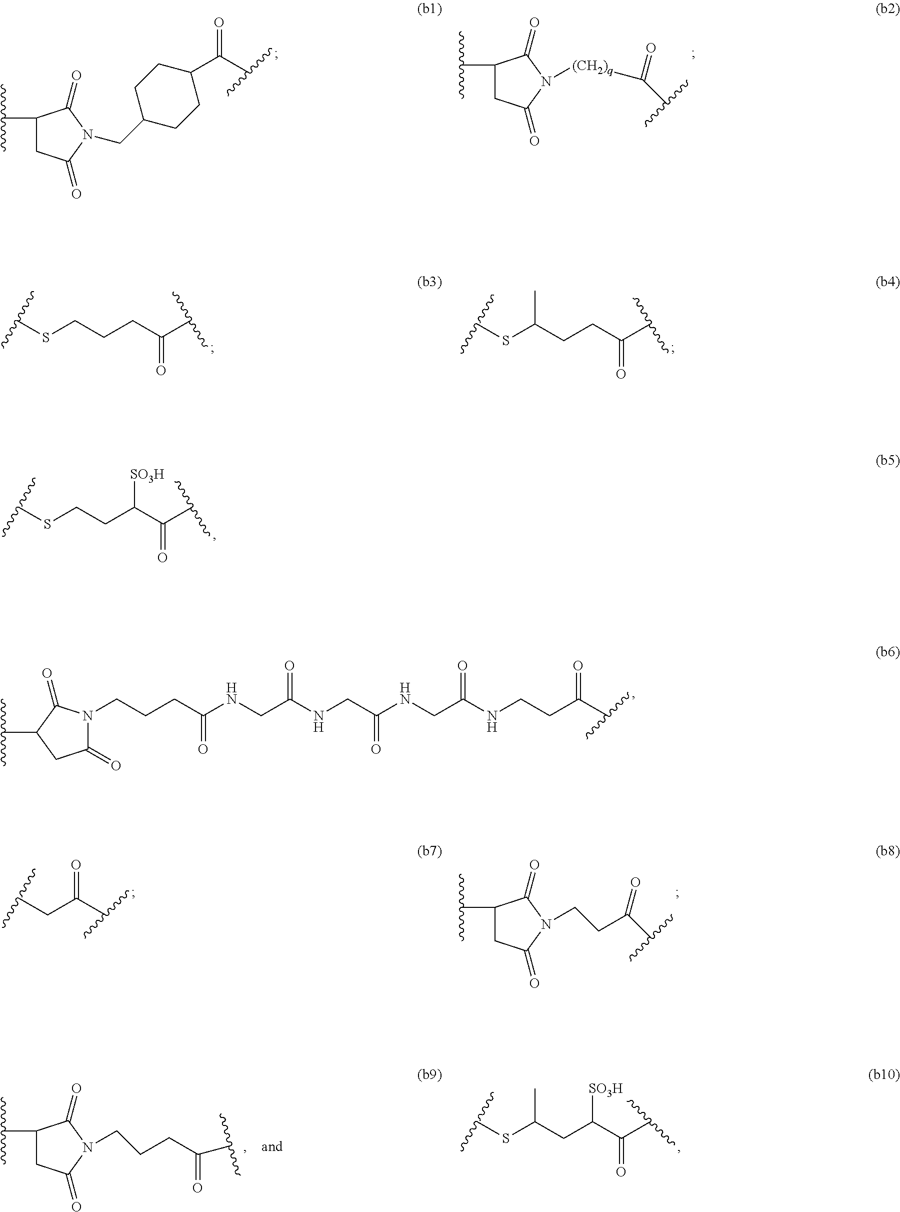

wherein q is an integer from 1 to 5.

In a 14^{th} specific embodiment, for immunoconjugates of formula (L3), m′ is 1, and R₁ and R₂ are both H; and the remaining variables are as described above in the 13^{th} specific embodiment.

In a 15^{th} specific embodiment, for immunoconjugates of formula (L3), m′ is 2, and R₁ and R₂ are both Me; and the remaining variables are as described above in the 13^{th} specific embodiment.

In a 16^{th} specific embodiment, the immunoconjugates of the first embodiment is represented by the following formula:

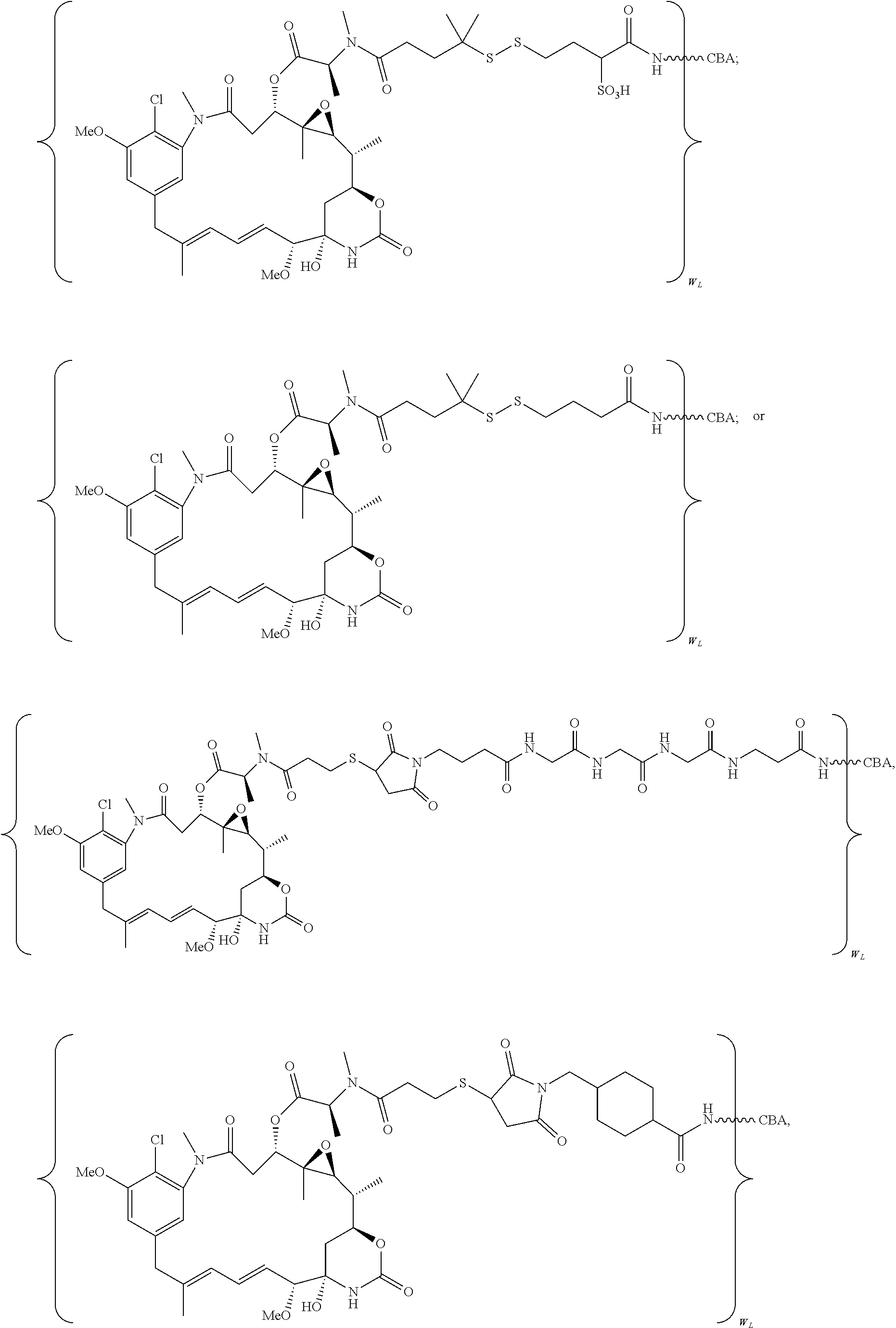

or a pharmaceutically acceptable salt thereof, wherein W_{L} is an integer from 1 to 10.

In a 17^{th} specific embodiment, for immunoconjugates of the first embodiment, Y is —SO₃H, —SO₃Na or —SO₃K; and the remaining variables are as described above in any one of the 1^{st} to 16^{th} specific embodiment or any more specific embodiments described therein. In one embodiment, Y is —SO₃Na.

In certain embodiments, for compositions (e.g., pharmaceutical compositions) comprising immunoconjugates of the first embodiment, or the 1st, 2nd, 3rd, 4th, 5th, 6th, 7th, 8th, 9th, 10th, 11th, 12th, 13th, 14th, 15th, 16th or 17th specific embodiment, the average number of the cytotoxic agent per antibody molecule (i.e., average value of wL), also known as Drug-Antibody Ratio (DAR) in the composition is in the range of 1.0 to 8.0. In some embodiments, DAR is in the range of 1.0 to 5.0, 1.0 to 4.0, 1.0 to 3.4, 1.0 to 3.0, 1.5 to 2.5, 2.0 to 2.5, or 1.8 to 2.2. In some embodiments, the DAR is less than 4.0, less than 3.8, less than 3.6, less than 3.5, less than 3.0 or less than 2.5.

In a second embodiment, the immunoconjugates of the present invention comprises an a MET-binding agent (e.g., an anti-MET antibody or an antibody fragment thereof) described herein above covalently linked to a cytotoxic agent described herein through the thiol group (—SH) of one or more cysteine residues located on the MET-binding agent.

In a 1^{st} specific embodiment, the immunoconjugate of the second embodiment is represented by the following formula:

CBACy^{C1})_{W}_{_{C}} (C1),

wherein:

CBA is a MET-binding agent (e.g., an anti-MET antibody or an antibody fragment thereof) described herein covalently linked to Cy^{C1} through a cysteine residue;

W_{C} is 1 or 2;

Cy^{C1} is represented by the following formula:

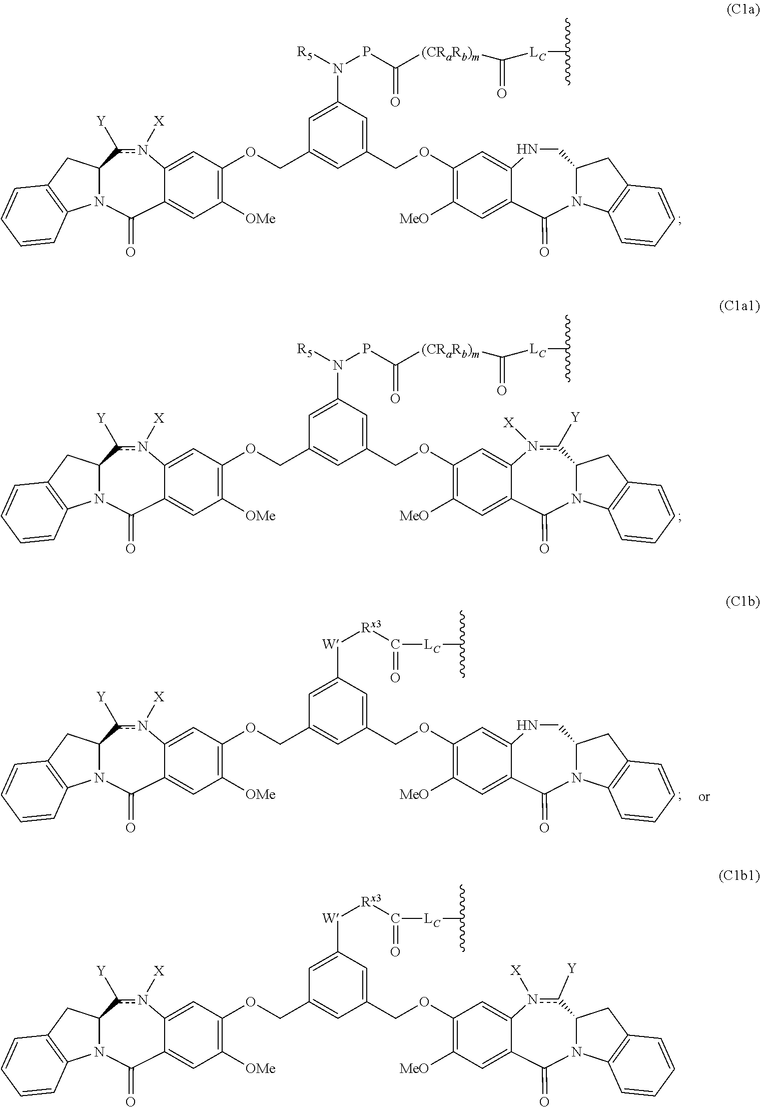

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃H or a pharmaceutically acceptable salt thereof;

R₅ is —H or a (C₁-C₃)alkyl;

P is an amino acid residue or a peptide containing 2 to 20 amino acid residues;

Rₐ and R_{b}, for each occurrence, are independently —H, (C₁-C₃)alkyl, or a charged substituent or an ionizable group Q;

m is an integer from 1 to 6;

W′ is —NR^{e′},

R^{e′} is —(CH₂—CH₂—O)ₙ—R^{k};

n is an integer from 2 to 6;

R^{k} is —H or -Me;

R^{x3} is a (C₁-C₆)alkyl; and,

L_{C} is represented by:

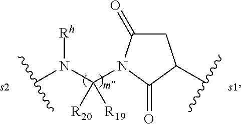

wherein s1 is the site covalently linked to CBA, and s2 is the site covalently linked to the —C(═O)— group on Cy^{C1}; wherein:

R₁₉ and R₂₀, for each occurrence, are independently —H or a (C₁-C₃)alkyl;

m″ is an integer between 1 and 10; and

R^{h} is —H or a (C₁-C₃)alkyl.

In a 2^{nd} specific embodiment, for immunoconjugate of formula (C1), Cy^{C1} is represented by formula (C1a) or (C1a1); and the remaining variables are as described above in the 1^{st} specific embodiment of the second embodiment.

In a 3^{rd} specific embodiment, for immunoconjugate of formula (C1), Cy^{C1} is represented by formula (C1b) or (C1b1); and the remaining variables are as described above in the 1st specific embodiment of the second embodiment.

In a 4^{th} specific embodiment, for immunoconjugate of formula (C1), Cy^{C1} is represented by formula (C1a) or (C1a1); Rₐ and R_{b} are both H; and R₅ is H or Me; and the remaining variables are as described above in the 1^{st} or 2^{nd} specific embodiment of the second embodiment.

In a 5^{th} specific embodiment, for immunoconjugate of formula (C1), P is a peptide containing 2 to 5 amino acid residues; and the remaining variables are as described above in the 1^{st}, 2^{nd} or 4^{th} specific embodiment of the second embodiment. In a more specific embodiment, P is selected from Gly-Gly-Gly, Ala-Val, Val-Ala, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:74), β-Ala-Leu-Ala-Leu (SEQ ID NO:75), Gly-Phe-Leu-Gly (SEQ ID NO:76), Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. In another more specific embodiment, P is Gly-Gly-Gly, Ala-Val, Ala-Ala, Ala-D-Ala, D-Ala-Ala, or D-Ala-D-Ala.

In a 6^{th} specific embodiment, for immunoconjugates of formula (C1), Q is —SO₃H or a pharmaceutically acceptable salt thereof; and the remaining variables are as describe above in the 1^{st}, 2^{nd}, 4^{th} or 5^{th} specific embodiment of the second embodiment or any more specific embodiments described therein.

In a 7^{th} specific embodiment, for immunoconjugates of formula (C1), R₁₉ and R₂₀ are both H; and m″ is an integer from 1 to 6; and the remaining variables are as described above in the 1^{st}, 2^{nd}, 3^{rd}, 4^{th}, 5^{th} or 6^{th} specific embodiment of the second embodiment or any more specific embodiments described therein.

In a 8^{th} specific embodiment, for immunoconjugates of formula (C1), -L-L_{C}- is represented by the following formula:

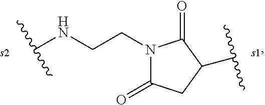 [SMILES: *NCCN1C(=O)CC(*)C1=O]

and the remaining variables are as described above in the 1^{st}, 2^{nd}, 3^{rd}, 4^{th}, 5^{th}, 6^{th} or 7^{th} specific embodiment of the second embodiment or any more specific embodiments described therein.

In a 9^{th} specific embodiment, the immunoconjugate of the second embodiment is represented by the following formula:

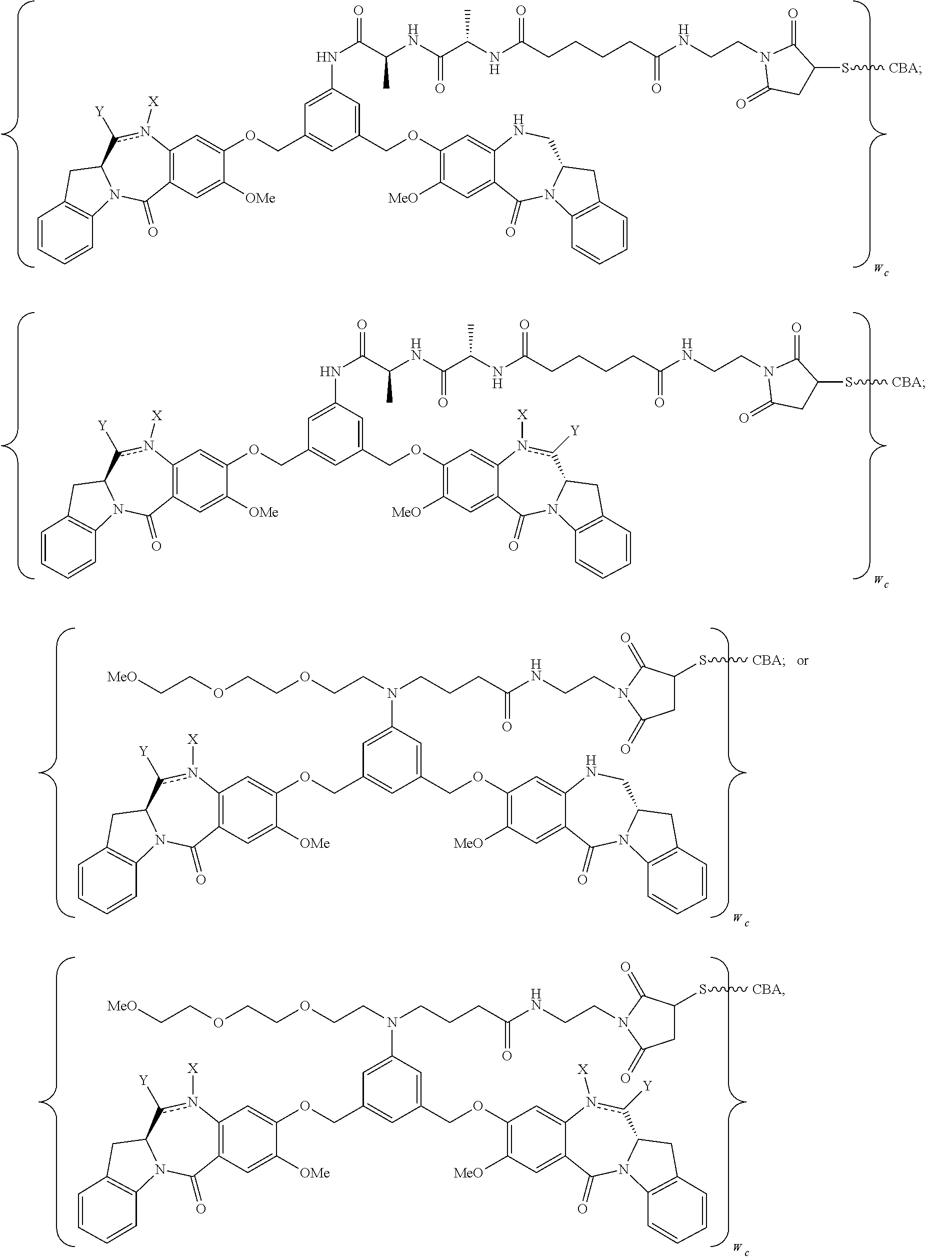

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO₃H or a pharmaceutically acceptable salt thereof. In a more specific embodiment, the double line [symbol] between N and C represents a double bond, X is absent and Y is —H. In another more specific embodiment, the double line [symbol] between N and C represents a single bond, X is —H and Y is —SO₃H or a pharmaceutically acceptable salt thereof.

In a 10^{th} specific embodiment, the immunoconjugate of the second embodiment is represented by the following formula:

CBACy^{C2})_{W}_{_{C}} (C2),

wherein:

CBA is a MET-binding agent (e.g., an anti-MET antibody or an antibody fragment thereof) described herein above that is covalently linked to Cy^{C2} through a cysteine residue;

W_{C} is 1 or 2;

Cy^{C2} is represented by the following formula:

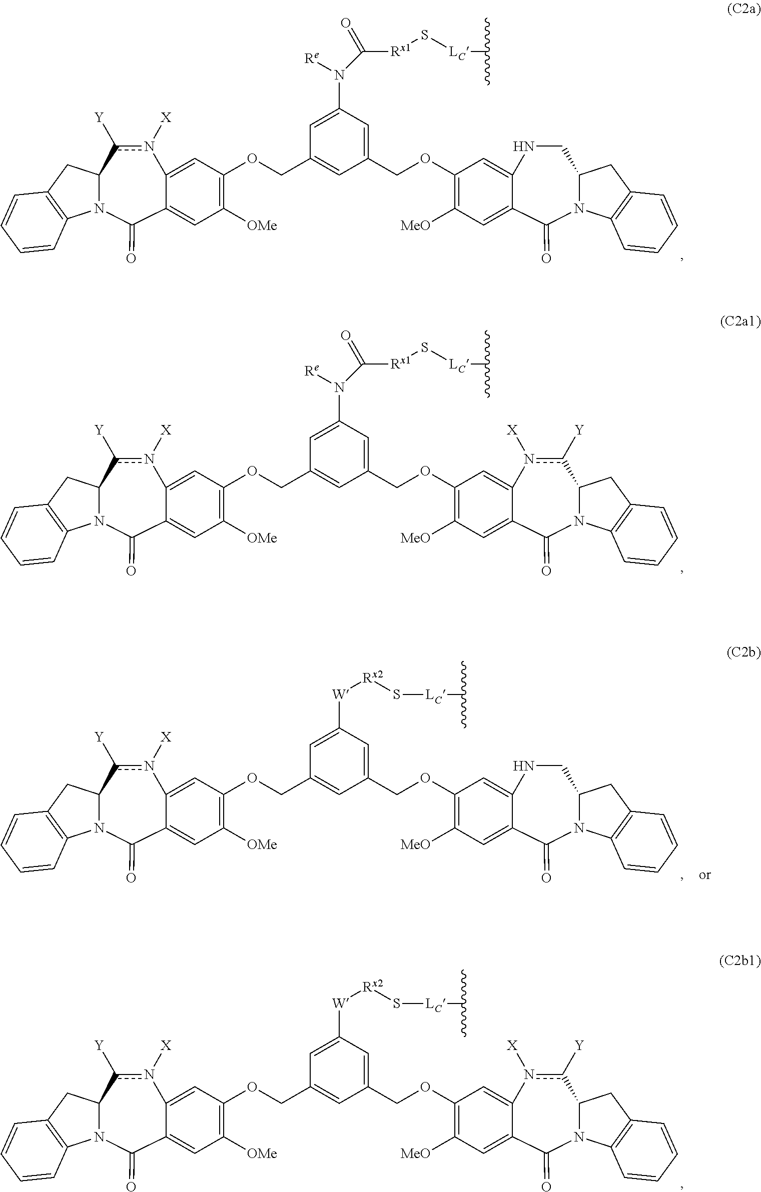

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃H or a pharmaceutically acceptable salt thereof;

R^{x1} is a (C₁-C₆)alkyl;

R^{e} is —H or a (C₁-C₆)alkyl;

W′ is —NR^{e′};

R^{e′} is —(CH₂—CH₂—O)ₙ—R^{k};

n is an integer from 2 to 6;

R^{k} is —H or -Me;

R^{x2} is a (C₁-C₆)alkyl;

L_{C}′ is represented by the following formula:

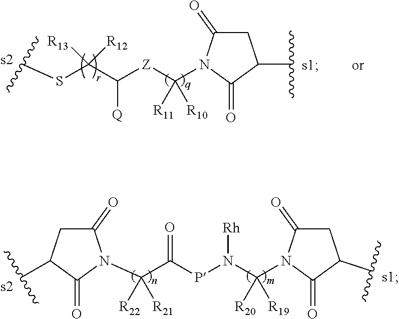

wherein:

s1 is the site covalently linked to the CBA and s2 is the site covalently linked to —S— group on Cy^{C2};

Z is —C(═O)—NR₉—, or —NR₉—C(═O)—;

Q is —H, a charged substituent, or an ionizable group;

R₉, R₁₀, R₁₁, R₁₂, R₁₃, R₁₉, R₂₀, R₂₁ and R₂₂, for each occurrence, are independently —H or a (C₁-C₃)alkyl;

q and r, for each occurrence, are independently an integer between 0 and 10;

m and n are each independently an integer between 0 and 10;

R^{h} is —H or a (C₁-C₃)alkyl; and

P′ is an amino acid residue or a peptide containing 2 to 20 amino acid residues.

In a more specific embodiment, q and r are each independently an integer between 1 to 6, more specifically, an integer between 1 to 3. Even more specifically, R₁₀, R₁₁, R₁₂ and R₁₃ are all H.

In another more specific embodiment, m and n are each independently an integer between 1 and 6, more specifically, an integer between 1 to 3. Even more specifically, R₁₉, R₂₀, R₂₁ and R₂₂ are all H.

In a 11^{th} specific embodiment, for immunoconjugates of formula (C2), Cy^{C2} is represented by formula (C2a) or (C2a1); and the remaining variables are as described above in the 10^{th} specific embodiment of the second embodiment or any more specific embodiments described therein.

In a 12^{th} specific embodiment, for immunoconjugates of formula (C2), Cy^{C2} is represented by formula (C2b) or (C2b1); and the remaining variables are as described above in the 10^{th} specific embodiment of the second embodiment.

In a 13^{th} specific embodiment, for immunoconjugates of formula (C2), P′ is a peptide containing 2 to 5 amino acid residues; and the remaining variables are as described in the 10^{th}, 11^{th} or 12^{th} specific embodiment of the second embodiment or any more specific embodiments described therein. In a more specific embodiment, P′ is selected from Gly-Gly-Gly, Ala-Val, Val-Ala, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:74), β-Ala-Leu-Ala-Leu (SEQ ID NO:75), Gly-Phe-Leu-Gly (SEQ ID NO:76), Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. In another more specific embodiment, P′ is Gly-Gly-Gly, Ala-Val, Ala-Ala, Ala-D-Ala, D-Ala-Ala, or D-Ala-D-Ala.

In a 14^{th} specific embodiment, for immunoconjugates of formula (C2), -L_{C}′- is represented by the following formula:

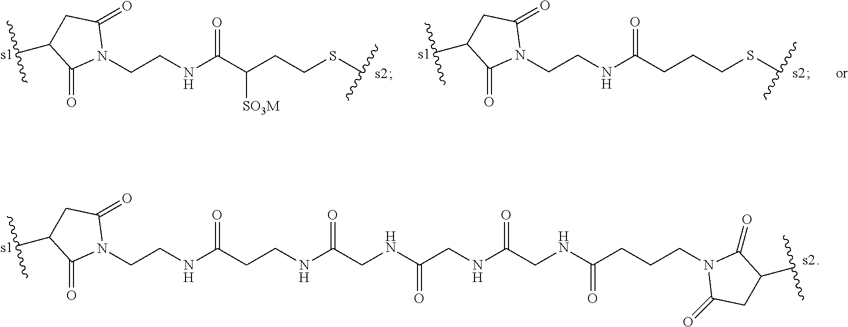

In a 15^{th} specific embodiment, for immunoconjugates of (C2), R^{e} is H or Me; R^{x1} is —(CH₂)ₚ—(CR^{f}R^{g})—, and R^{x2} is —(CH₂)ₚ—(CR^{f}R^{g})—, wherein R^{f} and R^{g} are each independently —H or a (C₁-C₄)alkyl; and p is 0, 1, 2 or 3; and the remaining variables are as described above in the 10^{th}, 11^{th}, 12^{th}, 13^{th}, or 14^{th} specific embodiment of the second embodiment. More specifically, R^{f} and R^{g} are the same or different, and are selected from —H and -Me.

In a 16^{th} specific embodiment, the immunoconjugate of the second embodiment is represented by the following formula:

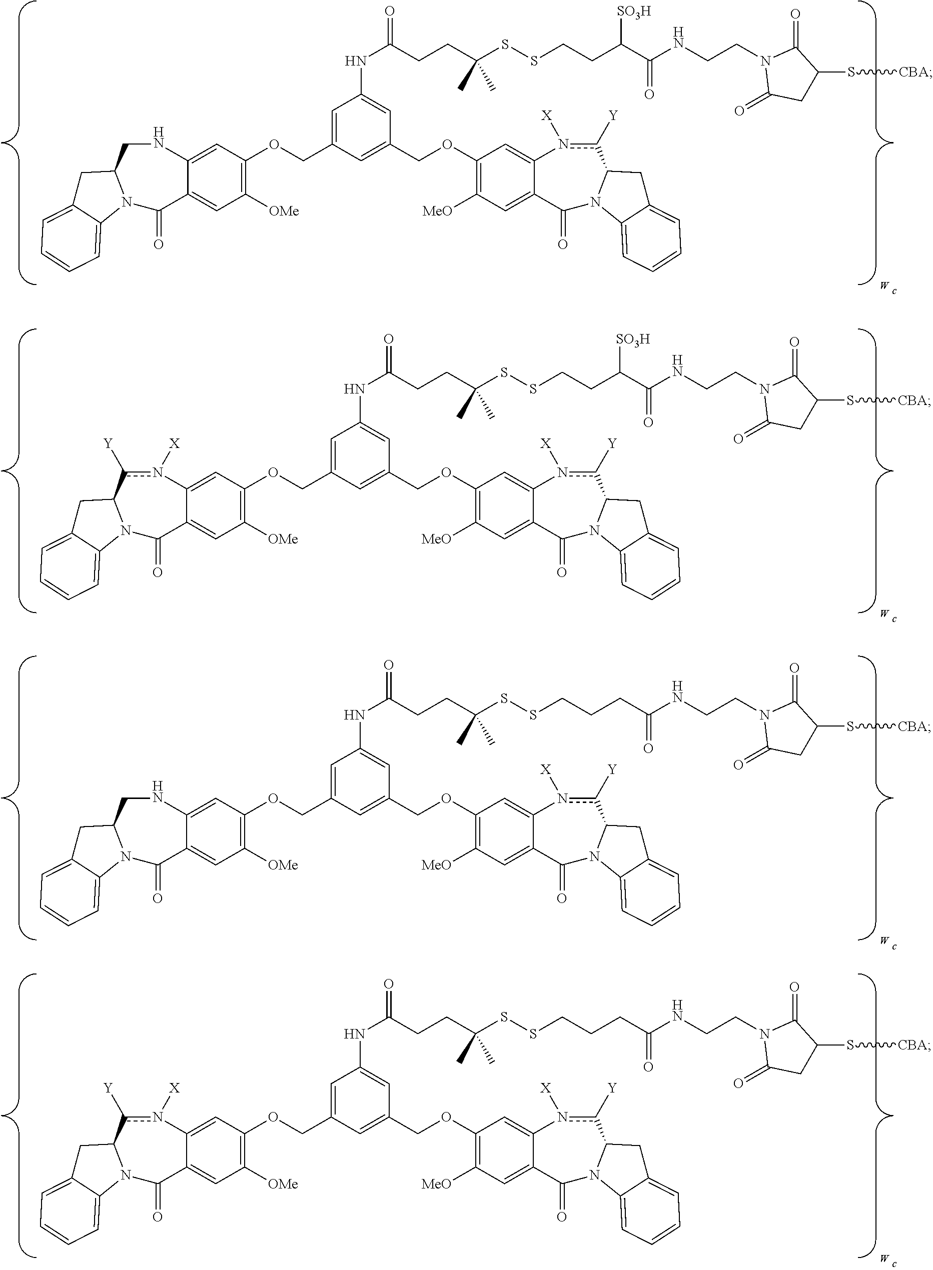

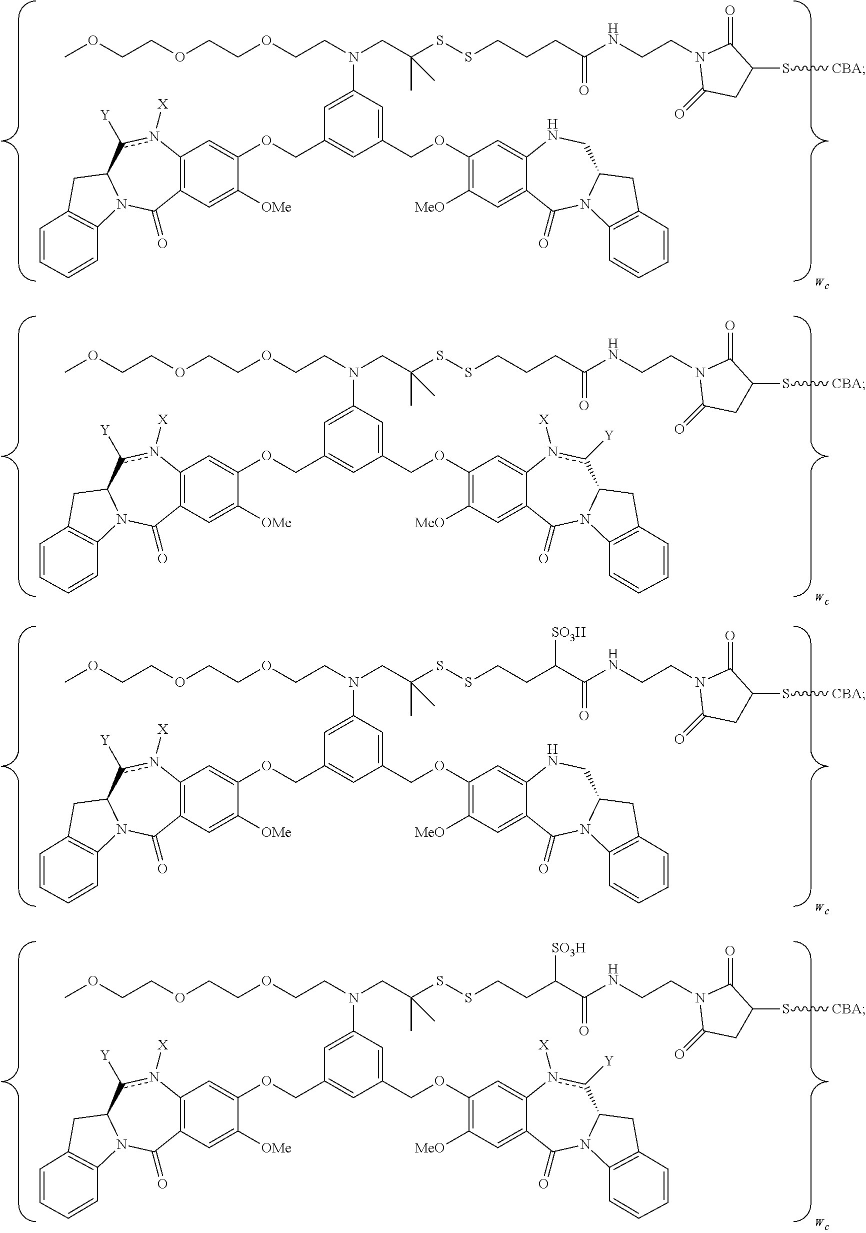

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO₃H or a pharmaceutically acceptable salt thereof. In a more specific embodiment, the double line [symbol] between N and C represents a double bond, X is absent and Y is —H. In another specific embodiment, the double line [symbol] between N and C represents a single bond, X is —H and Y is —SO₃H or a pharmaceutically acceptable salt thereof.

In a 17^{th} specific embodiment, the immunoconjugate of the second embodiment is represented by the following formula:

CBACy^{C3})_{W}_{_{C}} (C3),

wherein:

CBA is a MET-binding agent (e.g., an anti-MET antibody or an antibody fragment thereof) described herein above covalently linked to Cy^{C3} through a cysteine residue;

W_{C} is 1 or 2;

Cy^{C3} is represented by the following formula:

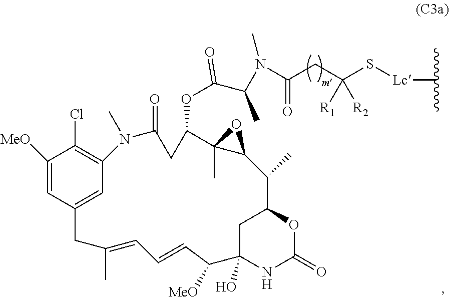

wherein:

m′ is 1 or 2;

R₁ and R₂, are each independently —H or a (C₁-C₃)alkyl;

L_{C}′ is represented by the following formula:

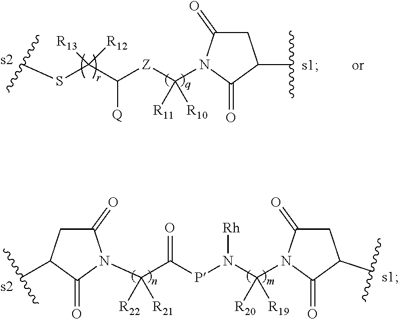

wherein:

s1 is the site covalently linked to the CBA and s2 is the site covalently linked to —S— group on Cy^{C3};

Z is —C(═O)—NR₉—, or —NR₉—C(═O)—;

Q is H, a charged substituent, or an ionizable group;

R₉, R₁₀, R₁₁, R₁₂, R₁₃, R₁₉, R₂₀, R₂₁ and R₂₂, for each occurrence, are independently —H or a (C₁-C₃)alkyl;

q and r, for each occurrence, are independently an integer between 0 and 10;

m and n are each independently an integer between 0 and 10;

R^{h} is —H or a (C₁-C₃)alkyl; and

P′ is an amino acid residue or a peptide containing 2 to 20 amino acid residues.

In a more specific embodiment, q and r are each independently an integer between 1 to 6, more specifically, an integer from 1 to 3. Even more specifically, R₁₀, R₁₁, R₁₂ and R₁₃ are all H.

In another more specific embodiment, m and n are each independently an integer between 1 and 6, more specifically, an integer from 1 to 3. Even more specifically, R₁₉, R₂₀, R₂₁ and R₂₂ are all H.

In a 18^{th} specific embodiment, for immunoconjugates of formula (C3), P′ is a peptide containing 2 to 5 amino acid residues; and the remaining variables are as described above in the 17^{th} specific embodiment of the second embodiment or any more specific embodiments described therein. In a more specific embodiment, P′ is selected from Gly-Gly-Gly, Ala-Val, Val-Ala, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:74), β-Ala-Leu-Ala-Leu (SEQ ID NO:75), Gly-Phe-Leu-Gly (SEQ ID NO:76), Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. In another more specific embodiment, P′ is Gly-Gly-Gly, Ala-Val, Ala-Ala, Ala-D-Ala, D-Ala-Ala, or D-Ala-D-Ala.

In a 19^{th} specific embodiment, for immunoconjugates of formula (C3), -L_{C}′- is represented by the following formula:

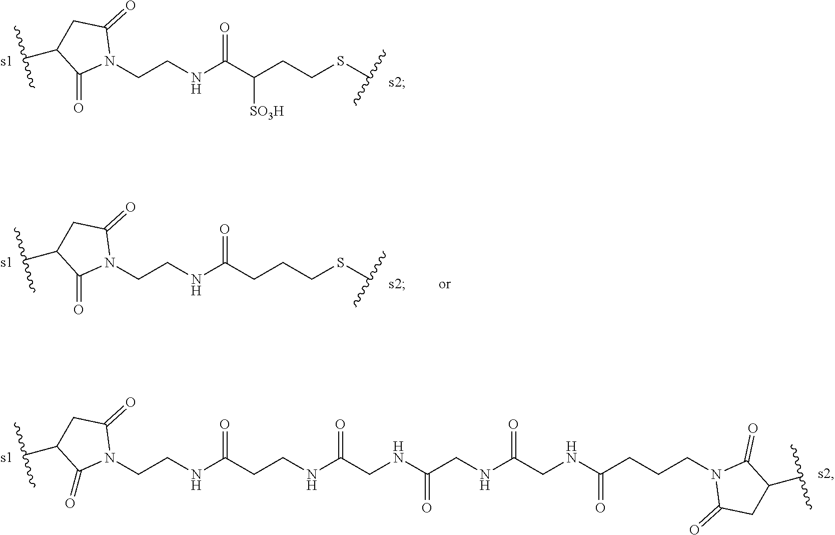

wherein M is H⁺ or a cation; and the remaining variables are as described above in the 17^{th} or 18^{th} specific embodiment of the second embodiment or any more specific embodiments described therein.

In a 20^{th} specific embodiment, for immunoconjugates of formula (C3), m′ is 1 and R₁ and R₂ are both H; and the remaining variables are as described above in the 17^{th}, 18^{th} or 19^{th} specific embodiment of the second embodiment or any more specific embodiments described therein.

In a 21^{st} specific embodiment, for immunoconjugates of formula (C3), m′ is 2 and R₁ and R₂ are both Me; and the remaining variables are as described above in the 17^{th}, 18^{th} or 19^{th} specific embodiment of the second embodiment or any more specific embodiments described therein.

In a 22^{nd} specific embodiment, the immunoconjugate of the second embodiment is represented by the following formula:

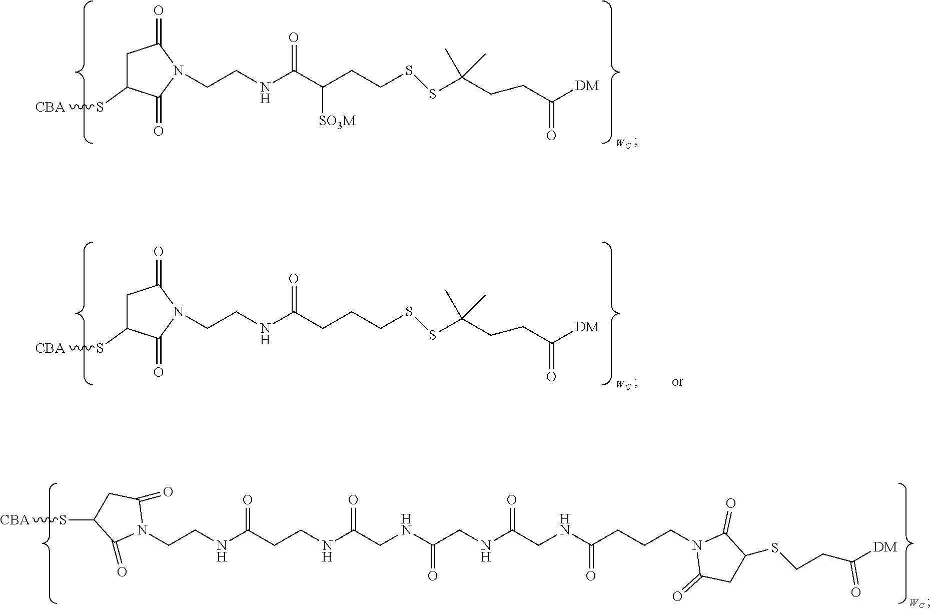

or a pharmaceutically acceptable salt thereof, wherein DM is a drug moiety represented by the following formula:

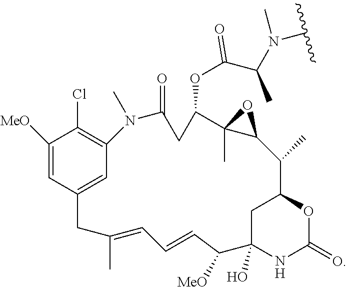 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In a 23^{rd} specific embodiment, for the immunoconjugates of the second embodiment, Y is —SO₃H, —SO₃Na or —SO₃K; and the remaining variables are as described in any one of the 1^{st} to 22^{nd} specific embodiments of the second embodiment or any more specific embodiments described therein. In one embodiment, Y is —SO₃Na.

B. Exemplary Linker Molecules

Any suitable linkers known in the art can be used in preparing the immunoconjugates of the present invention. In certain embodiments, the linkers are bifunctional linkers. As used herein, the term “bifunctional linker” refers to modifying agents that possess two reactive groups; one of which is capable of reacting with a cell binding agent while the other one reacts with the cytotoxic compound to link the two moieties together. Such bifunctional crosslinkers are well known in the art (see, for example, Isalm and Dent in Bioconjugation chapter 5, p218-363, Groves Dictionaries Inc. New York, 1999). For example, bifunctional crosslinking agents that enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that introduce maleimido groups or haloacetyl groups on to a cell binding agent are well known in the art (see US Patent Applications 2008/0050310, 20050169933, available from Pierce Biotechnology Inc. P.O. Box 117, Rockland, Ill. 61105, USA) and include, but not limited to, bis-maleimidopolyethyleneglycol (BMPEO), BM(PEO)₂, BM(PEO)₃, N-(β-maleimidopropyloxy)succinimide ester (BMPS), γ-maleimidobutyric acid N-succinimidyl ester (GMBS), ε-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), κ-maleimidoundecanoic acid N-succinimidyl ester (KMUA), N-succinimidyl 4-(p-maleimidophenyl)-butyrate (SMPB), succinimidyl-6-(β-maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(γ-maleimidobutryloxy)sulfosuccinimide ester (sulfo-GMBS), N-(ε-maleimidocaproyloxy)sulfosuccimido ester (sulfo-EMCS), N-(κ-maleimidoundecanoyloxy)sulfosuccinimide ester (sulfo-KMUS), and sulfosuccinimidyl 4-(p-maleimidophenyl)butyrate (sulfo-SMPB).

Heterobifunctional crosslinking agents are bifunctional crosslinking agents having two different reactive groups. Heterobifunctional crosslinking agents containing both an amine-reactive N-hydroxysuccinimide group (NHS group) and a carbonyl-reactive hydrazine group can also be used to link the cytotoxic compounds described herein with a cell-binding agent (e.g., antibody). Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present invention. Examples of bifunctional crosslinking agents that can be used include succinimidyl-p-formyl benzoate (SFB) and succinimidyl-p-formylphenoxyacetate (SFPA).

Bifunctional crosslinking agents that enable the linkage of cell binding agent with cytotoxic compounds via disulfide bonds are known in the art and include N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. Other bifunctional crosslinking agents that can be used to introduce disulfide groups are known in the art and are disclosed in U.S. Pat. Nos. 6,913,748, 6,716,821 and US Patent Publications 20090274713 and 20100129314, all of which are incorporated herein by reference. Alternatively, crosslinking agents such as 2-iminothiolane, homocysteine thiolactone or S-acetylsuccinic anhydride that introduce thiol groups can also be used.

In certain embodiments, the bifunctional linkers are represented by any one of the formula (a1L)-(a10L) described below.

C. Exemplary Cytotoxic Agents

1. Maytansinoid

In certain embodiments, the cytotoxic agent is a maytansinoid compound, such as those described in U.S. Pat. Nos. 5,208,020 and 7,276,497, incorporated herein by reference in its entirety. In certain embodiments, the maytansinoid compound is represented by the following formula:

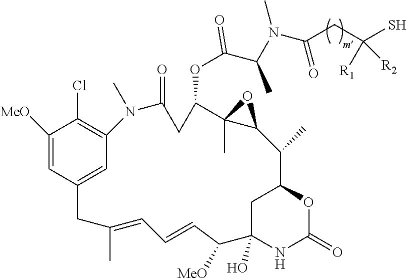

wherein the variables are as described above in any one of the 13^{th} to 15^{th} specific embodiments of the first embodiment above and any more specific embodiments described therein.

In a more specific embodiment, the maytansinoid compound is DM4:

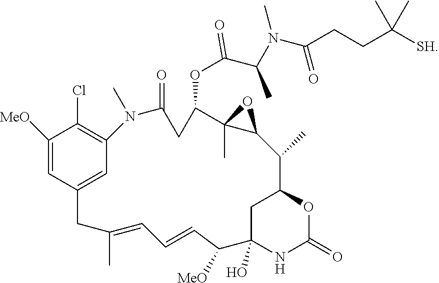 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)[C@@H]1C[C@@](O)(NC(=O)O1)[C@H](OC)/C=C/C=C(\C)C2]

In another embodiment, the maytansinoid compound is DM1:

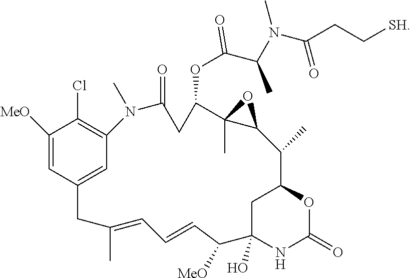 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

2. Benzodiazepine

In certain embodiments, the cytotoxic agent is a benzodiazepine compound, such as pyrrolobenzodiazepine (PBD) (such as those described in WO2010/043880, WO2011/130616, WO2009/016516, WO 2013/177481 and WO 2012/112708) and indolinobenzodiazepine (IGN) compounds (such as those described in WO/2010/091150, and WO 2012/128868 and U.S. application Ser. No. 15/195,269, filed on Jun. 28, 2016, entitled “CONJUGATES OF CYSTEINE ENGINEERED ANTIBODIES”. The entire teachings of all of these patents, patent publications and applications are incorporate herein by reference in their entireties.

As used herein, a “benzodiazepine” compound is a compound having a benzodiazepine core structure. The benzodiazepine core can be substituted or unsubstituted, and/or fused with one or more ring structures. It also includes a compound having two benzodiazepine core linked by a linker. The imine functionality (—C═N—) as part of benzodiazepine core can be reduced.

As used herein, a “pyrrolobenzodiazepine” (PBD) compound is a compound having a pyrrolobenzodiazepine core structure. The pyrrolobenzodiazepine can be substituted or unsubstituted. It also includes a compound having two pyrrolobenzodiazepine core linked by a linker. The imine functionality (—C═N—) as part of indolinobenzodiazepine core can be reduced.

In certain embodiments, the cytotoxic agent is an indolinobenzodiazepine compound represented by the following formula:

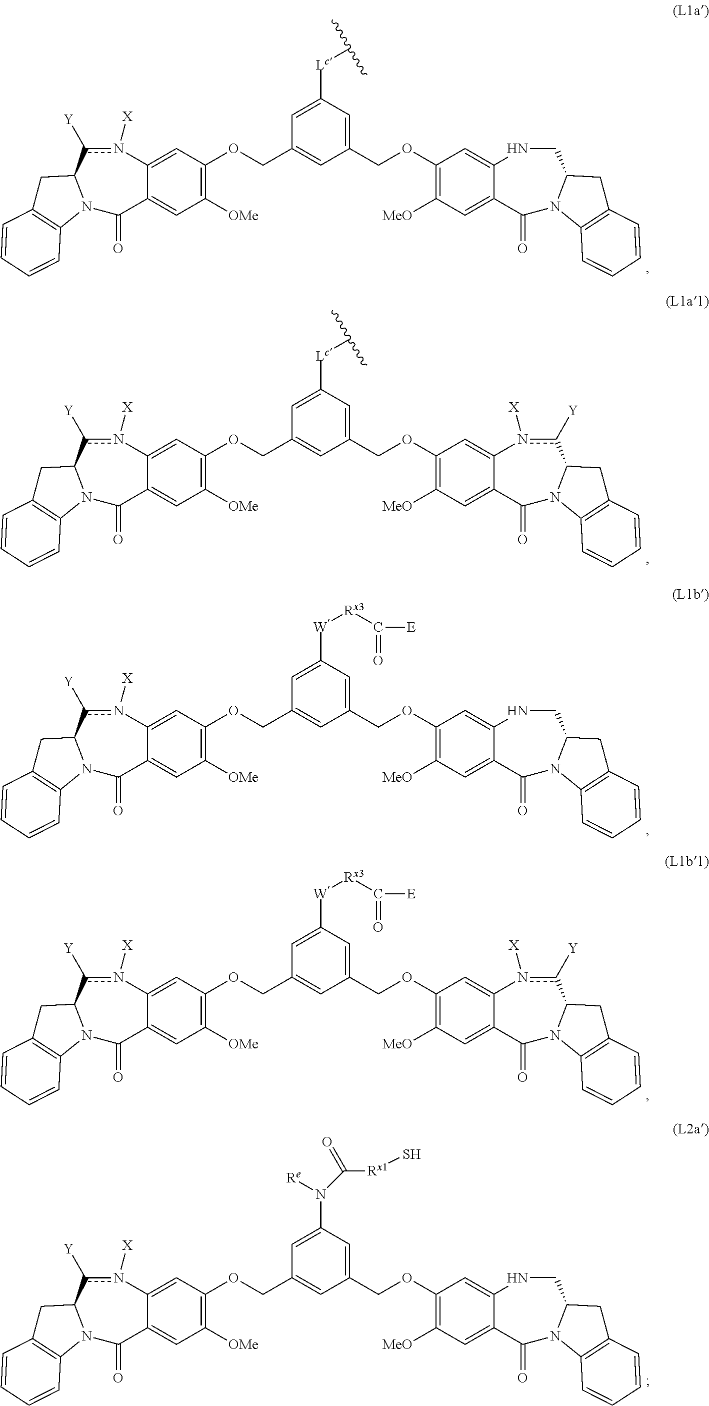

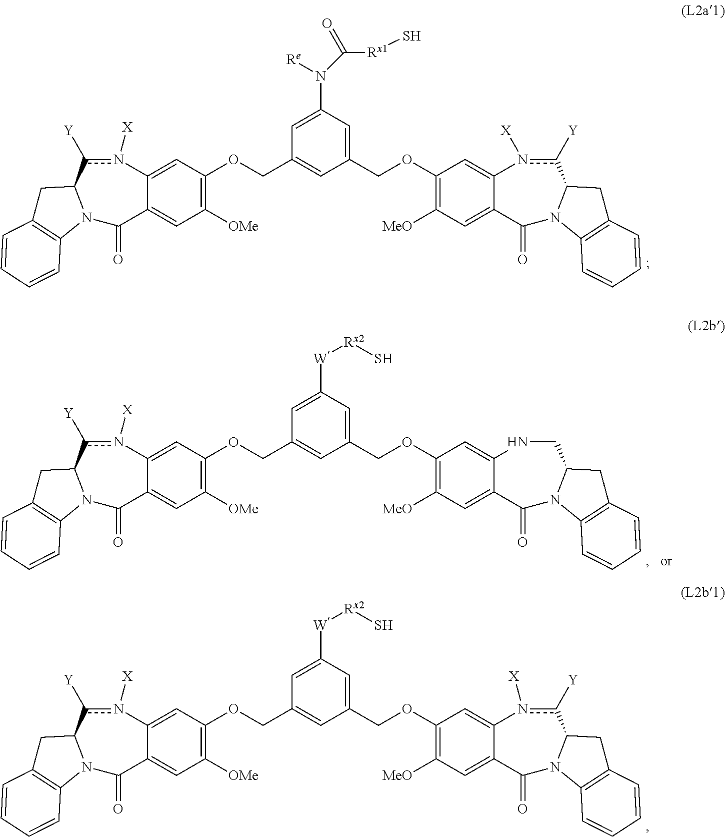

or a pharmaceutically acceptable salt thereof, wherein:

L^{c}′ is represented by the following formula:

—NR₅—P—C(═O)—(CRₐR_{b})ₘ—C(═O)E (B 1); or

—NR₅—P—C(═O)—(CRₐR_{b})ₘ—S—Z^{S} (B2)

C(═O)E is a reactive ester group, such as N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, or pentafluorophenyl ester, preferably N-hydroxysuccinimide ester;

Z^{S} is represented by the following formula:

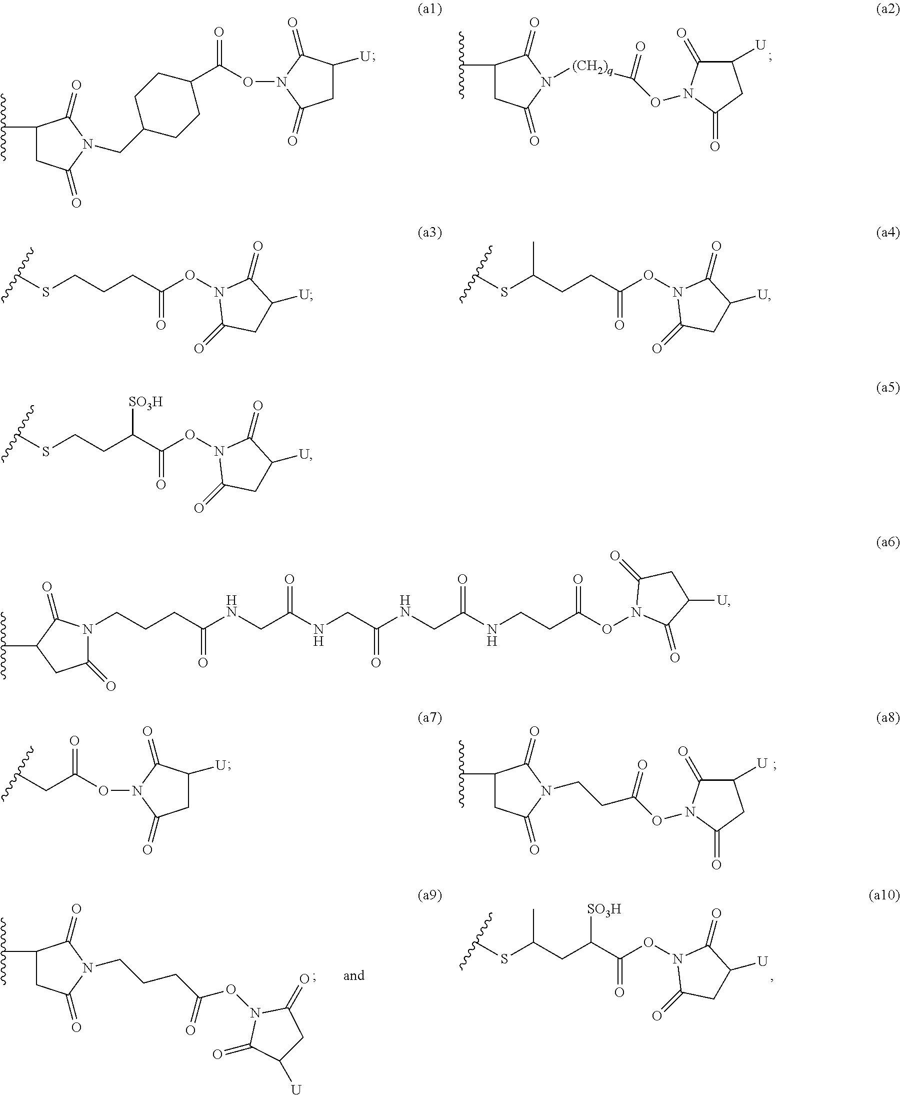

wherein:

q is an integer from 1 to 5; and

U is —H or SO₃H or a pharmaceutically acceptable salt thereof; and the remaining variables are as described in any one of the 1^{st} to 12^{th} and 17^{th} specific embodiments of the first embodiment described above or any more specific embodiments described therein.

In certain embodiments, the cytotoxic agent is an indolinobenzodiazepine compound represented by the following formula:

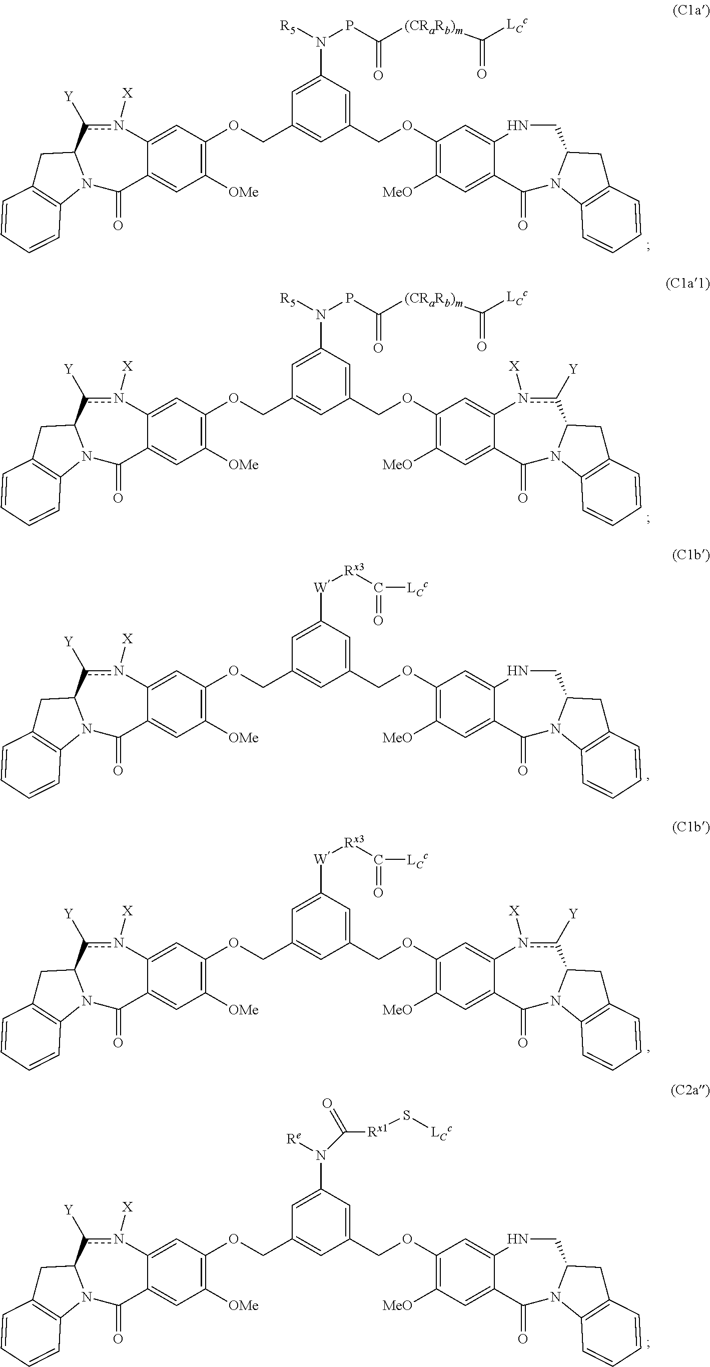

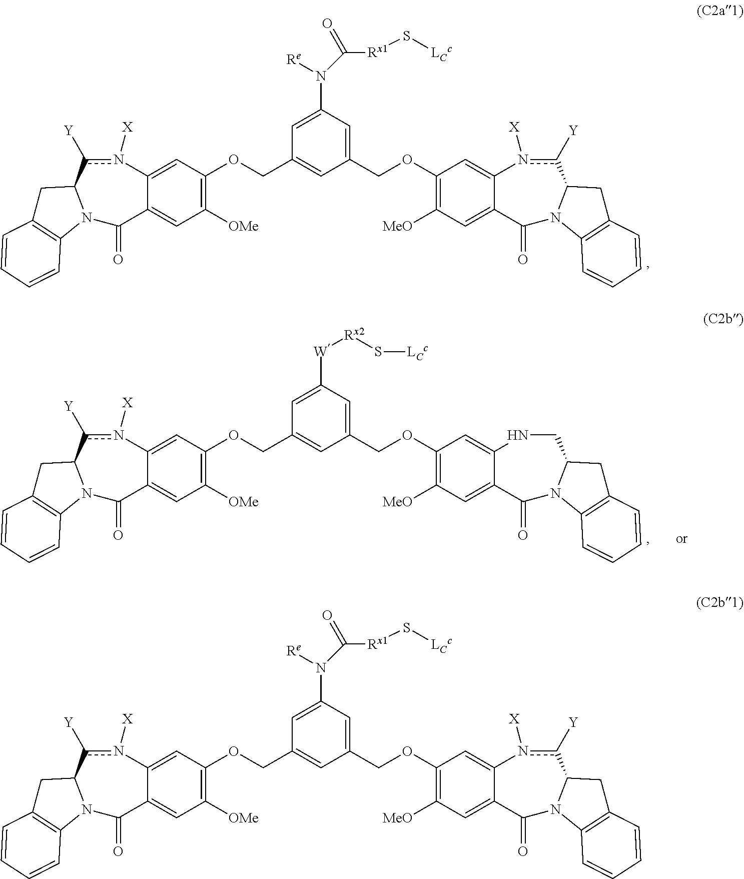

or a pharmaceutically acceptable salt thereof, wherein:

-L_{C}^{c} for formulas (C1a′), (C1a′1), (C1b′) and (C1b′1) is represented by the following formula:

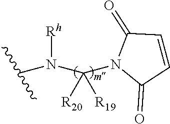

wherein the variables are as described above in any one of the 1^{st} to 9^{th} and 23^{rd} specific embodiments of the second embodiment or any more specific embodiments described therein; and

Lc^{c}′ for formulas (C2a″), (C2a″1), (C2b″) and (C2b″1) is represented by the following formula:

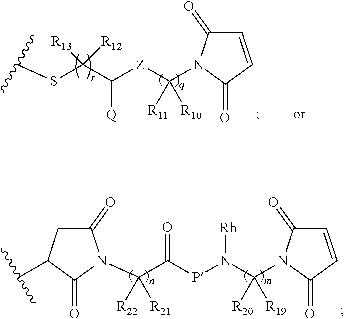

wherein the variables are as described above in any one of the 10^{th} to 16^{th} and 23^{rd} specific embodiment of the second embodiment or any more specific embodiments described therein.

In certain embodiments, the cytotoxic agent is an indolinobenzodiazepine compound of any one of the following or a pharmaceutically acceptable salt thereof:

| Com- | |
|---|---|
| pound | |
| No. | Structure |
| D1 | 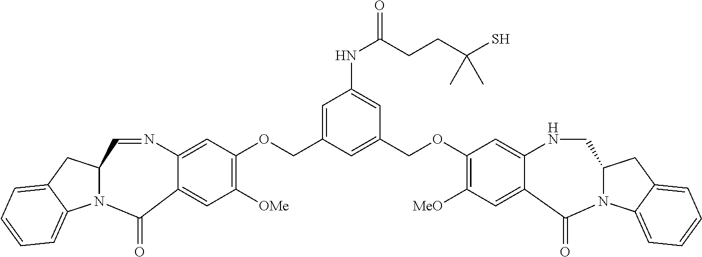 [SMILES: COc1cc2c(cc1OCc1cc(COc3cc4c(cc3OC)C(=O)N3c5ccccc5C[C@H]3CN4)cc(NC(=O)CCC(C)(C)S)c1)N=C[C@@H]1Cc3ccccc3N1C2=O] |
| sD1 | 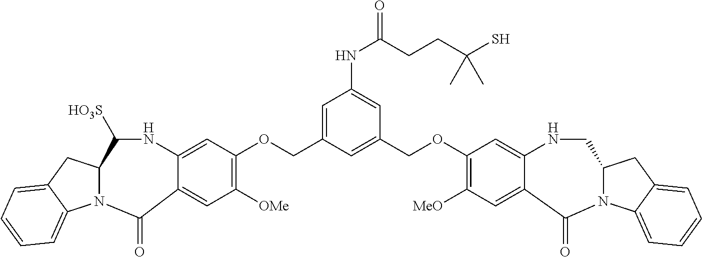 |
| D2 | 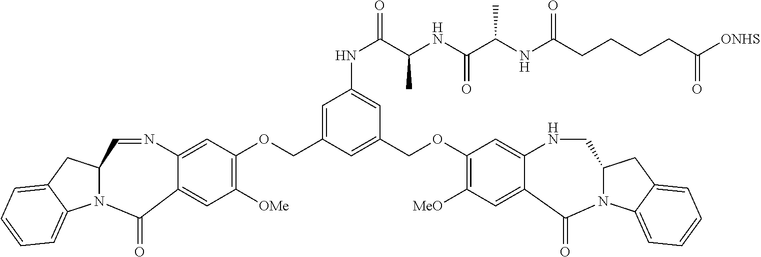 |
| sD2 | 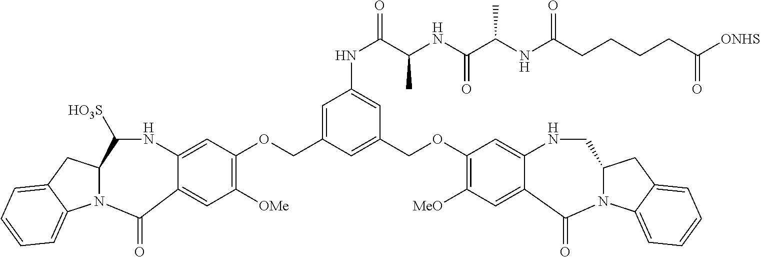 |
| DGN462 | 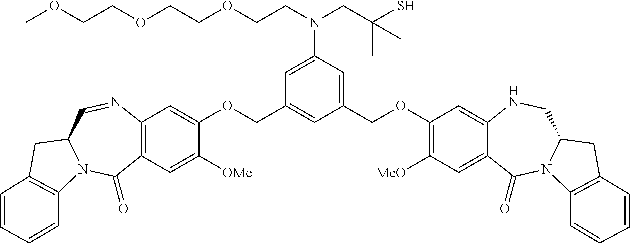 [SMILES: COCCOCCOCCN(CC(C)(C)S)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1] |
| sDGN462 | 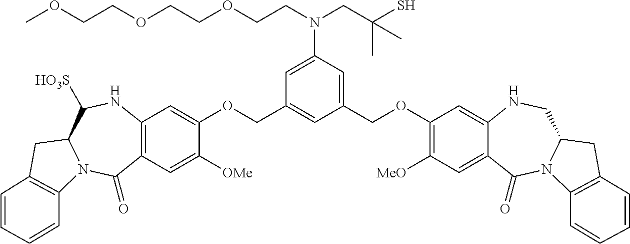 |
| D3 | 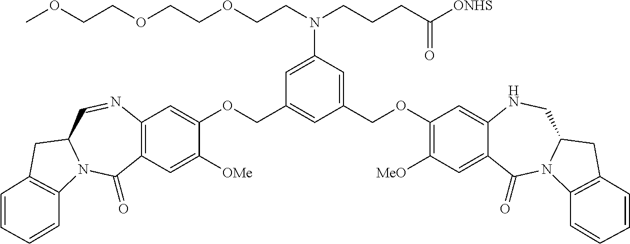 |
| sD3 | 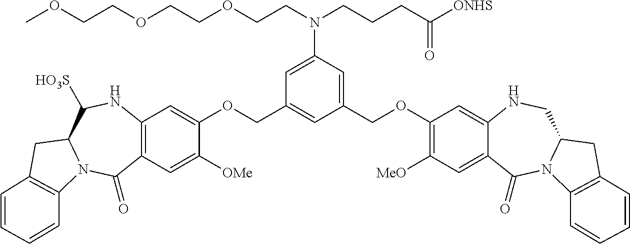 |
| D4 | 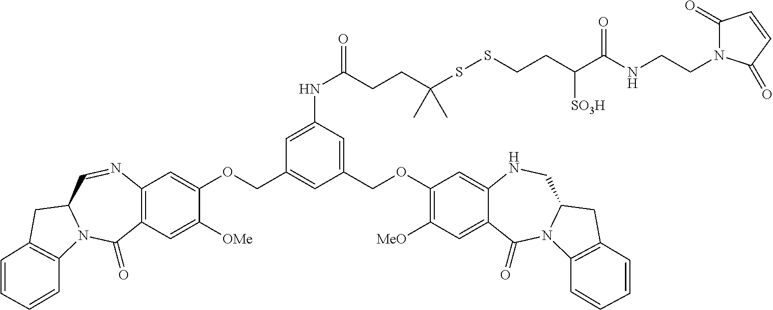 [SMILES: COc1cc2c(cc1OCc1cc(COc3cc4c(cc3OC)C(=O)N3c5ccccc5C[C@H]3CN4)cc(NC(=O)CCC(C)(C)SSCCC(C(=O)NCCN3C(=O)C=CC3=O)S(=O)(=O)O)c1)N=C[C@@H]1Cc3ccccc3N1C2=O] |
| sD4 | 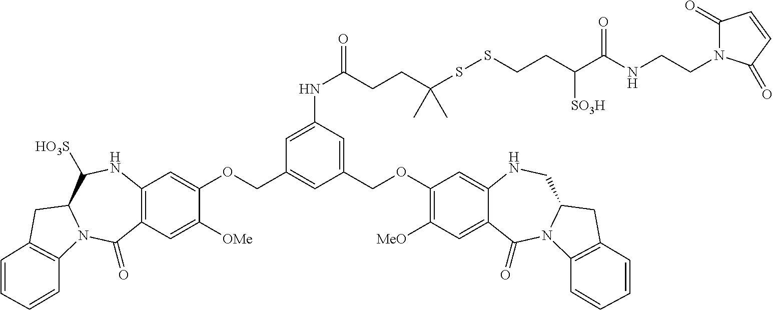 |
| D5 | 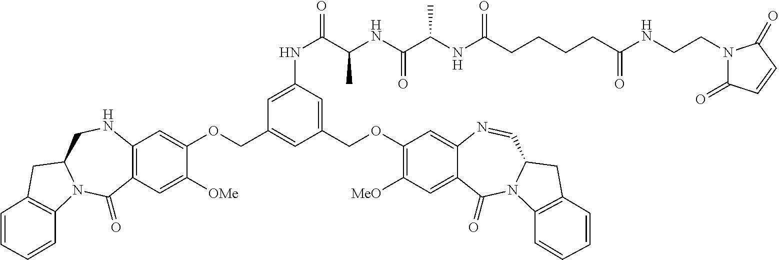 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O] |
| sD5 | 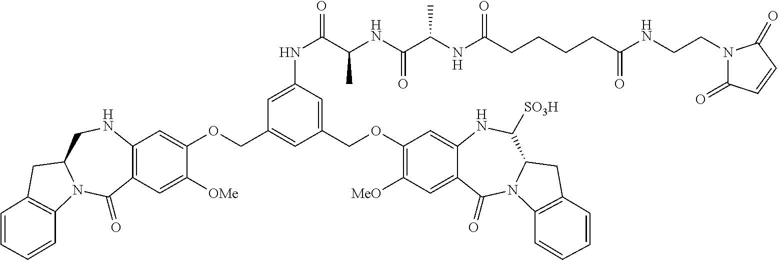 |
| D5′ | 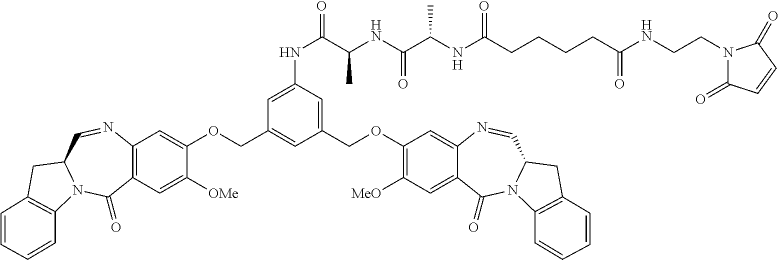 [SMILES: COc1cc2c(cc1OCc1cc(COc3cc4c(cc3OC)C(=O)N3c5ccccc5C[C@H]3C=N4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O] |
| sD5′ | 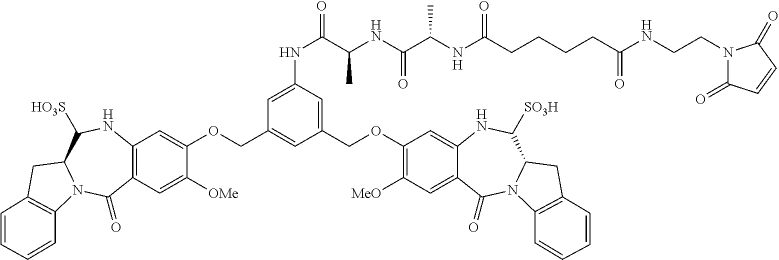 |
| D6 | 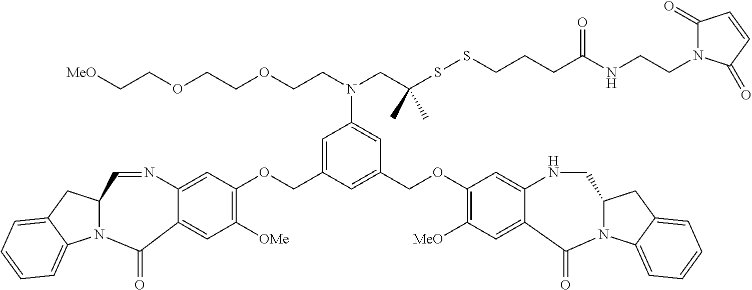 |
| sD6 | 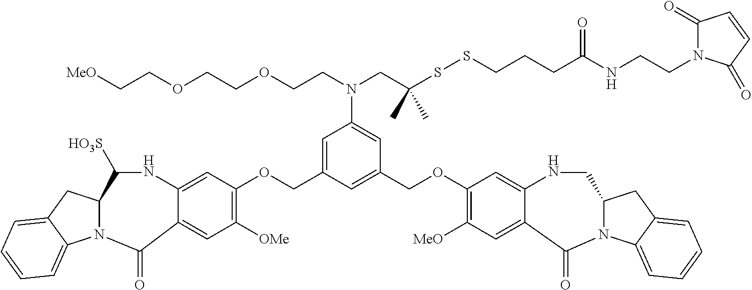 |
| D7 | 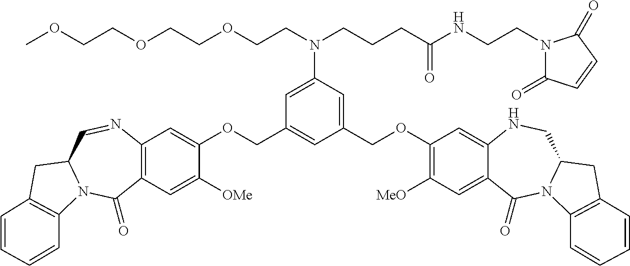 [SMILES: COCCOCCOCCN(CCCC(=O)NCCN1C(=O)C=CC1=O)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1] |
| sD7 | 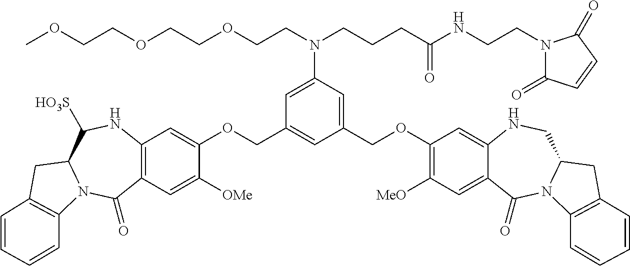 |

Compounds D1, sD1, D2, sD2, DGN462, sDGN462, D3 and sD3 shown above can be prepared according to procedures described in U.S. Pat. Nos. 9,381,256, 8,765,740, 8,426,402, and 9,353,127, and U.S. Application Publication US2016/0082114, all of which are incorporated herein by reference in their entireties.

In certain embodiments, the pharmaceutically acceptable salt of the compounds shown above (e.g., sD1, sD2, sD4, sDGN462, sD3, sD4, sD5, sD5′, sD6 or sD7) is a sodium or potassium salt. More specifically, the pharmaceutically acceptable salt is a sodium salt.

In a specific embodiment, the cytotoxic agent is represented by the following formula:

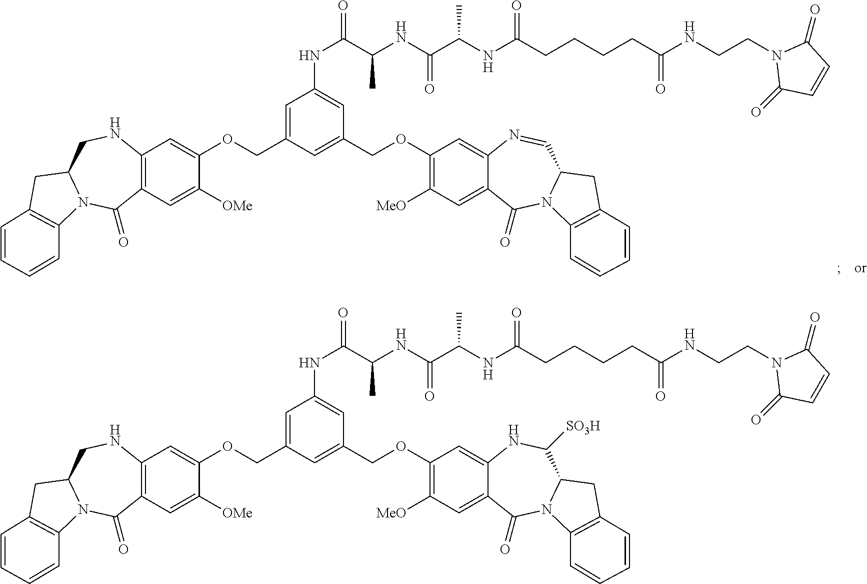

or a pharmaceutically acceptable salt thereof. In a specific embodiment, the pharmaceutically acceptable salt is a sodium or a potassium salt.

In another specific embodiment, the cytotoxic agent is represented by the following formula:

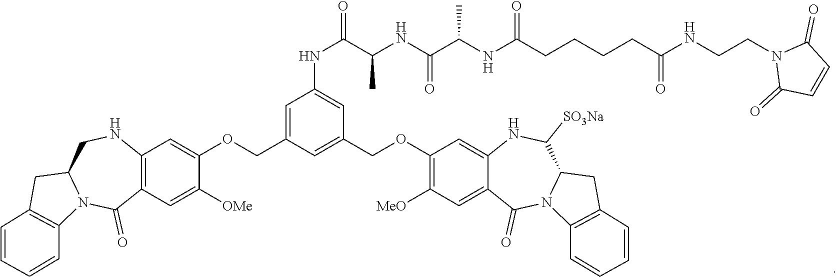

### IV. Drug Conjugation

The immunoconjugates comprising a MET-binding agent covalently linked to a cytotoxic agent through the ε-amino group of one or more lysine residues located on the MET-binding agent as described the first embodiment above or any specific embodiments described therein can be prepared according to any methods known in the art, see, for example, WO 2012/128868 and WO2012/112687, which are incorporate herein by reference.

In certain embodiments, the immunoconjugates of the first embodiment can be prepared by a first method comprising the steps of reacting the CBA (i.e. a MET-binding agent described herein) with the cytotoxic agent having an amine reactive group.

In one embodiment, for the first method described above, the reaction is carried out in the presence of an imine reactive reagent, such as NaHSO₃.

In one embodiment, for the first method described above the cytotoxic agent having an amine reactive group is represented by the following formula:

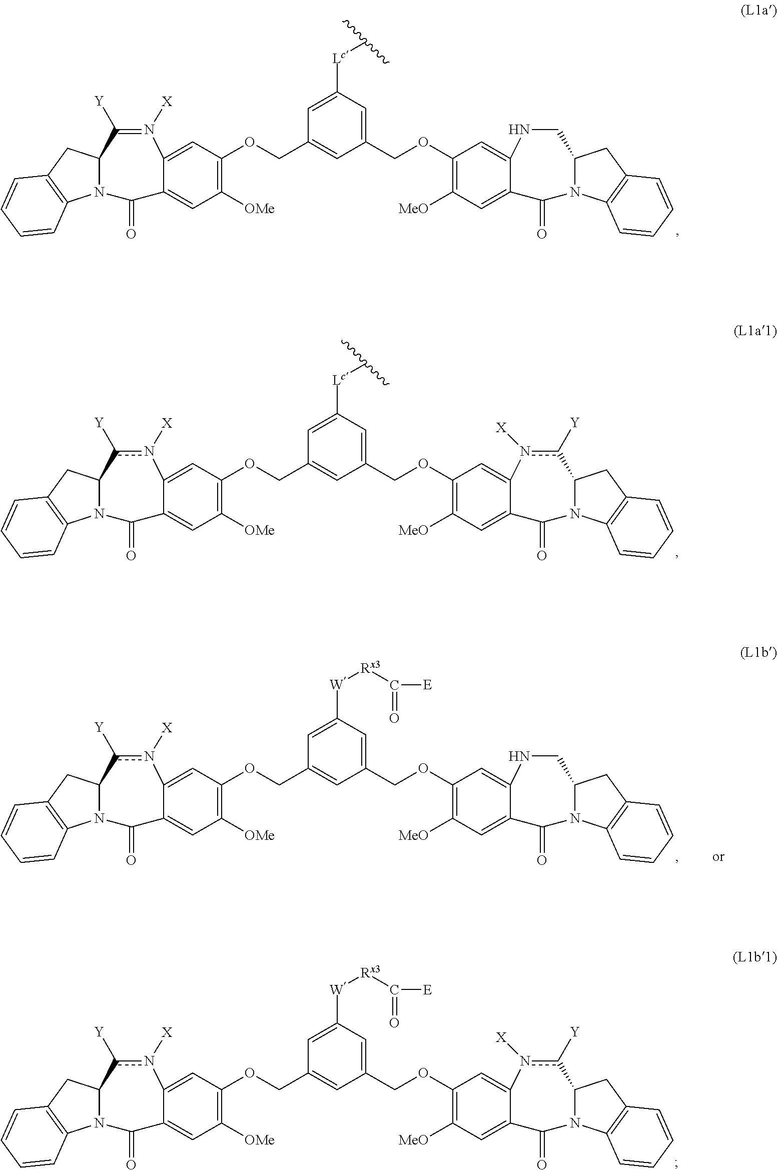

or a pharmaceutically acceptable salt thereof, wherein the definitions for the variables are described above for formulas (L1a′), (L1a′1), (L1b′) and (L1b′1).

In certain embodiments, the immunoconjugates of the first embodiment can be prepared by a second method comprising the steps of:

(a) reacting the cytotoxic agent with a linker compound having an amine reactive group and a thiol reactive group to form a cytotoxic agent-linker compound having the amine reactive group bound thereto; and

(b) reacting the CBA with the cytotoxic agent-linker compound.

In one embodiment, for the second method described above, the reaction in step (a) is carried out in the presence of an imine reactive reagent (e.g., NaHSO₃).

In one embodiment, for the second method described above, the cytotoxic agent-linker compound is reacted with the CBA without purification. Alternatively, the cytotoxic agent-linker compound is first purified before reacting with the CBA.

In certain embodiments, the immunoconjugates of the first embodiment can be prepared by a third method comprising the steps of:

(a) reacting the CBA with a linker compound having an amine reactive group and a thiol reactive group to form a modified CBA having a thiol reactive group bound thereto; and

(b) reacting the modified CBA with the cytotoxic agent.

In one embodiment, for the third method described above, the reaction in step (b) is carried out in the presence of an imine reactive reagent (e.g., NaHSO₃).

In certain embodiments, the immunoconjugates of the first embodiment can be prepared by a fourth method comprising the steps of reacting the CBA, a cytotoxic compound and a linker compound having an amine reactive group and a thiol reactive group.

In one embodiment, for the fourth method, the reaction is carried out in the presence of an imine reactive agent (e.g., NaHSO₃).

In certain embodiments, for the second, third or fourth method, described above, the linker compound having an amine reactive group and a thiol reactive group is represented by the following formula:

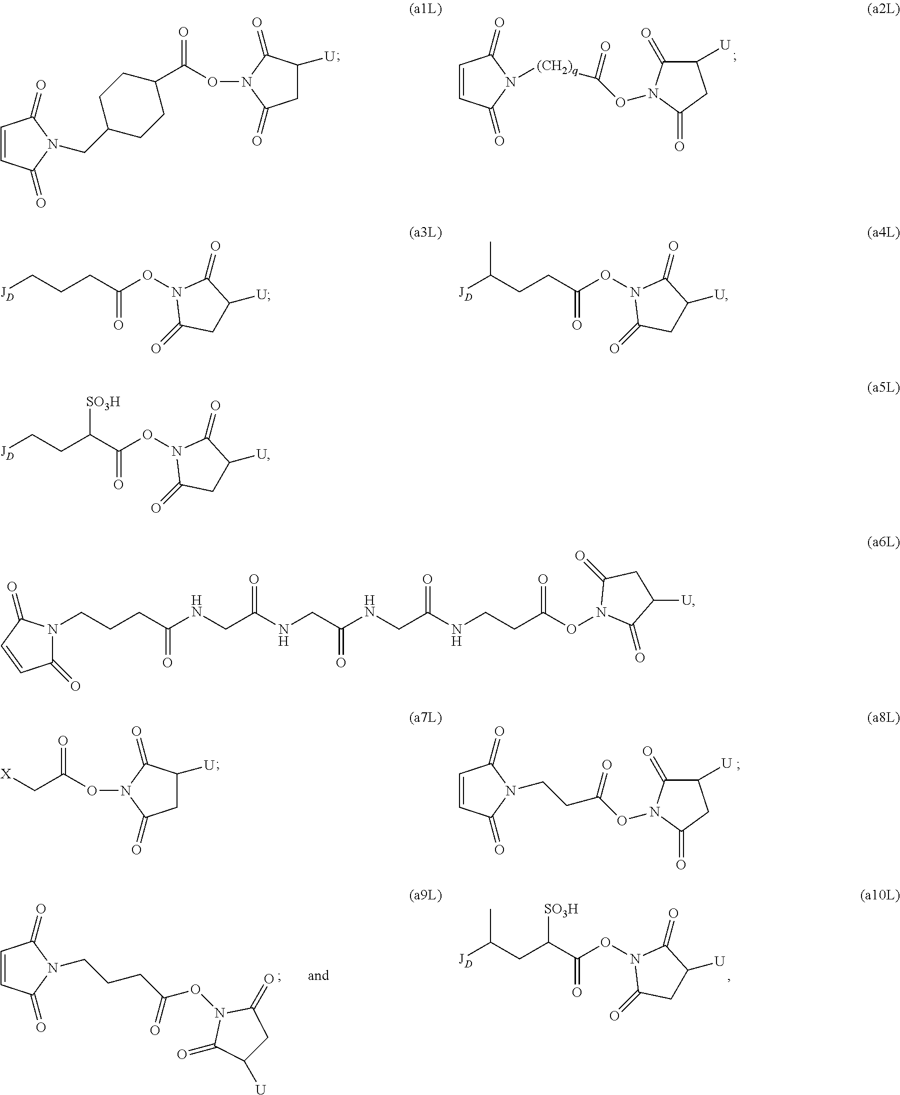

wherein X is halogen; J_{D}-SH, —SSR^{d}, or —SC(═O)R^{g}; R^{d} is phenyl, nitrophenyl, dinitrophenyl, carboxynitrophenyl, pyridyl or nitropyridyl; R^{g} is an alkyl; and the remaining variables are as described above for formula (a1)-(a10); and the cytotoxic agent is represented by the following formula:

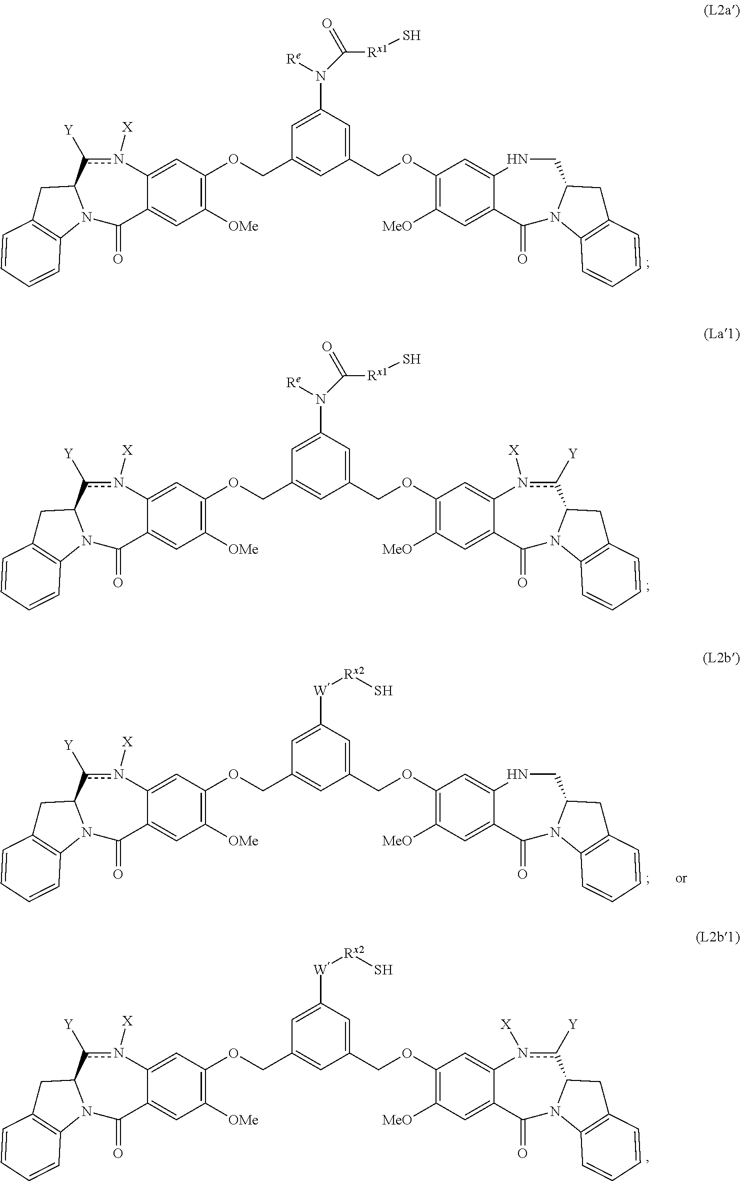

or a pharmaceutically acceptable salt thereof, wherein the variables are as described above for formulas (L1a′), (L1a′1), (L1b′), (L1b′1), (L2a′), (L2a′1), (L2b′) and (L2b′1).

In certain embodiments, for the second, third or fourth methods described above, the linker compound having an amine reactive group and a thiol reactive group is represented by any one of the formula (a1L)-(a10L) and the cytotoxic agent is represented by the following formula:

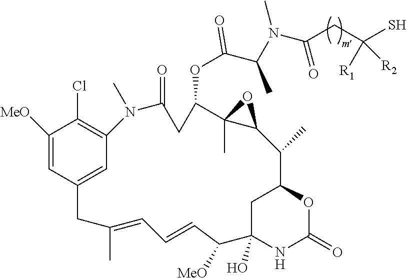

wherein the variables are as described above in any one of the 13^{th} to 15^{th} specific embodiments of the first embodiment described above and any more specific embodiments described therein.

In a specific embodiment, for the second, third or fourth methods described above, the linker is sulfo-SPDB, the cytotoxic agent is DM4 and the immunoconjugate is represented by the following formula:

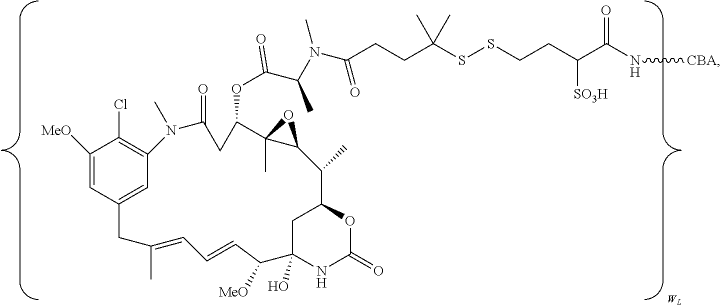

or a pharmaceutically acceptable salt thereof, wherein W_{L} is an integer from 1 to 10.

The immunoconjugates comprising a MET-binding agent covalently linked to a cytotoxic agent through the thiol group (—SH) of one or more cysteine residues located on the MET-binding agent as described in the second embodiment above (e.g., immunoconjugates of any one of the 1^{st} to 23^{rd} specific embodiments or any more specific embodiments described therein) can be prepared by reacting the CBA having one or more free cysteine with a cytotoxic agent having a thiol-reactive group described herein.

In one embodiment, the cytotoxic agent having a thiol-reactive group is represented by the following formula:

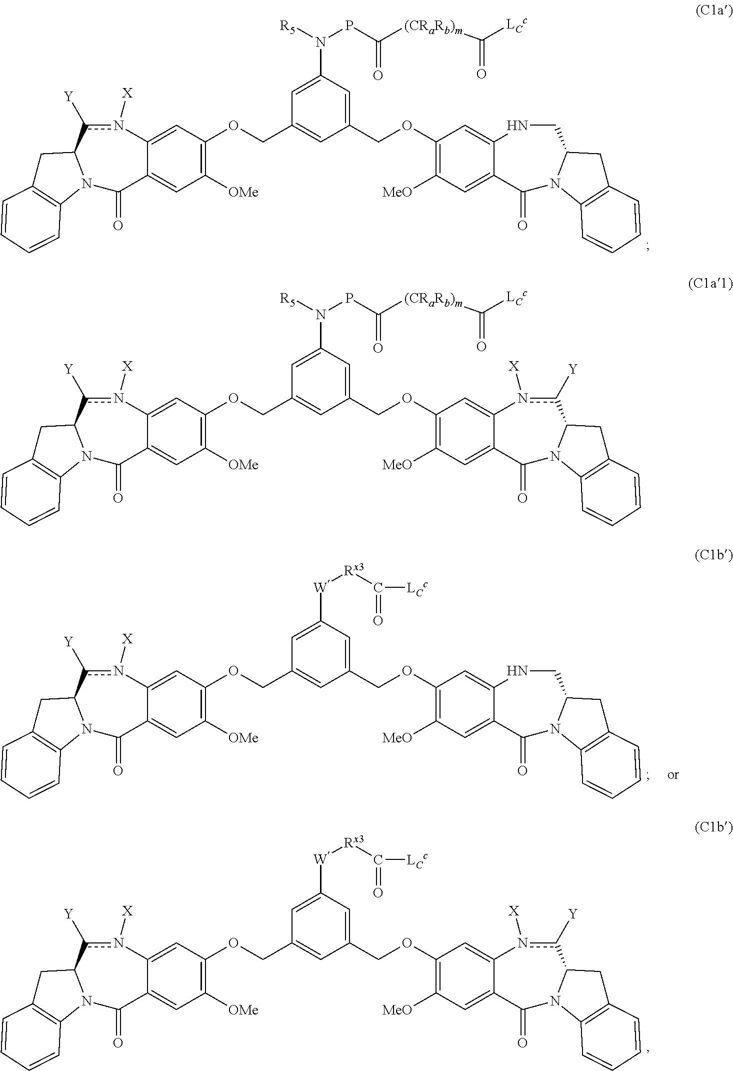

or a pharmaceutically acceptable salt thereof, wherein -L_{C}^{c} is represented by the following formula:

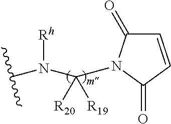

wherein the variables are as described above in any one of the 1^{st} to 9^{th} and 23^{rd} specific embodiments of the second embodiment or any more specific embodiments described therein.

In another embodiment, the cytotoxic agent having a thiol-reactive group is represented by the following formula:

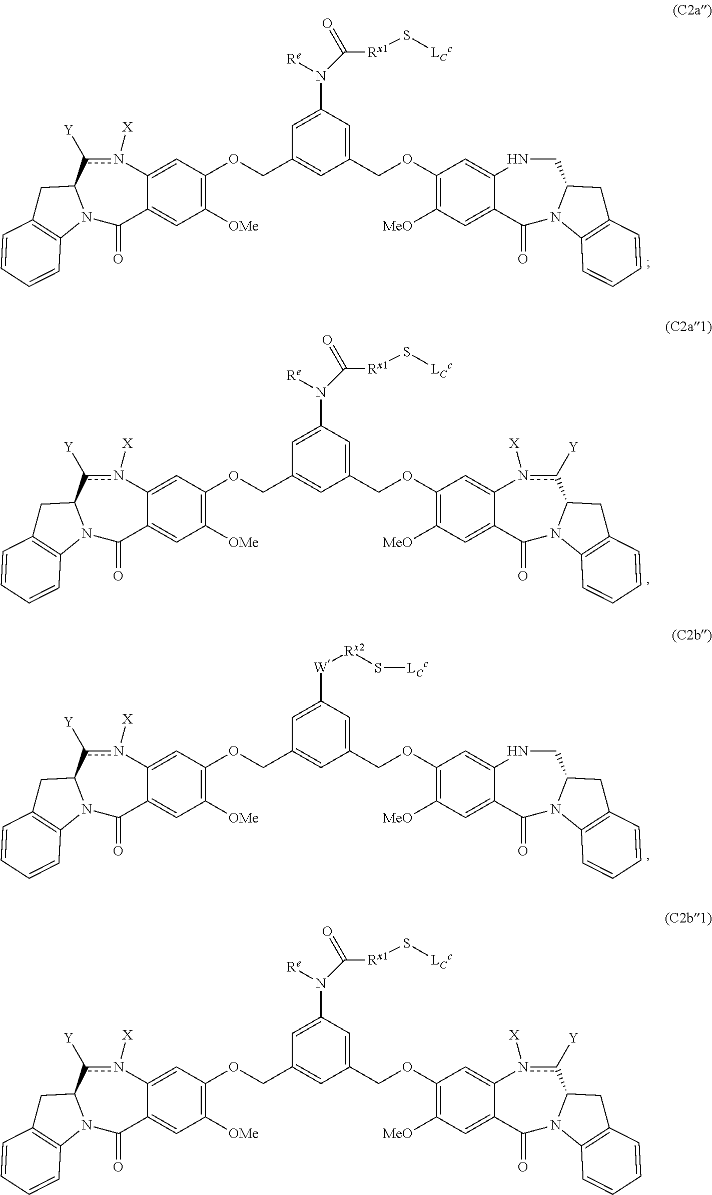

or a pharmaceutically acceptable salt thereof, wherein Lc^{c}′ is represented by the following formula:

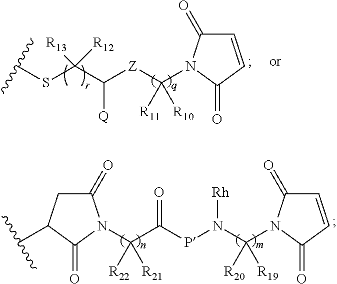

wherein the variables are as described above in any one of the 10^{th} to 16^{th} and 23^{rd} specific embodiment of the second embodiment or any more specific embodiments described therein.

In yet another embodiment, the cytotoxic agent having a thiol-reactive group is represented by the following formula:

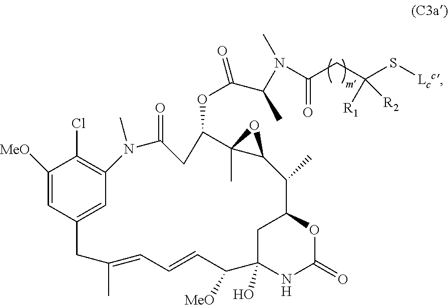

or a pharmaceutically acceptable salt thereof, wherein L_{C}^{c}′ is described above and the remaining variables are as described above in any one of the 17^{th} to 23^{rd} specific embodiments of the second embodiment or any more specific embodiments described therein.

In certain embodiments, organic solvents are used in the reaction of the CBA and the cytotoxic agent to solubilize the cytotoxic agent. Exemplary organic solvents include, but are not limited to, dimethylacetamide (DMA), propylene glycol, etc. In one embodiment, the reaction of the CBA and the cytotoxic agent is carried out in the presence of DMA and propylene glycol.

In a specific embodiment, the cytotoxic agent represented by the following formula:

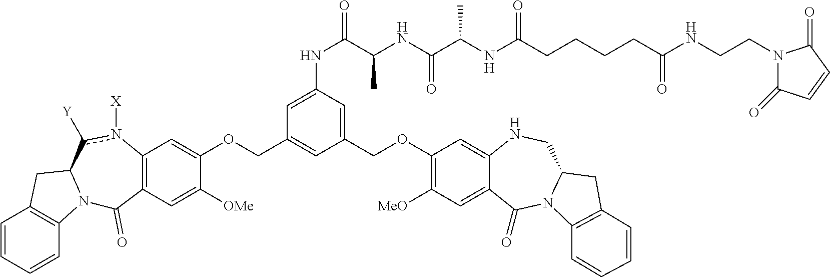

or a pharmaceutically acceptable salt thereof, is reacted with a CBA (e.g., an anti-MET antibody or an antibody fragment thereof)) to form the immunoconjugate represented by the following formula:

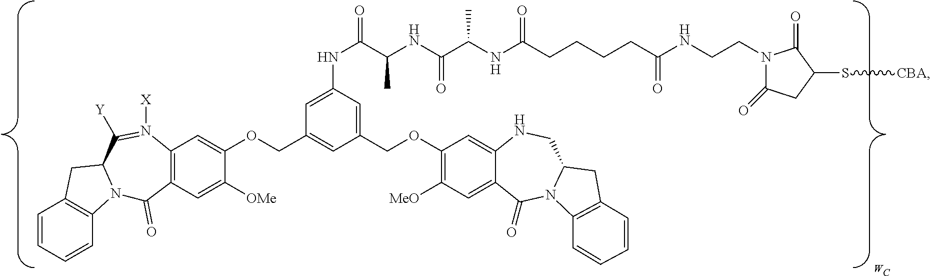

or a pharmaceutically acceptable salt thereof, wherein:

the double line [symbol] between N and C represents a single bond or a double bond, provided that when it is a double bond, X is absent and Y is —H, and when it is a single bond, X is —H, and Y is —SO₃H or a pharmaceutically acceptable salt thereof; and W_{C} is 1 or 2. In a more specific embodiment, the double line [symbol] between N and C represents a double bond, X is absent and Y is —H. In another more specific embodiment, the double line [symbol] between N and C represents a single bond, X is —H and Y is —SO₃H or a pharmaceutically acceptable salt thereof. Even more specifically, the pharmaceutically acceptable salt is a sodium or a potassium salt.

In certain embodiments, when Y is —SO₃H or a pharmaceutically acceptable salt thereof, the immunoconjugates can be prepared by (a) reacting the imine-moiety in the imine-containing cytotoxic agent having a thiol-reactive group described above (i.e., formula (C1a′), (C1a′1), (C1b′), (C1b′1), (C2a″), (C2a″1), (C2b″) or (C2b″1), wherein the double line [symbol] between N and C represents a double bond, X is absent and Y is —H) with a sulfur dioxide, bisulfite salt or a metabisulfite salt in an aqueous solution at a pH of 1.9 to 5.0 to form a modified cytotoxic agent comprising a modified imine moiety represented by the following formula:

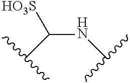 [SMILES: *NC(*)S(=O)(=O)O]

or a pharmaceutically acceptable salt thereof; and (b) reacting the modified cytotoxic agent with the MET-binding agent (e.g., an anti-MET antibody or an antibody fragment thereof) described herein to form the immunoconjugate.

In a 1^{st} aspect, for the method described above, the reaction of step (a) is carried out at a pH of 1.9 to 5.0. More specifically, the pH is 2.5 to 4.9, 1.9 to 4.8, 2.0 to 4.8, 2.5 to 4.5, 2.9 to 4.5, 2.9 to 4.0, 2.9 to 3.7, 3.1 to 3.5, or 3.2 to 3.4. In another specific embodiment, the reaction of step (a) is carried out at a pH of 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.5, 4.6, 4.7, 4.8, 4.9 or 5.0. In yet another specific embodiment, the reaction of step (a) is carried out at a pH of 3.3.

As used herein, a specific pH value means the specific value ±0.05.

In some embodiments, the reaction of step (a) is carried out in the presence of a buffer solution. Any suitable buffer solution known in the art can be used in the methods of the present invention. Suitable buffer solutions include, for example, but are not limited to, a citrate buffer, an acetate buffer, a succinate buffer, a phosphate buffer, a glycine-containing buffer (e.g., glycine-HCl buffer), a phthalate buffer (e.g., a buffer solution comprising sodium or potassium hydrogen phthalate), and a combination thereof. In some embodiments, the buffer solution is a succinate buffer. In some embodiments, the buffer solution is a phosphate buffer. In some embodiments, the buffer is a citrate-phosphate buffer. In some embodiments, the buffer is a citrate-phosphate buffer comprising citric acid and Na₂HPO₄. In other embodiments, the buffer is a citrate-phosphate buffer comprising citric acid and K₂HPO₄. In some embodiments, the concentration of the buffer solution described above can be in the range of 10 to 250 mM, 10 to 200 mM, 10 to 150 mM, 10 to 100 mM, 25 to 100 mM, 25 to 75 mM, 10 to 50 mM, or 20 to 50 mM.

In a 2^{nd} aspect, the reaction step (a) is carried out in the absence of a buffer solution (e.g., the buffers described in the 1^{st} aspect). In some embodiments, the present method comprises the steps of: (a) reacting the imine-moiety in the imine-containing cytotoxic agent having a thiol-reactive group described above (i.e., formula (C1a′), (C1a′1), (C1b′), (C1b′1), (C2a″), (C2a″1), (C2b″) or (C2b″1), wherein the double line [symbol] between N and C represents a double bond, X is absent and Y is —H) with sulfur dioxide, a bisulfite salt or a metabisulfite salt in an aqueous solution to form a modified cytotoxic agent comprising a modified imine moiety represented by the following formula:

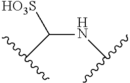 [SMILES: *NC(*)S(=O)(=O)O]

or a pharmaceutically acceptable salt thereof, wherein the aqueous solution does not comprise a buffer; and (b) reacting the modified cytotoxic agent with the MET-binding agent (e.g., an anti-MET antibody or an antibody fragment thereof) described herein to form the immunoconjugate. In some embodiments, the reaction of step (a) is carried out in a mixture of an organic solvent and water. More specifically, the reaction of step (a) is carried out in a mixture of dimethyacetamide (DMA) and water. In some embodiments, the mixture of DMA and water comprises less than 60% of DMA by volume. Even more specifically, the volume ratio of DMA and water is 1:1.

In a 3^{rd} aspect, for the methods described above or in the 1^{st} or 2^{nd} aspect, 0.5 to 5.0 equivalents of the bisulfite salt or 0.25 or 2.5 equivalents of the metabisulfite salt is used for every 1 equivalent of the imine-containing cytotoxic agent in the reaction of step (a). In some embodiments, 0.5 to 4.5, 0.5 to 4.0, 0.5 to 3.5, 0.5 to 4.0, 0.5 to 3.5, 0.5 to 3.0, 0.5 to 2.5, 0.8 to 2.0, 0.9 to 1.8, 1.0 to 1.7, 1.1 to 1.6, or 1.2 to 1.5 equivalents of the bisulfite salt or 0.25 to 2.25, 0.25 to 2.0, 0.25 to 1.75, 0.25 to 2.0, 0.25 to 1.75, 0.25 to 1.5, 0.25 to 1.25, 0.4 to 1.0, 0.45 to 0.9, 0.5 to 0.85, 0.55 to 0.8, or 0.6 to 0.75 equivalents of the metabisulfite salt is used for every 1 equivalent of the imine-containing cytotoxic agent. In other embodiments, 0.5, 0.6, 0.7, 0.8, 0.9, 1.0, 1.1, 1.2, 1.3 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4.0, 4.5 or 5.0 equivalents of the bisulfite salt or 0.25, 0.3, 0.35, 0.4, 0.45, 0.5, 0.55, 0.6, 0.65 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 2.0, 2.25 or 2.5 equivalents of the metabisulfite salt is used for every 1 equivalent of the imine-containing cytotoxic agent. In yet other embodiments, 1.4 equivalents of the bisulfite salt or 0.7 equivalent of the metabisulfite salt is used for every 1 equivalent of the imine-containing cytotoxic agent. In other embodiments, 1.2 equivalents of the bisulfite salt or 0.6 equivalent of the metabisulfite salt is used for every 1 equivalent of the imine-containing cytotoxic agent.

As used herein, a specific equivalent means the specific value ±0.05.

In a 4^{th} aspect, for methods described above, the reaction of step (a) is carried out at a pH of 2.9 to 3.7 and 1.0 to 1.8 equivalents of the bisulfite salt or 0.5 to 0.9 equivalents of the metabisulfite salt is reacted with 1 equivalent of the imine-containing cytotoxic agent. In some embodiments, the reaction of step (a) is carried out at a pH of 3.1 to 3.5 and 1.1 to 1.6 equivalents of the bisulfite salt or 0.55 to 0.8 equivalents of the metabisulfite salt is reacted with 1 equivalent of the imine-containing cytotoxic agent. In other embodiments, the reaction of step (a) is carried out at a pH of 3.2 to 3.4 and 1.3 to 1.5 equivalents of the bisulfite salt or 0.65 to 0.75 equivalents of the metabisulfite is reacted with 1 equivalent of the imine-containing cytotoxic agent. In other embodiments, the reaction of step (a) is carried out at a pH of 3.3 and 1.4 equivalents of the bisulfite salt or 0.7 equivalent of the metabisulfite salt is reacted with 1 equivalent of the imine-containing cytotoxic agent. In yet other embodiments, the reaction of step (a) is carried out at a pH of 3.3 and 1.4 equivalents of sodium bisulfite is reacted with 1 equivalent of the imine-containing cytotoxic agent.

In a 5^{th} aspect, for the methods described above or in the 1^{st}, 2^{nd}, 3^{rd} or 4^{th} aspect, the reaction of step (a) is carried out in a mixture of an organic solvent and water. Any suitable organic solvent can be used. Exemplary organic solvents include, but are not limited to, alcohols (e.g., methanol, ethanol, propanol, etc.), dimethylformamide (DMF), dimethylsulfoxide (DMSO), acetonitrile, acetone, methylene chloride, etc. In some embodiments, the organic solvent is miscible with water. In other embodiments, the organic solvent is not miscible with water, i.e., the reaction of step (a) is carried out in a biphasic solution. In some embodiments, the organic solvent is dimethylacetamide (DMA). The organic solvent (e.g., DMA) can be present in the amount of 1%-99%, 1-95%, 10-80%, 20-70%, 30-70%, 1-60%, 5-60%, 10-60%, 20-60%, 30-60%, 40-60%, 45-55%, 10-50%, or 20-40%, by volume of the total volume of water and the organic solvent. In some embodiments, the reaction of step (a) is carried out in a mixture of DMA and water, wherein the volume ratio of DMA and water is 1:1.

In a 6^{th} aspect, for the methods described above or in the 1^{st}, 2^{nd}, 3^{rd}, 4^{th} or 5^{th} aspect, the reaction of step (a) can be carried out at any suitable temperature. In some embodiments, the reaction is carried out at a temperature from 0° C. to 50° C., from 10° C. to 50° C., from 10° C. to 40° C., or from 10° C. to 30° C. In other embodiments, the reaction is carried out at a temperature from 15° C. to 30° C., from 20° C. to 30° C., from 15° C. to 25° C., from 16° C. to 24° C., from 17° C. to 23° C., from 18° C. to 22° C. or from 19° C. to 21° C. In yet other embodiments, the reaction can be carried out at 15° C., 16° C., 17° C., 18° C., 19° C., 20° C., 21° C., 22° C., 23° C., 24° C. or 25° C. In some embodiments, the reaction can be carried out from 0° C. to 15° C., from 0° C. to 10° C., from 1° C. to 10° C., 5° C. to 15° C., or from 5° C. to 10° C.

In a 7^{th} aspect, for the methods described above or in the 1^{st}, 2^{nd}, 3^{rd}, 4^{th}, 5^{th} or 6^{th} aspect, the reaction of step (a) is carried out for 1 minute to 48 hours, 5 minutes to 36 hours, 10 minutes to 24 hours, 30 minutes to 24 hours, 30 minutes to 20 hours, 1 hour to 20 hours, 1 hour to 15 hours, 1 hour to 10 hours, 2 hours to 10 hours, 3 hours to 9 hours, 3 hours to 8 hours, 4 hours to 6 hours, or 1 hour to 4 hours. In some embodiments, the reaction is allowed to proceed for 4 to 6 hours. In other embodiments, the reaction is allowed to proceed for 10 minutes, 15 minutes, 20 minutes, 30 minutes, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, etc. In other embodiments, the reaction is allowed to proceed for 4 hours. In yet other embodiments, the reaction is allowed to proceed for 2 hours.

In a 8^{th} aspect, for the methods of the present invention described herein or in the 1^{st}, 2^{nd}, 3^{rd}, 4^{th}, 5^{th}, 6^{th} or 7^{th} aspect, the reaction of step (b) is carried out at a pH of 4 to 9. In some embodiments, the reaction of step (b) is carried out at a pH of 4.5 to 8.5, 5 to 8.5, 5 to 8, 5 to 7.5, 5 to 7, 5 to 6.5, or 5.5 to 6.5. In other embodiments, the reaction of step (b) is carried out at pH 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In some embodiments, for the methods described above or in the 1^{st}, 2^{nd}, 3^{rd}, 4^{th}, 5^{th}, 6^{th}, 7^{th} or 8^{th} aspect, the reaction of step (b) is carried out in an aqueous solution comprising a mixture of water and an organic solvent. Any suitable organic solvent described above can be used. More specifically, the organic solvent is DMA. In some embodiments, the aqueous solution comprises less than 50%, less than 40%, less than 30%, less than 25%, less than 20%, less than 15%, less than 10%, less than 5%, less than 3%, less than 2%, or less than 1% of the organic solvent (e.g. DMA) by volume.

In some embodiments, for the methods described herein or in the 1^{st}, 2^{nd}, 3^{rd}, 4^{th}, 5^{th}, 6^{th}, 7^{th} or 8^{th} aspect, the bisulfite salt is sodium or potassium bisulfite and the metabisulfite salt is sodium or potassium metabisulfite. In a specific embodiment, the bisulfite salt is sodium bisulfite and the metabisulfite salt is sodium metabisulfite.

In some embodiments, for the methods described herein or in the 1^{st}, 2^{nd}, 3^{rd}, 4^{th}, 5^{th}, 6^{th}, 7^{th} or 8^{th} aspect, the modified cytotoxic agent is not purified before reacting with the cell-binding agent in step (b). Alternatively, the modified cytotoxic agent is purified before reacting with the cell-binding agent in step (b). Any suitable methods described herein can be used to purify the modified cytotoxic agent.

In some embodiments, for the methods described above, the reaction of step (a) results in no substantial sulfonation of the maleimide group. In some embodiments, less than 50%, 40%, 30%, 20%, 10%, 9%, 8%, 7%, 6%, 5%, 4%, 3%, 2%, or 1% of the maleimide group is sulfonated. The percentage of maleimide sulfonation is equal to the total amount of the maleimide-sulfonated cytotoxic agent (the cytotoxic agent having sulfonation on the maleimide only) and the di-sulfonated cytotoxic agent (the cytotoxic agent having sulfonation on both the maleimide and the imine moieties) divided by the starting amount of the imine-containing cytotoxic agent before its reaction with the bisulfite salt or the metabisulfite salt.

In some embodiments, the immunoconjugates prepared by any methods described above is subject to a purification step. In this regard, the immunoconjugate can be purified from the other components of the mixture using tangential flow filtration (TFF), non-adsorptive chromatography, adsorptive chromatography, adsorptive filtration, selective precipitation, or any other suitable purification process, as well as combinations thereof.

In some embodiments, the immunoconjugate is purified using a single purification step (e.g., TFF). Preferably, the conjugate is purified and exchanged into the appropriate formulation using a single purification step (e.g., TFF). In other embodiments of the invention, the immunoconjugate is purified using two sequential purification steps. For example, the immunoconjugate can be first purified by selective precipitation, adsorptive filtration, absorptive chromatography or non-absorptive chromatography, followed by purification with TFF. One of ordinary skill in the art will appreciate that purification of the immunoconjugate enables the isolation of a stable conjugate comprising the cell-binding agent chemically coupled to the cytotoxic agent.

Any suitable TFF systems may be utilized for purification, including a Pellicon type system (Millipore, Billerica, Mass.), a Sartocon Cassette system (Sartorius AG, Edgewood, N.Y.), and a Centrasette type system (Pall Corp., East Hills, N.Y.)

Any suitable adsorptive chromatography resin may be utilized for purification. Preferred adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepharose, Phenyl-Sepharose, and Octyl Sepharose resins (all from GE Healthcare, Piscataway, N.J.), as well as Macro-prep Methyl and Macro-Prep t-Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond ABx resin (JT Baker, Phillipsburg N.J.) Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g., Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, Calif.).

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

### V. Diagnostic and Research Applications

In addition to the therapeutic uses of the antibodies discussed herein, the antibodies and/or fragments of the present invention can be employed in many known diagnostic and research applications. Antibodies and or fragments of the present invention may be used, for example, in the purification, detection, and targeting of MET, included in both in vitro and in vivo diagnostic methods. For example, the antibodies and/or fragments may be used in immunoassays for qualitatively and quantitatively measuring levels of MET expressed by cells in biological samples. See, e.g., Harlow et al., Antibodies: A Laboratory Manual (Cold Spring Harbor Laboratory Press, 2nd ed. 1988), incorporated by reference herein in its entirety.

The antibodies of the present invention may be used in, for example, competitive binding assays, direct and indirect sandwich assays, and immunoprecipitation assays (Zola, Monoclonal Antibodies: A Manual of Techniques, pp.147-158 (CRC Press, Inc., 1987)).

For example, the present invention also provides the above anti-MET peptides and antibodies, detectably labeled, as described below, for use in diagnostic or prognostic or patient stratification methods for detecting MET in patients known to be or suspected of having a MET-mediated condition. Anti-MET peptides and/or antibodies of the present invention are useful for immunoassays which detect or quantitate MET, or anti-MET antibodies, in a sample. An immunoassay for MET typically comprises incubating a biological sample in the presence of a detectably labeled high affinity anti-MET peptide and/or antibody of the present invention capable of selectively binding to MET, and detecting the labeled peptide or antibody which is bound in a sample. Various clinical assay procedures are well known in the art, e.g., as described in Immunoassays for the 80's, A. Voller et al., eds., University Park, 1981. Thus, an anti-MET peptide or antibody or fragment thereof can be added to nitrocellulose, or another solid support which is capable of immobilizing cells, cell particles or soluble proteins. The support can then be washed with suitable buffers followed by treatment with the detectably labeled MET-specific peptide or antibody or fragment thereof. The solid phase support can then be washed with the buffer a second time to remove unbound peptide or antibody or fragment thereof. The amount of bound label on the solid support can then be detected by known method steps.

By “solid phase support” or “carrier” is intended any support capable of binding peptide, antigen or antibody or fragment thereof. Well-known supports or carriers, include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. Those skilled in the art will know many other suitable carriers for binding antibody or fragment thereof, peptide or antigen, or can ascertain the same by routine experimentation.

Well known method steps can determine binding activity of a given lot of anti-MET peptide and/or antibody or fragment thereof. Those skilled in the art can determine operative and optimal assay conditions by routine experimentation.

Detectably labeling a MET-specific peptide and/or antibody or fragment thereof can be accomplished by linking to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the MET-specific antibodies or fragment thereof of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

By radioactively labeling the MET-specific antibodies and/or fragment thereof, it is possible to detect MET through the use of a radioimmunoassay (RIA) (see, for example, Work, et al., Laboratory Techniques and Biochemistry in Molecular Biology, North Holland Publishing Company, N.Y. (1978)). The radioactive isotope can be detected by such means as the use of a gamma counter or a scintillation counter or by autoradiography. Isotopes which are particularly useful for the purpose of the present invention are: ³H, ¹²⁵I, ¹³¹I, ³⁵S, ¹⁴C, and, preferably, ¹²⁵I.

It is also possible to label the MET-specific antibodies and or fragments thereof with a fluorescent compound. When the fluorescent labeled antibody is exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine.

The MET-specific antibodies or fragments thereof can also be detectably labeled using fluorescence-emitting metals such as ¹²⁵Eu, or others of the lanthanide series. These metals can be attached to the MET-specific antibody or fragment thereof using such metal chelating groups as diethylenetriaminepentaacetic acid (DTPA) or ethylenediamine-tetraacetic acid (EDTA).

The MET-specific antibodies or fragments thereof also can be detectably labeled by coupling to a chemiluminescent compound. The presence of the chemiluminescently labeled antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the MET-specific antibody, fragment or derivative thereof of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection of the MET-specific antibody, fragment or derivative thereof can be accomplished by a scintillation counter, for example, if the detectable label is a radioactive gamma emitter, or by a fluorometer, for example, if the label is a fluorescent material. In the case of an enzyme label, the detection can be accomplished by colorometric methods which employ a substrate for the enzyme. Detection can also be accomplished by visual comparison of the extent of enzymatic reaction of a substrate in comparison with similarly prepared standards.

For the purposes of the present invention, the MET which is detected by the above assays can be present in a biological sample. Any sample containing MET can be used. Preferably, the sample is a biological fluid such as, for example, blood, serum, lymph, urine, inflammatory exudate, cerebrospinal fluid, amniotic fluid, a tissue extract or homogenate, and the like. However, the invention is not limited to assays using only these samples, it being possible for one of ordinary skill in the art to determine suitable conditions which allow the use of other samples.

In situ detection can be accomplished by removing a histological specimen from a patient, and providing the combination of labeled antibodies of the present invention to such a specimen. The antibody or fragment thereof is preferably provided by applying or by overlaying the labeled antibody or fragment thereof to a biological sample. Through the use of such a procedure, it is possible to determine not only the presence of MET but also the distribution of MET in the examined tissue. Using the present invention, those of ordinary skill will readily perceive that any of a wide variety of histological methods (such as staining procedures) can be modified in order to achieve such in situ detection.

The antibody or fragment thereof of the present invention can be adapted for utilization in an immunometric assay, also known as a “two-site” or “sandwich” assay. In a typical immunometric assay, a quantity of unlabeled antibody or fragment thereof is bound to a solid support that is insoluble in the fluid being tested and a quantity of detectably labeled soluble antibody is added to permit detection and/or quantitation of the ternary complex formed between solid-phase antibody, antigen, and labeled antibody.

Typical, and preferred, immunometric assays include “forward” assays in which the antibody bound to the solid phase is first contacted with the sample being tested to extract the MET from the sample by formation of a binary solid phase antibody-MET complex. After a suitable incubation period, the solid support is washed to remove the residue of the fluid sample, including unreacted MET, if any, and then contacted with the solution containing a known quantity of labeled antibody (which functions as a “reporter molecule”). After a second incubation period to permit the labeled antibody to complex with the MET bound to the solid support through the unlabeled antibody or fragment thereof, the solid support is washed a second time to remove the unreacted labeled antibody or fragment thereof. This type of forward sandwich assay can be a simple “yes/no” assay to determine whether MET is present or can be made quantitative by comparing the measure of labeled antibody or fragment thereof with that obtained for a standard sample containing known quantities of MET. Such “two-site” or “sandwich” assays are described by Wide (Radioimmune Assay Method, Kirkham, ed., Livingstone, Edinburgh, 1970, pp. 199-206).

Other type of “sandwich” assays, which can also be useful with MET, are the so-called “simultaneous” and “reverse” assays. A simultaneous assay involves a single incubation step wherein the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period, is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays. In one aspect, a combination of antibodies of the present invention specific for separate epitopes can be used to construct a sensitive three-site immunoradiometric assay.

The antibodies or fragments thereof of the invention also are useful for in vivo imaging, wherein an antibody or fragment thereof labeled with a detectable moiety such as a radio-opaque agent or radioisotope is administered to a subject, preferably into the bloodstream, and the presence and location of the labeled antibody in the host is assayed. This imaging technique is useful in the staging and treatment of malignancies. The antibody or fragment thereof may be labeled with any moiety that is detectable in a host, whether by nuclear magnetic resonance, radiology, or other detection means known in the art.

The label can be any detectable moiety that is capable of producing, either directly or indirectly, a detectable signal. For example, the label may be a biotin label, an enzyme label (e.g., luciferase, alkaline phosphatase, beta-galactosidase and horseradish peroxidase), a radio-label (e.g., ³H, ¹⁴C, ³²P, ³⁵S, and ¹²⁵I), a fluorophore such as fluorescent or chemiluminescent compound (e.g., fluorescein isothiocyanate, rhodamine), an imaging agent (e.g., Tc-m99 and indium (¹¹¹In)) and a metal ion (e.g., gallium and europium).

Any method known in the art for conjugating the antibody or fragment thereof to the label may be employed, including those exemplary methods described by Hunter, et al., 1962, Nature 144:945; David et al., 1974, Biochemistry 13:1014; Pain et al., 1981, J. Immunol. Meth. 40:219; Nygren, J., 1982, Histochem. and Cytochem. 30:407.

The antibodies or fragments thereof of the invention also are useful as affinity purification agents. In this process, the antibodies, for example, are immobilized on a suitable support, such a Sephadex resin or filter paper, using methods well known in the art. Thus, MET may be isolated and purified from a biological sample.

### VI. Therapeutic Applications

Also included in the present invention are methods for inhibiting the growth of cells expressing MET. As provided herein, the immunoconjugates of the present invention have the ability to bind MET present on the surface of a cell and mediate cell killing. In particular, the immunoconjugates of the present invention comprising a cytotoxic payload, e.g., a indolinobenzodiazepine DNA-alkylating agent, are internalized and mediate cell killing via the activity of the cytotoxic payload e.g., a benzodiazepine, e.g., an indolinobenzodiazepine DNA-alkylating agent. Such cell killing activity may be augmented by the immunoconjugate inducing antibody-dependent cell-mediated cytotoxicity (ADCC) and/or complement dependent cytotoxicity (CDC).

As used herein the terms “inhibit” and “inhibiting” should be understood to include any inhibitory effect on cell growth, including cell death. The inhibitory effects include temporary effects, sustained effects and permanent effects.

The therapeutic applications of the present invention include methods of treating a subject having a disease. The diseases treated with the methods of the present invention are those characterized by the expression (e.g., cMET overexpression in the presence or absence of gene amplification) and/or activation of MET (e.g., in the presence or absence of gene amplification). Such diseases include for example, glioblastoma, pancreatic cancer, gastric cancer, prostate cancer, ovarian cancer, breast cancer, hepatocellular carcinoma (HCC), melanoma, osteosarcoma, and colorectal cancer (CRC), lung cancer including small-cell lung cancer (SCLC) and non-small cell lung cancer (NSCLC), head and neck squamous cell carcinoma (HNSCC), kidney cancer, renal cancer, esophageal cancer and thyroid cancer. The skilled artisan will understand that the methods of the present invention may also be used to treat other diseases yet to be described but characterized by the expression of MET.

In other particular embodiments, immunoconjugates of the present invention may be useful in the treatment of non-small-cell lung cancer (squamous cell, adenocarcinoma, or large-cell undifferentiated carcinoma), colorectal cancer (adenocarcinoma, gastrointestinal carcinoid tumors, gastrointestinal stromal tumors, primary colorectal lymphoma, leiomyosarcoma, melanoma, or squamous cell carcinoma), and gastric cancer.

The therapeutic applications of the present invention can be also practiced in vitro and ex vivo.

The present invention also includes therapeutic applications of the antibodies or conjugates of the present invention wherein the antibodies or conjugates may be administered to a subject, in a pharmaceutically acceptable dosage form. They can be administered intravenously as a bolus or by continuous infusion over a period of time, by intramuscular, subcutaneous, parenteral, intra-articular, intrasynovial, intrathecal, oral, topical, or inhalation routes. They may also be administered by intratumoral, peritumoral, intralesional, or perilesional routes, to exert local as well as systemic therapeutic effects.

### VII. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of the immunoconjugates of the present invention, or a combination of such agents and a pharmaceutically acceptable carrier.

Preferably, compositions of the invention comprise a prophylactically or therapeutically effective amount of immunoconjugates of the present invention and a pharmaceutically acceptable carrier. The invention also encompasses such pharmaceutical compositions that additionally include a second therapeutic antibody (e.g., tumor-specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with an immunoconjugates of the present invention, alone or with such pharmaceutically acceptable carrier. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the immunoconjugates of the present invention.

### VIII. Methods of Administration

The compositions of the present invention may be provided for the treatment, prophylaxis, and amelioration of one or more symptoms associated with a disease, disorder by administering to a subject a therapeutically effective amount an immunoconjugate of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Methods of administering an immunoconjugate of the invention include, but are not limited to, parenteral administration (e.g., intradermal, intramuscular, intraperitoneal, intravenous and subcutaneous), epidural, and mucosal (e.g., intranasal and oral routes). In a specific embodiment, the immunoconjugates of the present invention are administered intramuscularly, intravenously, or subcutaneously. The compositions may be administered by any convenient route, for example, by infusion or bolus injection, and may be administered together with other biologically active agents. Administration can be systemic or local.

The invention also provides that preparations of the immunoconjugates of the present invention are packaged in a hermetically sealed container such as an ampoule or sachette indicating the quantity of the molecule. In one embodiment, such molecules are supplied as a dry sterilized lyophilized powder or water free concentrate in a hermetically sealed container and can be reconstituted, e.g., with water or saline to the appropriate concentration for administration to a subject. Preferably, the immunoconjugates of the present invention are supplied as a dry sterile lyophilized powder in a hermetically sealed container.

The lyophilized preparations of the immunoconjugates of the present invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such immunoconjugates when provided in liquid form are supplied in a hermetically sealed container.

As used herein, an “therapeutically effective amount” of a pharmaceutical composition is an amount sufficient to effect beneficial or desired results including, without limitation, clinical results such as decreasing symptoms resulting from the disease, attenuating a symptom of infection (e.g., viral load, fever, pain, sepsis, etc.) or a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

A therapeutically effective amount can be administered in one or more administrations. For purposes of this invention, a therapeutically effective amount of drug, compound, or pharmaceutical composition is an amount sufficient to reduce the proliferation of (or the effect of) viral presence and to reduce and/or delay the development of the viral disease, either directly or indirectly. In some embodiments, a therapeutically effective amount of a drug, compound, or pharmaceutical composition may or may not be achieved in conjunction with another drug, compound, or pharmaceutical composition.

The dosage and frequency of administration of an immunoconjugate of the present invention may be reduced or altered by enhancing uptake and tissue penetration of the molecule by modifications such as, for example, lipidation.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering an immunoconjugate of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 317-327).

Treatment of a subject with a therapeutically or prophylactically effective amount of an immunoconjugate of the present invention can include a single treatment or, preferably, can include a series of treatments. It will also be appreciated that the effective dosage of the molecules used for treatment may increase or decrease over the course of a particular treatment.

### EXAMPLES

### Example 1

### Production of Murine MET Antibodies and Hybridoma Screening

### Cell Lines and Growth

Cell lines used herein were grown in the appropriate media, for example DMEM or RPMI-1640 media supplemented with 10% fetal bovine serum, 2 mM glutamine and 1% penicillin-streptomycin (all reagents from Invitrogen) at 37° C. in a humidified 5% CO₂ incubator unless otherwise indicated. Cells were passaged twice per week and maintained between 0.2 to 1×10⁶ cells/ml.

### Production of Murine MET Antibodies

An expression plasmid pSRa-MET was constructed that contained the MET extracellular and transmembrane domain sequence flanked by KpnI and XhoI restriction sites that allowed expression of a truncated version of human MET which corresponds to the first 1077 amino acids of the 1390 amino acid protein described by GenBank Protein ID 188595716. This truncated version does not contain the intracellular receptor kinase domain which comprises the receptor autophosphorylation site and the adaptor protein docking site. However, it does contain the entire extracellular portion of MET including the ligand-binding site. 300-19 cells, a pre-B cell line derived from a Balb/c mouse (Reth et al., Nature, 317:353-355 (1985)), was transfected with this expression plasmid to stably express high levels of truncated human MET on the cell surface and used for immunization of Balb/c VAF mice. Mice were first immunized subcutaneously with 10 μg of recombinant human HGFR/cMET-Fc chimeric protein (R&D systems; 358-MT/CF) in complete Freund's adjuvant (CFA) followed by the same antigen in incomplete Freund's adjuvant (IFA) two weeks later. The mice were then boosted with three immunizations of 5×10⁶ MET-expressing 300-19 cells per mouse every 2 weeks by standard immunization protocols known to those of skill, for example, such as those used at ImmunoGen, Inc. Immunized mice were boosted one more time with 5×10⁶ MET-expressing 300-19 cells per mouse three days before being sacrificed for hybridoma generation. Spleens from mice was collected according to standard animal protocols, such as, for example grinding tissue between two sterile, frosted microscopic slides to obtain a single cell suspension in RPMI-1640 medium. The spleen cells were centrifuged, pelleted, washed, and fused with a murine myeloma, such as, for example P3X63Ag8.653 cells (Kearney et al., J. Immunol., 123:1548-1550 (1979)) using polyethylene glycol-1500 (Roche 783 641). The fused cells were resuspended in RPMI-1640 selection medium containing hypoxanthine-aminopterin-thymidine (HAT) (Sigma H-0262) and selected for growth in 96-well flat-bottomed culture plates (Corning-Costar 3596, 200 μL of cell suspension per well) at 37° C. with 5% carbon dioxide (CO₂). After 5 days of incubation, 100 μL of culture supernatant were removed from each well and replaced with 100 μL of RPMI-1640 medium containing hypoxanthine-thymidine (HT) supplement (Sigma H-0137). Incubation at 37° C. with 5% CO₂ was continued until hydridoma clones were ready for antibody screening. Other techniques of immunization and hybridoma production can also be used, including those described in Langone et al. (Eds., “Immunochemical Techniques, Part I”, Methods in Enzymology, Academic Press, volume 121, Florida) and Harlow et al. (“Antibodies: A Laboratory Manual”; Cold Spring Harbor Laboratory Press, New York (1988)).

### Hybridoma Screening for MET Binding

Culture supernatants from the hybridoma were screened by flow cytometry for secretion of mouse monoclonal antibodies that bind to antigen positive cells but not antigen negative cells. Antigen positive cells used were for example MET-expressing 300-19 cells or MKN45 gastric cells, while antigen negative cells used were for example the non-transfected 300-19 cells. 100 μl of hybridoma supernatants was incubated for 3 h with either MET-expressing cells or the non-transfected 300-19 cells (1×10⁵ cells per sample) in 100 μL FACS buffer (RPMI-1640 medium supplemented with 2% normal goat serum). Then, the cells were centrifuged, pelleted, washed, and incubated for 1 h with 100 μL of PE-conjugated goat anti-mouse IgG-antibody (such as obtainable from, for example Jackson Laboratory) at 6 μg/mL in FACS buffer. The cells were centrifuged, pelleted again, washed with FACS buffer and resuspended in 200 μL of PBS containing 1% formaldehyde. Cells were acquired using a FACSCalibur flow cytometer with the HTS multiwell sampler or a FACS array flow cytometer and analyzed using CellQuest Pro (all from BD Biosciences, San Diego, US). Hybridoma clones identified as secreting anti-MET antibodies were expanded and grown to collect antibody-containing supernatant for additional screening.

### Example 2

### Expression of Reference Antibodies

In order to compare the activity of the isolated antibodies, previously identified anti-MET antibodies were cloned and expressed. The amino acid sequence for the HC and LC variable region of the 224G11 antibody was derived from WO 2009007427 (Goetsch L.) using SEQ ID NO:18 for the HC variable region and SEQ ID NO:21 for the LC variable region. The amino acid sequence for the HC and LC variable region of the 5D5 antibody was derived from U.S. Ser. No. 07/476,724 using SEQ ID NOs 187 to 193 for the HC variable region and SEQ ID NOs 179 to 185 for the LC variable region.

The variable region sequences for both antibodies were codon-optimized and synthesized by Blue Heron Biotechnology. The sequences are flanked by restriction enzyme sites for cloning in-frame with the respective constant sequences in single chain mammalian expression plasmids. Cloning, expression and purification was carried out as described above.

In order to assess the activity of a monovalent version of 5D5, a Fab preparation was isolated from the whole IgG using the Pierce® Fab preparation kit (Thermo Fisher Scientific, Waltham, Mass.). Briefly, 0.5 ml of purified 5D5 IgG at a concentration of 4.7 mg/ml were buffer exchanged to the Fab digestion buffer containing 20 mM cysteine, pH 7.0, and mixed with 30 μg (0.88 BAEE unit) of immobilized papain that was equilibrated in the same digestion buffer. The digestion reaction was incubated for 6 hours with an end-over-end mixer at 37° C. to maintain constant mixing of resin. The digestion was then stopped by removing the IgG digest from the resin by centrifugation at 5000×g. The digested antibody solution was then incubated with pre-packed immobilized Protein A column that was equilibrated in phosphate buffered saline (PBS) for 10 mins. The Fab fragment was collected as the flowthrough fraction while the Fc fragments and undigested IgG bound to the column. The 5D5 Fab fragment was then buffer exchanged into PBS using an Amicon centrifugal filter unit (Millipore, Billerica, Mass.). Fab purity was assessed with size exclusion chromatography and SDS-PAGE, and its concentration was determined by absorbance measurement at 280 nm using an extinction coefficient of 1.66 ml mg⁻¹ cm⁻¹.

### Example 3

### Hybridoma Screening for Inhibition of HGF-Binding

The ability of antibodies to inhibit binding of the HGF ligand to MET was evaluated with a flow cytometry based assay using intact BxPC3 and MKN45 cells. Therefore, the ligand inhibition is measured in the context of MET present on the surface of a cell rather than using a recombinant source of the receptor. Briefly, target cells were harvested and re-suspended in at 400,000 cells/ml in binding buffer (1×PBS, 0.1% BSA, 0.05% sodium azide) and added to a 96-wel plate at 50 μL per well. Hybridoma supernatant is added to the cells at 50 μL per well and the mixture was incubated for 30 min on ice. Subsequently, 50 μL of HGF at 150 ng/mL was added to yield a final concentration of 50 ng/mL. The mixture was incubated for 30 min on ice and then washed three times with binding buffer. Biotinylated goat-anti-HGF-antibody was diluted to 0.4 μg/mL in binding buffer and added at 100 μL/well. Plates were incubated on ice for 45 minutes and then washed three times with binding buffer. Allophycocyanin (APC)-conjugated streptavidin (Jackson ImmunoResearch) was diluted to 1:200 in binding buffer, added at 100 μL/well and plates were incubated on ice for 45 minutes. Plates were washed three times with binding buffer and cells were re-suspended in 150 μL/well of fixation buffer (1% formaldehyde in 1×PBS). Samples were acquired using a FACSCalibur flow cytometer with the HTS multiwell sampler and analyzed using CellQuest Pro (BD Biosciences, San Diego, US). The mean fluorescence intensity (MFI) of FL4 was determined for each treated sample as well as cells in the presence of HGF but absence of antibody treatment. Controls included untreated cells incubated with HGF (0% inhibition) and untreated cells incubated without HGF (100% inhibition). Percent inhibition was calculated by normalizing MFI values of treated samples to that of control samples using the following formula: Percent inhibition=100×[1−(treated sample−untreated cells without HGF)/(untreated cell with HGF−untreated cells without HGF)]. The percent inhibition values were plotted for each treatment.

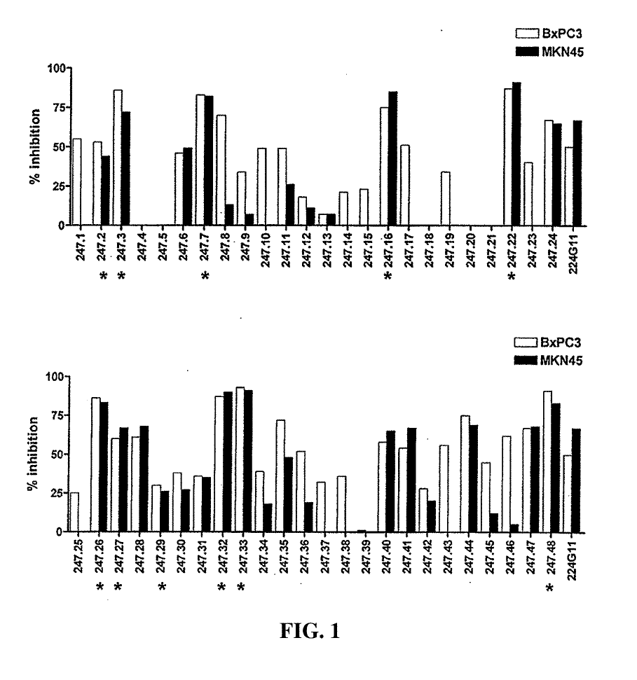

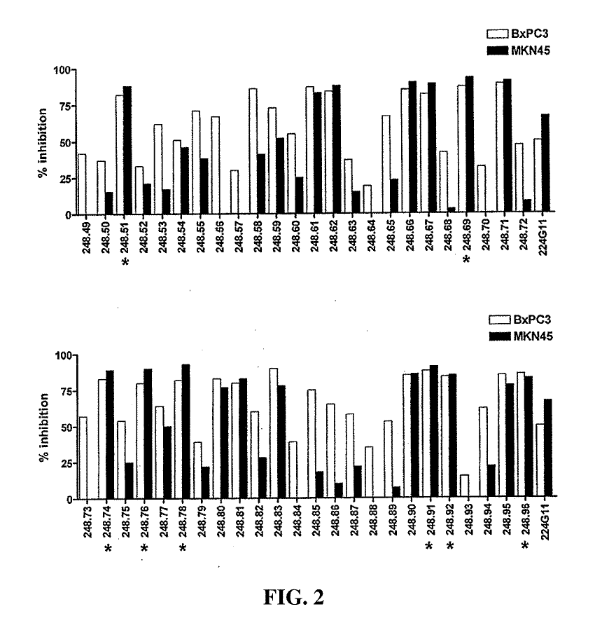

Supernatants from several isolated hybridoma clones showed strong activity in the flow cytometry based HGF-binding assay and were able to significantly inhibit HGF binding to BxPC3 as well as MKN45 cells (see FIG. 1 and FIG. 2). Clones were considered for further analysis if % inhibition of HGF binding to BxPC3 and MKN45 cells was at least 50% or above. The previously described anti-MET antibody 224G11 (Patent application WO 2009007427) was used in comparison and it resulted in % inhibition of HGF binding to BxPC3 and MKN45 cells of 50% and 67%, respectively. Several of the isolated hybridoma clones had more potent activity as compared to 224G11. Hybridoma clones such as 247.7, 247.22, 247.26, 247.32, 247.33, 247.48, 248.51, 248.61, 248.62, 248.66, 248.67, 248.69, 248.71, 248.74, 248.76, 248.78, 248.81, 248.83, 248.90, 248.91, 248.92, and 248.96 resulted in at least 80% inhibition of HGF binding to both BxPC3 and MKN45. Hybridoma clones 247.22, 247.48, and 248.69 are parental clones for hybridomas 247.22.2, 247.48.38 and 248.69.4, respectively, as described below in this and subsequent Examples.

The ability of exemplary antibodies to inhibit cell growth was measured using in vitro cytotoxicity assays. Briefly, target cells were plated at 4,000 cells per well in 100 μL in serum-free RPMI media (RPMI-1640, 2 mM glutamine, 1% penicillin-streptomycin, all reagents from Invitrogen). Antibodies were diluted into serum free media and 100 μL were added per well. Recombinant human HGF (R&D Systems) was diluted to 500 ng/ml in serum-free media and added at 50 μL per well to yield a final concentration of 100 ng/mL. Cells were incubated at 37° C. in a humidified 5% CO₂ incubator for 3 to 4 days. Viability of remaining cells was determined by colorimetric WST-8 assay (Dojindo Molecular Technologies, Inc., Rockville, Md., US). WST-8 is reduced by dehydrogenases in living cells to an orange formazan product that is soluble in tissue culture medium. The amount of formazan produced is directly proportional to the number of living cells. WST-8 was added to 10% of the final volume and plates were incubated at 37° C. in a humidified 5% CO₂ incubator for an additional 2-4 hours. Plates were analyzed by measuring the absorbance at 450 nm (A₄₅₀) in a multiwell plate reader. Controls included untreated cells incubated with HGF (0% inhibition) and untreated cells incubated without HGF (100% inhibition). Percent inhibition was calculated by normalizing MFI values of treated samples to that of control samples using the following formula: Percent inhibition=100×[1−(treated sample−untreated cells without HGF)/(untreated cell with HGF−untreated cells without HGF)]. The percent inhibition values were evaluated for each treatment.

Supernatants from several isolated hybridoma clones showed strong inhibition of HGF-induced proliferation of BxPC3. Clones were considered for further analysis if % inhibition of HGF-induced proliferation of BxPC3 cells was at least 40% or above.

### Hybridoma Subcloning and Subclone Screening

Desirable hybridoma clones were subcloned by limiting dilution. Hybridoma supernatant from subclones were screened again for binding to MET-expressing cell by flow cytometry as outlined above. One or two subclones from each parental hybridoma clone, which showed the same reactivity against MET as the parental clone by flow cytometry, was chosen for subsequent analysis.

Hybridoma supernatant from positive subclones was tested for inhibition of HGF binding to MKN45 and BxPC3 cells as outlined above. Percent inhibition was determined for each sample. Typically, subclones showed substantial inhibition of HGF binding to MKN45 and BxPC3 cells as expected.

One subclone from each parental hybridoma that showed substantial inhibition of HGF binding to MKN45 and BxPC3 cells was selected for subsequent analysis. Stable subclones were cultured and the isotype of each secreted anti-MET antibody was identified using commercial isotyping reagents (Roche #1493027 or EY Laboratories, Inc. #IC-IS-002-20).

### Example 4

### Antibody Purification

Antibodies were purified from hybridoma subclone supernatants using standard methods such as, for example, Protein A or G chromatography.

For purification of antibody desired standard methods such as for example chromatography with MabSelectSuRe, HiTrap Protein A or G HP (Amersham Biosciences) was used. Briefly, supernatant was prepared for chromatography by the addition of 1/10 volume of 1 M Tris/HCl buffer, pH 8.0. The pH-adjusted supernatant was filtered through a 0.22 μm filter membrane and loaded onto column equilibrated with binding buffer (PBS, pH 7.3). The column was washed with binding buffer until a stable baseline was obtained with no absorbance at 280 nm. Antibody was eluted with 0.1 M acetic acid buffer containing 0.15 M NaCl, pH 2.8, using a flow rate of 0.5 mL/min. Fractions of approximately 0.25 mL were collected and neutralized by the addition of 1/10 volume of 1M Tris/HCl, pH 8.0. The peak fraction(s) was dialyzed overnight twice against 1×PBS and sterilized by filtering through a 0.2 μm filter membrane. Purified antibody was quantified by absorbance at A280.

Protein A purified fractions were further polished using ion exchange chromatography (IEX) with quaternary ammonium (Q) chromatography for murine antibodies. Briefly, samples from protein A purification were buffer exchanged into binding buffer (10 mM Tris, 10 mM sodium chloride, pH 8.0) and filtered through 0.22 μm filer. The prepared sample was then loaded onto a Q fast flow resin (GE Lifesciences) that was equilibrated with binding buffer at a flow rate of 120 cm/hr. Column size was chosen to have sufficient capacity to bind all the MAb in the sample. The column was then washed with binding buffer until a stable baseline was obtained with no absorbance at 280 nm. Antibody was eluted by initiating a gradient from 10 mM to 500 mM sodium chloride in 20 column volume (CV). Peak fractions were collected based on absorbance measurement at 280 nm (A280). The percentage of monomer was assessed with size exclusion chromatography (SEC) on a TSK gel G3000SWXL, 7.8×300 mm with a SWXL guard column, 6.0×40 mm (Tosoh Bioscience, Montgomeryville, Pa.) using an Agilent HPLC 1100 system (Agilent, Santa Clara, Calif.). Fractions with monomer content above 95% were pooled, buffer exchanged to PBS (pH 7.4) using a TFF system, and sterilized by filtering through a 0.2 μm filter membrane. The IgG concentration of purified antibody was determined by A280 using an extinction coefficient of 1.47. Alternative methods such as ceramic hydroxyapatite (CHT) were also used to polish antibodies with good selectivity. Type II CHT resin with 40 μm particle size (Bio-Rad Laboratories) were used with a similar protocol as described for IEX chromatography. The binding buffer for CHT corresponds to 20 mM sodium phosphate, pH 7.0 and antibody was eluted with a gradient of 20-160 mM sodium phosphate over 20 CV.

### Example 5

### Sequencing, Chimerization, and Humanization of Anti-MET Antibodies

### Sequencing and Chimerization of the Anti-MET Antibodies

Total cellular RNA was prepared from 5×10⁶ cells of the MET hybridomas using an RNeasy kit (QIAgen) according to the manufacturer's protocol. cDNA was subsequently synthesized from total RNA using the SuperScript II cDNA synthesis kit (Invitrogen).

The procedure for degenerate PCR reactions on the cDNA derived from hybridoma cells was based on methods described in Wang et al. ((2000) J Immunol Methods. 233:167-77) and Co et al. ((1992) J Immunol. 148:1149-54). The primers and vectors were modified to facilitate directly cloning the hybridoma RT-PCR products in-frame with human constant region sequences in a mammalian expression vector capable of expressing chimeric versions of the murine antibodies. In this scheme, the PCR products themselves were initially sequenced with the PCR primers, and then following cloning, the variable regions were resequenced with vector specific primers. Since degenerate PCR primers were used at both the 5′ and 3′ ends of the antibody variable regions, germline sequence information obtained by searching NCBI IgBlast site (www.ncbi.nlm.nih.gov/igblast/) for the murine germline sequences was used to predict the N and C terminal murine sequences, though the primer generated residues remained in the chimeric expression plasmids.

These expression plasmids were then used to express chimeric antibodies in suspension HEK-293T cells using a modified Poly Ethyl Immine (PEI) procedure (Durocher, Y., et al., Nucleic Acids Res. 30:E9 (2002)). Supernatant was purified using standard Protein A chromatography procedures as described above, but the polishing chromatography steps were performed using either carboxymethyl (CM) fast flow ion exchange (IEX) resin (GE Lifesciences) and 10 mM potassium phosphate, 10 mM sodium chloride binding buffer (pH 7.5) or the alternative CHT methods described above. Binding experiments were performed with the chimeric antibodies to confirm that the cloned sequences preserve the expected binding properties of the murine antibodies.

All procedures related to antibody cloning and expression followed conventional molecular biology methods such as those described in standard laboratory manuals (Ausubel, F., et al, Wiley, 2010) or were performed according to the manufacturer's instructions.

### Humanization by Resurfacing Methods

The 247.22.2 and 247.27.16 antibodies were humanized following resurfacing methods previously described, such as, for example in Roguska et al., Proc. Natl. Acad. Sci., USA, 91(3):969-973 (1994) and Roguska et al., Protein Eng. 9(10):895-904 (1996), which are incorporated in their entirety herein by reference. Resurfacing generally involves identification of the variable region surface residues in both light and heavy chains and replacing them with human equivalents. Surface residue positions are defined as any position with its relative accessibility of 30% or greater (Pedersen et al., J. Mol. Biol., 235(3):959-973 (1994)). Surface residues are aligned with human germline surface sequences to identify the most homologous human surface sequence and replacements with human equivalent residues are made based on these alignments.

Exemplary CDRs for 247.22.2 are defined as indicated in the table below.

TABLE 7
Exemplary 247.22.2 (cMET-22) CDRs
Light Chain

| | |
|---|---|
| LC CDR1: | RASENIYSTLA |
| | (SEQ ID NO: 1) |
| LC CDR2: | AATNLAD |
| | (SEQ ID NO: 2) |
| LC CDR3: | QHFWGTPYT |
| | (SEQ ID NO: 3) |

Heavy Chain

| | |
|---|---|
| HC CDR1: | DYNMD |
| | (SEQ ID NO: 8) |
| HC CDR2: | DLNPNNGATI |
| | (SEQ ID NO: 12) |
| HC CDR3: | GNYYGNYYYLMDY |
| | (SEQ ID NO: 10) |

Kabat Defined HC CDR2

| | |
|---|---|
| Murine HC CDR2: | DLNPNNGATIYNQKFKG |
| | (SEQ ID NO: 9) |
| Human HC CDR2: | DLNPNNGATIYNEKFQG |
| | (SEQ ID NO: 73) |

Exemplary CDRs for 247.27.16 are defined as indicated in the table below.

TABLE 8
Exemplary 247.27.16 (cMET-27) CDRs
Light Chain

| | |
|---|---|
| LC CDR1: | RASESVDSYGNSFI |
| | (SEQ ID NO: 4) |
| LC CDR2: | RASNLES |
| | (SEQ ID NO: 5) |
| LC CDR3 1.0: | QQSNEDPLT |
| | (SEQ ID NO: 6) |
| LC CDR3 1.2: | QQSNEEPLT |
| | (SEQ ID NO: 7) |
| LC CDR3 1.3: | QQSNENPLT |
| | (SEQ ID NO: 8) |

Heavy Chain

| | |
|---|---|
| HC CDR1: | SYDMS |
| | (SEQ ID NO: 13) |
| HC CDR2: | TINSNGVSIY |
| | (SEQ ID NO: 17) |
| HC CDR3: | EEITTEMDY |
| | (SEQ ID NO: 15) |

Kabat Defined HC CDR2

| | |
|---|---|
| Murine and human HC CDR2: | TINSNGVSIYYPDSVKG |
| | (SEQ ID NO: 14) |

The light and heavy chain CDR's as defined for the resurfacing are given by way of example in Table 7 and Table 8. The Kabat definition for heavy chain CDR2 is also given for both the murine and human sequence. The underlined sequence marks the portion of the Kabat heavy chain CDR2 not considered a CDR for resurfacing.

The CDR3 of the 247.27.16 light chain contains a potential protease cleavage site. Therefore two alternate resurfaced versions LC CDR3 1.2 and LC CDR3 1.3 were generated to remove this site.

### Humanization by CDR-Grafting Methods

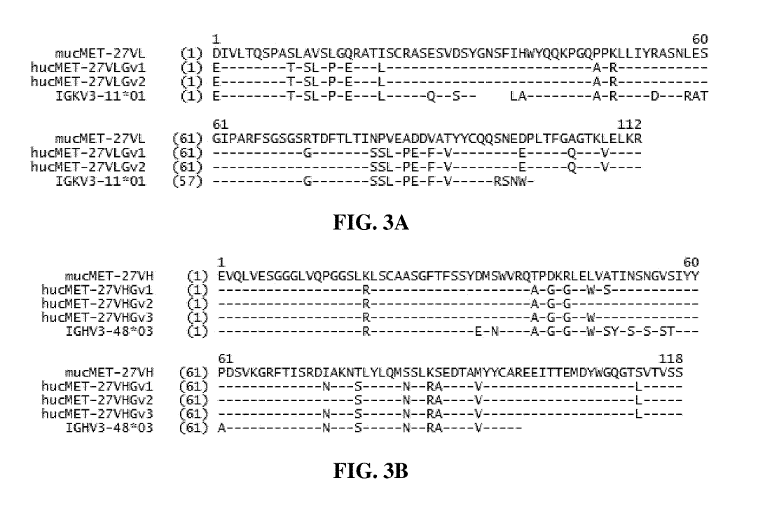

The murine CMET-27 antibody was humanized following complementary determining region (CDR) grafting procedures described in Jones et al., Nature 321: 604-608 (1986), Verhoeyen et al., Science 239: 1534-1536 (1988), U.S. Pat. No. 5,225,539 A (1993), and U.S. Pat. No. 5,585,089 A (1996). CDR grafting consists of replacing the Fv framework regions (FRs) of a mouse antibody with human antibody Fv framework regions while preserving the mouse CDR residues. Exemplary CDRs of the CMET-27 antibody following the Kabat numbering scheme and the Kabat CDR definitions are as indicated in Table 9. The CDR-grafting process begins by selecting appropriate human acceptor frameworks, typically those derived from human antibody genes sharing the highest sequence homology to the parent murine antibody utilizing the interactive tool, DomainGapAlign, of the International ImMunoGeneTics information system® (IMGT, http://www.imgt.org/) as described in Ehrenmann et al, Nucleic Acids Res. 38: D301-307 (2010). The human germline sequences selected as the acceptor frameworks for the V_{L} and V_{H} domains of cMET-27 antibody were IGKV3-11*01 and IGHV3-48*03, respectively (FIGS. 3A and 3B and in Table 3).

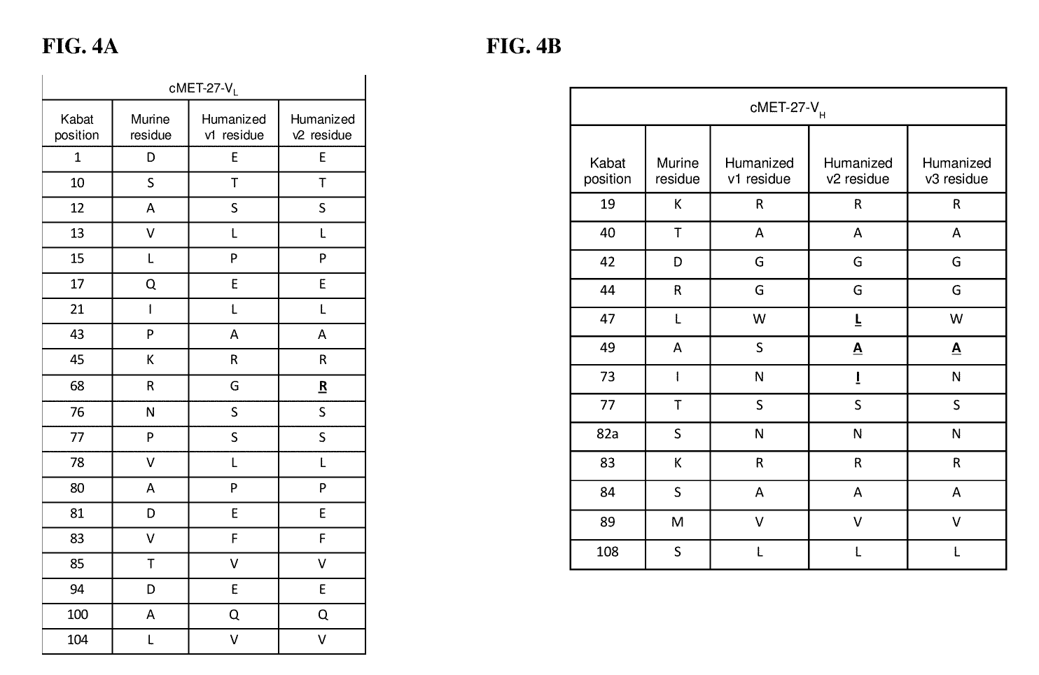

Further, the two consecutive LC CDR3 residues, Aspartate at position L94 and Proline at position L95, were considered as potential cleavage sites. Such potential sequence liability was successfully removed by replacing Aspartate with an homologous residue Glutamate at position L94 without impacting the binding affinity compared to the parent antibody. Further, it is well established that framework residues can make critical structural contributions to antigen binding and may need be to re-introduced as backmutations to restore antigen-binding affinity, Foote and Winter, J. Mol. Biol. 224: 487-499 (1992). Instead of introducing backmutations sequentially after the initial CDR grafted CMET-27 construct was made and evaluated, one additional humanized V_{L} version (VLGv2) containing backmutations at position L68 and one additional humanized V_{H} version (VHGv2) containing three backmutations at positions H47, H49, and H73 were constructed at the same time as the initial constructs were made (FIGS. 4A and 4B). All the four backmutations in both the V_{L} domain (G68R) and the V_{H} domain (W47L, S49A, and N73I) belong to the Vernier zone residues.

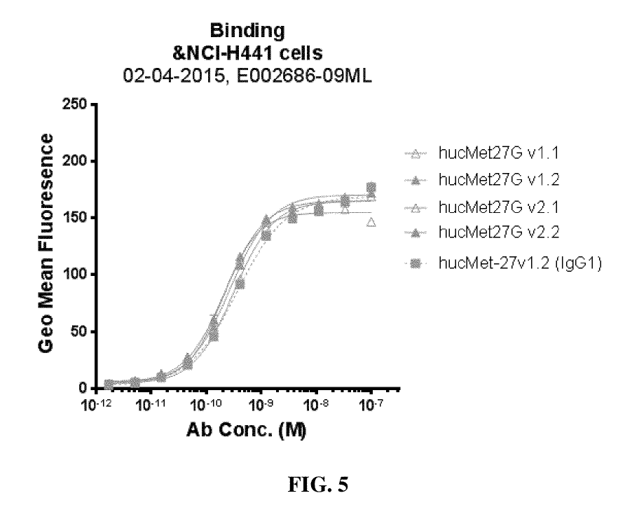

The humanized DNA constructs were synthesized, expressed via transient transfection of HEK 293T cells, and the recombinant antibodies purified with standard methods for subsequent cMET binding analysis compared with the parent antibody. As demonstrated in FIG. 5, all the humanized versions tested, including v1.1, contains no backmutation, v1.2, contains backmutations only in the V_{H} domain, v2.1, contains backmutation only in the V_{L} domain, and v2.2, contains backmutations in both V_{L} and V_{H} domains, retained parent binding to the cell line expressing human cMET antigen in direct FACS binding. It would be intuitive to pick v1.1 as the final humanized version since it contains no backmutation thereby keeping the CDR grafted antibody as “human” as possible. However, direct comparison of the transient expression titers of the four versions revealed that v1.1 expressed at a low level, 6 mg/L (Table 10). Low yield from transient expression makes research material less accessible; additionally, based on our experience, low transient titer is indicative of the difficulty in obtaining high expressing stable cell lines. Meanwhile, while version 1.2 expressed at an acceptable transient yield, two of the three backmutations in the V_{H} domain (W47L and N731) have very low relative frequency in human antibody molecules based on Abysis database (http://www.abysis.org/), which raised potential immunogenicity concerns. Consequently, one more humanized V_{H} version (VHGv3) removing the two low frequency backmutations was constructed (FIG. 4B). The hucMET27G v1.3, which contains one S49A backmutation in the V_{H} domain was transiently expressed at an intermediately level, and selected as the preferred CDR grafted cMET-27 construct. The huCMET27Gv1.3 antibodies containing hinge modifications (i.e., anti-MET antibodies comprising a light chain having the amino acid sequence of SEQ ID NO:49 and a heavy chain having the amino acid sequence of SEQ ID NO:77, SEQ ID NO:78; SEQ ID NO:79; SEQ ID NO:80, SEQ ID NO:81; SEQ ID NO:82; SEQ ID NO:83 or SEQ ID NO:84) are made as outlined above. In a specific embodiment, anti-MET antibody containing hinge modifications has a light chain having the amino acid sequence of SEQ ID NO:49 and a heavy chain having the amino acid sequence of SEQ ID NO:82.

TABLE 9
Exemplary cMET-27 CDRs
CMET-27 CDRs (CDR grafting)
Light Chain

| | |
|---|---|
| LC CDR1: | RASESVDSYGNSFIH |
| | (SEQ ID NO: 4) |
| LC CDR2: | RASNLES |
| | (SEQ ID NO: 5) |
| LC CDR3: | |
| Murine: | QQSNEDPLT |
| | (SEQ ID NO: 6) |
| Humanized versions: | QQSNEEPLT |
| | (SEQ ID NO: 7) |

Heavy Chain

| | |
|---|---|
| HC CDR1: | SYDMS |
| | (SEQ ID NO: 13) |
| HC CDR2: | TINSNGVSIYYPDSVKG |
| | (SEQ ID NO: 14) |
| HC CDR3: | EEITTEMDY |
| | (SEQ ID NO: 15) |

TABLE 10
Transient transfection titer comparison
for humanized cMET-27 versions

| Humanized versions | Transient expression titer (mg/mL) |
|---|---|
| hucMET27Gv1.1 | 6.81 |
| hucMET27Gv1.2 | 18.7 |
| hucMET27Gv2.1 | 10.5 |
| hucMET27Gv2.2 | 17.2 |

### Expression of Human Antibodies

The variable region sequences for hu247.22.2 and hu247.27.16 were codon-optimized and synthesized by Blue Heron Biotechnology. The sequences are flanked by restriction enzyme sites for cloning in-frame with the respective constant sequences in single chain mammalian expression plasmids. The light chain variable region is cloned into EcoRI and BsiWI sites in the LC expression plasmids. The heavy chain variable region is cloned into the HindIII and Apa1 sites in the HC expression plasmid. These plasmids can be used to express human antibodies in either transient or stable transfections in mammalian cells. Transient transfections to express human antibodies in HEK-293T cells can be performed using a modified PEI procedure (Durocher, Y. et al., Nucleic Acids Res. 30(2):E9 (2002)). Supernatant can be purified by Protein A and polishing chromatography steps using standard procedures as described above for chimerized antibodies.

The activity of chimeric or humanized antibodies can be evaluated as described for murine antibodies in the above examples.

### Example 6

### Target Expression Analysis

A preliminary prevalence analysis of MET expression was performed in gastric and non-small cell lung cancer (NSCLC).

All samples analyzed were FFPE (Formalin fixed & paraffin embedded) samples. The NSCLC (105) and Gastric cancer samples (15) were purchased from Avaden Biosciences. Immunohistochemical staining for cMet was carried out using the Ventana Discovery Ultra auto stainer. The primary antibody for cMET (SP44) was a commercially available rabbit monoclonal antibody. The IHC assay was developed at ImmunoGen for preliminary research use.

All samples were evaluated and scored by a board certified pathologist trained in the scoring algorithm. The presence of at least 100 viable tumor cells was required for scoring. Staining intensity was scored on a semi-quantitative integer scale from 0 to 3, with 0 representing no staining, 1 representing weak staining, 2 representing moderate and 3 representing strong staining. The percentage of cells staining positively at each intensity level was recorded. Scoring was based on localization of cMET to the cell membrane only, as well as evaluation of localization to both cytoplasm and membrane. The staining results were analyzed by H score, which combines components of staining intensity with the percentage of positive cells. It has a value between 0 and 300 and is defined as: 1*(percentage of cells staining at 1+intensity); +2*(percentage of cells staining at 2+intensity); +3*(percentage of cells staining at 3+intensity).

For NSCLC, 86 adenocarcinoma whole tissue sections and 19 squamous cell carcinoma whole tissue sections were stained and evaluated. For gastric cancer, 15 whole tissue sections of adenocarcinoma were analyzed. All of these samples were scored for membrane staining, and the results are summarized in the table below.

TABLE 11
Prevalence of MET in NSCLC and Gastric Cancer

| | % Positive | H score: | H score: | H score |
|---|---|---|---|---|
| Tumor type | (H score >=1) | 1-100 | 101-200 | 201-300 |
| NSCLC | 95% | 55% | 34% | 7% |
| Adenocarcinoma (n = 86) | | | | |
| NSCLC Squamous Cell | 100% | 95% | 5% | 0% |
| Carcinoma (n = 19) | | | | |
| Gastric | 93% | 53% | 40% | 0% |
| Adenocarcinoma (n = 15) | | | | |

### Example 7

### Cross-Species Reactivity and Binding Affinity Studies of MET Antibodies and Conjugates

The relative binding affinity of the humanized cMet targeting antibodies to human cMet (hu cMet) and cynomolgus monkey cMet (cyno cMet) was investigated using ForteBio analysis, in which soluble recombinant hu cMet or cyno cMet protein (containing the extracellular domain of cMet fused to a histidine-containing peptide) was incubated with biosensors loaded with immobilized anti-cMet antibody. Briefly, each antibody was bound and immobilized onto an anti-hIgG Fc Capture biosensor and then incubated in the presence of different concentrations (2.6-30 nM) of His-tagged soluble cMet. The kinetics of binding were determined via ForteBio analysis binding using a 1:1 binding fit model. The calculated ka, kd and KD from these studies are presented in Table 12. The results of these studies demonstrate that the humanized anti-cMet antibodies have similar binding affinity to human and cynomolgus monkey cMet, which will allow for toxicology and safety studies to validate the use of anti-cMet immunoconjugates as drug therapies.

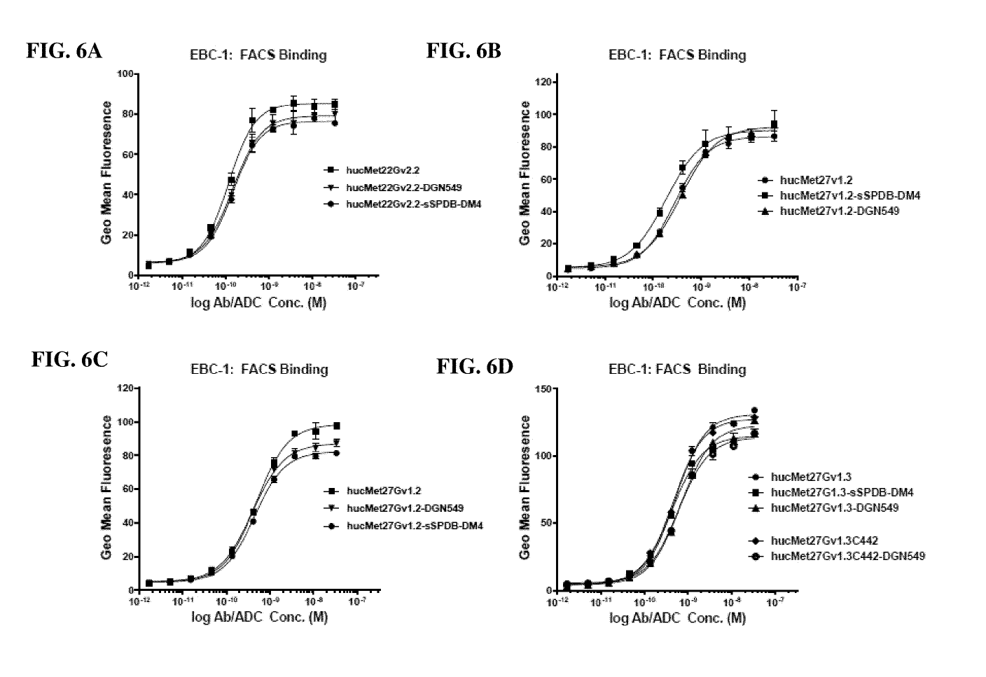

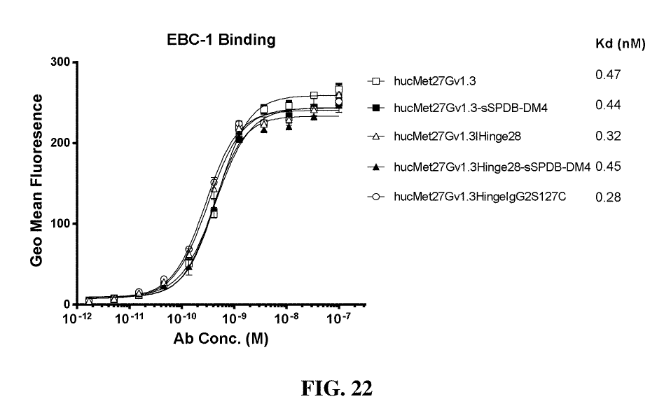

To evaluate the consequence of conjugation on antigen binding, the relative binding affinity of each anti-cMet immunoconjugate and its respective unconjugated antibody to cMet was determined by FACS analysis on EBC-1 cells, which endogenously express human cMet. Briefly, EBC-1 cells were incubated with dilution series of anti-cMet antibodies or immunoconjugates for 30 min @ 4° C. in FACS buffer (PBS, 0.1% BSA, 0.01% NaN₃). Samples were then washed and incubated with fluorescently-labeled secondary antibody for 30 minutes at 4° C. The geometric mean fluorescent intensity at each concentration was plotted and the EC50 of binding was calculated using a nonlinear regression analysis (GraphPad Prims 6). All of the anti-cMet antibodies and immunoconjugates tested bound with similar affinity to human cMet with an EC50 of approximately 0.4 nM measured by flow cytometry, indicating that conjugation did not appreciably alter antibody binding affinity (FIG. 6A-FIG. 6D). Similarly, the anti-cMET antibodies and immunoconjugates containing hinge modifications bound with similar affinity to human cMet (FIG. 22).

TABLE 12
Summary for binding of hucMet antibodies to the Extracelluar domain (ECD) of
cyno-cMet and human-cMet

| Anti-cMet | Human cMet-ECD-his | | | Cyno cMet-ECD-his | | |
|---|---|---|---|---|---|---|
| Antibodies | KD (M) | kon (1/Ms) | kdis (1/s) | KD (M) | kon (1/Ms) | kdis (1/s) |
| hucMet22Gv2.2 | 4.77E−09 | 4.45E+05 | 2.13E−03 | 3.62E−09 | 3.53E+05 | 1.28E−03 |
| hucMet27v1.2 | 9.89E−10 | 2.36E+05 | 2.33E−04 | 9.28E−10 | 2.12E+05 | 1.96E−04 |
| hucMet27Gv1.2 | 9.41E−10 | 2.66E+05 | 1.54E−04 | 6.14E−10 | 2.41E+05 | 1.48E−04 |
| hucMet27Gv1.3 | 1.81E−09 | 2.64E+05 | 4.78E−04 | 1.94E−09 | 2.14E+05 | 4.15E−04 |
| hucMet27Gv1.3C442 | 1.64E−09 | 2.72E+05 | 4.47E−04 | 1.60E−09 | 2.73E+05 | 4.39E−04 |
| hucMet27Gv1.3Hinge26C442 | 1.90E−09 | 2.62E+05 | 4.97E−04 | 1.57E−09 | 3.17E+05 | 4.96E−04 |
| hucMet27Gv1.3Hinge28 | 1.96E−09 | 2.30E+05 | 4.51E−04 | 1.68E−09 | 3.36E+05 | 5.64E−04 |
| hucMet27Gv1.3HingeIgG2S127C | 1.67E−09 | 2.35E+05 | 4.36E−04 | 1.47E−09 | 3.07E+05 | 4.50E−04 |

### Example 8

### Evaluation of Agonistic Activity of Anti-cMet Antibodies

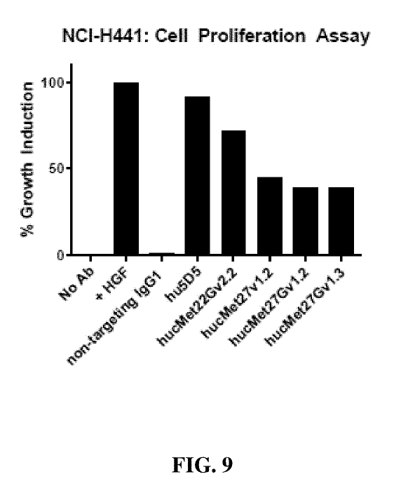

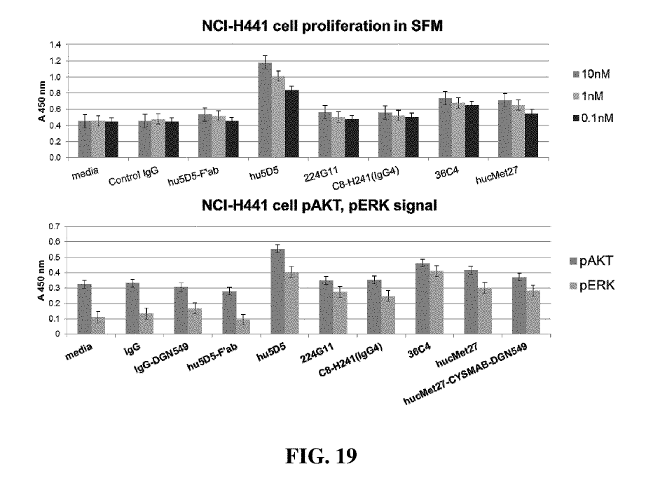

Exemplary antibodies were evaluated for potential induction of cell growth in the absence of HGF under serum-free conditions. Briefly, 3,000 NCI-H441 cells were plated in serum free media (SFM; 0.1%BSA in RPMI1640 medium). The following day cells were incubated with 1 nM of the indicated anti-cMet antibodies in SFM or 100 ng/mL of HGF at 37° C. in a humidified 5% CO₂ incubator for 4 days. Viability was tested using WST-8 which was added to 10% of the final volume and the samples were incubated at 37° C. in a humidified 5% CO₂ incubator for an additional 2-4 hours. Samples were analyzed by measuring the absorbance at 450 nm (A₄₅₀) in a multiwell plate reader. Background A₄₅₀ absorbance of wells with media and WST-8 only was subtracted from all values. Controls included untreated cells in grown in the presence of 100 ng/mL HGF (100% induction) and untreated cells grown in the absence of HGF (0% induction). Percent induction was calculated by normalizing A₄₅₀ values of treated samples to that of control samples using the following formula: Percent induction=100×(treated sample−untreated cells without HGF)/(cell with HGF−untreated cells without HGF). The results are shown in FIG. 9. The percent induction value was plotted for each treatment. The known agonisitic antibody, 5D5, alone induced cell growth to 92% of that observed by HGF treatment. However, the hucMet22Gv2.2 showed a 72% growth induction and the hucMet27 antibodies resulted in less than 45% induction at 1 nM. Therefore, all of the exemplary anti-cMet antibodies induced significantly less proliferation of NCI-H441 cells than cells treated with the cMet ligand, HGF, or a known agonistic antibody and comparable levels of proliferation to other cMET targeting antibodies with known low agonist activity (see, FIG. 19).

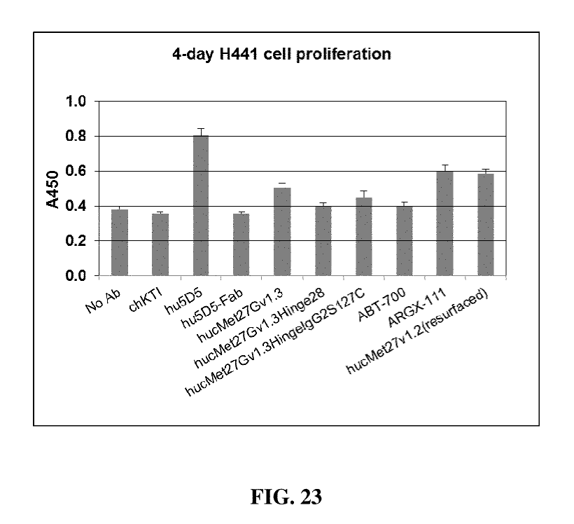

Exemplary antibodies containing hinge modifications were also evaluated for potential induction of cell growth in the absence of HGF under serum-free conditions. Briefly, 3,000 NCI-H441 cells were plated in serum free media (SFM; 0.1%BSA in RPMI1640 medium). The following day cells were incubated with 10 μg/mL of the indicated anti-cMet antibodies in SFM at 37° C. in a humidified 5% CO₂ incubator for 4 days. Viability was tested using WST-8 which was added to 10% of the final volume and the samples were incubated at 37° C. in a humidified 5% CO₂ incubator for an additional 2-4 hours. Samples were analyzed by measuring the absorbance at 450 nm (A₄₅₀) in a multi-well plate reader. Background A₄₅₀ absorbance of wells with media and WST-8 only was subtracted from all values. Controls included untreated cells grown in SFM. The results are shown in FIG. 23. The A450 absorbance value was plotted for each treatment. The known agonistic antibody, 5D5, alone induced cell growth as indicated by the increased A450 value. However, the hucMet27Gv1.3, and particularly hucMet27Gv1.3Hinge28 and hucMet27Gv1.3HingeIgG2S127C antibodies resulted in significantly less induction at 10 ug/mL than 5D5 and ARGX-111, with signal similar to ABT-700 and 5D5-F′ab.

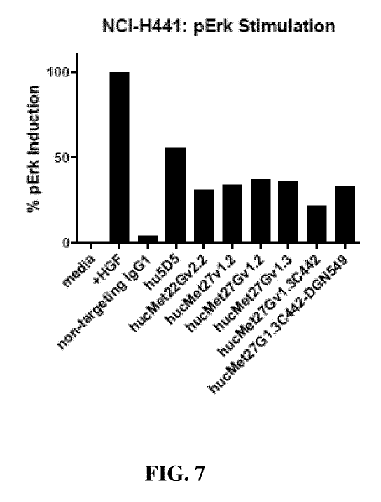

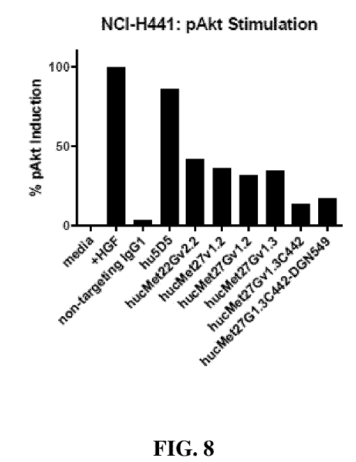

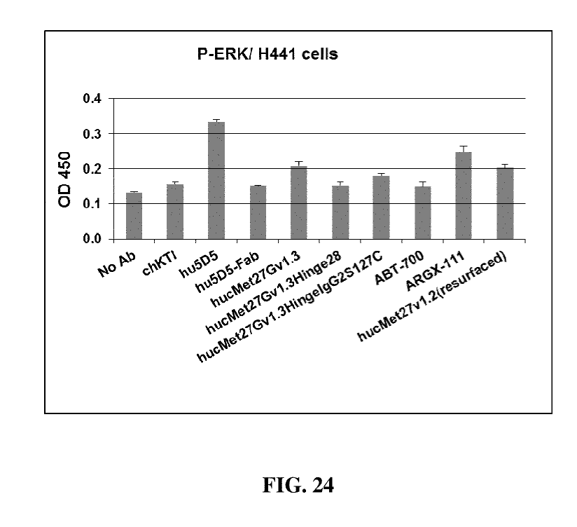

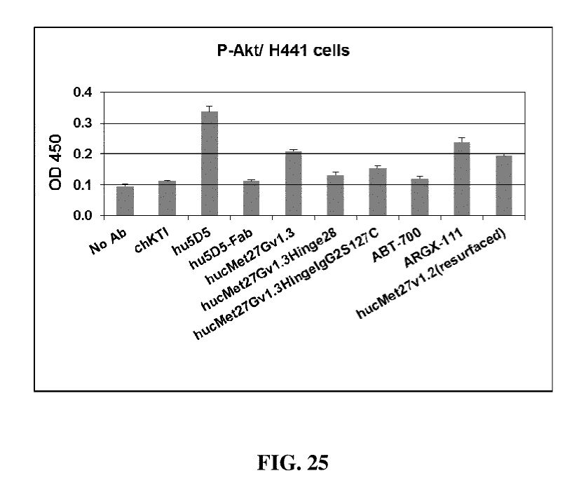

In order to determine the effect of anti-cMet antibodies on the activation of the tyrosine kinase activity of c-Met, ELISA-based assays were used to quantify downstream signaling events triggered by cMet activation. As described above, NCI-H441 cells were plated in SFM. The next day cells were incubated with 1 nM of the indicated anti-cMet antibodies/ADC in SFM or 100 ng/mL of HGF for 15 minutes. Samples were lysed and clarified lysates were assayed by ELISA for phophorylated-Erk and phosphorylated-Akt. Briefly, an immobilized capture antibody binds both phosphorylated and unphosphorylated of either Erk or Akt. After washing away unbound material, a biotinylated detection antibody is used to detect only phosphorylated protein, utilizing a standard HRP format. Samples were analyzed by measuring the absorbance at 450 nm (A₄₅₀) in a multiwell plate reader. Controls included cells treated with 100 ng/mL HGF (100% induction) and untreated cells treated in media alone (0% induction). Percent induction was calculated by normalizing A₄₅₀ values of treated samples to that of control samples using the following formula: Percent induction=100×(treated sample−untreated cells without HGF)/(cell with HGF−untreated cells without HGF). The percent induction value was plotted for each treatment. The results are shown in FIG. 7, FIG. 8, FIG. 24, and FIG. 25. While treatment with the agonistic cMet antibody 5D5 resulted in moderate phosphorylation of Erk, 5D5 induced elevated levels of phosphorylated Akt that mimic the activity of the native ligand, HGF. In contrast, hucMet22Gv2.2 and hucMet27 antibodies, and particularly hucMet27Gv1.3Hinge28 and hucMet27Gv1.3HingeIgG2S127C antibodies induced significantly lower levels of phosphorylated Erk and in particular phosphorylated Akt. hucMET27 antibody and conjugate showed similar levels of downstream signaling as compared to other cMET targeting antibodies with less agonistic activity compared to 5D5 (see, FIG. 19).

### Example 9

### Synthesis of N-(2-(2,5-dioxo-2,5-dihydro-1H-pyrrol-1-yl)ethyl)-11-(3-((((S)-8-methoxy-6-oxo-11,12,12a,13-tetrahydro-6H-benzo[5,6][1,4]diazepino[1,2-a]indol-9-yl)oxy)methyl)-5-((((S)-8-methoxy-6-oxo-12a,13-dihydro-6H-benzo[5,6][1,4]diazepino[1,2-a]indol-9-yl)oxy)methyl)phenyl)-13,13-dimethyl-2,5,8-trioxa-14,15-dithia-11-azanonadecan-19-amide, Compound D6

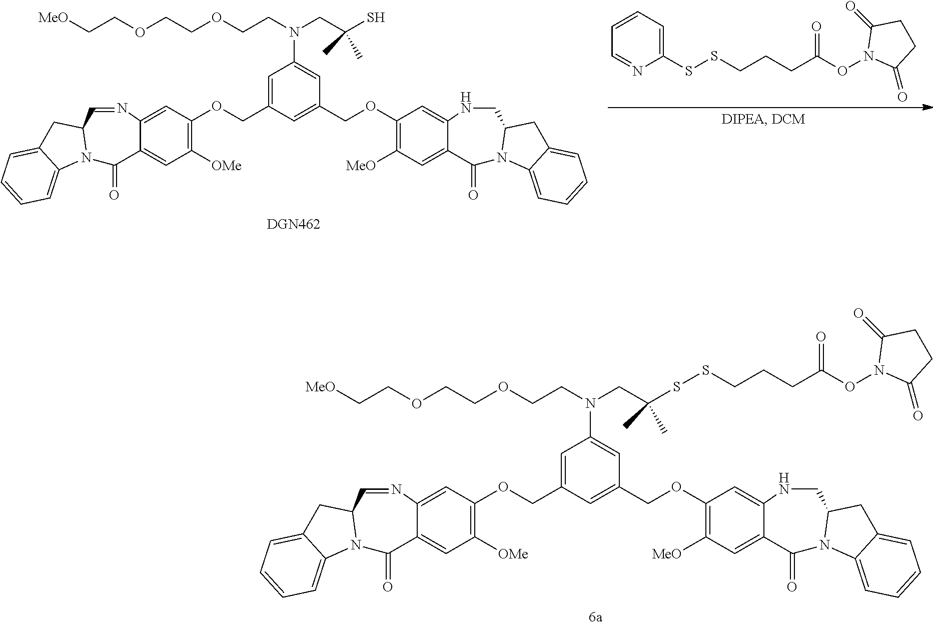

Step 1: To a solution of the free thiol DGN462 (40 mg, 0.042 mmol) and NHS 4-(2-pyridyldithio)butanate (35 mg, 80% purity, 0.085 mmol) in anhydrous dichloromethane (0.5 mL) was added anhydrous diisopropylethylamine (0.015 mL, 0.085 mmol) and was stirred at room temperature for 16 hours. The reaction mixture was quenched with saturated ammonium chloride and diluted with dichloromethane. The obtained mixture was separated in a separatory funnel. The organic layer was washed with brine, dried over anhydrous sodium sulfate, filtered and stripped under reduced pressure. The residue was purified by semi-preparative reverse phase HPLC (C18 column, CH₃CN/H₂O). The fractions that contained pure product were combined, frozen and lyophilized to give the desired NHS ester, compound 6a (29.7 mg, 60% yield). LCMS=9.1 min (15 min method). MS (m/z): 1157.3 (M+1)⁺.

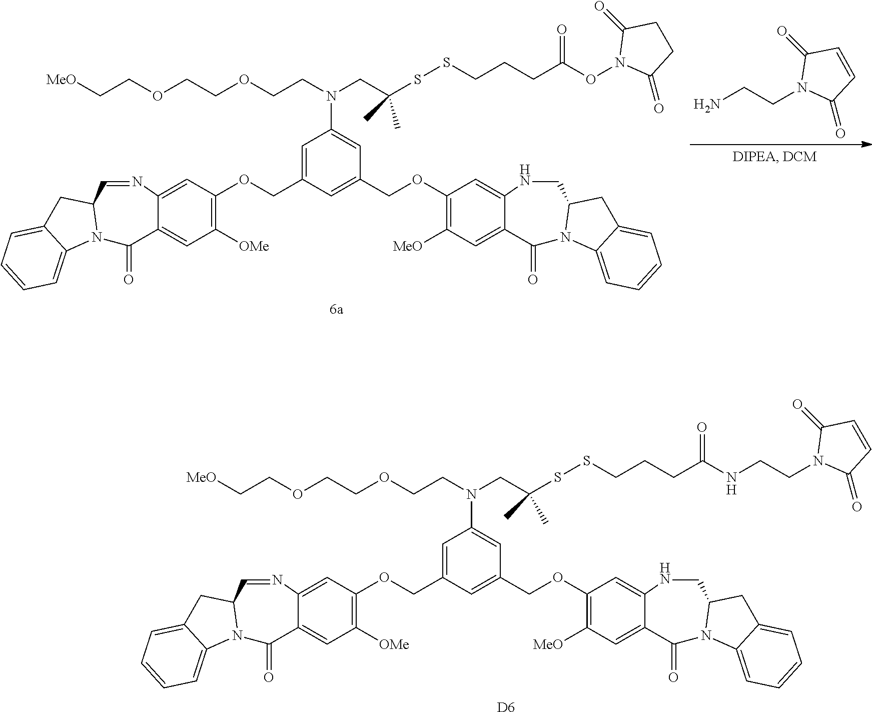

Step 2: To a solution of the NHS ester, compound 6a (12.3 mg, 0.011 mmol) and N-(2-aminoethyl)maleimide hydrochloride (2.0 mg, 0.011 mmol) in anhydrous dichloromethane (0.3 mL) was added DIPEA (0.0022 mL, 0.013 mmol). The mixture was stirred at room temperature for 3 hours then it was stripped under reduced pressure. The residue was purified by semi-preparative reverse phase HPLC (C18 column, CH₃CN/H₂O). The fractions that contained pure product were combined, frozen and lyophilized to give the desired maleimide, compound D6 (10 mg, 80% yield). LCMS=8.3 min (15 min method). MS (m/z): 1181.8 (M+1)⁺.

### Example 10

### Synthesis of N1-(2-(2,5-dioxo-2,5-dihydro-1H-pyrrol-1-yl)ethyl)-N6-((S)-1-(((S)-1-((3-((((S)-8-methoxy-6-oxo-11,12,12a,13-tetrahydro-6H-benzo[5,6][1,4]diazepino[1,2-a]indol-9-yl)oxy)methyl)-5-(4(S)-8-methoxy-6-oxo-12a,13-dihydro-6H-benzo[5,6][1,4]diazepino[1,2-a]indol-9-yl)oxy)methyl)phenyl)amino)-1-oxopropan-2-yl)amino)-1-oxopropan-2-yl)adipamide, compound D5

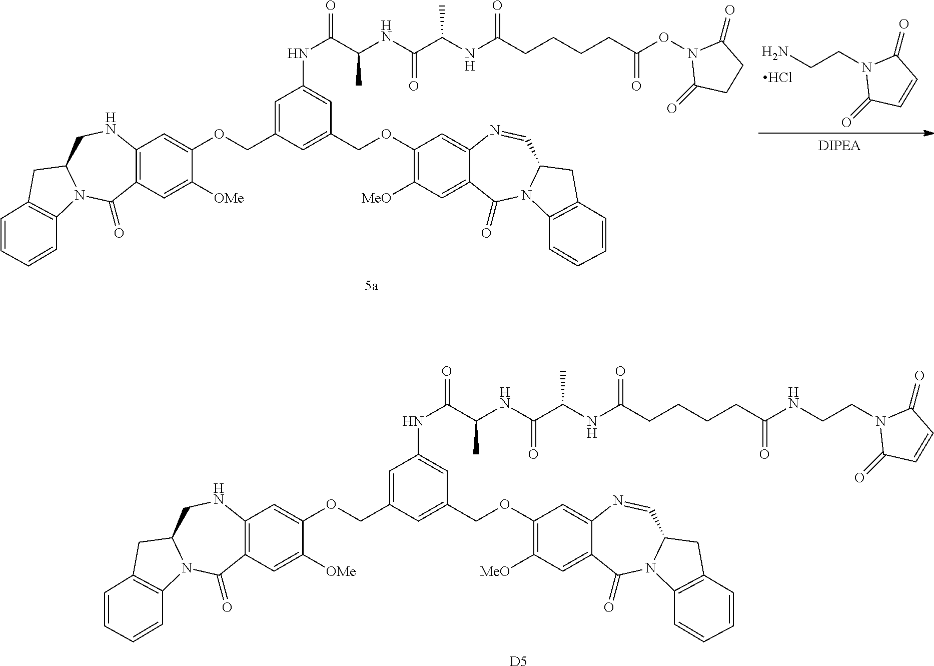

NHS ester, compound 5a (8.2 mg, 7.6 μmol) and 1-(2-aminoethyl)-1H-pyrrole-2,5-dione hydrochloride (2.2 mg, 0.011 mmol) were dissolved in anhydrous dichloromethane (305 μL) at room temperature. DIPEA (2.66 μL, 0.015 mmol) was added and the reaction and was stirred for 3.5 hours. The reaction mixture was concentrated and was purified by RPHPLC (C18 column, CH₃CN/H₂O, gradient, 35% to 55%). The desired product fractions were frozen and lyophilized to give maleimide, compound D5 as a solid white powder (5.3 mg, 58% yield). LCMS=5.11 min (8 min method). MS (m/z): 1100.6 (M+1)⁺.

### Example 11

### Synthesis of 1-((2-(2,5-dioxo-2,5-dihydro-1H-pyrrol-1-yl)ethyl)amino)-4-((5-((3-((((S)-8-methoxy-6-oxo-11,12,12a,13-tetrahydro-6H-benzo[5,6][1,4]diazepino[1,2-a]indol-9-yl)oxy)methyl)-5-((((S)-8-methoxy-6-oxo-12a,13-dihydro-6H-benzo[5,6][1,4]diazepino[1,2-a]indol-9-yl)oxy)methyl)phenyl)amino)-2-methyl-5-oxopentan-2-yl)disulfanyl)-1-oxobutane-2-sulfonic acid, compound D4

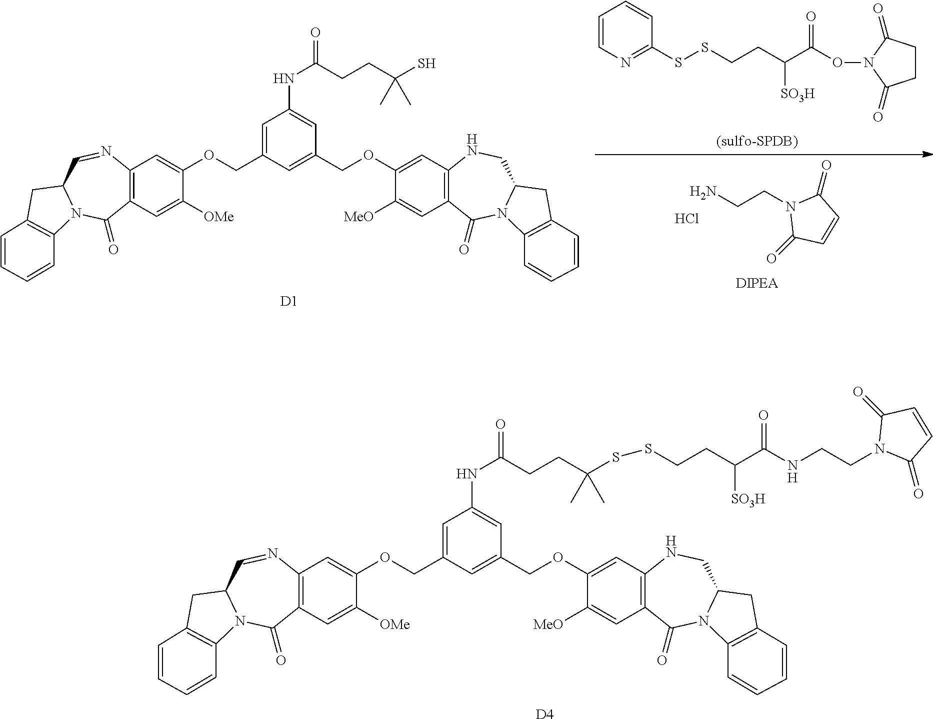

To a suspension of the free thiol, D1 (88 mg, 0.105 mmol) and 1-((2,5-dioxopyrrolidin-1-yl)oxy)-1-oxo-4-(pyridin-2-yldisulfanyl)butane-2-sulfonic acid (sulfo-SPDB) (64.0 mg, 0.158 mmol) in anhydrous dichloromethane (2.10 mL) was added DIPEA (55.0 μL, 0.315 mmol) under nitrogen at room temperature. The mixture stirred for 16 hours and then 1-(2-aminoethyl)-1H-pyrrole-2,5-dione hydrochloride (55.6 mg, 0.315 mmol), anhydrous dichloromethane (1.0 mL) and DIPEA (0.055 mL, 0.315 mmol) were added. The mixture stirred for an additional 5 hours at room temperature upon which the reaction was concentrated in vacuo. The resulting residue was purified by RP-HPLC (C18, CH₃CN/H₂O). Fractions containing desired product were frozen and lyophilized to give maleimide, D4 (20 mg, 16% yield) as a white solid. LCMS=4.92 min (8 min method). MS (m/z): 1158.6 (M+1)⁺.

### Example 12

### Synthesis of N-(2-(2,5-dioxo-2,5-dihydro-1H-pyrrol-1-yl)ethyl)-11-(3-((((S)-8-methoxy-6-oxo-11,12,12a,13-tetrahydro-6H-benzo[5,6][1,4]diazepino[1,2-a]indol-9-yl)oxy)methyl)-5-((((S)-8-methoxy-6-oxo-12a,13-dihydro-6H-benzo[5,6][1,4]diazepino[1,2-a]indol-9-yl)oxy)methyl)phenyl)-2,5,8-trioxa-11-azapentadecan-15-amide, compound D7

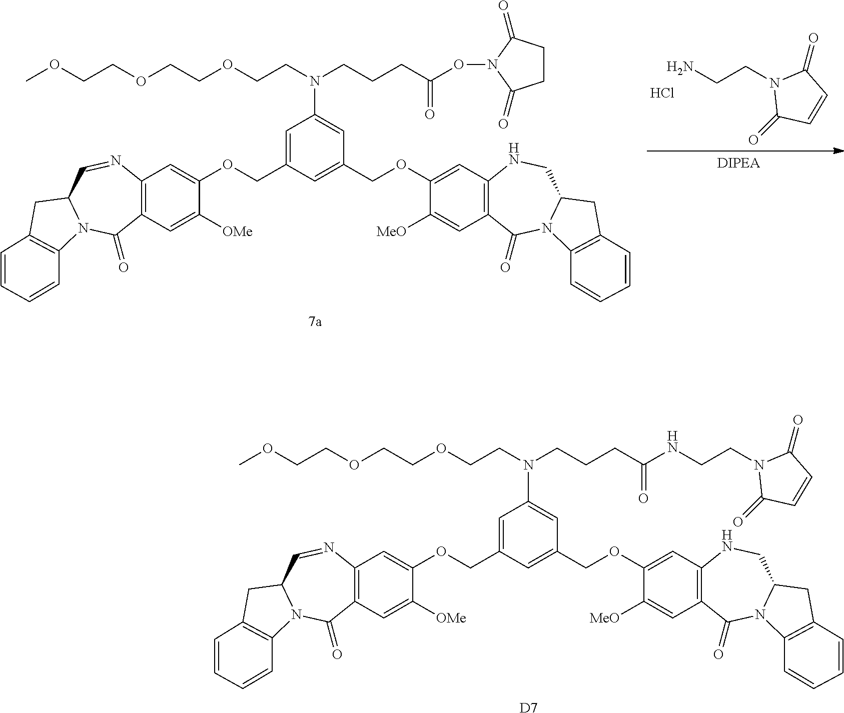 [SMILES: COCCOCCOCCN(CCCC(=O)ON1C(=O)CCC1=O)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1 ; Cl ; NCCN1C(=O)C=CC1=O ; COCCOCCOCCN(CCCC(=O)NCCN1C(=O)C=CC1=O)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1 ; "D7"]

To a solution of NHS ester, 7a (5 mg, 4.82 μmol) and 1-(2-aminoethyl)-1H-pyrrole-2,5-dione hydrochloride (1.7 mg, 9.64 μmol) in anhydrous dichloromethane (200 μL) was added DIPEA (1.512 μL, 8.68 μmol) under nitrogen. The mixture was stirred at room temperature for 4 hours and then concentrated in vacuo. The resulting residue was purified by RP-HPLC (C18, CH₃CN/H₂O). Fractions containing desired product were frozen and lyophilized to give maleimide, compound D7 (3.5 mg, 68% yield). LCMS=4.61 min (15 min method). MS (m/z): 1062.8 (M+1)⁺.

### Example 13

### Selective Sulfonation of Imine-Containing Cytotoxic Agent Bearing Maleimide Group

To a mixture of 50 mM sodium succinate pH 3.3 (116.5 mL) and DMA (98.5 mL) was added compound D5 (263.6 mg) dissolved in 21.4 mL of DMA. Subsequently 3.4 mL of a 100 mM sodium bisulfite solution (1.4 equivalents) in water containing 1 v/v % DMA was introduced into the reaction. The homogenous mixture was allowed to react for 2 h at 25° C., at which time completeness of the reaction was assayed by UPLC-MS. The reaction mixture is suitable for conjugation without further purification. UPLC-MS analysis of the reaction mixture shows 92.5% imine-sulfo D5, 1.9% unreacted D5, 0.8% maleimide-sulfo D5, and 4.8% di-sulfo D5. ESI-MS negative ion mode [M−H]⁻ calculated for imine-sulfo D5 (C₆₀H₆₂N₉O₁₅S⁻):1180.41; found:1180.03.

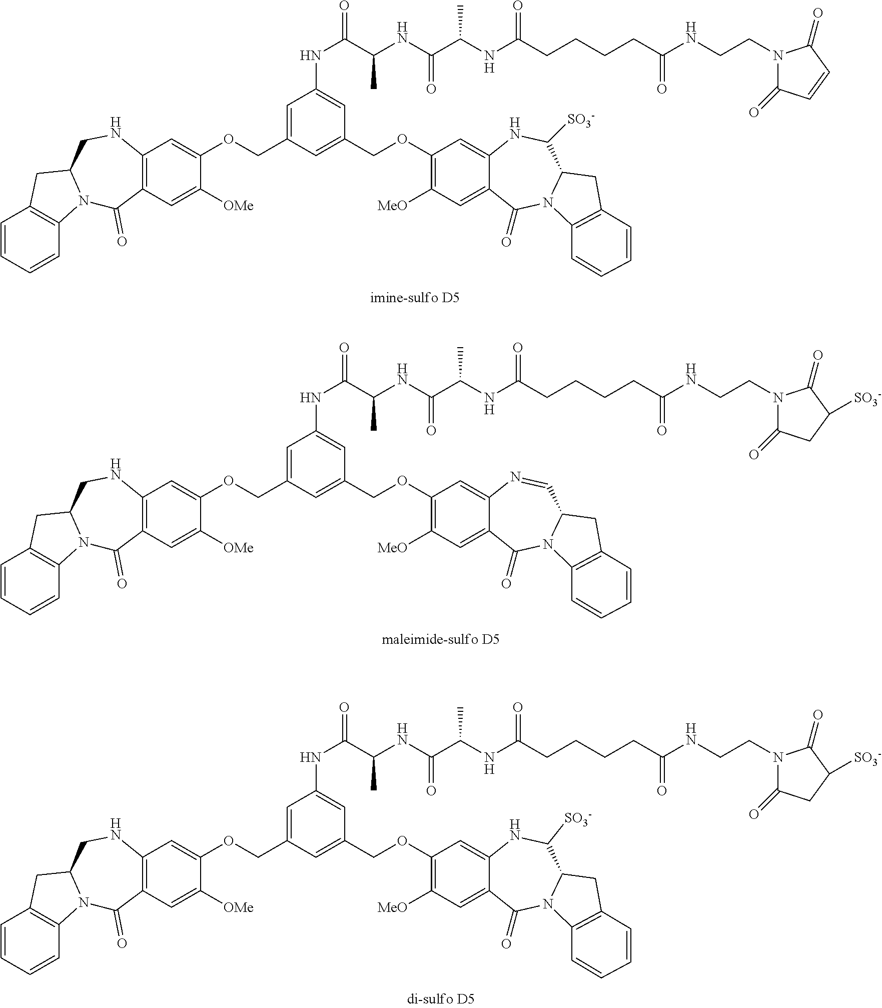

### Example 14

### Preparation of Antibody-Cytotoxic Agent Conjugates Using Cytotoxic Agent Prepared by Selective Sulfonation

The sulfonation reaction mixture (240 mL, 3.5 equiv.) prepared according to Example 13 was subsequently introduced into a 50 mM potassium phosphate pH 6.0 solution containing 10 g of hucMet27Gv1.3-C442 antibody with 2 engineered cysteines. At a final concentration of 2 mg/mL antibody and 15 v/v % DMA, the conjugation reaction was allowed to proceed for 18 h at 25° C. SEC analysis of the reaction product gives ADC with a DAR (drug to antibody ratio) of 1.9 and a % HMW (percentage of high molecule weight species) of 4.4% vs. 3.7% prior to conjugation.

### Example 15

### Preparation of MET Antibody Conjugates

Preparation of hucMET27v1.2-sulfo-SPDB-DM4 Conjugates

The sSPDB linker was dissolved in DMA to a concentration of 32.0 mM. The antibody was incubated at 3.8 mg/mL with a 21.5-fold molar excess of sSPDB linker for approximately 2 hours in a 25° C. water bath in a buffer of 60 mM EPPS pH 8.0, with 50 mM sodium chloride, 2 mM EDTA and 5% final DMA. The modified antibody was purified via Sephadex G-25 column into 50 mM EPPS, 50 mM sodium chloride, 2 mM EDTA, pH 8.0 buffer. The ratio of linker to antibody (LAR) was calculated by reducing and quantifying the released thiopyridine groups and assuming one thiopyridine per linked sSPDB. The conjugation reaction was then set up at 1.5 mg/mL antibody concentration containing 5% DMA and a 1.5-fold molar excess of DM4 over calculated LAR. After 15-20 hour incubation in a 25° C. water bath, the reaction mixture was purified via Sephadex G-25 column equilibrated in 10 mM succinate, 250 mM glycine, 0.5% sucrose, 0.01% Tween 20, pH 5.5 buffer and filtered through a 0.22 μm PVDF syringe filter. The number of DM4 molecules linked per antibody and the percentage of total free maytansinoid species were determined as described below under “analysis”. Conjugates with an average of 3-4 DM4 molecules per antibody were obtained with <2% present as unconjugated maytansinoid.

For DM conjugates, the molar concentration of antibody and linker were calculated according to Beer's law using the UV/Vis absorbance values at 280 and 343 nm and their extinction co-efficients. A 1:20 dilution of Ab-linker was used for the 280 nm value whereas a 1:5 dilution in a pH 7.5 buffer with 50 mM DTT was used for the 343 nm value. The DTT-treated sample represents sSPDB linker assuming a 1:1 ratio of released thiopyridine. The final ratio of linker to antibody was calculated from the resulting [Ab] and [sSPDB] values. The number of DM4 molecules per antibody was determined by measuring the UV/Vis absorbance at 252 and 280 nm and calculating the [Ab] and [DM4] using binomial equations that account for contribution of each component. The amount of unbound maytansinoid present in final cMet-DM4 conjugates was calculated from the resulting peak areas seen in samples analyzed via HISEP column (Supelco #58919 25 cm×4.6 mm, 5 μm). The percent free maytansinoid (% FM) present in the conjugate sample was calculated using the following equation: % Free Maytansinoid=(Reverse-phase PA 252 due to DM1)/(Reverse-phase PA 252 due to DM1+Flow through PA 252 due to DM1)×100%.

### Preparation of SMCC-DM1 Conjugates

The sulfosuccinimidyl-4-(N-maleimidomethyl)cyclohexane-1-carboxylate (sulfo-SMCC, Thermo Scientific Pierce) linker was dissolved in dimethylacetamide (DMA) and reacted at 1.3-molar excess to N2′-deacetyl-N-2′(3-mercapto-1-oxopropyl)-maytansine (DM1) in a 60:40 mixture of 50 mM succinate, pH 5.0 to DMA. After 10 minutes 0.2 mM of an N-ethylmaleimide (NEM) solution in ethanol was added to quench unreacted thiols. After 15 minutes 5-6 equivalents of this linker-drug mixture was added to the antibody in a buffer of 60 mM 4-(2-hydroxyethyl)-1-piperazinepropanesolfonic acid (EPPS), 10 mM phosphate, 139 mM sodium chloride, pH 8 containing 10% DMA at 2.5 mg/mL antibody concentration. After 15-20 hour incubation in a 25° C. water bath, the reaction mixture was purified using a SEPHADEX™ G25 columns equilibrated in a buffer containing 10 mM Tris-HCl, 80 mM sodium chloride, 3.5% sucrose 0.01% polysorbate 20, pH 7.5.

The number of DM1 molecules linked per antibody molecule was determined using the previously reported extinction coefficients for antibody and DM1 (Liu et al., Proc. Natl. Acad. Sci. USA, 93, 8618-8623 (1996)). The percentage of free maytansinoid present after the conjugation reaction was determined by injecting 50-200 μg conjugate onto a SUPELCOSIL™ Hisep™ column (Sigma-Aldrich) equilibrated in 25% acetonitrile in 100 mM ammonium acetate buffer, pH 7.0, and eluting in acetonitrile. The peak area of total free maytansinoid species (eluted in the gradient and identified by comparison of elution time with known standards) was measured using an absorbance detector set to a wavelength of 252 nm and compared with the peak area related to bound maytansinoid (eluted in the conjugate peak in the column flow-through fractions) to calculate the percentage of total free maytansinoid species. Conjugates with an average of 3-4 DM1 molecules per antibody were obtained with <3% present as unconjugated maytansinoid.

The binding affinity of anti-MET antibodies and conjugates to MET-expressing BxPC3 cells was measure using flow cytometry based methods. Primary antibodies and conjugates were used at concentrations from 1.5 μg/mL to 0.03 ng/mL. Secondary antibody used was FITC-conjugated goat anti-human IgG antibody (from Jackson ImmunoResearch) at 5 μg/mL in FACS buffer. Humanized antibody hu247.22.2 and its -SMCC-DM1 and -SPDB-DM4 conjugates bound BxPC3 cells with an affinity of 0.09 nM, 0.08 nM and 0.07 nM, respectively. Humanized antibody hu247.27.16, and its -SMCC-DM1 and -SPDB-DM4 conjugates bound BxPC3 cells with an affinity of 0.39 nM, 0.44 nM and 0.84 nM, respectively. This result indicates that maytansinoid conjugation of these antibodies does not significantly change the binding affinity to the MET antigen.

Preparation of hucMet27Gv1.3-Sulfo-SPDB-DM4 Conjugates

The sSPDB linker was dissolved in DMA to a concentration of 22.8 mM. The antibody was incubated at 4 mg/mL with a 21.5-fold molar excess of sSPDB linker for approximately 2 hours in a 25° C. water bath in a buffer of 60 mM EPPS, pH 8.0 with 50 mM sodium chloride, 2 mM EDTA and 5% final DMA. The modified antibody was purified via Sephadex G-25column into 50 mM EPPS, 50 mM sodium chloride, 2 mM EDTA, pH 8.0 buffer. The ratio of linker to antibody (LAR) was calculated by reducing and quantifying the released thiopyridine groups and assuming one thiopyridine per linked sSPDB. The conjugation reaction was then set up at 1.4-1.5 mg/mL antibody concentration containing 5% DMA and a 1.5-fold molar excess of DM4 over calculated LAR. After 15-20 hour incubation in a 25° C. water bath, the reaction mixture was purified via Sephadex G-25 columns equilibrated in 10 mM succinate, 250 mM glycine, 0.5% sucrose, 0.01% Tween 20, pH 5.5 buffer and filtered through a 0.22 μm PVDF syringe filter. The number of DM4 molecules linked per antibody and the percentage of total free maytansinoid species were determined as described below under “analysis”. Conjugates with an average of 3-4 DM4 molecules per antibody were obtained with <2% present as unconjugated maytansinoid.

Preparation of hucMet22-Sulfo-SPDB-DM4 Conjugates

The sSPDB linker was dissolved in DMA to a concentration of 22.8 mM. The antibody was incubated at 4 mg/mL with a 10-fold molar excess of sSPDB linker for 15-20 hours in a 25° C. water bath in 60 mM EPPS pH 8.5 buffer, with 50 mM potassium phosphate, 50 mM sodium chloride, 2 mM EDTA, pH 7.5, 5% final DMA and a 1.5-fold molar excess of DM4 over linker. After 15-20 hour incubation in a 25° C. water bath, the reaction mixture was purified via Sephadex G-25 column equilibrated in 10 mM succinate, 250 mM glycine, 0.5% sucrose, 0.01% Tween 20, pH 5.5 buffer and filtered through a 0.22 μm PVDF syringe filter. The number of DM4 molecules linked per antibody and the percentage of total free maytansinoid species were determined as described below under “analysis”. Conjugates with an average of 3-4 DM4 molecules per antibody were obtained with <2% present as unconjugated maytansinoid.

Preparation of hucMet27Gv1.3-DGN549 Conjugates (Lysine Linked)

cMet-DGN549 conjugates were made using a pre-sulfonated DGN549-NHS reagent (or D2). Stocks of DGN549-NHS were prepared in DMA (9.4-15 mM) and the reactive imines were sulfonated by incubating with 5-fold molar excess of sodium bisulfite (1M stock in 50 mM succinate, pH 5.0) in a final composition of ˜90% organic, 10% aqueous at 25° C. for three hours followed by 15-20 hours of incubation at 4° C. The antibody was buffer exchanged from PBS, pH 7.4 to 15 mM HEPES, pH 8.5 prior to conjugation. The conjugation reaction was carried out for 4 hours in a water bath at 25° C. at an Ab concentration of 2.0 mg/mL in 15 mM HEPES, pH 8.5 buffer containing 10% DMA and 3.1-3.5 molar excess of sulfonated DGN549-NHS over Ab. The reaction mixture was purified via Sephadex G-25 column equilibrated in a buffer containing 20 mM histidine, 50 mM sodium chloride, 8.5% sucrose, 0.01% Tween-20, 50 μM sodium bisulfite, pH 6.2, filtered using 0.22 um PVDF syringe filter, and assayed. The number of DGN549 molecules linked per antibody and the percentage of total free DGN549 species were determined as described below under “analysis”. Conjugates with an average of 2-3 DGN549 molecules per antibody were obtained with <1% present as unconjugated DGN549.

For DGN conjugates, the number of DGN549 molecules per antibody was determined by measuring the UV/Vis absorbance at 280 and 330 nm and calculating the [Ab] and [DGN549] according to Beer's law. Samples were read neat or at a 1:2 dilution. To determine the amount of unbound DGN549, conjugates were analyzed by a dual-column system (TOSOH SEC QC-PAK GFC 300 and Agilent Zorbax C18 columns) to calculate total AUC for free DGN549. The free DGN549 concentration was determined by using the resulting peak AUC against a pre-established standard curve.

Preparation of hucMet27Gv1.3-C442-DGN549 Conjugates

HucMet27Gv1.3 antibody bearing two unpaired cysteine residues in the reduced state was prepared using standard procedures. The conjugation reaction was carried out using this intermediate at a final antibody concentration of 1 mg/mL in PBS containing 5 mM EDTA, pH 6.0 and 10 molar equivalents of Mal-DGN549 (or D5, as a 6.8 mM stock solution in DMA) with 2% v/v DMA and 38% v/v propylene glycol. The conjugation reaction was carried out for 15-20 hours in a water bath at 25° C. The conjugate was purified into 20 mM succinate, 8.5% sucrose, 50 μM sodium bisulfite, 0.01% Tween 20, pH 4.2 buffer via Sephadex G-25column, concentrated by ultrafiltration via regenerated cellulose membrane (Amicon Ultracel, 10,000 Da molecular weight cutoff), and filtered through a 0.22 μm PVDF syringe filter. The number of DGN549 molecules linked per antibody and the percentage of total free DGN549 species were determined as described below under “analysis”. Conjugates with an average of 2-3 DGN549 molecules per antibody were obtained with <1% present as unconjugated DGN549.

### Example 16

### In vitro Cytotoxicity of SMCC-DM1 MET Antibody Conjugates

### In Vitro Cytotoxic Activity in MKN45 Cells

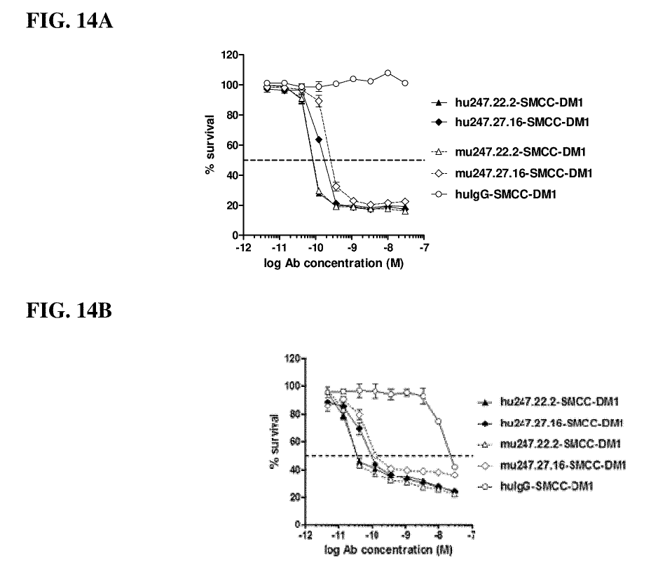

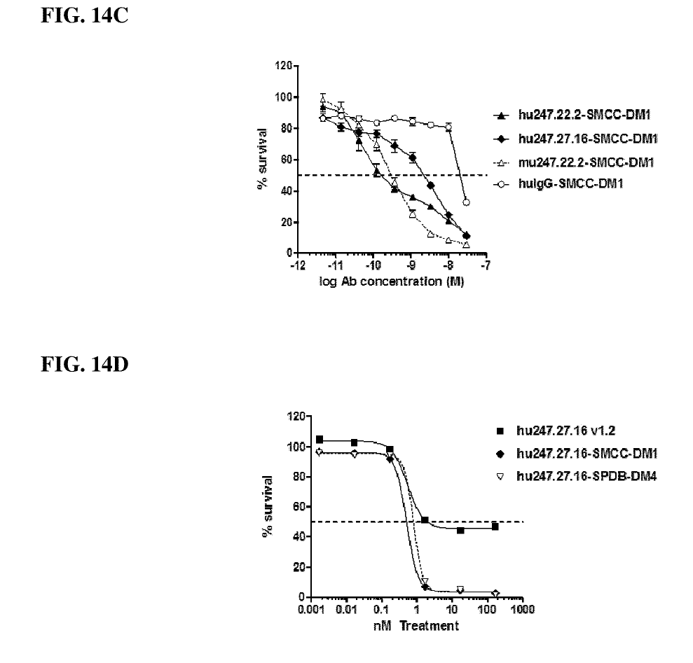

The in vitro cytotoxicity of SMCC-DM1 conjugates made with anti-MET antibodies hu247.22.2, hu247.27.16, mu247.22.2, and mu247.27.16 was compared to the activity of a non-specific huIgG-SMCC-DM1 conjugate in MET-expressing MKN45 cells and the results from a typical cytotoxicity assay are shown in FIG. 14A. All anti-MET antibody conjugates resulted in specific cell killing as compared to the huIgG control conjugate. The EC50 values correspond to 0.07 nM, 0.15 nM, 0.08 nM, and 0.27 nM, for SMCC-DM1 conjugates of hu247.22.2, hu247.27.16, mu247.22.2, and mu247.27.16, respectively. In contrast, SMCC-DM1 conjugates of a non-binding huIgG control antibody resulted in cell killing with an EC50 value of >30 nM.

### In Vitro Cytotoxic Activity in NCI-H441 Cells

The in vitro cytotoxicity of SMCC-DM1 conjugates made with anti-MET antibodies hu247.22.2, hu247.27.16, mu247.22.2, and mu247.27.16 was compared to the activity of a non-specific huIgG-SMCC-DM1 conjugate in MET-expressing NCI-H441 cells and the results from a typical cytotoxicity assay are shown in FIG. 14B. All anti-MET antibody conjugates resulted in specific cell killing as compared to the huIgG control conjugate. The EC50 values correspond to 0.04 nM, 0.08 nM, 0.04 nM, and 0.09 nM, for SMCC-DM1 conjugates of hu247.22.2, hu247.27.16, mu247.22.2, and mu247.27.16, respectively. In contrast, SMCC-DM1 conjugates of a non-binding huIgG control antibody resulted in cell killing with an EC50 value of approximately 25 nM.

### In Vitro Cytotoxic Activity in BxPC3 Cells

The in vitro cytotoxicity of SMCC-DM1 conjugates made with anti-MET antibodies hu247.22.2, hu247.27.16, and mu247.22.2 was compared to the activity of a non-specific huIgG-SMCC-DM1 conjugate in MET-expressing BxPC3 cells and the results from a typical cytotoxicity assay are shown in FIG. 14C. All anti-MET antibody conjugates resulted in specific cell killing as compared to the huIgG control conjugate. The EC50 values correspond to 0.15 nM, 2.0 nM, and 0.27 nM, for SMCC-DM1 conjugates of hu247.22.2, hu247.27.16, and mu247.22.2, respectively. In contrast, SMCC-DM1 conjugates of a non-binding huIgG control antibody resulted in cell killing with an EC50 value of approximately 26 nM.

In Vitro Cytotoxic Activity in SNU-5 Cells

The in vitro cytotoxicity of hu247.27.16 antibody was compared to the activity of hu247.27.16-SMCC-DM1 and -SPDB-DM4 conjugates in MET-expressing SNU-5 cells and the results from a typical cytotoxicity assay are shown in FIG. 14D.

SNU-5 cells were obtained from American Type Culture Collection (ATCC) and maintained in culture media (IMDM with 20% fetal bovine serum) at 37° C. in a humidified atmosphere containing 5% CO₂. SNU-5 cells were plated at 10,000 cells per well in the same culture media in a 96 well plate and incubated overnight at 37° C. The next day, antibodies or conjugates were diluted into the same culture media and added to wells at various concentrations in a total volume of 200 μL per well. Cells were incubated at 37° C. in a humidified 5% CO₂ incubator for 5 days. Subsequently, cells were lysed and viability was assessed using CellTiter Glo reagent (Promega), which quantitates total cellular ATP content. A Trilux luminometer was utilized to measure relative luminescence units (RLU) in each well and percent viability was calculated by dividing each treated sample value by the average value of wells with untreated cells. The percent viability value was plotted against the antibody concentration in a semi-log plot for each treatment. A dose-response curve was generated by non-linear regression and the EC50 value of each curve was calculated using GraphPad Prism (GraphPad software, San Diego, Calif.).

The hu247.27.16-SMCC-DM1 and -SPDB-DM4 conjugates resulted in complete killing of SNU-5 cells with EC50 values of approximately 0.5 and 0.8 nM, respectively. The unconjugated hu247.27.16 antibody also resulted in cell killing and reduced the SNU-5 viability to approximately 50% with an EC50 of approximately 0.5 nM. This demonstrates that the hu247.27.16 antibody can have inhibitory activity against MET expressing cells and conjugation to maytansinoids, such as for example DM1 or DM4, enhances this cell killing activity.

### Example 17

### In Vitro Cytotoxicity of MET Antibody Conjugates

The ability of anti-cMet antibody conjugates to kill tumor cells was measured using in vitro cytotoxicity assays. Target cells were plated at 2,000 cells per well in 100 μL in complete RPMI media (RPMI-1640, 10% fetal bovine serum, 2 mM glutamine,1% penicillin-streptomycin, all reagents from Invitrogen). Conjugates were diluted in complete RPMI media using dilution series and 100 μL of each dilution was added per well. The final concentration typically ranged from 3×10⁻⁸ M to 4.6×10⁻¹² M for DM4 conjugates and 1×10⁻⁸ M to 1.5×10⁻¹³ M for DGN549 conjugates. Cells were incubated at 37° C. in a humidified 5% CO₂ incubator for 4 to 5 days. Viability of remaining cells was determined by colorimetric WST-8 assay (Dojindo Molecular Technologies). WST-8 is reduced by dehydrogenases in living cells to an orange formazan product that is soluble in tissue culture medium. The amount of formazan produced is directly proportional to the number of living cells. WST-8 was added to 10% of the final volume and plates were incubated at 37° C. in a humidified 5% CO₂ incubator for an additional 2-4 hours. Plates were analyzed by measuring the absorbance at 450 nm (A₄₅₀) in a multiwell plate reader. Background A₄₅₀ absorbance of wells with media and WST-8 only was subtracted from all values. The percent viability was calculated by dividing each treated sample value by the average value of wells with untreated cells. Percent viability=100*(A₄₅₀ treated sample−A₄₅₀ background)/(A₄₅₀ untreated sample−A₄₅₀ background). The percent viability value was plotted against the antibody concentration in a semi-log plot for each treatment. A dose-response curve was generated by non-linear regression and the EC50 value of each curve was calculated using GraphPad Prism 6.

### In Vitro Cytotoxic Activity in Gastic Carcinoma Cell Lines

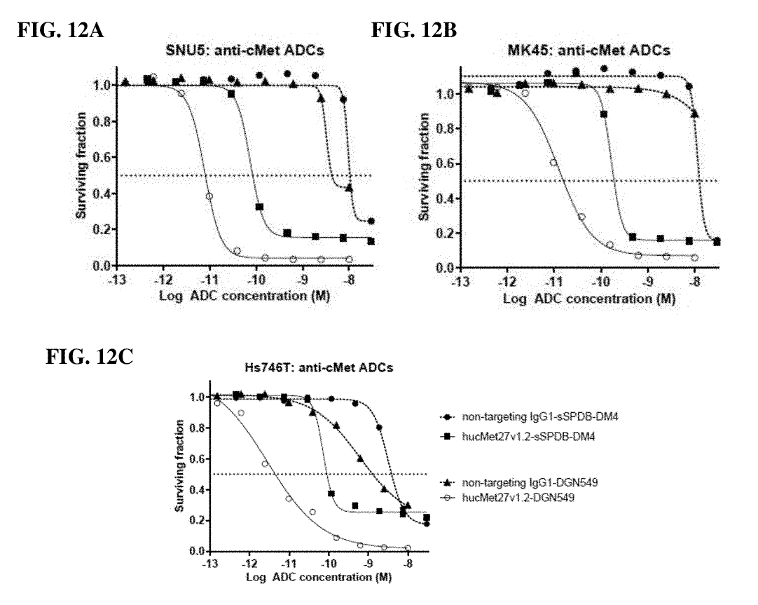

The in vitro cytotoxicity of sSBDP-DM4 and DGN549 conjugates made with anti-cMet antibody hucMet27v1.2 was compared to the activity of non-targeting IgG1 conjugates in the MET-amplified, cMet-overexpressing gastric carcinoma cell lines: SNU5, MKN45 and Hs746T. The results from typical cytotoxicity assays are shown in FIG. 12A through FIG. 12C. All anti-cMet antibody conjugates resulted in specific cell killing as compared to the IgG1 control conjugates. The EC50 values for hucMet27v1.2-sSPDB-DM4 conjugates was 0.08 nM in SNU5 cells, 0.17 nM in MKN45 cells and 0.07 nM in Hs746T cells. In contrast, sSPDB-DM4 conjugates of a non-targeting IgG1 control antibody resulted in cell killing with an EC50 value of 10 nM, 12 nM and 3 nM respectively. Strikingly, the hucMet27v1.2-DGN549 conjugate was extremely potent resulting in the complete reduction in cell viability at even low concentrations. The EC50 values for hucMet27v1.2-sSPDB-DM4 conjugates was 0.008 nM in SNU5 cells, 0.013 nM in MKN45 cells and 0.003 nM in Hs746T cells and in all cases was at least 3 logs more active than a non-targeting conjugate.

### In Vitro Cytotoxic Activity in NSCLC Cell Lines

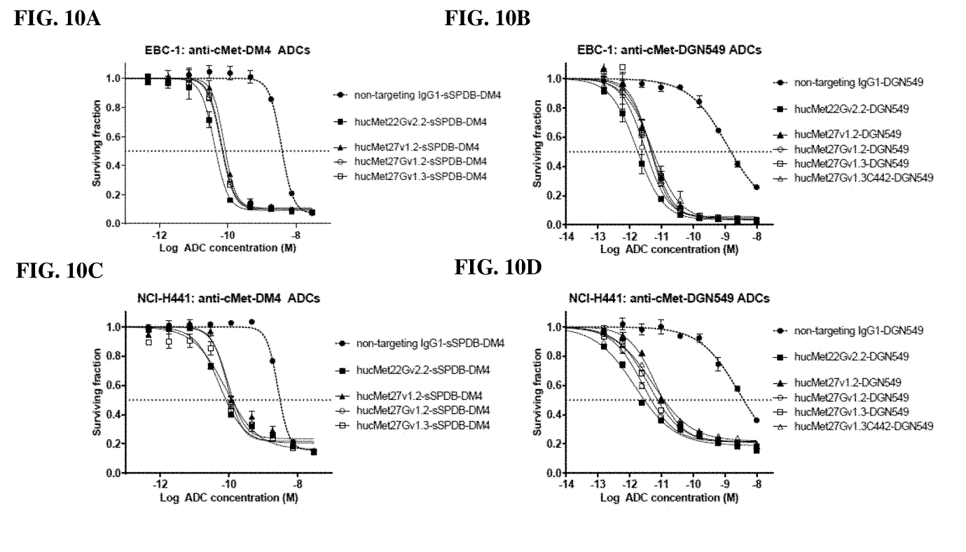

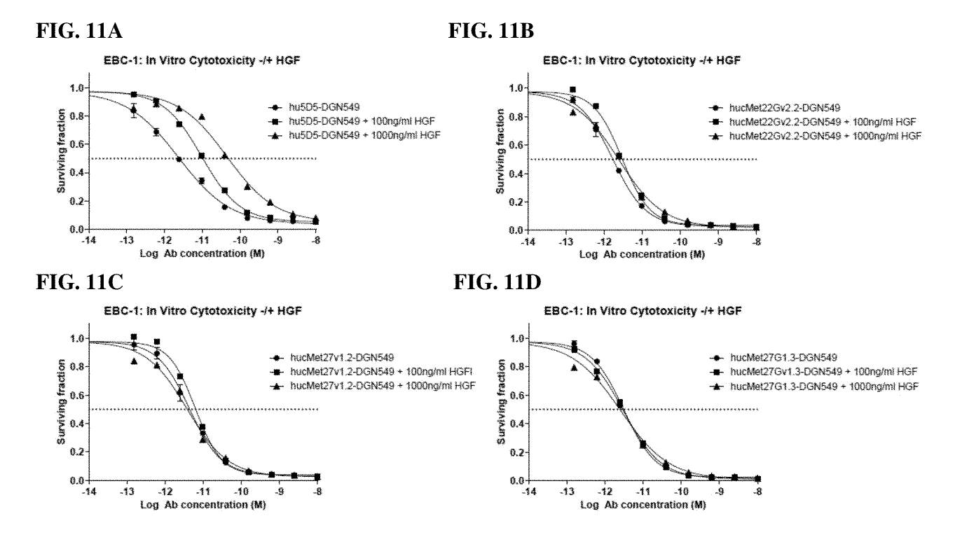

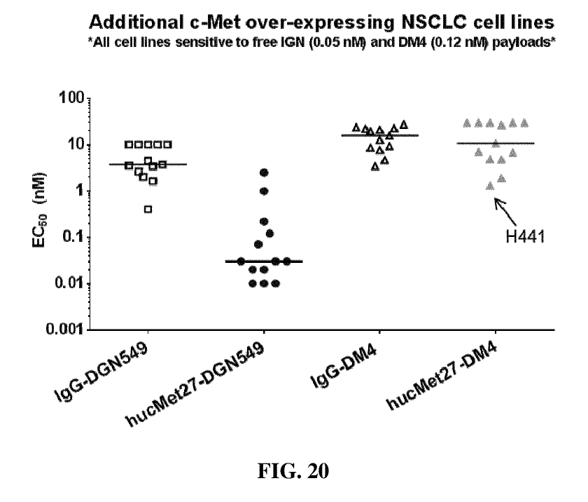

To expand upon the in vitro cytotoxicity activity seen with the hucMet27v1.2 conjugates in gastric carcinoma cell lines, additional anti-cMet sSBDP-DM4 and DGN549 conjugates were compared to the activity of non-targeting IgG1 conjugates in cMet-overexpressing NSCLC cell lines: EBC-1 (MET-amplified, cMet-overexpressed) and NCI-H411 (MET non-amplified, cMet-overexpressed). The results from typical cytotoxicity assays are shown in FIG. 10A through FIG. 10D. In each case tested, a good specificity window is observed, suggesting that cytotoxicity is a result of anti-cMet antibody binding to target cells. For the anti-cMet-sSPDB-DM4 conjugates, the EC50 values ranged from 41 to 95 pM. The anti-cMet-DGN549 conjugates in particular were very potent with EC50 values from 1 to 5 pM and were ˜3 logs more active than a non-targeting IgG1 conjugate. Surprisingly, the anti-cMet-DGN549 conjugates were also very potent in the non-amplified, overexpressed setting, whereas the huCMET27-sSPDB-DM4 conjugate did not show targeted potency on a majority of c-MET over-expressing NSCLC cell lines (see, FIG. 20).

In Vitro Cytotoxicity Activity of huCMet27Gv1.3 Hinge Modified Conjugates in NSCLC and Gastric Carcinoma Cell Lines

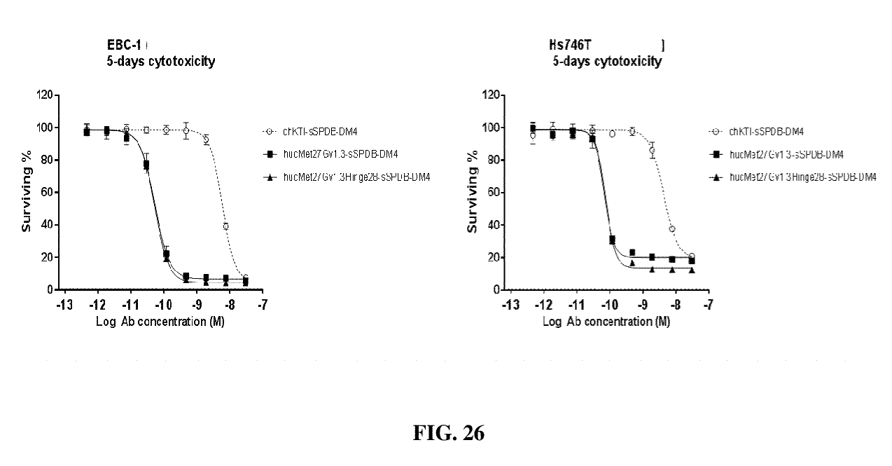

To expand upon the in vitro cytotoxicity activity seen with the hucMet27Gv1.3 conjugates in NSCLC and gastric carcinoma cell lines, additional anti-cMet-sSPDB-DM4 conjugates were compared to the activity of non-targeting IgG1 conjugates (chKTI-sSPDB-DM4) in EBC-1 (MET-amplified NSCLC cell line) and Hs746T (MET-amplified gastric carcinoma cell line). The results from typical cytotoxicity assays are shown in FIG. 26. In each case tested, a good specificity window is observed, suggesting that cytotoxicity is a result of anti-cMet antibody binding to target cells. Both hucMet27Gv1.3-sSPDB-DM4 and hucMet27Gv1.3Hinge28-sSPDB-DM4 conjugates were very potent with EC50 values ranging from 0.05 nM to 0.07 nM and were ˜2 logs more active than a non-targeting IgG1 conjugates.

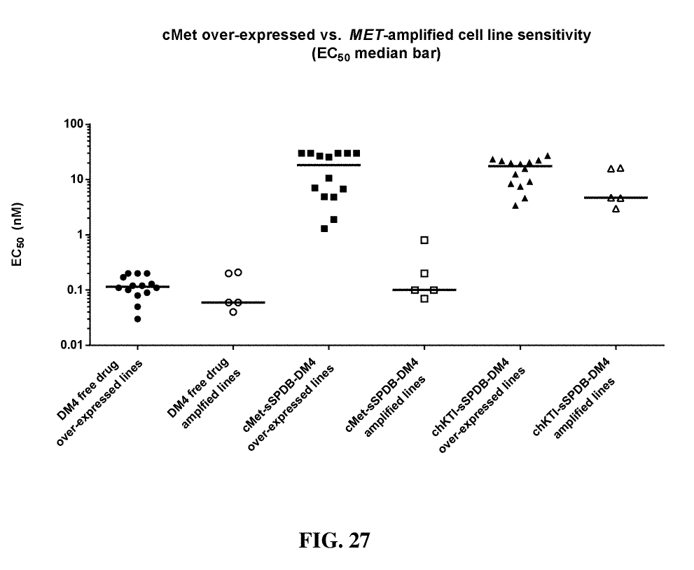

When the EC50 values of anti-cMet sSPDB-DM4 conjugates were analyzed, it was observed that the MET-amplified cell lines had lower EC50 values than over-expressed cell lines. Both sets of lines were equally sensitive to the DM4 free payload and equally insensitive to the non-targeting chKTI-sSPDB-DM4 controls (FIG. 27).

### In Vitro Cytotoxic Activity in Hep3B Cells

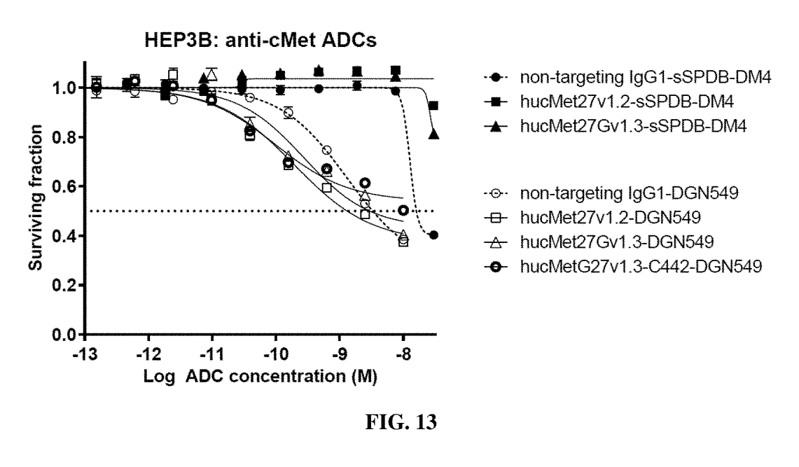

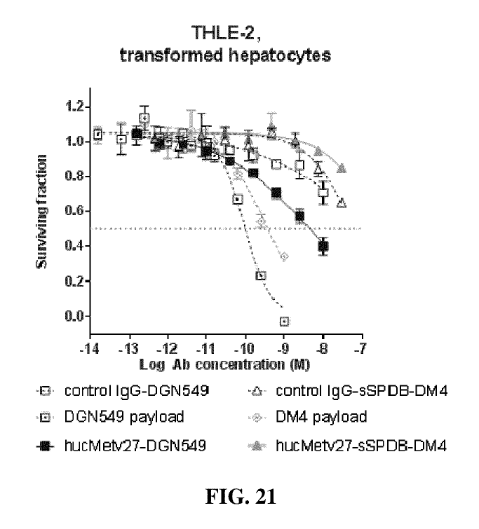

To evaluate the potency of anti-cMet conjugates in cells expressing a low level of cMet, we compared the in vitro cytotoxicity activity of anti-cMet conjugates to non-targeting IgG1 conjugates in the hepatocellular carcinoma Hep3B cell line. Hep3B cells have a normal MET gene copy number and express less than 30,000 cell surface receptors per cell. The results from typical cytotoxicity assays are shown in FIG. 13. Even at high concentrations sSPDB-DM4 and DGN54 conjugates only showed marginal levels of cytotoxicity and there was no specificity window observed, indicating that the low level of activity was non-targeted. Similarly, even at high concentrations, huCMET27 conjugates are 1000× less potent on transformed liver cells (see, FIG. 21).

### Example 18

### In Vitro Cytotoxicity of Anti-cMet ADCs in Presence of the cMet Ligand, HGF

Activation of cMET in tumors can occur through both HGF-dependent autocrine and paracrine mechanisms. Either HGF is released from the surrounding stromal cells, resulting in a constitutive paracrine cMET activation; or HGF and cMET can be coexpressed in tumors leading to autocrine activation, as found in carcinomas, sarcomas, gliomas, and B-cell tumors. To test the potency of anti-cMet antibody conjugates in the presence of the native c-Met ligand, in vitro cytotoxicity assays were performed in the presence of HGF. Briefly, 2,000 EBC-1 cells/well were plated in 96-well plates and a dilution series of anti-cMet conjugates was added in complete RPMI media alone or supplemented with 100 ng/mL or 1000 ng/mL of HGF. Control wells containing cells but lacking conjugate, as well as wells contained medium only, were included in each assay plate. Assays were performed in triplicate for each data point. The plates were incubated at 37° C. in a humidified 5% CO₂ incubator for 5 days. Then the relative number of viable cells in each well was determined using the WST-8 based Cell Counting Kit-8 (Dojindo Molecular Technologies). The surviving fraction of cells in each well was calculated by first correcting for the medium background absorbance, and then dividing each value by the average of the values in the control wells (non-treated cells). The percentage of surviving cells was plotted against conjugate concentration and the EC50 of activity was calculated using a nonlinear regression analysis (GraphPad Prims 6).

All anti-cMet-DGN549 conjugates showed similar potency in the absence of ligand (FIG. 11B through FIG. 11D). Interestingly, the results show that the potency of hucMetGv2.2-DGN549, hucMetv1.2-DGN549 & hucMetGv1.3-DGN549 were uneffected by even high concentrations of HGF. In contrast, the potency of the hu5D5-DGN549 conjugate was dramatically reduced with increasing concentrations of HGF (FIG. 11A). This data suggests that unlike hu5D5-DGN549, the hucMetGv2.2-DGN549, hucMetv1.2-DGN549 & hucMetGv1.3-DGN549 conjugates would likely retain their full potency even if HGF levels are locally high at the tumor site.

### Example 19

### Anti-Tumor Activity of MET Antibody SMCC-DM1 Conjugates

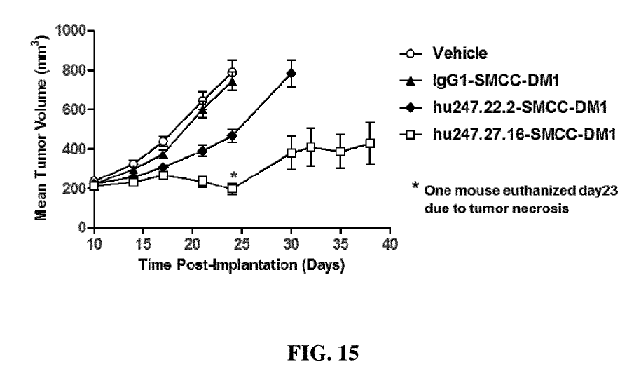

Anti-MET antibodies and their respective SMCC-DM1 conjugates were tested in an EBC-1 xenograft model established in female athymic nu/nu mice (Harlan, Livermore, Calif.) as described above. Animals were randomized according to tumor volume into treatment groups (n=10/group) when tumors reached a mean tumor volume of approximately 200 mm³ and treated once on day 11 post tumor implantation with 10 mg/kg of either vehicle, hu247.22.2-SMCC-DM1, hu247.27.16-SMCC-DM1 or a non-binding huIgG-SMCC-DM1 control conjugate. Following randomization and the start of dosing, tumor xenografts were measured as described above. Statistical significance in differences in tumor volume was determined by ANOVA and pair wise comparisons were made by Tukey's post-test using SigmaPlot. The mean tumor volume of the different treatment groups is plotted against time post tumor implantation in FIG. 15. It is apparent that treatment with a non-binding huIgG-SMCC-DM1 control conjugate did not reduce the tumor volume as compared to the vehicle control. In contrast, treatment with the hu247.22.2-SMCC-DM1 or hu247.27.16-SMCC-DM1 conjugates resulted in a significant reduction in mean tumor volume (p<0.001 as compared to huIgG-SMCC-DM1 control on day 24).

### Example 20

### Anti-Tumor Activity of MET Antibody Conjugates in Nude Mice Bearing Ebc-1 Human Non-Small Cell Lung Squamous Cell Carcinoma Xenografts

The antitumor activity of varying doses of hucMet27v1.2-DGN549 and a single dose of hucMet27v1.2-sSPDB-DM4 conjugates were evaluated in female Athymic Nude-Foxn1^{nu} mice bearing Ebc-1 cells, a human non-small cell lune squamous cell carcinoma xenograft model.

Ebc-1 cells were harvested for inoculation with 99% cell viability determined by trypan blue exclusion. Mice were inoculated with 5×10⁶ Ebc-1 cells in 0.1 ml of serum free medium by subcutaneous injection on the right flank using a 27-gauge needle. Eighty female Nude mice (6-7 weeks of age) were randomized into 8 groups (10-mice per group) by tumor volume. The mean tumor volumes ranged were betw. 108-115 mm³. The mice were measured, randomized, and dosed based on the tumor volume on day 7 post implantation. Administration of the test agents and vehicle were carried out intravenously by using a 1.0 ml syringe fitted with a 27-gauge needle. The groups included were a control group dosed with vehicle (PBS), chKTI-sSPDB-DM4 at 5 mg/kg, hucMet27v1.2-sSPDB-DM4 at 5 mg/kg, chKTI-DGN549 at 3 μg/kg (by payload; 0.18 mg/kg by antibody), chKTI-DGN549 at 10 μg/kg (by payload; 0.6 mg/kg by antibody) hucMet27v1.2-DGN549 at 3 μg/kg (by payload; 0.18 mg/kg by antibody), hucMet27v1.2-DGN549 at 10 μg/kg (by payload, 0.6 mg/kg by antibody). All test agents were administered as a single i.v. dose.

Tumor measurements were recorded 3 times weekly. Tumor burden (mm³) was estimated from caliper measurements by the formula for the volume measurement as: Tumor burden (mm³)=(L×W2)/2, where L and W are the respective orthogonal tumor length and width measurements (in mm). The primary endpoints that were used to evaluate efficacy were tumor growth inhibition, % T/C, complete and partial tumor response, and the number of tumor-free survivors at the end of the study. In this experiment, % T/C was evaluated when the median Control tumor burden reached to 1080 mm³ (on Day 24).

Body weights (BW) of mice were expressed as percent change in body weight from the pre-treatment body weight as follows: % BW change=[(BW post/BW pre)−1]×100, where BW post is weight after treatment and BW pre is the starting body weight prior to treatment. Percent body weight loss (BWL) was expressed as the mean change in body weight post treatment. Animals were sacrificed when the tumor volume was larger than 1000 mm³ or necrotic, or if body weight dropped by 20% more at any point in the study.

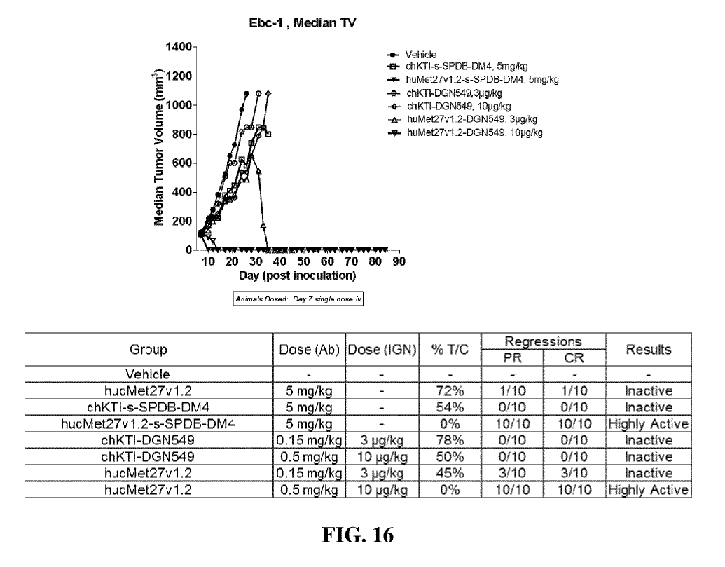

The results of the study are shown in FIG. 16. hucMet27v1.2-sSPDB-DM4, conjugate was highly active at 5 mg/kg dose. hucMet27v1.2-sSPDB-DM4 conjugate had a tumor growth inhibition (T/C) value of 0%, at 5 mg/kg dose (with 10/10 complete regressions). hucMet27v1.2-DGN549 was highly active at 10 μg/kg (by payload; 0.6 mg/kg by antibody) (with 10/10 complete regressions) and had a tumor growth inhibition (T/C) value of 0. While hucMet27v1.2-DGN549 was inactive at 3 μg/kg (by payload; 0.18 mg/kg by antibody) with a tumor growth inhibition (T/C) value of 45%, there were 3/10 complete regressions.

Treatments with chKTI-sSPDB-DM4, hucMet27v1.2-sSPDB-DM4, chKTI-DGN549 and hucMet27v1.2-DGN549 were all well tolerated at the indicated doses, and no significant body weight loss was observed in this study. Two regimens, i.e. hucMet27v1.2-sSPDB-DM4 at 5 mg/kg and hucMet27v1.2-DGN549 at 10 μg/kg (by payload; 0.6 mg/kg by antibody), showed potent activity inducing a 100% incidence of tumor regressions, with all mice remaining tumor free at study termination on Day 91. Tumor regressions on both regimens were immediate, starting at early time points following the dosing on Day7. All the rest of the treatment regimens were inactive in this study.

### Example 21

### Anti-Tumor Activity of Anti-cMet Antibody Drug Conjugates in SCID Mice Bearing HSC-2 Human Head and Neck Squamous Cell Carcinoma (HNSCC) Xenograft

The antitumor activity of varying doses of hucMet27Gv1.3-C442-DGN549 and a single dose of hMucMet27Gv1.3-sSPDB-DM4 conjugates were evaluated in female SCID mice bearing HSC2 cells, a HNSCC xenograft model.

HSC2 cells were harvested for inoculation on Sep. 16, 2016, with 100% viability determined by trypan blue exclusion. Mice were inoculated with 5×10⁶ HSC2 cells in 0.1 ml 50% Matrigel/50% serum free medium by subcutaneous injection in the area on the right hind flank. Thirty-six female SCID mice (6 weeks of age) were obtained from Charles River Laboratories. Upon receipt, the animals were observed for 4 days prior to study initiation. Animals showed no sign of disease or illness upon arrival, or prior to treatment.

Thirty-six mice were randomized into 6 groups (6 mice per group) by tumor volume. The tumor volumes ranged from 84.77 to 118.74 mm³ (Mean TV for groups was betw. 95.57-101.91 mm³). The mice were randomized and dosed based on the tumor volume on Day 6 post implantation. Body weight of the mice ranged from 16.85 to 20.76 grams (Mean BW for groups were betw. 18.24-19.31 grams). Mice in each group were identified by ear punch method. Administration of the test agents and vehicle were carried out intravenously by using a 1.0 ml syringe fitted with a 27 gauge, ½ inch needle. The groups included: a control group dosed with vehicle (PBS) at 150 μL/mouse, chKTI-sSPDB-DM4 at 5 mg/kg, hucMet27Gv1.3-sSPDB-DM4 at 5 mg/kg, huKTI-C442-DGN549 at 10 μ/kg (by payload; 0.5 mg/kg by antibody), and hucMetGv271.3-C442-DGN549 at 3 μ/kg and 10 μg/kg (by payload; 0.15 mg/kg and 0.5 mg/kg by antibody respectively). All test agents were administered as a single i.v. dose.

Tumor size was measured two times per week in three dimensions using a caliper. The tumor volume was expressed in mm³ using the formula V=Length×Width×Height×½. A mouse was considered to have a partial regression (PR) when tumor volume was reduced by 50% or greater, complete tumor regression (CR) when no palpable tumor could be detected and tumor-free survivor (TFS) is the number of mice tumor free at the end of the study. Body weight of all the mice was measured twice per week as a rough index of drug toxicity. Tumor volume and body weight were determined by StudyLog software.

Body weights (BW) of mice were expressed as percent change in body weight from the pre-treatment body weight as follows: % BW change=[(BW post/BW pre)−1]×100, where BW post is weight after treatment and BW pre is the starting body weight prior to treatment. Percent body weight loss (BWL) was expressed as the mean change in body weight post treatment. Animals were sacrificed when the tumor volume was larger than 1000 mm³ or necrotic, or if body weight dropped by 20% more at any point in the study.

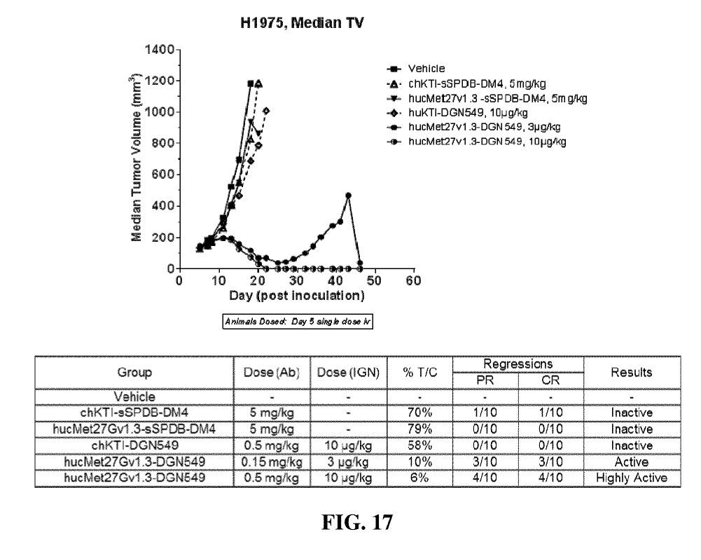

The results of the study are shown in FIG. 17. hMucMet27Gv1.3-sSPDB-DM4 conjugate was active at 5 mg/kg dose. hucMet27Gv1.3-sSPDB-DM4 conjugate had a tumor growth inhibition (T/C) value of 11% at 5 mg/kg dose (with 1/6 partial regressions and 1/6 complete regressions). hucMet27Gv1.3-C442-DGN549 was highly active at 3 μg/kg (by payload; 0.15 mg/kg by antibody) (with 6/6 partial regressions and 4/6 complete regressions and had a had a tumor growth inhibition (T/C) value of 4%. hucMet27Gv1.3-C442-DGN549 was highly active at 10 μg/kg (by payload; 0.5 mg/kg by antibody) (with 6/6 partial regressions and 4/6 complete regressions) and had a tumor growth inhibition (T/C) value of 4%.

Treatments with chKTI-sSPDB-DM4, hucMet27Gv1.3-sSPDB-DM4, huKTI-C442-DGN549 and hucMet27Gv1.3-C442-DGN549 were all well tolerated at the indicated doses, and no significant body weight loss was observed in this study. Two regimens, i.e., hucMet27Gv1.3-C442-DGN549 at 3 ug/kg (by payload; 0.15 mg/kg by antibody) and 10 μg/kg (by payload; 0.5 mg/kg by antibody), showed potent activity inducing a 66.7% incidence of tumor regressions, with all 4 mice out of 6 mice remaining tumor free at Day 72. Tumor regressions on both regimens were immediate, starting at early time points following the dosing on Day6. One additional treatment group, hucMet27Gv1.3-sSPDB-DM4 at 5 mg/kg also showed relatively good anti-tumor activity inducing a 16.7% incidence of tumor regressions with one mouse staying at complete regression at Day 72. All the rest of the treatment regimens other than these three treatment groups were inactive in this study.

### Example 22

### Anti-Tumor Activity of Anti-cMet Antibody Drug Conjugates in Nude Mice Bearing H1975 Human Non-Small Cell Lung Squamous Cell Carcinoma Xenografts

The antitumor activity of varying doses of hucMet27Gv1.3-DGN549 (lysine linked) and a single dose of hucMet27Gv1.3-sSPDB-DM4 conjugates were evaluated in female Athymic Nude-Foxn1nu mice bearing H1975 cells, a human non-small cell lune squamous cell carcinoma xenograft model.

H1975 cells were harvested for inoculation with 99% cell viability determined by trypan blue exclusion. Mice were inoculated with 5×106 H1975 cells in 0.1 ml of 50% Matrigel/50% serum free medium by subcutaneous injection on the right flank using a 27-gauge needle. Sixty female Nude mice (6-7 weeks of age) were randomized into 6 groups (10-mice per group) by tumor volume. The mean tumor volumes ranged were betw. 113-144 mm³. The mice were measured, randomized, and dosed based on the tumor volume on day 5 post implantation. Administration of the test agents and vehicle were carried out intravenously by using a 1.0 ml syringe fitted with a 27-gauge needle. The groups included were a control group dosed with vehicle (PBS), chKTI-sSPDB-DM4 at 5 mg/kg, hucMet27Gv1.3-sSPDB-DM4 at 5 mg/kg, chKTI-DGN549 at 10 μg/kg (by payload; 0.5 mg/kg by antibody), hucMet27Gv1.3-DGN549 at 3 μg/kg (by payload; 0.15 mg/kg by antibody), hucMet27Gv1.3-DGN549 at 10 μg/kg (by payload; 0.5 mg/kg by antibody). All test agents were administered as a single i.v. dose.

Tumor measurements were recorded 3 times weekly. Tumor burden (mm³) was estimated from caliper measurements by the formula for the volume measurement as: Tumor burden (mm3)=(L×W2)/2, where L and W are the respective orthogonal tumor length and width measurements (in mm). The primary endpoints that were used to evaluate efficacy were tumor growth inhibition, % T/C, complete and partial tumor response, and the number of tumor-free survivors at the end of the study. In this experiment, % T/C was evaluated when the median Control tumor burden reached to 1183 mm³ (on Day 18).

Body weights (BW) of mice were expressed as percent change in body weight from the pre-treatment body weight as follows: % BW change=[(BW post/BW pre)−1]×100, where BW post is weight after treatment and BW pre is the starting body weight prior to treatment. Percent body weight loss (BWL) was expressed as the mean change in body weight post treatment. Animals were sacrificed when the tumor volume was larger than 1000 mm³ or necrotic, or if body weight dropped by 20% more at any point in the study.

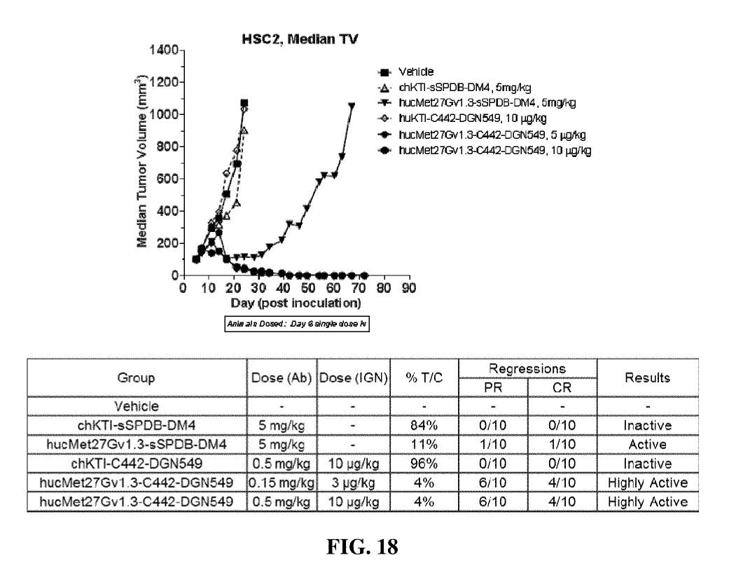

The results of the study are shown in FIG. 18. hucMet27Gv1.3-sSPDB-DM4, conjugate was inactive at 5 mg/kg dose. hucMet27Gv1.3-sSPDB-DM4 conjugate had a tumor growth inhibition (T/C) value of 79%, at 5 mg/kg dose (with No PR or CRs). hucMet27Gv1.3-DGN549 was highly active at 10 μg/kg (by payload; 0.5 mg/kg by antibody) (with 4 out of10 complete regressions) and had a tumor growth inhibition (T/C) value of 6%. hucMet27Gv1.3-DGN549 was active at 3 μg/kg (by payload; 0.15 mg/kg by antibody) (with 3 out 10 complete regressions) and had a tumor growth inhibition (T/C) value of 10%.

Treatments with chKTI-sSPDB-DM4, hucMet27Gv1.3-sSPDB-DM4, chKTI-DGN549 and hucMet27Gv1.3-DGN549 were all well tolerated at the indicated doses, and no significant body weight loss was observed in this study.

While the invention has been described in detail and with reference to specific aspects thereof, it is apparent to one of skill in the art that various changes and modifications can be made thereto without departing from the spirit and scope thereof.

All references mentioned herein are incorporated by reference in their entireties.

1. An isolated monoclonal antibody, or antigen-binding fragment thereof, that specifically binds to an epitope in the extracellular region of human cMET, wherein said antibody or antigen-binding fragment thereof comprises light chain complementary determining regions LC CDR1, LC CDR2, and LC CDR3 and heavy chain complementary determining regions HC CDR1, HC CDR2, and HC CDR3 having the sequences selected from the group consisting of:
(a) SEQ ID NOs:4, 5, and 7 and SEQ ID NOs:13, 14, and 15, respectively;
(b) SEQ ID NOs:1, 2, and 3 and SEQ ID NOs:8, 9, and 10, respectively;
(c) SEQ ID NOs: 1, 2, and 3 and SEQ ID NOs: 8, 12, and 10, respectively;
(d) SEQ ID NOs:4, 5, and 6 and SEQ ID NOs:13, 14, and 15, respectively;
(e) SEQ ID NOs:4, 5, and 6 and SEQ ID NOs:13, 17, and 15, respectively;
(f) SEQ ID NOs:4, 5, and 7 and SEQ ID NOs:13, 17, and 15, respectively; and
(g) SEQ ID NOs:4, 5, and 8 and SEQ ID NOs:13, 17, and 15, respectively.

2-5. (canceled)

6. The antibody or antigen-binding fragment thereof of claim 1, wherein said antibody or antigen-binding fragment thereof comprises a light chain variable domain (VL) and a heavy chain variable domain (VH) having sequences that are at least 95%, 96%, 97%, 98%, 99%, or 100% identical to sequences selected from the group consisting of:
(a) SEQ ID NO:32 and SEQ ID NO:36, respectively;
(b) SEQ ID NO:18 and SEQ ID NO:19, respectively;
(c) SEQ ID NO:20 and SEQ ID NO:21, respectively;
(d) SEQ ID NO:22 and SEQ ID NO:23, respectively;
(e) SEQ ID NO:24 and SEQ ID NO:25, respectively;
(f) SEQ ID NO:26 and SEQ ID NO:27, respectively;
(g) SEQ ID NO:28 and SEQ ID NO:31, respectively;
(h) SEQ ID NO:29 and SEQ ID NO:31, respectively;
(i) SEQ ID NO:30 and SEQ ID NO:31, respectively;
(j) SEQ ID NO:32 and SEQ ID NO:35, respectively;
(k) SEQ ID NO:32 and SEQ ID NO:36, respectively;
(l) SEQ ID NO:33 and SEQ ID NO:36, respectively;
(m) SEQ ID NO:33 and SEQ ID NO:35, respectively; and
(n) SEQ ID NO:33 and SEQ ID NO:34, respectively.

7. The antibody or antigen-binding fragment thereof of claim 1, wherein said antibody or antigen-binding fragment thereof comprises a light chain and a heavy chain having the sequences selected from the group consisting of:
(a) SEQ ID NO:49 and SEQ ID NO:54, respectively;
(b) SEQ ID NO:39 and SEQ ID NO:40, respectively;
(c) SEQ ID NO:41 and SEQ ID NO:42, respectively;
(d) SEQ ID NO:43 and SEQ ID NO:44, respectively;
(e) SEQ ID NO:45 and SEQ ID NO:48, respectively;
(f) SEQ ID NO:46 and SEQ ID NO:48, respectively;
(g) SEQ ID NO:47 and SEQ ID NO:48, respectively;
(h) SEQ ID NO:49 and SEQ ID NO:53, respectively;
(i) SEQ ID NO:49 and SEQ ID NO:52, respectively;
(j) SEQ ID NO:49 and SEQ ID NO:51, respectively;
(k) SEQ ID NO:50 and SEQ ID NO:53, respectively;
(l) SEQ ID NO:50 and SEQ ID NO:52, respectively;
(m) SEQ ID NO:50 and SEQ ID NO:51, respectively;
(n) SEQ ID NO:49 and SEQ ID NO:77, respectively;
(o) SEQ ID NO:49 and SEQ ID NO:78, respectively;
(p) SEQ ID NO:49 and SEQ ID NO:79, respectively;
(q) SEQ ID NO:49 and SEQ ID NO:80, respectively;
(r) SEQ ID NO:49 and SEQ ID NO:81, respectively;
(s) SEQ ID NO:49 and SEQ ID NO:82, respectively;
(t) SEQ ID NO:49 and SEQ ID NO:83, respectively; and
(u) SEQ ID NO:49 and SEQ ID NO:84, respectively.

8. The antibody or antigen-binding fragment thereof of claim 1, wherein said antibody comprises a light chain having the sequence of SEQ ID NO:49 and a heavy chain having the sequence of SEQ ID NO:53.

9. The antibody or antigen-binding fragment thereof of claim 1, wherein said antibody comprises a light chain having the sequence of SEQ ID NO:49 and a heavy chain having the sequence of SEQ ID NO:82.

10. An isolated antibody, or antigen-binding fragment thereof, produced by any of hybridomas 247.27.16, 247.2.26, 247.48.38, 247.3.14, 247.22.2, 248.69.4, and 247.16.8.

11. A polypeptide comprising the VL and VH sequences of claim 6.

12. A cell producing the antibody or antigen-binding fragment thereof of claim 1.

13. A method of producing the antibody or antigen-binding fragment thereof of claim 1, comprising:
(a) culturing a cell producing the antibody or antigen-binding fragment thereof; and,
(b) isolating said antibody, antigen-binding fragment thereof, or polypeptide from said cultured cell.

14. (canceled)

15. A diagnostic reagent comprising the antibody or antigen-binding fragment thereof of claim 1.

16-17. (canceled)

18. A polynucleotide encoding the antibody or antigen-binding fragment of claim 1, wherein said polynucleotide has a sequence selected from the group consisting of SEQ ID NOs:55-72 and 109-116.

19. A vector comprising the polynucleotide of claim 18.

20. A host cell comprising the expression vector of claim 19.

21. An immunoconjugate represented by the following formula:
CBACy^{L1})_{W}_{_{L}},
CBACy^{L2})_{W}_{_{L}},
CBACy^{L3})_{W}_{_{L}},
CBACy^{C1})_{W}_{_{C}}, or
CBACy^{C2})_{W}_{_{C}},
wherein:
CBA is the antibody or antigen-binding fragment thereof of claim 1 that is covalently linked to Cy^{L1} through a lysine residue;
W_{L} is an integer from 1 to 20;
W_{C} is 1 or 2;
Cy^{L1} is represented by the following formula: 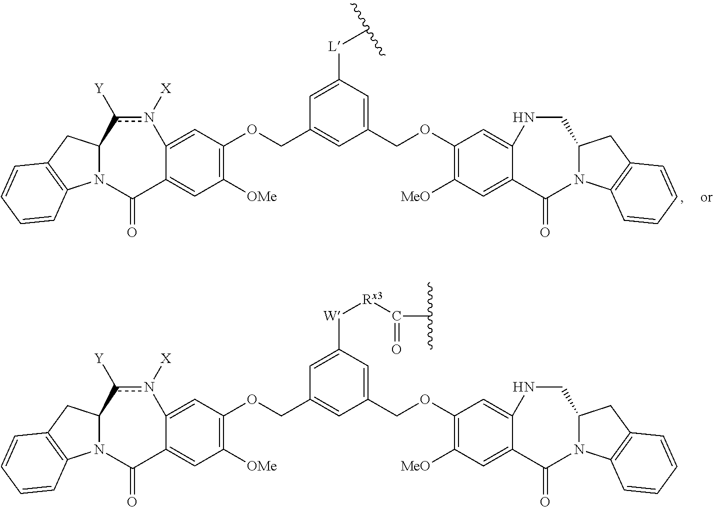
or a pharmaceutically acceptable salt thereof, wherein:
the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃H or a pharmaceutically acceptable salt thereof;
W′ is —NR^{e′},
R^{e′} is —(CH₂—CH₂—O)ₙ—R^{k};
n is an integer from 2 to 6;
R^{k} is —H or -Me;
R^{x3} is a (C₁-C₆)alkyl;
L′ is represented by the following formula:
—NR₅—P—C(═O)—(CRₐR_{b})ₘ—C(═O)— (B 1′); or
—NR₅—P—C(═O)—(CRₐR_{b})ₘ—S—Z^{S1}— (B2′);
R₅ is —H or a (C₁-C₃)alkyl;
P is an amino acid residue or a peptide containing between 2 to 20 amino acid residues;
Rₐ and R_{b}, for each occurrence, are each independently —H, (C₁-C₃)alkyl, or a charged substituent or an ionizable group Q;
m is an integer from 1 to 6; and
Z^{S1} is selected from any one of the following formulas: 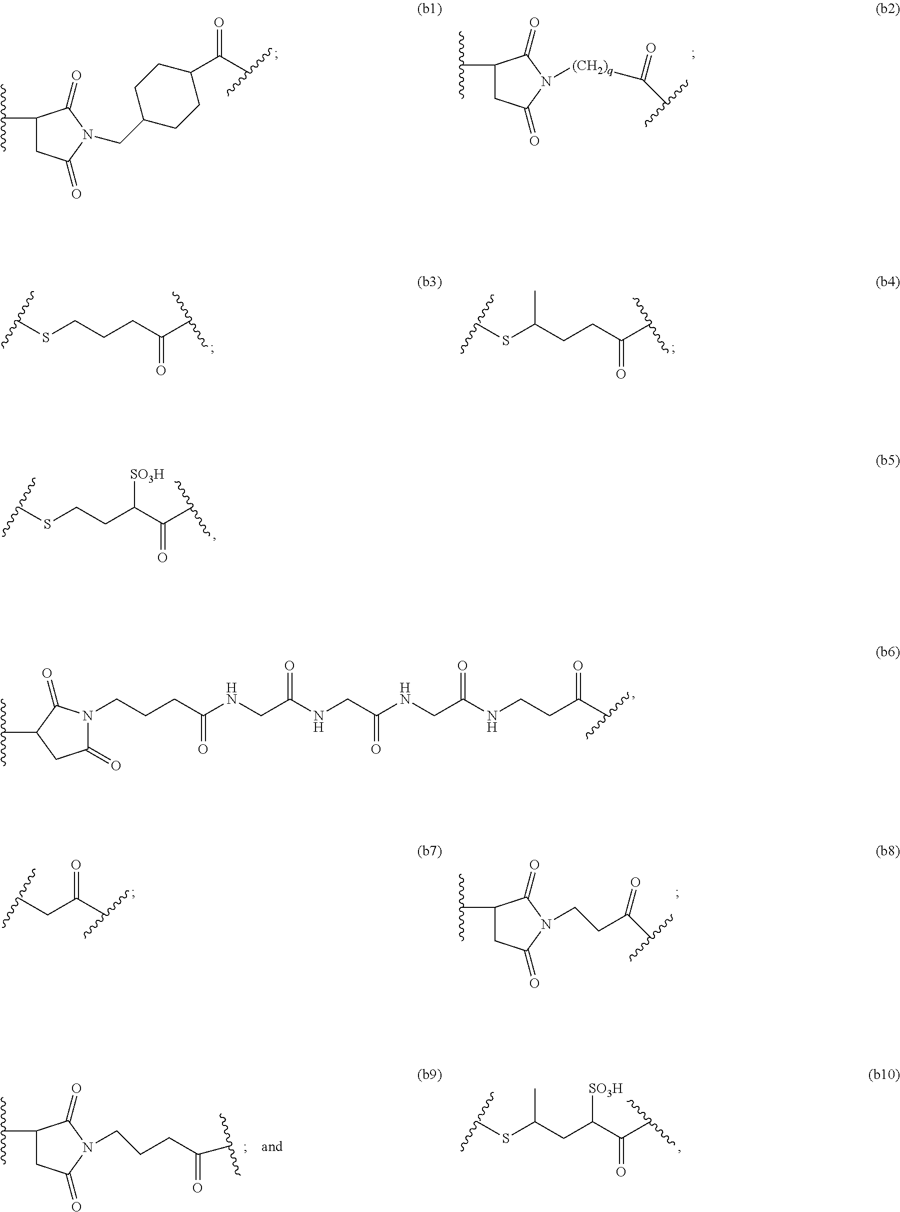
wherein q is an integer from 1 to 5;
Cy^{L2} is represented by the following formula: 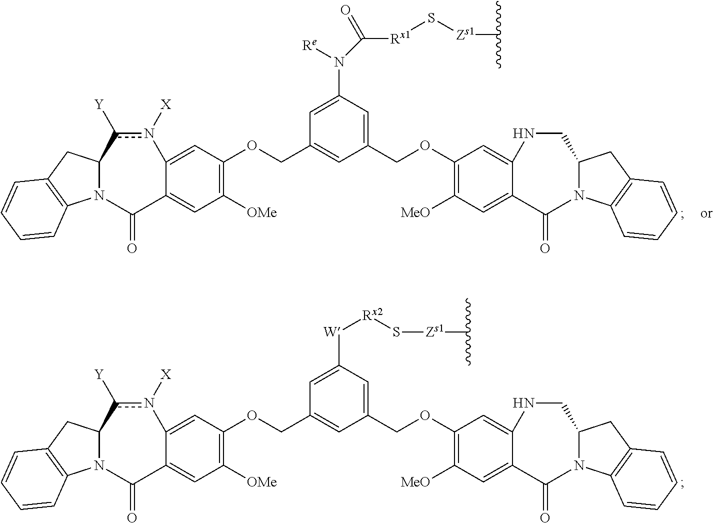
or a pharmaceutically acceptable salt thereof, wherein:
the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃H;
R^{x1} and R^{x2} are independently (C₁-C₆)alkyl;
R^{e} is —H or a (C₁-C₆)alkyl;
W′ is —NR^{e′},
R^{e} is —(CH₂—CH₂—O)ₙ—R^{k};
n is an integer from 2 to 6;
R^{k} is —H or -Me;
Z^{S1} is selected from any one of the following formulas: 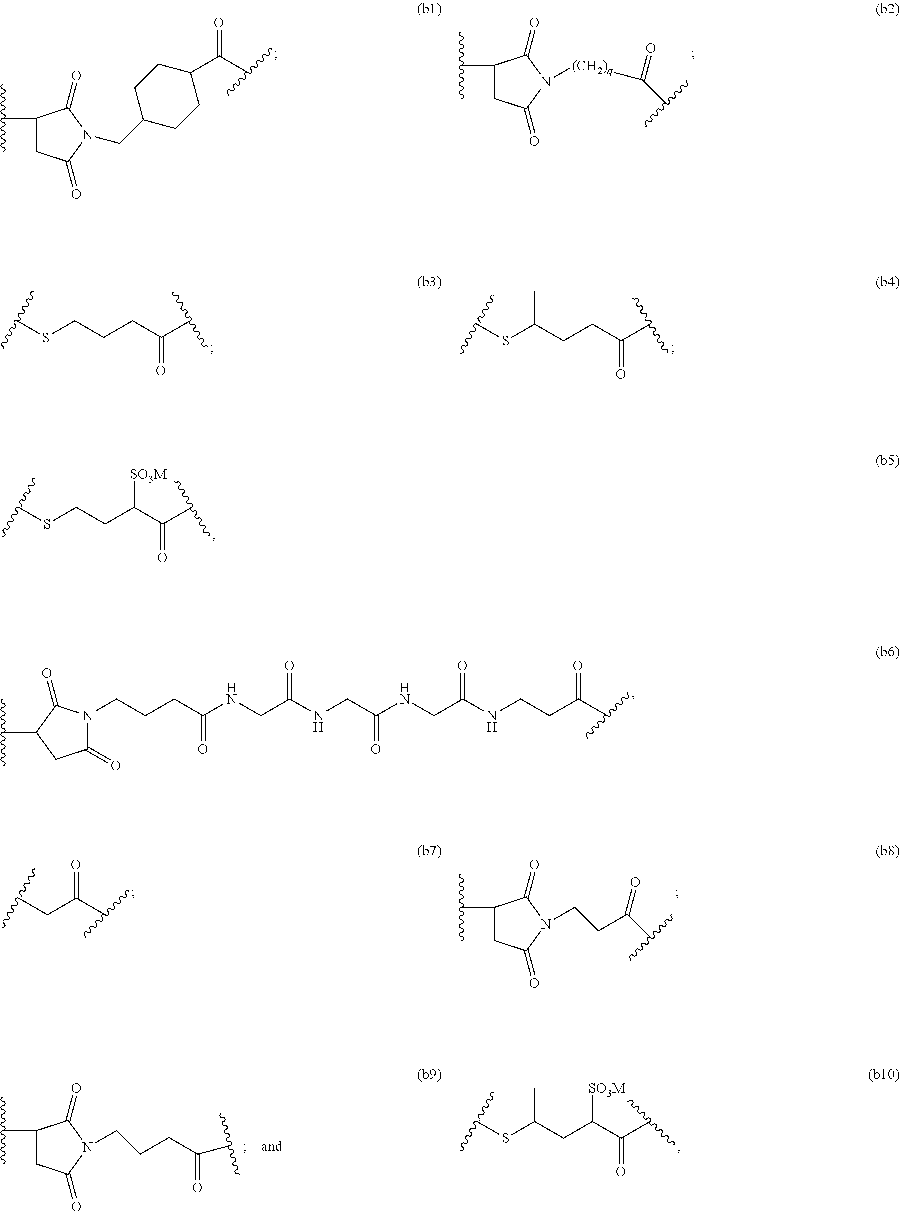
wherein q is an integer from 1 to 5;
Cy^{L3} is represented by the following formula: 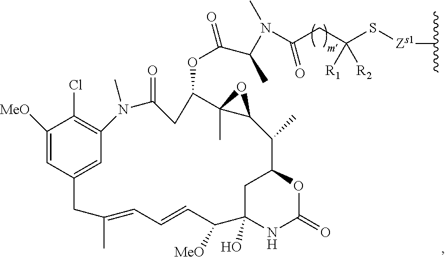
m′ is 1 or 2;
R₁ and R₂, are each independently H or a (C₁-C₃)alkyl; and
Z^{S1} is selected from any one of the following formulas: 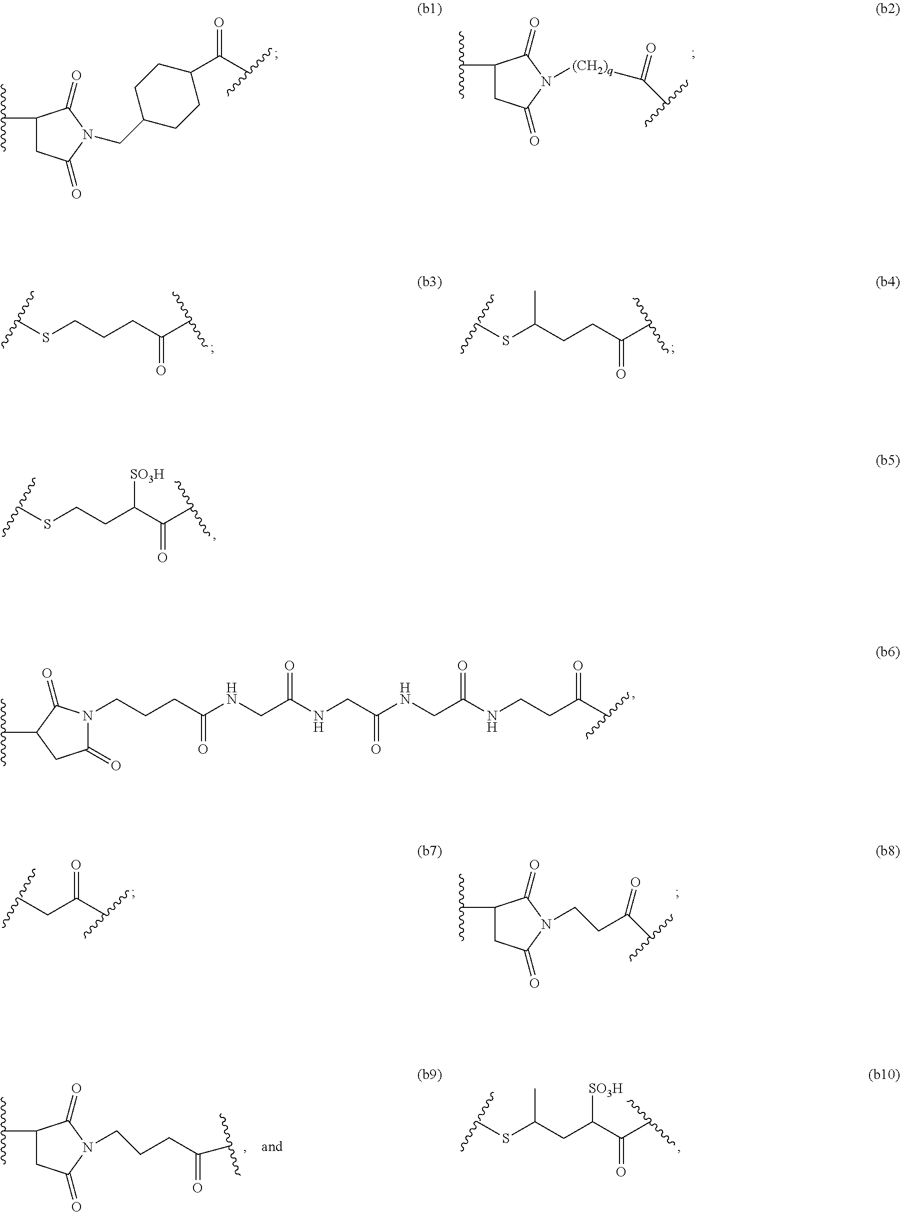
wherein q is an integer from 1 to 5;
Cy^{C1} is represented by the following formula: 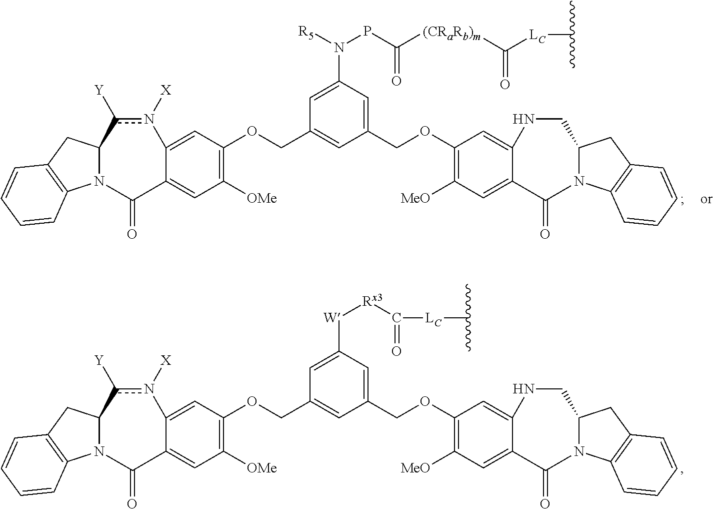
or a pharmaceutically acceptable salt thereof, wherein:
the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃H or a pharmaceutically acceptable salt thereof;
R₅ is —H or a (C₁-C₃)alkyl;
P is an amino acid residue or a peptide containing 2 to 20 amino acid residues;
Rₐ and R_{b}, for each occurrence, are independently —H, (C₁-C₃)alkyl, or a charged substituent or an ionizable group Q;
m is an integer from 1 to 6;
W′ is —NR^{e′},
R^{e′} is —(CH₂—CH₂—O)ₙ—R^{k};
n is an integer from 2 to 6;
R^{k} is —H or -Me;
R^{x3} is a (C₁-C₆)alkyl; and,
L_{C} is represented by 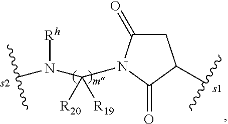
s1 is the site covalently linked to CBA, and s2 is the site covalently linked to the —C(═O)— group on Cy^{C1}; wherein:
R₁₉ and R₂₀, for each occurrence, are independently —H or a (C₁-C₃)alkyl;
m″ is an integer between 1 and 10; and
R^{h} is —H or a (C₁-C₃)alkyl;
Cy^{C2} is represented by the following formula: 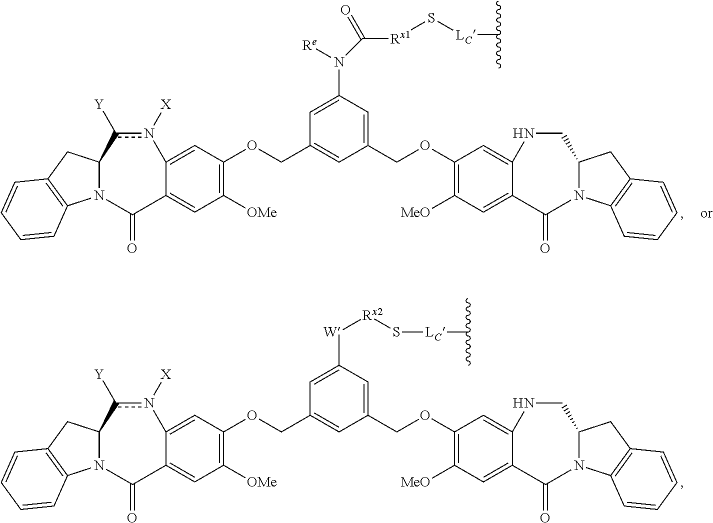
or a pharmaceutically acceptable salt thereof, wherein:
the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃H or a pharmaceutically acceptable salt thereof;
R^{x1} is a (C₁-C₆)alkyl;
R^{e} is —H or a (C₁-C₆)alkyl;
W′ is —NR^{e′};
R^{e′} is —(CH₂—CH₂—O)ₙ—R^{k};
n is an integer from 2 to 6;
R^{k} is —H or -Me;
R^{x2} is a (C₁-C₆)alkyl;
L_{C}′ is represented by the following formula: 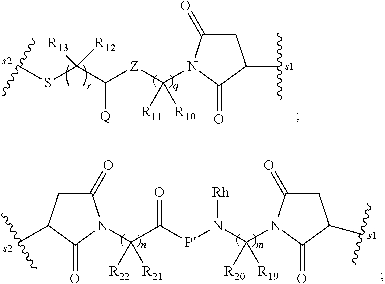
wherein:
s1 is the site covalently linked to the CBA and s2 is the site covalently linked to —S— group on Cy^{C2};
Z is —C(═O)—NR₉—, or —NR₉—C(═O)—;
Q is —H, a charged substituent, or an ionizable group;
R₉, R₁₀, R₁₁, R₁₂, R₁₃, R₁₉, R₂₀, R₂₁ and R₂₂, for each occurrence, are independently —H or a (C₁-C₃)alkyl;
q and r, for each occurrence, are independently an integer between 0 and 10;
m and n are each independently an integer between 0 and 10;
R^{h} is —H or a (C₁-C₃)alkyl; and
P′ is an amino acid residue or a peptide containing 2 to 20 amino acid residues.

22-72. (canceled)

73. A pharmaceutical composition comprising the antibody or antigen-binding fragment of claim 1 and a pharmaceutically acceptable carrier.

74. A pharmaceutical composition comprising the immunoconjugate of claim 21 and a pharmaceutically acceptable carrier.

75. A method for inhibiting aberrant cell proliferation comprising contacting a MET-expressing cell with the isolated monoclonal antibody or antigen-binding fragment of claim 1, wherein said contacting inhibits the aberrant proliferation of said cells.

76. A method for inhibiting aberrant cell proliferation comprising contacting a MET-expressing cell with the immunoconjugate of claim 21, wherein said contacting inhibits the aberrant proliferation of said cells.

77-80. (canceled)

81. A method for treating a cell proliferation disorder in a patient, comprising administering to the patient a therapeutically effective amount of the isolated, monoclonal antibody or antigen-binding fragment thereof of claim 1.

82. A method for treating a cell proliferation disorder in a patient, comprising administering to the patient a therapeutically effective amount of the immunoconjugate of claim 21.

83-86. (canceled)


SEQUENCE LISTING (117 sequences)

SEQ ID NO: 1 - protein, 11 aa, Artificial Sequence
  Description of Artificial Sequence Synthetic peptide
RASENIYSTLA

SEQ ID NO: 2 - protein, 7 aa, Artificial Sequence
  Description of Artificial Sequence Synthetic peptide
AATNLAD

SEQ ID NO: 3 - protein, 9 aa, Artificial Sequence
  Description of Artificial Sequence Synthetic peptide
QHFWGTPYT

SEQ ID NO: 4 - protein, 15 aa, Artificial Sequence
  Description of Artificial Sequence Synthetic peptide
RASESVDSYGNSFIH

SEQ ID NO: 5 - protein, 7 aa, Artificial Sequence
  Description of Artificial Sequence Synthetic peptide
RASNLES

SEQ ID NO: 6 - protein, 9 aa, Artificial Sequence
  Description of Artificial Sequence Synthetic peptide
QQSNEDPLT

SEQ ID NO: 7 - protein, 9 aa, Artificial Sequence
  Description of Artificial Sequence Synthetic peptide
QQSNEEPLT

SEQ ID NO: 8 - protein, 5 aa, Artificial Sequence
  Description of Artificial Sequence Synthetic peptide
DYNMD

SEQ ID NO: 9 - protein, 17 aa, Artificial Sequence
  Description of Artificial Sequence Synthetic peptide
DLNPNNGATIYNQKFKG

SEQ ID NO: 10 - protein, 13 aa, Artificial Sequence
  Description of Artificial Sequence Synthetic peptide
GNYYGNYYYLMDY

SEQ ID NO: 11 - protein, 10 aa, Artificial Sequence
  Description of Artificial Sequence Synthetic peptide
GYTFTDYNMD

SEQ ID NO: 12 - protein, 10 aa, Artificial Sequence
  Description of Artificial Sequence Synthetic peptide
DLNPNNGATI

SEQ ID NO: 13 - protein, 5 aa, Artificial Sequence
  Description of Artificial Sequence Synthetic peptide
SYDMS

SEQ ID NO: 14 - protein, 17 aa, Unknown
  Description of Unknown Murine or human HC CDR2 sequence
TINSNGVSIYYPDSVKG

SEQ ID NO: 15 - protein, 9 aa, Artificial Sequence
  Description of Artificial Sequence Synthetic peptide
EEITTEMDY

SEQ ID NO: 16 - protein, 10 aa, Artificial Sequence
  Description of Artificial Sequence Synthetic peptide
GFTFSSYDMS

SEQ ID NO: 17 - protein, 10 aa, Artificial Sequence
  Description of Artificial Sequence Synthetic peptide
TINSNGVSIY

SEQ ID NO: 18 - protein, 109 aa, Artificial Sequence
  Description of Artificial Sequence Synthetic polypeptide
DIVMTQSPASLSVSVGETVTITCRASENIYSTLAWYQQKQGKSPQLLVYAATNLADGVPSRFSGSGSGTQYSLKINSLQSEDFGSYYCQHFWGTPYTFGGGTKLEIKRT

SEQ ID NO: 19 - protein, 122 aa, Artificial Sequence
  Description of Artificial Sequence Synthetic polypeptide
EVQLEESGPELVKPGASVKIPCKASGYTFTDYNMDWVRQSHGKSLEWIGDLNPNNGATIYNQKFKGKATLTVDMSSSTAYMELRSLTSEDTAVYYCARGNYYGNYYYLMDYWGQGTSVTVSS

SEQ ID NO: 20 - protein, 108 aa, Artificial Sequence
  Description of Artificial Sequence Synthetic polypeptide
DIQMTQSPSSLSVSVGERVTITCRASENIYSTLAWYQQKPGKSPKLLVYAATNLADGVPSRFSGSGSGTEYSLKINSLQPDDFGSYYCQHFWGTPYTFGGGTKLEIKR

SEQ ID NO: 21 - protein, 122 aa, Artificial Sequence
  Description of Artificial Sequence Synthetic polypeptide
EVQLVQSGAEVVKPGASVKIPCKASGYTFTDYNMDWVRQSPGKSLEWIGDLNPNNGATIYNEKFQGKATLTVDTSSSTAYMELRSLTSEDTAVYYCARGNYYGNYYYLMDYWGQGTSVTVSS

SEQ ID NO: 22 - protein, 108 aa, Artificial Sequence
  Description of Artificial Sequence Synthetic polypeptide
DIQMTQSPSSLSASVGDRVTITCRASENIYSTLAWYQQKPGKAPKLLVYAATNLADGVPSRFSGSGSGTDYTLTISSLQPEDFATYYCQHFWGTPYTFGQGTKVEIKR

SEQ ID NO: 23 - protein, 122 aa, Artificial Sequence
  Description of Artificial Sequence Synthetic polypeptide
QVQLVQSGAEVKKPGASVKVSCKASGYTFTDYNMDWVRQAPGQRLEWIGDLNPNNGATIYNQKFKGRATLTVDMSASTAYMELSSLRSEDTAVYYCARGNYYGNYYYLMDYWGQGTSVTVSS

SEQ ID NO: 24 - protein, 112 aa, Artificial Sequence
  Description of Artificial Sequence Synthetic polypeptide
DIVLTQSPASLAVSLGQRATISCRASESVDSYGNSFIHWYQQKPGQPPKLLIYRASNLESGIPARFSGSGSRTDFTLTINPVEADDVATYYCQQSNEDPLTFGAGTKLELKR

SEQ ID NO: 25 - protein, 118 aa, Artificial Sequence
  Description of Artificial Sequence Synthetic polypeptide
EVQLVESGGGLVQPGGSLKLSCAASGFTFSSYDMSWVRQTPDKRLELVATINSNGVSIYYPDSVKGRFTISRDIAKNTLYLQMSSLKSEDTAMYYCAREEITTEMDYWGQGTSVTVSS

SEQ ID NO: 26 - protein, 113 aa, Artificial Sequence
  Description of Artificial Sequence Synthetic polypeptide
DIVMTQSPASLAVSLGQRATISCRASESVDSYGNSFIHWYQQKPGQPPKLLIYRASNLESGIPARFSGSGSRTDFTLTINPVEADDVATYYCQQSNEDPLTFGAGTKLELKRT

SEQ ID NO: 27 - protein, 118 aa, Artificial Sequence
  Description of Artificial Sequence Synthetic polypeptide
EVQLEESGGGLVQPGGSLKLSCAASGFTFSSYDMSWVRQTPDKRLELVATINSNGVSIYYPDSVKGRFTISRDIAKNTLYLQMSSLKSEDTAMYYCAREEITTEMDYWGQGTSVTVSS

SEQ ID NO: 28 - protein, 111 aa, Artificial Sequence
  Description of Artificial Sequence Synthetic polypeptide
DIVLTQSPASLAVSPGQRATISCRASESVDSYGNSFIHWYQQKPGQPPKLLIYRASNLESGIPARFSGSGSRTDFTLTINPVEANDVATYYCQQSNEDPLTFGGGTKLELK

SEQ ID NO: 29 - protein, 111 aa, Artificial Sequence
  Description of Artificial Sequence Synthetic polypeptide
DIVLTQSPASLAVSPGQRATISCRASESVDSYGNSFIHWYQQKPGQPPKLLIYRASNLESGIPARFSGSGSRTDFTLTINPVEANDVATYYCQQSNEEPLTFGGGTKLELK

SEQ ID NO: 30 - protein, 111 aa, Artificial Sequence
  Description of Artificial Sequence Synthetic polypeptide
DIVLTQSPASLAVSPGQRATISCRASESVDSYGNSFIHWYQQKPGQPPKLLIYRASNLESGIPARFSGSGSRTDFTLTINPVEANDVATYYCQQSNENPLTFGGGTKLELK

SEQ ID NO: 31 - protein, 118 aa, Artificial Sequence
  Description of Artificial Sequence Synthetic polypeptide
EVQLVESGGGLVQPGGSLRLSCAASGFTFSSYDMSWVRQTPGKGLELVATINSNGVSIYYPDSVKGRFTISRDIAKNTLYLQMSSLRAEDTAMYYCAREEITTEMDYWGQGTSVTVSS

SEQ ID NO: 32 - protein, 112 aa, Artificial Sequence
  Description of Artificial Sequence Synthetic polypeptide
EIVLTQSPATLSLSPGERATLSCRASESVDSYGNSFIHWYQQKPGQAPRLLIYRASNLESGIPARFSGSGSGTDFTLTISSLEPEDFAVYYCQQSNEEPLTFGQGTKVELKR

SEQ ID NO: 33 - protein, 112 aa, Artificial Sequence
  Description of Artificial Sequence Synthetic polypeptide
EIVLTQSPATLSLSPGERATLSCRASESVDSYGNSFIHWYQQKPGQAPRLLIYRASNLESGIPARFSGSGSRTDFTLTISSLEPEDFAVYYCQQSNEEPLTFGQGTKVELKR

SEQ ID NO: 34 - protein, 118 aa, Artificial Sequence
  Description of Artificial Sequence Synthetic polypeptide
EVQLVESGGGLVQPGGSLRLSCAASGFTFSSYDMSWVRQAPGKGLEWVSTINSNGVSIYYPDSVKGRFTISRDNAKNSLYLQMNSLRAEDTAVYYCAREEITTEMDYWGQGTLVTVSS

SEQ ID NO: 35 - protein, 118 aa, Artificial Sequence
  Description of Artificial Sequence Synthetic polypeptide
EVQLVESGGGLVQPGGSLRLSCAASGFTFSSYDMSWVRQAPGKGLELVATINSNGVSIYYPDSVKGRFTISRDIAKNSLYLQMNSLRAEDTAVYYCAREEITTEMDYWGQGTLVTVSS

SEQ ID NO: 36 - protein, 118 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSS

SEQ ID NO: 37 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 38 - protein, 98 aa, Homo sapiens
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCAR

SEQ ID NO: 39 - protein, 214 aa, Artificial Sequence
  Description of Artificial Sequence Synthetic polypeptide
DIQMTQSPSSLSVSVGERVTITCRASENIYSTLAWYQQKPGKSPKLLVYAATNLADGVPSRFSGSGSGTEYSLKINSLQPDDFGSYYCQHFWGTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 451 aa, Artificial Sequence
  Description of Artificial Sequence Synthetic polypeptide
EVQLVQSGAEVVKPGASVKIPCKASGYTFTDYNMDWVRQSPGKSLEWIGDLNPNNGATIYNEKFQGKATLTVDTSSSTAYMELRSLTSEDTAVYYCARGNYYGNYYYLMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 214 aa, Artificial Sequence
  Description of Artificial Sequence Synthetic polypeptide
DIQMTQSPSSLSASVGDRVTITCRASENIYSTLAWYQQKPGKAPKLLVYAATNLADGVPSRFSGSGSGTDYTLTISSLQPEDFATYYCQHFWGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 451 aa, Artificial Sequence
  Description of Artificial Sequence Synthetic polypeptide
QVQLVQSGAEVKKPGASVKVSCKASGYTFTDYNMDWVRQAPGQRLEWIGDLNPNNGATIYNQKFKGRATLTVDMSASTAYMELSSLRSEDTAVYYCARGNYYGNYYYLMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 218 aa, Artificial Sequence
  Description of Artificial Sequence Synthetic polypeptide
DIVLTQSPASLAVSLGQRATISCRASESVDSYGNSFIHWYQQKPGQPPKLLIYRASNLESGIPARFSGSGSRTDFTLTINPVEADDVATYYCQQSNEDPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 44 - protein, 442 aa, Artificial Sequence
  Description of Artificial Sequence Synthetic polypeptide
EVQLVESGGGLVQPGGSLKLSCAASGFTFSSYDMSWVRQTPDKRLELVATINSNGVSIYYPDSVKGRFTISRDIAKNTLYLQMSSLKSEDTAMYYCAREEITTEMDYWGQGTSVTVSSAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 45 - protein, 218 aa, Artificial Sequence
  Description of Artificial Sequence Synthetic polypeptide
DIVLTQSPASLAVSPGQRATISCRASESVDSYGNSFIHWYQQKPGQPPKLLIYRASNLESGIPARFSGSGSRTDFTLTINPVEANDVATYYCQQSNEDPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 218 aa, Artificial Sequence
  Description of Artificial Sequence Synthetic polypeptide
DIVLTQSPASLAVSPGQRATISCRASESVDSYGNSFIHWYQQKPGQPPKLLIYRASNLESGIPARFSGSGSRTDFTLTINPVEANDVATYYCQQSNEEPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 218 aa, Artificial Sequence
  Description of Artificial Sequence Synthetic polypeptide
DIVLTQSPASLAVSPGQRATISCRASESVDSYGNSFIHWYQQKPGQPPKLLIYRASNLESGIPARFSGSGSRTDFTLTINPVEANDVATYYCQQSNENPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 447 aa, Artificial Sequence
  Description of Artificial Sequence Synthetic polypeptide
EVQLVESGGGLVQPGGSLRLSCAASGFTFSSYDMSWVRQTPGKGLELVATINSNGVSIYYPDSVKGRFTISRDIAKNTLYLQMSSLRAEDTAMYYCAREEITTEMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 218 aa, Artificial Sequence
  Description of Artificial Sequence Synthetic polypeptide
EIVLTQSPATLSLSPGERATLSCRASESVDSYGNSFIHWYQQKPGQAPRLLIYRASNLESGIPARFSGSGSGTDFTLTISSLEPEDFAVYYCQQSNEEPLTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 218 aa, Artificial Sequence
  Description of Artificial Sequence Synthetic polypeptide
EIVLTQSPATLSLSPGERATLSCRASESVDSYGNSFIHWYQQKPGQAPRLLIYRASNLESGIPARFSGSGSRTDFTLTISSLEPEDFAVYYCQQSNEEPLTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 447 aa, Artificial Sequence
  Description of Artificial Sequence Synthetic polypeptide
EVQLVESGGGLVQPGGSLRLSCAASGFTFSSYDMSWVRQAPGKGLEWVSTINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 447 aa, Artificial Sequence
  Description of Artificial Sequence Synthetic polypeptide
EVQLVESGGGLVQPGGSLRLSCAASGFTFSSYDMSWVRQAPGKGLELVATINSNGVSIYYPDSVKGRFTISRDIAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 447 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 447 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 55 - DNA, 396 bp, Artificial Sequence
  Description of Artificial Sequence Synthetic polynucleotide
gaattcgccaccatgggatggtcctgtattatcctgttcctggtcgctaccgctactggggtgcatagtgatattcagatgactcagtcccctagttcactgtccgcctctgtgggcgaccgggtgaccatcacatgcagagcctctgagaacatctacagcaccctggcttggtatcagcagaagccaggcaaggcccccaagctgctggtgtacgctgctaccaacctggccgatggcgtgccatcccggttcagcggctccggctctggcaccgactataccctgacaatcagctccctgcaaccagaggatttcgccacatactattgccagcacttctggggcaccccatacacctttggccagggcacaaaggtggagatcaagcgtacg

SEQ ID NO: 56 - DNA, 452 bp, Artificial Sequence
  Description of Artificial Sequence Synthetic polynucleotide
aagcttgccaccatgggttggtcatgtattattctgtttctggtcgctaccgctaccggggtccatagtcaggtgcagctggtgcagtctggagccgaagtgaagaagccaggcgccagcgtgaaggtgtcttgcaaggcttccggctacaccttcacagactataacatggattgggtgaggcaggctccaggacagcggctggagtggatcggcgacctgaaccctaacaatggcgccaccatctacaatcagaagtttaagggcagggctaccctgacagtggacatgtctgcctccaccgcttatatggagctgtccagcctgagaagcgaggatacagccgtgtactattgtgctcgcggcaactactatggcaattactattatctgatggattactggggacaggggacttccgtgaccgtgagttccgcctctacaaagggccc

SEQ ID NO: 57 - DNA, 408 bp, Artificial Sequence
  Description of Artificial Sequence Synthetic polynucleotide
gaattcgccaccatgggatggtcatgcattatcctgtttctggtggccacagctacaggcgtccactccgaaattgtcctcacacagagccccgcaactctctccctctcccctggagaacgggcaactttgtcatgtcgcgccagtgagtctgttgattcatacggcaacagttttattcattggtatcaacagaagccaggacaagctccccggttgctgatctacagggccagtaatctggagagtggcatccccgcccgattctccggctctggcagcggcaccgactttacattgaccatctctagcctcgaacccgaagacttcgctgtgtattattgccagcagtcaaatgaggaacctctgacctttggacagggaaccaaggtggaactgaagcgtacg

SEQ ID NO: 58 - DNA, 408 bp, Artificial Sequence
  Description of Artificial Sequence Synthetic polynucleotide
gaattcgccaccatggggtggtcttgcataatccttttcctggtggcaaccgcaactggcgttcactctgaaatcgtattgacacaaagccccgccacactgtctctgagccccggagaacgagccaccctgtcttgtagagcttctgaaagcgtggactcctacgggaatagctttatccactggtatcagcaaaagccaggtcaagcccccaggctcttgatttaccgggcctcaaacctggaatctggcatcccagcaaggttttctggctccggcagtaggacagacttcacacttacaatcagttccttggagccagaagattttgcagtatattattgtcagcagtcaaatgaggagcctctgaccttcggccagggaactaaagttgaattgaagcgtacg

SEQ ID NO: 59 - DNA, 440 bp, Artificial Sequence
  Description of Artificial Sequence Synthetic polynucleotide
aagcttgccaccatgggctggagctgtataatactgttcctggtcgctacagcaaccggcgttcactcagaggtacagcttgtggagtccggtggaggactcgttcaacccgggggctcactgcgtctgagctgtgctgcaagcgggttcacattttcttcttatgacatgtcatgggtaaggcaagctcccggcaagggtttggaatgggttagcactatcaattccaatggtgtgtccatctattacccagatagcgttaaaggacgttttactataagcagggataatgccaaaaactcactgtatctgcagatgaactctctccgagctgaagacaccgcagtgtattattgtgcacgggaggagattacaactgagatggattattggggacaggggactctcgtaactgtgtcctccgctagtacaaagggccc

SEQ ID NO: 60 - DNA, 440 bp, Artificial Sequence
  Description of Artificial Sequence Synthetic polynucleotide
aagcttgccaccatgggctggagctgtatcatcctgttcctggtcgccaccgcaacaggcgttcactccgaagtacagctcgtggaatctggcggcggccttgtgcagcccggcggctccctgagactgtcttgtgccgcctccggctttaccttcagcagctacgatatgtcctgggttaggcaagctcccggaaaaggcttggaactggtcgccacaatcaattctaatggcgtgtctatctattaccccgacagtgtgaagggacgcttcacaatcagtagagacattgctaaaaactctctctatttgcagatgaactcactcagggctgaagacactgctgtctactactgtgcccgagaggaaattaccaccgaaatggactattggggtcagggaaccctggttactgtgtcctctgcctctaccaagggccc

SEQ ID NO: 61 - DNA, 440 bp, Artificial Sequence
  Description of Artificial Sequence Synthetic polynucleotide
aagcttgccaccatgggttggtcttgtattatcctgttcctggtcgctactgctaccggcgtccactcagaagtccagctggtcgagagcggcgggggtctggtgcagccaggaggctctctgaggctgtcctgcgccgctagcggcttcaccttttccagctacgacatgagctgggtgagacaggctccaggcaagggcctggagtgggtggctaccatcaactctaatggcgtgtccatctactatcctgactctgtgaagggcaggttcacaatcagccgggataacgccaagaactccctgtatctgcagatgaactccctgagagccgaggatacagccgtgtactattgtgctcgcgaggagatcacaaccgaaatggactattggggacagggaactctggtgaccgtctcatccgcaagcactaagggccc

SEQ ID NO: 62 - DNA, 396 bp, Artificial Sequence
  Description of Artificial Sequence Synthetic polynucleotide
gaattcgccaccatgggctggtcatgcattatcctgttcctggtggccacagctaccggcgtccactcagacattcagatgacccaatcaccatcctctctgagcgtgtcagtcggtgaaagggttactattacctgccgtgcatccgaaaatatttactccactctggcttggtaccaacaaaagccaggcaagtcccctaaactgctggtatatgcagccaccaatttggcagatggagtcccttcccgattttccgggagtggcagtggaactgagtactcactcaaaatcaacagcctccagcccgacgatttcggatcttactactgtcagcacttctggggcacaccttacaccttcggcgggggaactaagctggagatcaagcgtacg

SEQ ID NO: 63 - DNA, 452 bp, Artificial Sequence
  Description of Artificial Sequence Synthetic polynucleotide
aagcttgccaccatgggctggtcctgcatcatacttttcctggtcgccaccgctacaggtgtgcactcagaggtgcagctggtgcagtcaggcgccgaagtggttaaacccggagcctcagtgaagatcccttgcaaagcatccggctatacattcaccgactataatatggattgggtaagacagtcccctgggaagtctcttgaatggatcggtgaccttaatcccaacaatggtgcaacaatctacaatgaaaaatttcaggggaaagctactctgaccgtggatacttcatccagtaccgcctatatggaactgaggtctcttacatccgaggatactgcagtgtattactgcgcccggggcaactactacggaaattactactatctgatggactactgggggcagggcacttctgtgactgtttcctccgcttccacaaagggccc

SEQ ID NO: 64 - DNA, 408 bp, Artificial Sequence
  Description of Artificial Sequence Synthetic polynucleotide
gaattcgccaccatgggctggtcttgtatcattttgttcctggttgccaccgcaacaggtgtacactctgacatagtccttacacaaagcccagcatcactcgcagtcagcccaggccagagagctacaatctcctgtcgggcttccgagtccgtcgattcctatggaaacagcttcatacactggtaccagcagaaacctgggcagcccccaaaacttctgatttatcgggcaagtaatttggagtcaggtatccctgccaggttcagtggttctggctcccgaaccgattttacactcactattaaccccgtggaagccaatgacgtcgcaacttactactgccaacagagtaacgaggaccccttgaccttcggcggcggcactaagctggagctcaagcgtacg

SEQ ID NO: 65 - DNA, 408 bp, Artificial Sequence
  Description of Artificial Sequence Synthetic polynucleotide
gaattcgccaccatgggttggtcctgcattatcctctttttggtggctacagcaaccggtgttcattctgacattgttctcactcagtcacctgcaagtttggccgtcagtccaggacagcgggcaaccatctcctgtcgcgctagcgaatccgtagatagctatggaaactcctttattcactggtaccagcaaaagccagggcagcctcccaaactgctgatttacagagcctccaacctggaaagtggcatccccgcccggttcagtggctcaggttctcggaccgattttacactcaccattaatcccgtggaagctaacgatgtggctacatactattgtcagcagagcaatgaggaacctctcaccttcggggggggcactaagctggagctgaagcgtacg

SEQ ID NO: 66 - DNA, 408 bp, Artificial Sequence
  Description of Artificial Sequence Synthetic polynucleotide
gaattcgccaccatgggctggtcttgcatcatactgttcctggtcgcaactgccacaggcgttcactctgatatcgtgttgacccagtcccctgccagcctcgcagtcagccctggccagcgcgctaccatatcttgtcgtgcttctgaaagcgtggattcttacggcaacagtttcatacactggtaccagcagaaacctgggcagccacctaaactgctgatctatcgagcttcaaaccttgaatccggcattcctgcccggttttcaggctccggctccagaaccgatttcaccttgaccattaatcctgtagaggctaatgacgttgccacctactattgccagcaatccaatgaaaaccctctcacctttggcggcgggacaaagctggagctgaagcgtacg

SEQ ID NO: 67 - DNA, 440 bp, Artificial Sequence
  Description of Artificial Sequence Synthetic polynucleotide
aagcttgccaccatgggttggtcctgtattatcctgtttttggtggctactgcaaccggcgtacatagtgaggtccagttggttgagtccggcggcggtctggtccagcccggcggtagcctgcggctgagttgcgctgcctcaggctttactttctccagctacgacatgagttgggttcgacagacaccaggcaagggcctggaactcgtggcaacaatcaatagtaacggtgtcagcatatactaccccgacagtgtcaaggggaggtttaccataagtagagatatcgccaaaaacacattgtacctgcagatgtccagtctgcgtgccgaggatacagctatgtactactgtgcacgcgaagagatcaccacagagatggactactgggggcagggtacaagcgtcaccgtcagctctgctagtaccaagggccc

SEQ ID NO: 68 - DNA, 726 bp, Artificial Sequence
  Description of Artificial Sequence Synthetic polynucleotide
gaattcgccaccatgggatggtcatgcattatcctgtttctggtggccacagctacaggcgtccactccgaaattgtcctcacacagagccccgcaactctctccctctcccctggagaacgggcaactttgtcatgtcgcgccagtgagtctgttgattcatacggcaacagttttattcattggtatcaacagaagccaggacaagctccccggttgctgatctacagggccagtaatctggagagtggcatccccgcccgattctccggctctggcagcggcaccgactttacattgaccatctctagcctcgaacccgaagacttcgctgtgtattattgccagcagtcaaatgaggaacctctgacctttggacagggaaccaaggtggaactgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 69 - DNA, 726 bp, Artificial Sequence
  Description of Artificial Sequence Synthetic polynucleotide
gaattcgccaccatggggtggtcttgcataatccttttcctggtggcaaccgcaactggcgttcactctgaaatcgtattgacacaaagccccgccacactgtctctgagccccggagaacgagccaccctgtcttgtagagcttctgaaagcgtggactcctacgggaatagctttatccactggtatcagcaaaagccaggtcaagcccccaggctcttgatttaccgggcctcaaacctggaatctggcatcccagcaaggttttctggctccggcagtaggacagacttcacacttacaatcagttccttggagccagaagattttgcagtatattattgtcagcagtcaaatgaggagcctctgaccttcggccagggaactaaagttgaattgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 70 - DNA, 1419 bp, Artificial Sequence
  Description of Artificial Sequence Synthetic polynucleotide
aagcttgccaccatgggctggagctgtataatactgttcctggtcgctacagcaaccggcgttcactcagaggtacagcttgtggagtccggtggaggactcgttcaacccgggggctcactgcgtctgagctgtgctgcaagcgggttcacattttcttcttatgacatgtcatgggtaaggcaagctcccggcaagggtttggaatgggttagcactatcaattccaatggtgtgtccatctattacccagatagcgttaaaggacgttttactataagcagggataatgccaaaaactcactgtatctgcagatgaactctctccgagctgaagacaccgcagtgtattattgtgcacgggaggagattacaactgagatggattattggggacaggggactctcgtaactgtgtcctccgctagtacaaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 71 - DNA, 1419 bp, Artificial Sequence
  Description of Artificial Sequence Synthetic polynucleotide
aagcttgccaccatgggctggagctgtatcatcctgttcctggtcgccaccgcaacaggcgttcactccgaagtacagctcgtggaatctggcggcggccttgtgcagcccggcggctccctgagactgtcttgtgccgcctccggctttaccttcagcagctacgatatgtcctgggttaggcaagctcccggaaaaggcttggaactggtcgccacaatcaattctaatggcgtgtctatctattaccccgacagtgtgaagggacgcttcacaatcagtagagacattgctaaaaactctctctatttgcagatgaactcactcagggctgaagacactgctgtctactactgtgcccgagaggaaattaccaccgaaatggactattggggtcagggaaccctggttactgtgtcctctgcctctaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 72 - DNA, 1419 bp, Artificial Sequence
  Description of Artificial Sequence Synthetic polynucleotide
aagcttgccaccatgggttggtcttgtattatcctgttcctggtcgctactgctaccggcgtccactcagaagtccagctggtcgagagcggcgggggtctggtgcagccaggaggctctctgaggctgtcctgcgccgctagcggcttcaccttttccagctacgacatgagctgggtgagacaggctccaggcaagggcctggagtgggtggctaccatcaactctaatggcgtgtccatctactatcctgactctgtgaagggcaggttcacaatcagccgggataacgccaagaactccctgtatctgcagatgaactccctgagagccgaggatacagccgtgtactattgtgctcgcgaggagatcacaaccgaaatggactattggggacagggaactctggtgaccgtctcatccgcaagcactaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 73 - protein, 17 aa, Homo sapiens
DLNPNNGATIYNEKFQG

SEQ ID NO: 74 - protein, 4 aa, Artificial Sequence
  Description of Artificial Sequence Synthetic peptide
ALAL

SEQ ID NO: 75 - protein, 4 aa, Artificial Sequence
  Description of Artificial Sequence Synthetic peptide
  MOD_RES (1)..(1): Beta-Ala
ALAL

SEQ ID NO: 76 - protein, 4 aa, Artificial Sequence
  Description of Artificial Sequence Synthetic peptide
GFLK

SEQ ID NO: 77 - protein, 444 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 78 - protein, 444 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 79 - protein, 444 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 444 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 445 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 82 - protein, 445 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 446 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPRDCGCKPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 84 - protein, 446 aa, Artificial Sequence
  Description of Artificial Sequence Synthetic polypeptide
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPRDCGCKPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 13 aa, Artificial Sequence
  Description of Artificial Sequence Synthetic peptide
PRDCGCKPCPPCP

SEQ ID NO: 86 - protein, 12 aa, Artificial Sequence
  Description of Artificial Sequence Synthetic peptide
PKSCDCHCPPCP

SEQ ID NO: 87 - protein, 11 aa, Artificial Sequence
  Description of Artificial Sequence Synthetic peptide
RKCCVECPPCP

SEQ ID NO: 88 - protein, 12 aa, Artificial Sequence
  Description of Artificial Sequence Synthetic peptide
PRDCGCKPCICT

SEQ ID NO: 89 - protein, 12 aa, Artificial Sequence
  Description of Artificial Sequence Synthetic peptide
PKSCGCKPCICT

SEQ ID NO: 90 - protein, 12 aa, Artificial Sequence
  Description of Artificial Sequence Synthetic peptide
PKSCGCKPCICP

SEQ ID NO: 91 - protein, 13 aa, Artificial Sequence
  Description of Artificial Sequence Synthetic peptide
PRDCGCHTCPPCP

SEQ ID NO: 92 - protein, 13 aa, Artificial Sequence
  Description of Artificial Sequence Synthetic peptide
PRDCGCHTCPPCP

SEQ ID NO: 93 - protein, 14 aa, Artificial Sequence
  Description of Artificial Sequence Synthetic peptide
CKSCDKTHTCPPCP

SEQ ID NO: 94 - protein, 14 aa, Artificial Sequence
  Description of Artificial Sequence Synthetic peptide
PCSCDKTHTCPPCP

SEQ ID NO: 95 - protein, 14 aa, Artificial Sequence
  Description of Artificial Sequence Synthetic peptide
PKCCDKTHTCPPCP

SEQ ID NO: 96 - protein, 14 aa, Artificial Sequence [sequence omitted]

SEQ ID NO: 97 - protein, 14 aa, Artificial Sequence
  Description of Artificial Sequence Synthetic peptide
PKSCDCTHTCPPCP

SEQ ID NO: 98 - protein, 14 aa, Artificial Sequence
  Description of Artificial Sequence Synthetic peptide
PKSCDKCHTCPPCP

SEQ ID NO: 99 - protein, 14 aa, Artificial Sequence
  Description of Artificial Sequence Synthetic peptide
PKSCDKTHCCPPCP

SEQ ID NO: 100 - protein, 12 aa, Artificial Sequence
  Description of Artificial Sequence Synthetic peptide
KCDKTHTCPPCP

SEQ ID NO: 101 - protein, 13 aa, Artificial Sequence
  Description of Artificial Sequence Synthetic peptide
PKSCDCHTCPPCP

SEQ ID NO: 102 - protein, 13 aa, Artificial Sequence
  Description of Artificial Sequence Synthetic peptide
PKSCDCTHCPPCP

SEQ ID NO: 103 - protein, 12 aa, Artificial Sequence
  Description of Artificial Sequence Synthetic peptide
PCSCKHTCPPCP

SEQ ID NO: 104 - protein, 12 aa, Artificial Sequence
  Description of Artificial Sequence Synthetic peptide
PSCCTHTCPPCP

SEQ ID NO: 105 - protein, 12 aa, Artificial Sequence
  Description of Artificial Sequence Synthetic peptide
PSCDKHCCPPCP

SEQ ID NO: 106 - protein, 10 aa, Artificial Sequence
  Description of Artificial Sequence Synthetic peptide
PKSCTCPPCP

SEQ ID NO: 107 - protein, 14 aa, Artificial Sequence
  Description of Artificial Sequence Synthetic peptide
PKSCDKCVECPPCP

SEQ ID NO: 108 - protein, 11 aa, Artificial Sequence
  Description of Artificial Sequence Synthetic peptide
RKCCVECPPCP

SEQ ID NO: 109 - DNA, 1389 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccatccgtgttcccactggctccctcatccaaaagtacttcagggggtaccgcagccctgggatgtctggtgaaggactacttcccagagcccgtcaccgtgtcttggaacagtggggctctgacctccggtgtccacacatttccagcagtgctgcagagctctggcctgtactccctgagttcagtggtcacagtgccctccagctctctgggaacacagacttatatctgcaacgtgaatcataagccttccaatactaaagtcgataagaaagtggagcgaaagtgctgcgtggaatgccccccttgtcctgcaccagaactgctgggcggaccctccgtgttcctgtttccacccaagcctaaagacactctgatgatttcccggacacctgaggtcacttgcgtggtcgtggacgtgtcccacgaggaccccgaagtcaagttcaactggtacgtggatggagtcgaagtgcataatgctaagacaaaacctagagaggaacagtacaacagtacatatagagtcgtgtcagtcctgactgtgctgcaccaggactggctgaacgggaaggagtataagtgcaaagtgagcaataaggctctgcccgcacctatcgagaaaaccatttctaaggctaaaggccagcctagggaaccacaggtgtacacactgcctccatctcgggacgagctgactaagaaccaggtcagtctgacctgtctggtgaaagggttctatccatccgatatcgcagtggagtgggaaagcaatggtcagcccgagaacaattacaagactaccccccctgtgctggactcagatgggtccttctttctgtatagtaagctgaccgtggataaatcaaggtggcagcagggtaatgtcttttcctgtagcgtgatgcacgaagccctgcataaccactacactcagaaaagcctgtgcctgtcccctgga

SEQ ID NO: 110 - DNA, 1389 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccaagcgtcttccccctggctccatgctcaaagtcaacaagcggtggtactgctgctctgggttgcctggtcaaggattattttcccgagcctgtcaccgtgtcatggaactccggggcactgaccagcggtgtccacacattcccagccgtgctgcagtccagcgggctgtactctctgtctagtgtggtcactgtgccatcatccagcctgggtactcagacctatatctgcaacgtgaatcataagccctccaataccaaagtcgacaagaaagtggagcgaaagtgctgcgtggaatgcccaccttgtccagcaccagaactgctgggcggaccatccgtgttcctgtttccacccaagcccaaagacacactgatgattagcaggacacccgaggtcacttgcgtggtcgtggacgtgtctcacgaggaccccgaagtcaagtttaactggtacgtggatggcgtcgaagtgcataatgctaagactaaacccagggaggaacagtacaacagtacatatcgggtcgtgtcagtcctgactgtgctgcaccaggattggctgaacgggaaggagtataagtgcaaagtgagtaataaggccctgcctgctccaatcgagaaaaccatttccaaggctaaaggccagcccagagaacctcaggtgtacacactgcctccatcacgcgacgagctgactaagaaccaggtctccctgacctgtctggtgaaaggcttctatccttctgatatcgcagtggagtgggaaagtaatggacagccagagaacaattacaagaccacaccccctgtgctggacagcgatggctctttctttctgtattccaagctgacagtcgacaaaagcagatggcagcagggaaacgtgttctcctgcagtgtgatgcacgaagccctgcataaccattacactcagaaaagcctgtgcctgtcccctggg

SEQ ID NO: 111 - DNA, 1389 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccatccgtgttcccactggctccctcatccaaaagtacttcagggggtaccgcagccctgggatgtctggtgaaggactacttcccagagcccgtcaccgtgtcttggaacagtggggctctgacctccggtgtccacacatttccagcagtgctgcagagctctggcctgtactccctgagttcagtggtcacagtgccctccagctctctgggaacacagacttatatctgcaacgtgaatcataagccttccaatactaaagtcgataagaaagtggagcgaaagtgctgcgtggaatgccccccttgtcctgcaccagaactgctgggcggaccctccgtgttcctgtttccacccaagcctaaagacactctgatgatttcccggacacctgaggtcacttgcgtggtcgtggacgtgtcccacgaggaccccgaagtcaagttcaactggtacgtggatggagtcgaagtgcataatgctaagacaaaacctagagaggaacagtacaacagtacatatagagtcgtgtcagtcctgactgtgctgcaccaggactggctgaacgggaaggagtataagtgcaaagtgagcaataaggctctgcccgcacctatcgagaaaaccatttctaaggctaaaggccagcctagggaaccacaggtgtacacactgcctccatctcgggacgagctgactaagaaccaggtcagtctgacctgtctggtgaaagggttctatccatccgatatcgcagtggagtgggaaagcaatggtcagcccgagaacaattacaagactaccccccctgtgctggactcagatgggtccttctttctgtatagtaagctgaccgtggataaatcaaggtggcagcagggtaatgtcttttcctgtagcgtgatgcacgaagccctgcataaccactacactcagaaaagcctgtccctgtcccctgga

SEQ ID NO: 112 - DNA, 1389 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccaagcgtcttccccctggctccatgctcaaagtcaacaagcggtggtactgctgctctgggttgcctggtcaaggattattttcccgagcctgtcaccgtgtcatggaactccggggcactgaccagcggtgtccacacattcccagccgtgctgcagtccagcgggctgtactctctgtctagtgtggtcactgtgccatcatccagcctgggtactcagacctatatctgcaacgtgaatcataagccctccaataccaaagtcgacaagaaagtggagcgaaagtgctgcgtggaatgcccaccttgtccagcaccagaactgctgggcggaccatccgtgttcctgtttccacccaagcccaaagacacactgatgattagcaggacacccgaggtcacttgcgtggtcgtggacgtgtctcacgaggaccccgaagtcaagtttaactggtacgtggatggcgtcgaagtgcataatgctaagactaaacccagggaggaacagtacaacagtacatatcgggtcgtgtcagtcctgactgtgctgcaccaggattggctgaacgggaaggagtataagtgcaaagtgagtaataaggccctgcctgctccaatcgagaaaaccatttccaaggctaaaggccagcccagagaacctcaggtgtacacactgcctccatcacgcgacgagctgactaagaaccaggtctccctgacctgtctggtgaaaggcttctatccttctgatatcgcagtggagtgggaaagtaatggacagccagagaacaattacaagaccacaccccctgtgctggacagcgatggctctttctttctgtattccaagctgacagtcgacaaaagcagatggcagcagggaaacgtgttctcctgcagtgtgatgcacgaagccctgcataaccattacactcagaaaagcctgagcctgtcccctggg

SEQ ID NO: 113 - DNA, 1392 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccaagcgtctttccactggctccaagttccaagtctacaagcggcggtactgctgctctggggtgtctggtgaaggattatttccctgaaccagtcaccgtgtcatggaactccggggctctgacctccggtgtccacacattccccgcagtgctgcagtccagcgggctgtactccctgtctagtgtggtcactgtgccttcatccagcctgggtactcagacctatatctgtaacgtgaatcacaagccaagcaataccaaggtcgacaaacgagtggaacccaaatcttgcgattgtcattgcccaccttgcccagctcctgagctgctgggcggacccagcgtgttcctgtttccacccaagcctaaagacacactgatgattagtaggacacccgaagtcacttgcgtggtcgtggacgtgtcccacgaggaccccgaagtcaagtttaactggtacgtggatggcgtcgaggtgcataatgcaaagactaaaccaagggaggaacagtacaacagtacatatcgggtcgtgtcagtcctgactgtgctgcatcaggactggctgaacgggaaggaatataagtgtaaagtgagcaataaggcactgccagcccccatcgagaaaaccatttctaaggccaaaggccagcctagagaaccacaggtgtacacactgcctccatcacgcgacgagctgactaagaaccaggtctccctgacctgcctggtgaaaggcttctatccttctgatatcgctgtggagtgggaaagtaatggacagccagagaacaattacaagaccacaccccctgtgctggacagcgatggctctttctttctgtattccaagctgacagtggataaaagcagatggcagcagggaaacgtgttctcctgcagtgtgatgcacgaagccctgcataaccattacactcagaagagcctgtgcctgtcccctggg

SEQ ID NO: 114 - DNA, 1392 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccaagcgtgttcccactggctcccagctccaagagcacctccggaggaacagccgctctgggctgtctggtgaaggactacttcccagagcccgtgaccgtgtcctggaactctggcgccctgacctccggagtgcacacatttccagctgtgctgcagtctagcggcctgtactctctgtcctctgtggtgaccgtgcccagctcctctctgggcacccagacatatatctgtaacgtgaatcacaagccatccaatacaaaggtggacaagcgggtggagcccaagtcttgcgattgtcactgcccaccttgccctgctccagagctgctgggcggcccttccgtgttcctgtttccacccaagcctaaggacaccctgatgatcagcagaacccccgaggtgacatgcgtggtggtggacgtgtcccacgaggaccccgaggtgaagtttaactggtacgtggatggcgtggaggtgcacaatgctaagacaaagcctcgggaggagcagtacaactctacctatagggtggtgagcgtgctgacagtgctgcaccaggactggctgaacggcaaggagtataagtgtaaggtgtctaataaggccctgcccgctcctatcgagaagaccatcagcaaggccaagggccagcctagagagccacaggtgtacacactgcctccatctcgcgacgagctgaccaagaaccaggtgagcctgacatgcctggtgaagggcttctatcctagcgatatcgctgtggagtgggagtccaatggccagccagagaacaattacaagaccacaccccctgtgctggacagcgatggctccttctttctgtattccaagctgaccgtggataagtctcggtggcagcagggcaacgtgttttcttgtagcgtgatgcacgaggctctgcacaatcactatacacagaagtccctgtctctgagccccggc

SEQ ID NO: 115 - DNA, 1395 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccaagcgtcttccctctggctccatcaagcaaatcaacttctggcggcacagcagcactggggtgtctggtcaaggactactttcccgagcctgtcaccgtgtcatggaactccggggctctgaccagcggtgtccacacattcccagcagtgctgcagtccagcgggctgtactctctgtctagtgtggtcactgtgccatcatccagcctgggtactcagacctatatctgcaacgtgaatcataagccctccaataccaaggtcgacaaaagggtggaacccagggactgcggctgtaaaccttgcccaccttgtccagctcctgagctgctgggcggaccatccgtgttcctgtttccacccaagcccaaagacacactgatgattagccggacacccgaagtcacttgcgtggtcgtggacgtgtctcacgaggaccccgaagtcaagtttaactggtacgtggatggagtcgaggtgcataatgcaaagactaaacctagagaggaacagtacaacagtacatatagagtcgtgtcagtcctgactgtgctgcaccaggactggctgaacgggaaggaatataagtgcaaagtgagtaataaggcactgccagcccccatcgagaaaaccatttccaaggccaaaggccagcccagggagccacaggtgtacacactgcctccatcacgtgacgagctgactaagaaccaggtctccctgacctgtctggtgaaaggcttctatccttctgatatcgctgtggagtgggaaagtaatggacagccagagaacaattacaagaccacaccccctgtgctggacagcgatggctctttctttctgtattccaagctgacagtggataaaagccgctggcagcagggaaacgtgttctcctgcagtgtgatgcacgaagccctgcataaccactacactcagaagagcctgtgcctgtcccctggc

SEQ ID NO: 116 - DNA, 1395 bp, Artificial Sequence
  Description of Artificial Sequence Synthetic polynucleotide
atgggttggtcctgtattattctgtttctggtcgctaccgctactggggtccattccgaagtgcagctggtcgagagtgggggagggctggtgcagcctggcggaagcctgagactgtcttgcgccgctagtggcttcaccttttccagctacgacatgagctgggtgcgccaggcaccagggaagggtctggagtgggtcgccactatcaactcaaatggcgtgtccatctactatcccgactctgtcaagggaaggttcaccatctccagggacaacgcaaaaaatagcctgtacctgcagatgaactctctgcgagccgaagacaccgccgtgtactattgcgcccgtgaggaaattaccacagagatggattattggggccagggaaccctggtcacagtgtctagtgccagcacaaagggcccaagcgtcttccctctggctccatcaagcaaatcaacttctggcggcacagcagcactggggtgtctggtcaaggactactttcccgagcctgtcaccgtgtcatggaactccggggctctgaccagcggtgtccacacattcccagcagtgctgcagtccagcgggctgtactctctgtctagtgtggtcactgtgccatcatccagcctgggtactcagacctatatctgcaacgtgaatcataagccctccaataccaaggtcgacaaaagggtggaacccagggactgcggctgtaaaccttgcccaccttgtccagctcctgagctgctgggcggaccatccgtgttcctgtttccacccaagcccaaagacacactgatgattagccggacacccgaagtcacttgcgtggtcgtggacgtgtctcacgaggaccccgaagtcaagtttaactggtacgtggatggagtcgaggtgcataatgcaaagactaaacctagagaggaacagtacaacagtacatatagagtcgtgtcagtcctgactgtgctgcaccaggactggctgaacgggaaggaatataagtgcaaagtgagtaataaggcactgccagcccccatcgagaaaaccatttccaaggccaaaggccagcccagggagccacaggtgtacacactgcctccatcacgtgacgagctgactaagaaccaggtctccctgacctgtctggtgaaaggcttctatccttctgatatcgctgtggagtgggaaagtaatggacagccagagaacaattacaagaccacaccccctgtgctggacagcgatggctctttctttctgtattccaagctgacagtggataaaagccgctggcagcagggaaacgtgttctcctgcagtgtgatgcacgaagccctgcataaccactacactcagaagagcctgtccctgtcccctggc

SEQ ID NO: 117 - protein, 9 aa, Artificial Sequence
  Description of Artificial Sequence Synthetic peptide
QQSNENPLT